(19) United States
(12) Patent Application Publication
(10) Pub. No.: US 2022/0069233 A1
(43) Pub. Date: Mar. 3, 2022

(54) COMPOUND, ORGANIC OPTOELECTRONIC DIODE, AND DISPLAY DEVICE

(71) Applicant: LT Materials Co.,Ltd., Yongin-si, Gyeonggi-do (KR)

(72) Inventors: Mi Jin KIM, Yongin-si (KR); Jun Tae MO, Yongin-si (KR); Jong Su LEE, Yongin-si (KR); Ji Yoon BYUN, Yongin-si (KR); Yong Hui LEE, Yongin-si (KR); Dong Jun KIM, Yongin-si (KR)

(73) Assignee: LT Materials Co.,Ltd., Yongin-si, Gyeonggi-do (KR)

(21) Appl. No.: 17/418,728

(22) PCT Filed: Dec. 17, 2019
(86) PCT No.: PCT/KR2019/017906

§ 371 Date: Jun. 25, 2021

(30) Foreign Application Priority Data
Dec. 26, 2018 (KR) 10-2018-0169376

(51) Int. Cl. H01L 51/00; C07D 409/14; C09K 11/06

(52) U.S. Cl. CPC H01L 51/0072; C07D 409/14; C09K 11/06; H01L 51/5012; H01L 51/0074; H01L 51/0073; H01L 51/0052; H01L 51/0067

(57) ABSTRACT

The present invention relates to compounds represented by specific formulas, organic optoelectronic diodes, and display devices.

### TECHNICAL FIELD OF THE INVENTION

A compound, an organic optoelectronic diode, and a display device are disclosed.

### BACKGROUND OF THE INVENTION

An organic optoelectronic diode is a diode that converts electrical energy into photoenergy, and vice versa. An organic optoelectronic diode may be classified as follows in accordance with its driving principles. One is a photoelectric diode in which excitons generated by photoenergy, are separated into electrons and holes, and electrons and holes are transferred to different electrodes respectively to generate electrical energy, and the other is a light emitting diode where a voltage or a current is supplied to an electrode to generate photoenergy from electrical energy.

Examples of the organic optoelectronic diode may be an organic photoelectric diode, an organic light emitting diode, an organic solar cell, and an organic photoconductor drum.

Of these, an organic light emitting diode (OLED) has recently drawn attention due to an increase in demand for flat panel displays. The organic light emitting diode (OLED) converts electrical energy into light, and the performance of the organic light emitting diode (OLED) may be mainly affected by characteristics of organic materials located between the electrodes.

The organic light emitting diode (OLED) has a structure in which an organic thin film is disposed between two electrodes. When a voltage is applied to the organic light emitting diode (OLED) having the structure, electrons and holes injected from the two electrodes combine with each other in an organic thin film to make a pair, and then emit light while being extinguished. The organic thin film may be composed of a single layer or multilayers as necessary.

A material of the organic thin film may have a light emitting function as necessary. For example, as the material for the organic thin film, it is also possible to use a compound which may itself constitute a light emitting layer alone, or it is also possible to use a compound which may serve as a host or dopant of a host-dopant-based light emitting layer.

In addition, as a material for the organic thin film, it is also possible to use a compound which may perform a function such as hole injection, hole transport, electron blocking, hole blocking, electron transport, or electron injection.

In order to improve the performance, life span, or efficiency of an organic light emitting diode (OLED), development of materials of an organic thin film is continuously required.

### SUMMARY OF THE INVENTION

### Technical Problem

An embodiment provides a compound for an organic optoelectronic diode capable or realizing an organic optoelectronic diode having high efficiency and long life span.

Another embodiment provides an organic optoelectronic diode including the compound.

Another embodiment provides a display device including the organic optoelectronic diode.

### Technical Solution

According to an embodiment, a compound represented by the following formulas is provided.

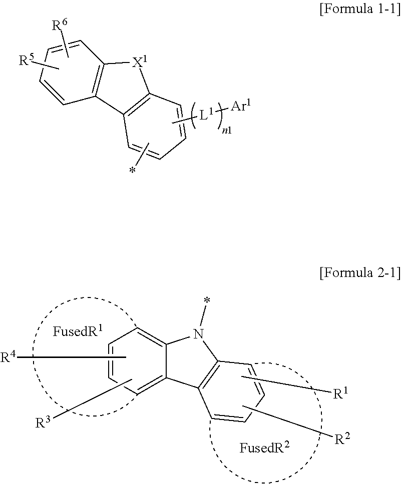

In Formula 1-1 and Formula 2-1,

X¹ is —O— or —S—, Ar¹ is a substituent having electron characteristics or a substituent having hole characteristics,

R¹ to R⁶ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof,

L¹ is a single bond, a substituted or unsubstituted C6 to C60 arylene group, or a substituted or unsubstituted C2 to C60 heteroarylene group,

n1 is one of integers of 0 to 2,

* is a linking point of Formula 1-1 and Formula 2-1, and

FuseR¹ and FusedR² are each independently a substituted or unsubstituted C3 to C60 fused ring.

According to another embodiment, an organic optoelectronic diode includes an anode and a cathode facing each other and at least one organic layer disposed between the anode and the cathode, wherein the organic layer includes the compound.

According to another embodiment, a display device including the organic optoelectronic diode is provided.

### Effect of the Invention

An organic optoelectronic diode having high efficiency and a long life span may be realized.

### BRIEF DESCRIPTION OF THE DRAWINGS

FIGS. 1 to 3 are each cross-sectional views showing organic light emitting diodes according to embodiments.

### DETAILED DESCRIPTION OF THE INVENTION

Hereinafter, embodiments of the present invention are described in detail. However, these embodiments are exemplary, the present invention is not limited thereto, and the present invention is defined by the scope of claims.

In the present invention, “substituted or unsubstituted” is substituted or unsubstituted with one or more substituents selected from the group consisting deuterium; a halogen; —CN; a C1 to C60 linear or branched alkyl group; a C2 to C60 linear or branched alkenyl group; a C2 to C60 linear or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O) RR′; a C1 to C20 alkylamine group; a C6 to C60 monocyclic or polycyclic arylamine group; a C2 to C60 monocyclic or polycyclic heteroarylamine group; and a substituted or unsubstituted alkoxy group, or a substituent to which two or more of the substituents selected from the substituents are combined, or a substituent to which two or more substituents selected from the substituents are linked.

These substituents can additionally form rings with adjacent substituents. For example, “a substituent formed by linking two or more substituents” may be a biphenyl group.

That is, the biphenyl group may refer to an aryl group or a substituent to which two phenyl groups are linked.

The additional substituents may be further substituted.

The R, R′, and R″ are the same as or different from each other, and are each independently hydrogen; deuterium; —CN; a substituted or unsubstituted C1 to C60 linear or branched alkyl; a substituted or unsubstituted C3 to C60 monocyclic or polycyclic cycloalkyl group; a substituted or unsubstituted C6 to C60 monocyclic or polycyclic aryl group; or a substituted or unsubstituted C2 to C60 monocyclic or polycyclic heteroaryl group.

According to an embodiment of the present invention, the “substituted or unsubstituted” is substituted or unsubstituted with one or more substituents selected from the group consisting of deuterium, a halogen, —CN, —SiRR′R″, —P(═O)RR′, a C1 to C20 linear or branched alkyl group, a C6 to C60 monocyclic or polycyclic aryl group, and a C2 to C60 monocyclic or polycyclic hetero aryl group, and the R, R′, and R″ are the same as or different from each other, and are each independently hydrogen, deuterium, —CN, a C1 to C60 alkyl group substituted or unsubstituted with deuterium, a halogen, —CN, a C1 to C20 alky group, a C6 to C60 aryl group, and a C2 to C60 hetero aryl group; a C3 to C60 cycloalkyl group substituted or unsubstituted with deuterium, a halogen, —CN, a C1 to C20 alky group, a C6 to C60 aryl group, and a C2 to C60 hetero aryl group; a C6 to C60 aryl group substituted or unsubstituted with deuterium, a halogen, —CN, a C1 to C20 alky group, a C6 to C60 aryl group, and a C2 to C60 hetero aryl group; or a C2 to C60 hetero aryl group substituted or unsubstituted with deuterium, a halogen, —CN, a C1 to C20 alky group, a C6 to C60 aryl group, and a C2 to C60 hetero aryl group.

The term “substituted” refers to when a hydrogen atom bonded to a carbon atom of a compound is replaced with another substituent, and the position to be substituted is a position at which a hydrogen atom is substituted, that is, the position is not limited as long as the substituent can be substituted, when substituted with two or more, two or more substituents may be the same as or different from each other.

In the present specification, the halogen may be fluorine, chlorine, bromine, or iodine.

In the present specification, the alkyl group may include a C1 to C60 linear or branched chain, and may be further substituted by other substituents.

The carbon number of the alkyl group may be 1 to 60, specifically 1 to 40, and more specifically 1 to 20.

Specific examples of the alky group are methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cylcopentylmethyl, cylcohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present specification, the alkenyl group may include a C2 to C60 linear or branched chain, and may be further substituted by other substituents.

The carbon number of the alkenyl group may be 2 to 60, specifically 2 to 40, and more specifically 2 to 20.

Specific examples of the alkenyl group are vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, stilbenyl, styrenyl, and the like, but are not limited thereto.

In the present specification, the alkynyl group may include a C2 to C60 linear or branched chain, and may be further substituted by another substituent. The carbon number of the alkynyl group may be 2 to 60, specifically 2 to 40, and more specifically 2 to 20.

In the present specification, the cycloalkyl group may include a C3 to C60 monocyclic or polycyclic ring, and may be further substituted by another substituent.

In here, the polycyclic ring refers to a group in which a cycloalkyl group is directly connected or condensed with another ring group.

In here, the other ring group may be a cycloalkyl group, but may be another type of ring group, such as a heterocycloalkyl, an aryl, a heteroaryl, or the like.

The carbon number of the cycloalkyl group may be 3 to 60, specifically 3 to 40, and more specifically 5 to 20.

Specifically, the cycloalkyl group is cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but is not limited thereto.

In the present specification, the alkoxy group may be a C1 to C10 alkoxy group, and more specifically, may be methoxy, ethoxy, propoxy, butoxy, pentoxy, and the like.

In the present specification, the silyl group may be represented by —SiRR′R″, and the definition of R is as described above. More specifically, the silyl group may be dimethylsilyl, diethylsilyl, methylethylsilyl, or the like.

In the present specification, the phosphine oxide group may be represented by —P(═O)RR′, and the definitions of R and R′ are as described above. More specifically, the phosphine oxide group may be a dimethyl phosphine oxide, a diethyl phosphine oxide, a methyl ethyl phosphine oxide, or the like.

In the present specification, the fluorenyl group refers to a substituent including various substituents at position 9. Specifically, the fluorenyl group may be used in a concept including a fluorenyl group substituted with two hydrogens, two alkyl groups, two aryl groups, or two heteroaryl groups at position 9. More specifically, the fluorenyl group may be a 9-di-H-fluorenyl group, a 9-di-methyl-fluorenyl group, a 9-di-phenyl-fluorenyl group, or the like.

In the present specification, the heterocycloalkyl group may include O, S, Se, N, or Si as a hetero atom, may include a C2 to C60 monocyclic or polycyclic ring, and may be further substituted by another substituent. Here, the polycyclic ring refers a group in which a heterocycloalkyl is directly connected or condensed with another ring group. Here, the other ring group may be a heterocycloalkyl group, but may be another type of ring group, such as a cycloalkyl group, an aryl group, a heteroaryl group, or the like. The carbon number of the heterocycloalkyl group may be 2 to 60, specifically 2 to 40, and more specifically 3 to 20.

In the present specification, the aryl group may include a C6 to C60 monocyclic or polycyclic ring, and may be further substituted by another substituent. Here, the polycyclic ring refers a group in which an aryl is directly connected or condensed with another ring group. Here, the other ring group may be an aryl group, but may be another type of ring group, such as a cycloalkyl group, a heterocycloalkyl group, a heteroaryl group, or the like. The aryl group may include a spiro group. The carbon number of the aryl group may be 6 to 60, specifically 6 to 40, and more specifically 6 to 25. Specific examples of the aryl group are phenyl, biphenyl, triphenyl, naphthyl, anthryl, chrysenyl, phenanthrenyl, perylenyl, fluoranthenyl, triphenylenyl, phenalenyl, pyrenyl, tetrasenyl, pentacenyl, fluorenyl, indenyl, acenaphthylenyl, benzofluorenyl, spirobifluorenyl, 2,3-dihydro-1H-indenyl, and condensed rings thereof, or the like, but are not limited thereto.

In the present specification, the spiro group may include a spiro structure, and may be C15 to C60. For example, the spiro group may include a structure in which a 2,3-dihydro-1H-indene group or a cyclohexane group is spiro bonded to a fluorenyl group. Specifically, the spiro group may include any one of following structural formulas.

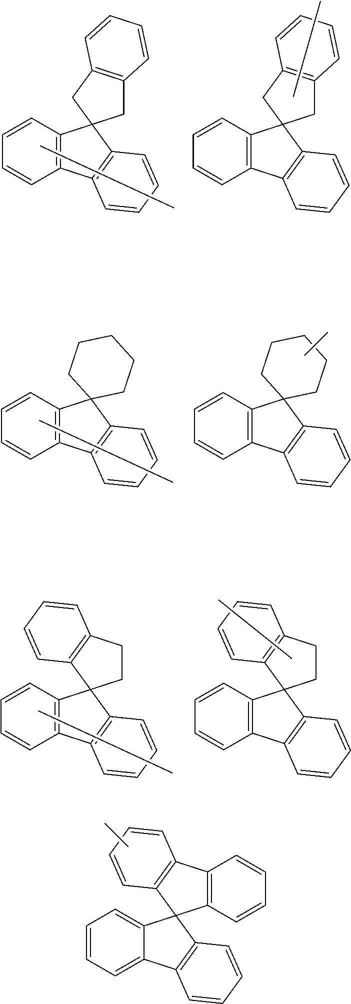

In the present specification, the heteroaryl may include S, O, Se, N, or Si as a hetero atom, and may include a C2 to C60 monocyclic or polycyclic ring, and may be further substituted by other substituents. Here, the polycyclic ring refers a group in which a heteroaryl group is directly connected or condensed with another ring group. Here, the other ring group may be a heteroaryl group, but may be another type of ring group, such as a cycloalkyl group, an heterocycloalkyl group, an aryl group, or the like. The carbon number of the heteroaryl group may be 2 to 60, specifically 2 to 40, and more specifically 3 to 25.

Specific examples of the heteroaryl group are pyridyl, pyrrolyl, pyrimidyl, pyridazinyl, furanyl, thiophene, imidazolyl, pyrazolyl, oxazolyl, isoxazolyl, thiazolyl, isothiazolyl, and triazolyl groups, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a deoxyl group, a triazinyl group, a tetrazinyl group, a quinolyl group, an isoquinolyl group, a quinazolinyl group, an isoquinazolinyl group, a quinozolinyl group, a naphthyridyl group, an acridinyl group, a phenantridinyl group, an imidazopyridinyl group, a diazanaphthalenyl group, a triazaindene group, an indolyl group, an indolinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilol group, a spirobi(dibenzosilol) group, a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b, f]azepine group, a 9,10-dihydroacridinyl group, a phenanthrazinyl group, a phenothiathiazinyl group, a phthalazinyl group, a naphthyridinyl group, a phenanthrolinyl group, a benzo[c][1,2,5]thiadiazolyl group, a 5,10-dihydrodibenzo[b, e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, or the like, but are not limited thereto.

In the present specification, the amine group may be selected from the group consisting of: a monoalkylamine group; a monoarylamine group; a monoheteroarylamine group; —NH2; a dialkylamine group; a diarylamine group; a diheteroarylamine group; an alkylarylamine group; an alkylheteroarylamine group; and an arylheteroarylamine group, and the carbon number is not particularly limited, but is preferably 1 to 30.

Specific examples of the heteroaryl group are a methylamine group, a dimethylamine group, an ethylamine group, a diethylamine group, phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenyrenylamine group, a biphenyltriphenylenylamine group, or the like, but are not limited thereto.

In the present specification, an arylene group means one having two bonding positions, that is, a divalent group. The description of the aryl groups described above may be applied except that they are each divalent. In addition, a heteroarylene group means having two bond positions in a heteroaryl group, that is, a divalent group. The description of the heteroaryl group described above may be applied except that they are each divalent.

In the present specification, hole characteristics refer to an ability to donate an electron to form a hole when an electric field is applied, and that a hole formed in the anode may be easily injected into the light emitting layer and transported in the light emitting layer due to conductive characteristics according to a highest occupied molecular orbital (HOMO) level.

As the substituent having hole characteristics, a substituted or unsubstituted C6 to C60 aryl group having hole characteristics, a substituted or unsubstituted C2 to C60 heteroaryl group having hole characteristics, a substituted or unsubstituted arylamine group, or a substituted or unsubstituted heteroarylamine group may be used.

More specifically, the substituted or unsubstituted C6 to C60 aryl group having the above hole characteristics may be a substituted or unsubstituted phenyl group, a substituted or unsubstituted naphthyl group, a substituted or unsubstituted phenanthrenyl group, a substituted or unsubstituted anthracenyl group, a substituted or unsubstituted fluorenyl group, a substituted or unsubstituted triphenylenyl group, a substituted or unsubstituted Spiro-fluorenyl group, a substituted or unsubstituted terphenyl group, a substituted or unsubstituted pyrenyl group, a substituted or unsubstituted perrylenyl group, or a combination thereof.

More specifically, the substituted or unsubstituted C2 to C60 heteroaryl group having the hole characteristics may be a substituted or unsubstituted carbazolyl group, a substituted or unsubstituted dibenzofuranyl group, a substituted or unsubstituted dibenzothiophenyl group, a substituted or unsubstituted indole carbazolyl group, and the like.

More specifically, the aryl group or heteroaryl group which is a substituent bonded to the nitrogen of the substituted or unsubstituted arylamine group and substituted or unsubstituted heteroarylamine group may be a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenylyl group, a substituted or unsubstituted p-terphenyl group, a substituted or unsubstituted m-terphenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, or a combination thereof.

In addition, electron characteristics refer to an ability to accept an electron when an electric field is applied and that electrons formed in the cathode may be easily injected into the light emitting layer and transported in the light emitting layer due to conductive characteristics according to a lowest unoccupied molecular orbital (LUMO) level.

The substituted or unsubstituted C2 to C60 heteroaryl group having electron characteristics may be a substituted or unsubstituted imidazolyl group, a substituted or unsubstituted tetrazolyl group, a substituted or unsubstituted quinolinyl group, a substituted or unsubstituted isoquinolinyl group, a substituted or unsubstituted pyridinylene group, a substituted or unsubstituted pyrimidinylene group, a substituted or unsubstituted triazinylene group, a substituted or unsubstituted furanyl group, a substituted or unsubstituted benzofuranyl group, a substituted or unsubstituted isofuranyl group, a substituted or unsubstituted benzoisofuranyl group, a substituted or unsubstituted oxazoline group, a substituted or unsubstituted benzooxazoline group, a substituted or unsubstituted oxadiazoline group, a substituted or unsubstituted benzooxadiazoline group, a substituted or unsubstituted oxatriazolyl group, a substituted or unsubstituted thiophenyl group, a substituted or unsubstituted benzothiophenyl group, a substituted or unsubstituted isothiazoline group, a substituted or unsubstituted benzoisothiazoline group, a substituted or unsubstituted thiazoline group, a substituted or unsubstituted benzothiazoline group, a substituted or unsubstituted pyridazinyl group, a substituted or unsubstituted benzopyridazinyl group, a substituted or unsubstituted pyrazinyl group, a substituted or unsubstituted benzopyrazinyl group, a substituted or unsubstituted phthalazinyl group, a substituted or unsubstituted benzoquinolinyl group, a substituted or unsubstituted quinoxalinyl group, a substituted or unsubstituted quinazolinyl group, a substituted or unsubstituted acridinyl group, a substituted or unsubstituted phenanthrolinyl group, a substituted or unsubstituted phenazinyl group, or a combination thereof.

More specifically, the substituted or unsubstituted C2 to C60 heteroaryl group having the above electron characteristics may be any one of the following formulas X-1 to X-5.

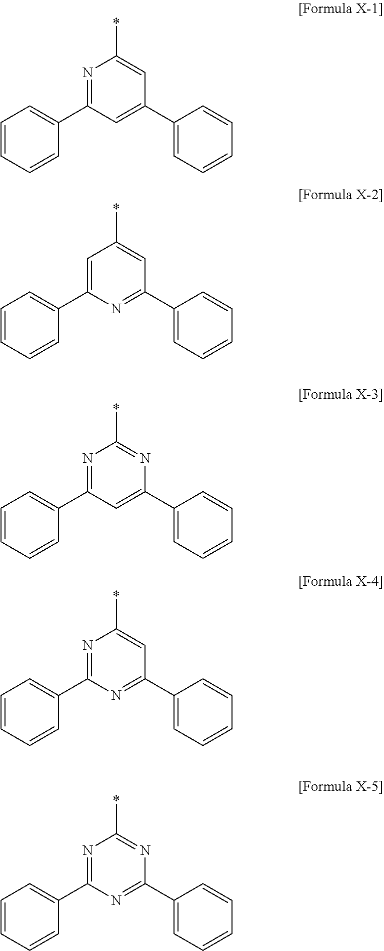 [SMILES: *c1cc(-c2ccccc2)cc(-c2ccccc2)n1 ; *c1cc(-c2ccccc2)nc(-c2ccccc2)c1 ; *c1nc(-c2ccccc2)cc(-c2ccccc2)n1 ; *c1cc(-c2ccccc2)nc(-c2ccccc2)n1 ; *c1nc(-c2ccccc2)nc(-c2ccccc2)n1]

In one embodiment of the present application, Lⁿ may be a direct bond (or a single bond), a substituted or unsubstituted arylene group, or a substituted or unsubstituted heteroarylene group.

In another embodiment, Lⁿ may be a direct bond, a substituted or unsubstituted C6 to C60 arylene group, or a substituted or unsubstituted C2 to C60 heteroarylene group.

In another embodiment, Lⁿ may be a direct bond, a substituted or unsubstituted C6 to C40 arylene group, or a substituted or unsubstituted C2 to C40 heteroarylene group.

The “n” in the Lⁿ means a number for distinguishing a substituent.

Hereinafter, a compound according to an embodiment is described.

The compound according to an embodiment is represented by the following formulas.

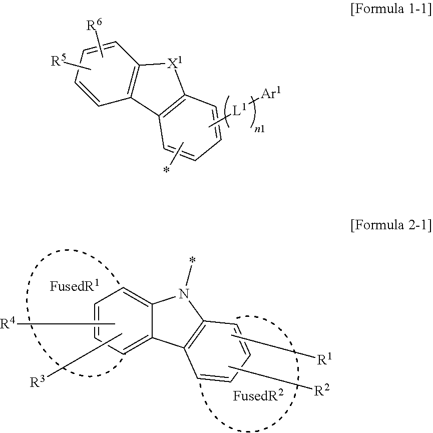

In Formulas 1-1 and 2-1, X¹ may be —O—, or —S—, Ar¹ may be a substituent having electron characteristics or a substituent having hole characteristics, R¹ to R⁶ may each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, may be a single bond, a substituted or unsubstituted C6 to C60 arylene group, or a substituted or unsubstituted C2 to C60 heteroarylene group, n1 may be one of integers of 0 to 2, * may be a linking point of Formulas 1-1 and 2-1, and FuseR¹ and FusedR² may each independently be a substituted or unsubstituted C3 to C60 fused ring. More specifically, FuseR¹ and FusedR² may each independently be a substituted or unsubstituted C3 to C20 fused ring.

The compound is a structure in which at least one fused ring is formed in the carbazole core. A dibenzofuranyl group or a dibenzothiophenyl group may be bonded to the core structure, and a substituent having electron characteristics or a substituent having hole characteristics may be further bonded.

In addition, by introducing a variety of substituents in the structure of the formulas, it is possible to synthesize a compound having a unique characteristic of the introduced substituents. For example, it is possible to synthesize a material that satisfies the conditions required for each organic layer by introducing a substituent mainly used in the hole injection layer material, the hole transport material, the light emitting layer material, the electron transport layer material, and the charge generating layer material used in the manufacture of the organic light emitting diode to the core structure

In addition, it is possible to finely control the energy band gap to improve the interfacial properties between the organic material and to vary the use of the material by introducing a variety of substituents in the structure of the formulas.

On the other hand, the compound has high thermal stability because of the high glass transition temperature (Tg). Such high thermal stability is an important factor in providing driving stability to the device.

Formula 1-1 may be represented by Formula 1-2.

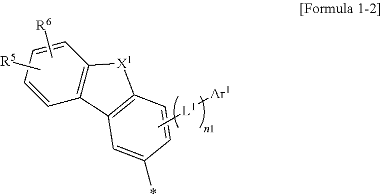

In Formula 1-2, X¹ may be —O— or —S—, Ar¹ may be a substituent having electron characteristics or a substituent having hole characteristics, R⁵ and R⁶ may each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, may be a singly bond, a substituted or unsubstituted C6 to C60 arylene group, or a substituted or unsubstituted C2 to C60 heteroarylene group, n1 may be one of integers of 0 to 2, and * may be a linking point of Formula 1-2 and 2-1.

Formula 1-2 specifically describes the binding position, considering the ease of synthesis and the efficiency of the expansion of the electron cloud.

Hereinafter, a carbazole core structure including the fused ring is described with more specific examples.

Formula 2-1 may be represented by Formula 2-2.

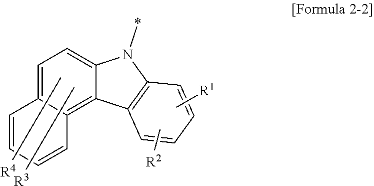

In Formula 2-2, R¹ to R⁴ may be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and * may be a linking point of Formula 1-1 and 2-2.

Alternatively, Formula 2-1 may be represented by Formula 2-3.

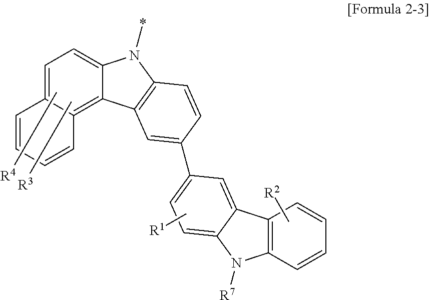

In Formula 2-3, R¹ to R⁴ and R⁷ may each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and * may be a linking point of Formula 1-1 and 2-3.

Alternatively, Formula 2-1 may be represented by Formula 2-4.

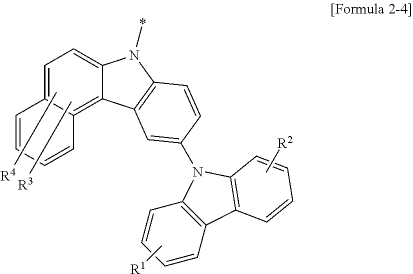

In Formula 2-4, R¹ to R⁴ may be each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and * may be a linking point of Formula 1-1 and 2-4.

Formula 2-1 may be represented by Formula 2-5.

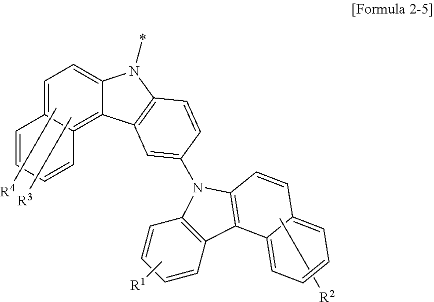

In Formula 2-5, R¹ to R⁴ may each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and * may be a linking point of Formula 1-1 and 2-5.

Formula 2-1 may be represented by Formula 2-6.

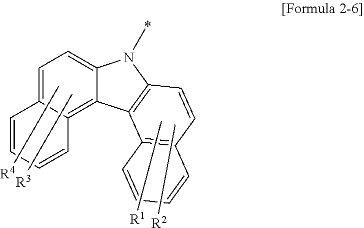

In Formula 2-6, R¹ to R⁴ may each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and * may be a linking point of Formula 1-1 and 2-6.

Formula 2-1 may be represented by Formula 2-7.

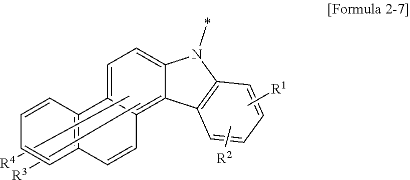

In Formula 2-7, R¹ to R⁴ may each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and * may be a linking point of Formula 1-1 and 2-7.

Formula 2-1 may be represented by Formula 2-8.

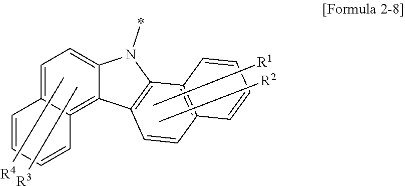

In Formula 2-8, R¹ to R⁴ may each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and * may be a linking point of Formula 1-1 and 2-8.

Formula 2-1 may be represented by Formula 2-9.

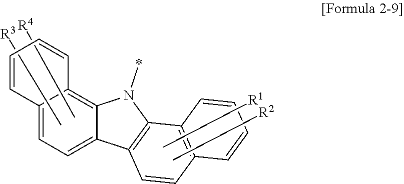

In Formula 2-9, R¹ to R⁴ may each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and * may be a linking point of Formula 1-1 and 2-9.

Formula 2-1 may be represented by Formula 2-10.

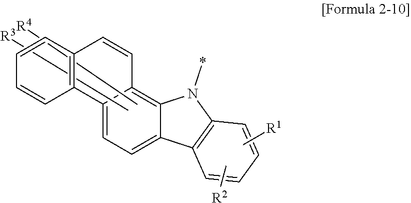

In Formula 2-10, R¹ to R⁴ may each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and * may be a linking point of Formula 1-1 and 2-10.

Formula 2-1 may be represented by Formula 2-11.

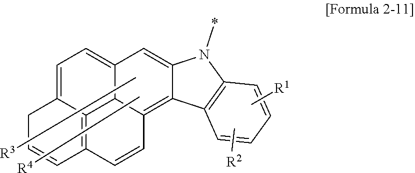

In Formula 2-11, R¹ to R⁴ may each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and * may be a linking point of Formula 1-1 and 2-11.

The carbazole cores of Formulas 2-2 to 2-11 may be selected in consideration of a substituent additionally bonded to the compound. The various carbazole structures can satisfy the thermal stability and various energy levels of the compound.

More specifically, the Ar¹ may be a substituted or unsubstituted C6 to C60 aryl group, or a substituted or unsubstituted C2 to C60 heteroaryl group.

More specifically, for example, the Ar¹ may be the following Formula 3-1 or Formula 3-2.

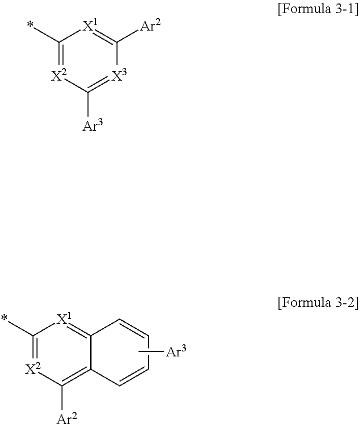

In Formula 3-1 and Formula 3-2, the X¹ to X³ may be —CR′—, or —N—, at least one of X¹ to X³ may be —N—, Ar² and Ar³ may each independently be hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and R′ may be hydrogen, deuterium, a cyano group, or a substituted or unsubstituted C1 to C60 alkyl group.

As in the case of Formula 3-1 or 3-2, when the substituent with enhanced electron characteristics is introduced, the distribution of HOMO-LUMO with the carbazole core becomes clearer, thereby providing a bi-polar compound.

In Formula 3-1 and Formula 3-2, at least one of Ar² and Ar³ may be any one of Formula 4-1 to Formula 4-5.

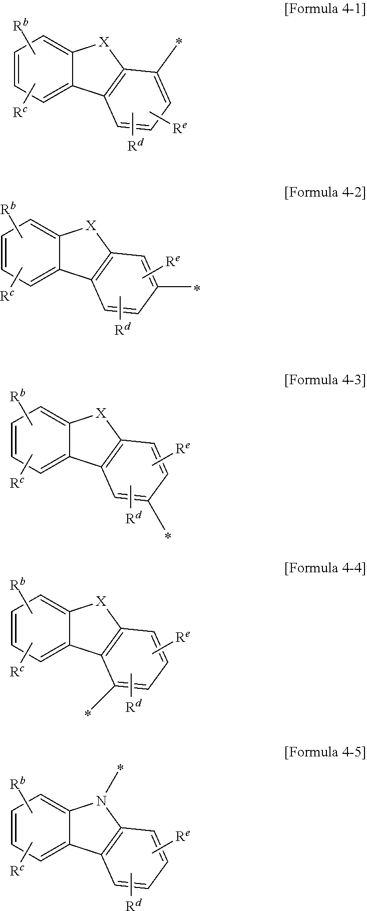

In Formula 4-1 to Formula 4-5, X may be —NR^{x}—, —O—, —S—, or —CR^{x}R^{y}—, R^{x} and R^{y} may each independently be hydrogen, deuterium, a cyano group, a substituted or unsubstituted C1 to C60 alkyl group, or a C6 to C60 aryl group, and R^{b} to R^{e} may each independently be hydrogen, deuterium, a cyano group, a substituted or unsubstituted C1 to C60 alkyl group, or a C6 to C60 aryl group.

By including the above Formulas 4-1 to 4-5, it can be expected that the strength and heat resistance properties of the compound can be improved, and the electric field strength of the compound is lowered, thereby improving the hole movement velocity.

The R¹ to R⁶ may each independently be any one of substituents of the following Group I.

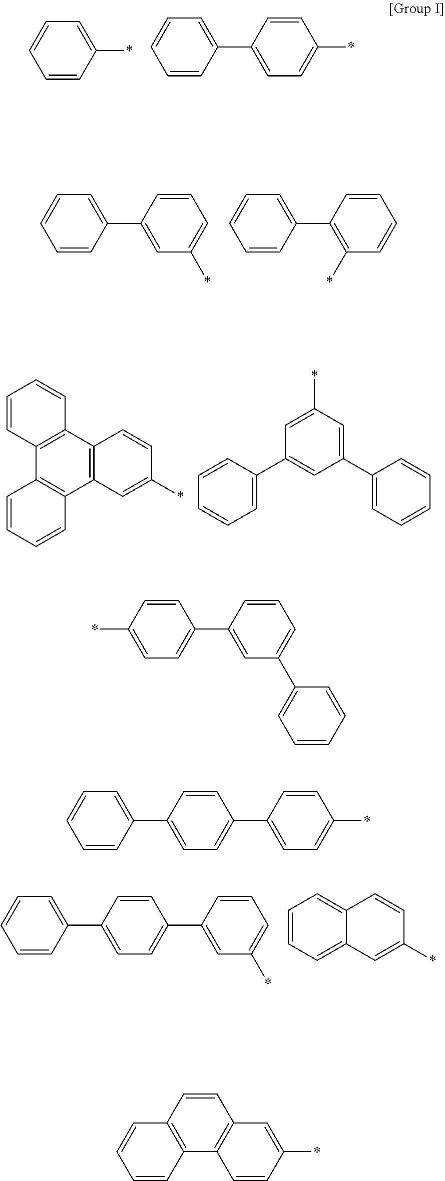

In Group I, * may be a linking point.

The compound of one example described above may be represented by any one of the compounds of Group II.

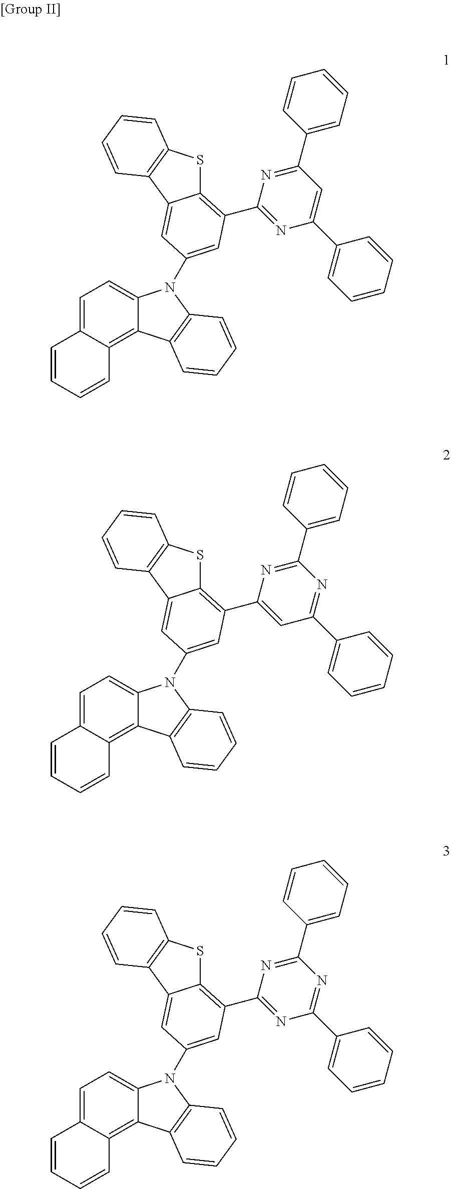 [SMILES: c1ccc(-c2cc(-c3ccccc3)nc(-c3cc(-n4c5ccccc5c5c6ccccc6ccc54)cc4c3sc3ccccc34)n2)cc1 ; c1ccc(-c2cc(-c3cc(-n4c5ccccc5c5c6ccccc6ccc54)cc4c3sc3ccccc34)nc(-c3ccccc3)n2)cc1 ; c1ccc(-c2nc(-c3ccccc3)nc(-c3cc(-n4c5ccccc5c5c6ccccc6ccc54)cc4c3sc3ccccc34)n2)cc1]

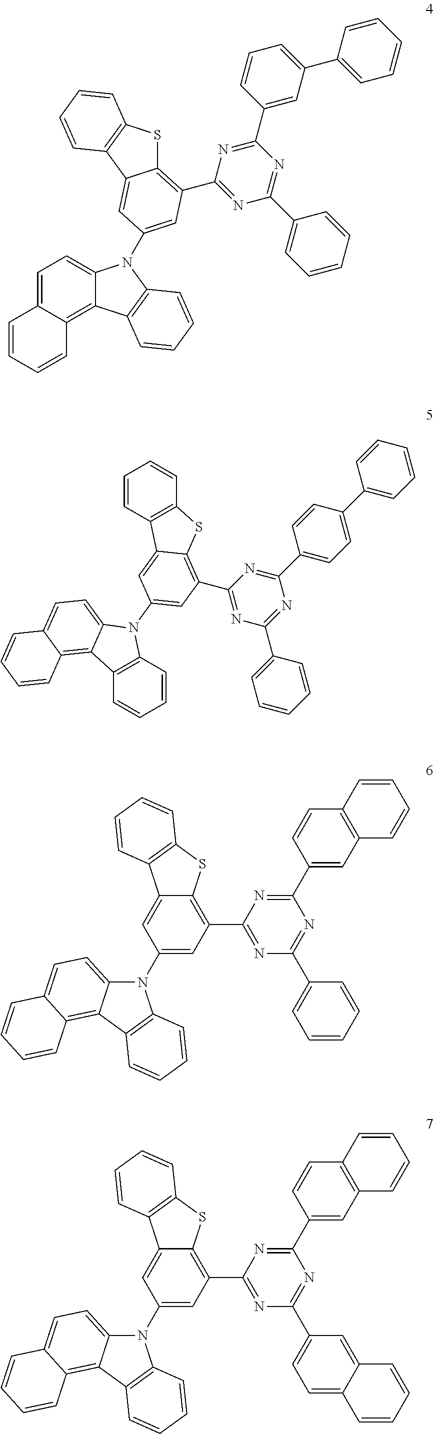 [SMILES: c1ccc(-c2cccc(-c3nc(-c4ccccc4)nc(-c4cc(-n5c6ccccc6c6c7ccccc7ccc65)cc5c4sc4ccccc45)n3)c2)cc1 ; c1ccc(-c2ccc(-c3nc(-c4ccccc4)nc(-c4cc(-n5c6ccccc6c6c7ccccc7ccc65)cc5c4sc4ccccc45)n3)cc2)cc1 ; c1ccc(-c2nc(-c3ccc4ccccc4c3)nc(-c3cc(-n4c5ccccc5c5c6ccccc6ccc54)cc4c3sc3ccccc34)n2)cc1 ; c1ccc2cc(-c3nc(-c4ccc5ccccc5c4)nc(-c4cc(-n5c6ccccc6c6c7ccccc7ccc65)cc5c4sc4ccccc45)n3)ccc2c1]

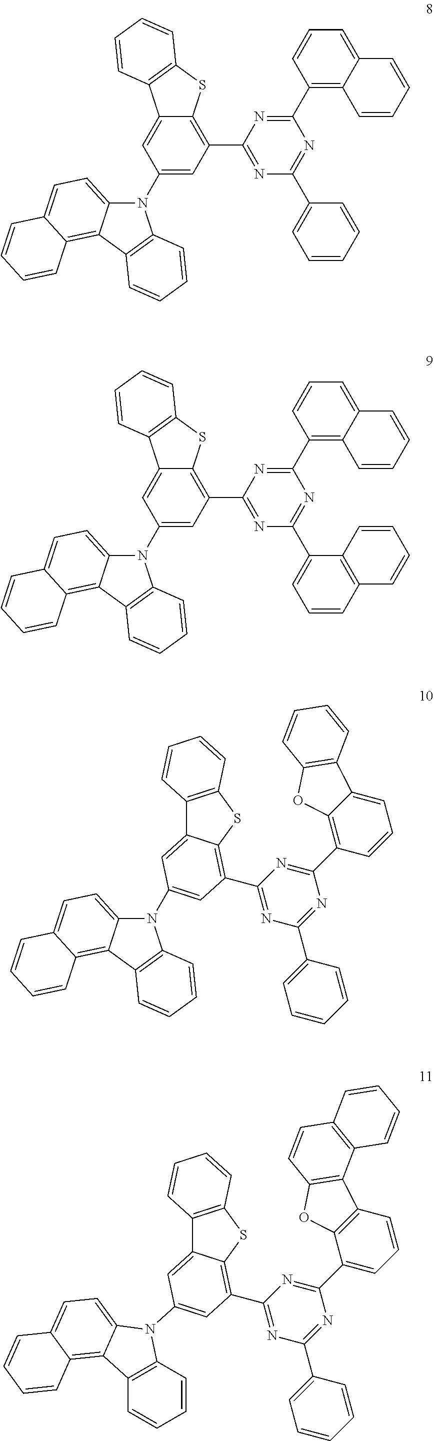 [SMILES: c1ccc(-c2nc(-c3cccc4ccccc34)nc(-c3cc(-n4c5ccccc5c5c6ccccc6ccc54)cc4c3sc3ccccc34)n2)cc1 ; c1ccc2c(-c3nc(-c4cccc5ccccc45)nc(-c4cc(-n5c6ccccc6c6c7ccccc7ccc65)cc5c4sc4ccccc45)n3)cccc2c1 ; c1ccc(-c2nc(-c3cccc4c3oc3ccccc34)nc(-c3cc(-n4c5ccccc5c5c6ccccc6ccc54)cc4c3sc3ccccc34)n2)cc1 ; c1ccc(-c2nc(-c3cccc4c3oc3ccc5ccccc5c34)nc(-c3cc(-n4c5ccccc5c5c6ccccc6ccc54)cc4c3sc3ccccc34)n2)cc1]

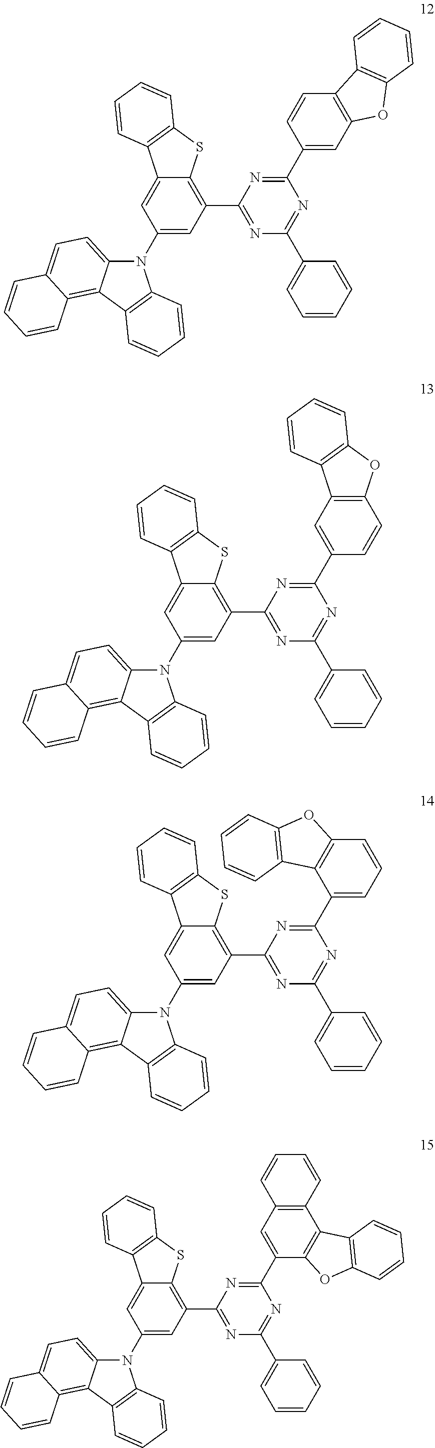 [SMILES: c1ccc(-c2nc(-c3ccc4c(c3)oc3ccccc34)nc(-c3cc(-n4c5ccccc5c5c6ccccc6ccc54)cc4c3sc3ccccc34)n2)cc1 ; c1ccc(-c2nc(-c3ccc4oc5ccccc5c4c3)nc(-c3cc(-n4c5ccccc5c5c6ccccc6ccc54)cc4c3sc3ccccc34)n2)cc1 ; c1ccc(-c2nc(-c3cc(-n4c5ccccc5c5c6ccccc6ccc54)cc4c3sc3ccccc34)nc(-c3cccc4oc5ccccc5c34)n2)cc1 ; c1ccc(-c2nc(-c3cc4ccccc4c4c3oc3ccccc34)nc(-c3cc(-n4c5ccccc5c5c6ccccc6ccc54)cc4c3sc3ccccc34)n2)cc1]

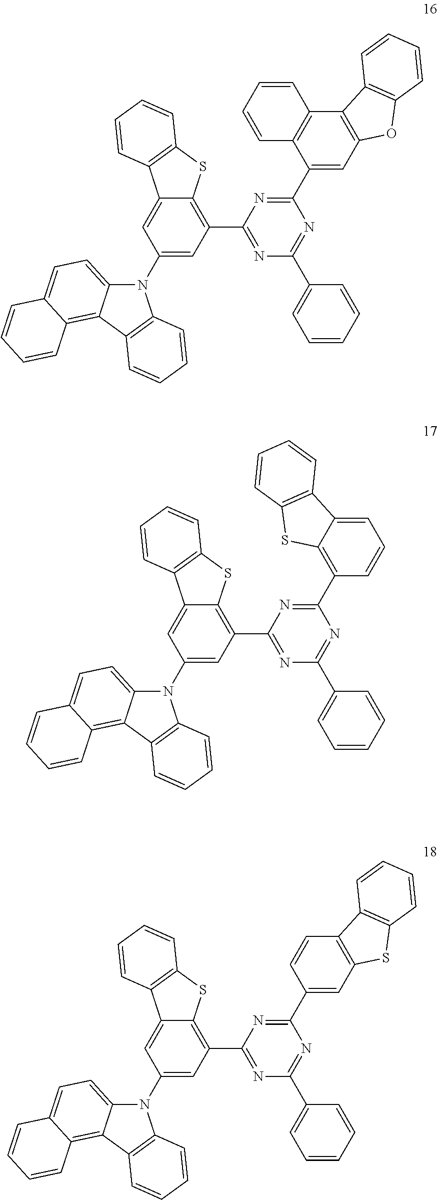 [SMILES: c1ccc(-c2nc(-c3cc4oc5ccccc5c4c4ccccc34)nc(-c3cc(-n4c5ccccc5c5c6ccccc6ccc54)cc4c3sc3ccccc34)n2)cc1 ; c1ccc(-c2nc(-c3cccc4c3sc3ccccc34)nc(-c3cc(-n4c5ccccc5c5c6ccccc6ccc54)cc4c3sc3ccccc34)n2)cc1 ; c1ccc(-c2nc(-c3ccc4c(c3)sc3ccccc34)nc(-c3cc(-n4c5ccccc5c5c6ccccc6ccc54)cc4c3sc3ccccc34)n2)cc1]

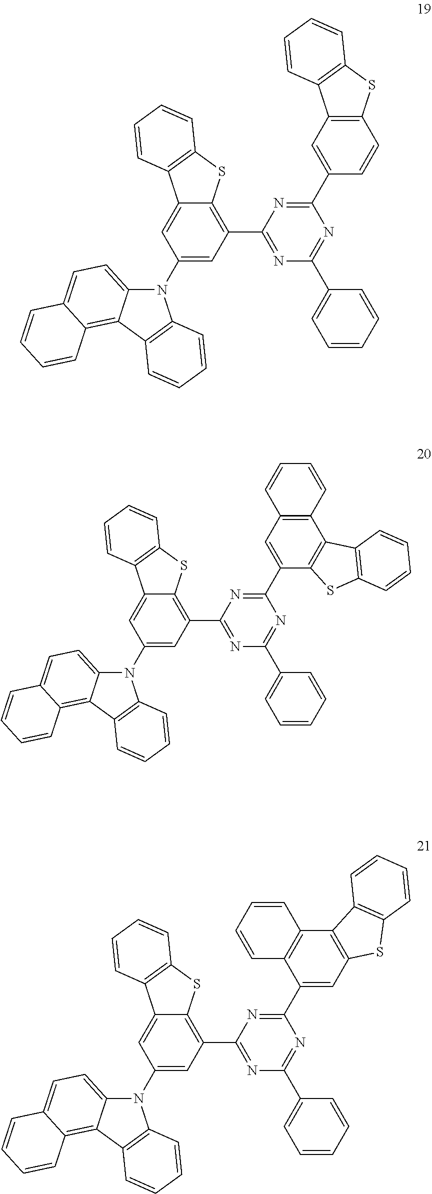 [SMILES: c1ccc(-c2nc(-c3ccc4sc5ccccc5c4c3)nc(-c3cc(-n4c5ccccc5c5c6ccccc6ccc54)cc4c3sc3ccccc34)n2)cc1 ; c1ccc(-c2nc(-c3cc(-n4c5ccccc5c5c6ccccc6ccc54)cc4c3sc3ccccc34)nc(-c3cc4ccccc4c4c3sc3ccccc34)n2)cc1 ; c1ccc(-c2nc(-c3cc4sc5ccccc5c4c4ccccc34)nc(-c3cc(-n4c5ccccc5c5c6ccccc6ccc54)cc4c3sc3ccccc34)n2)cc1]

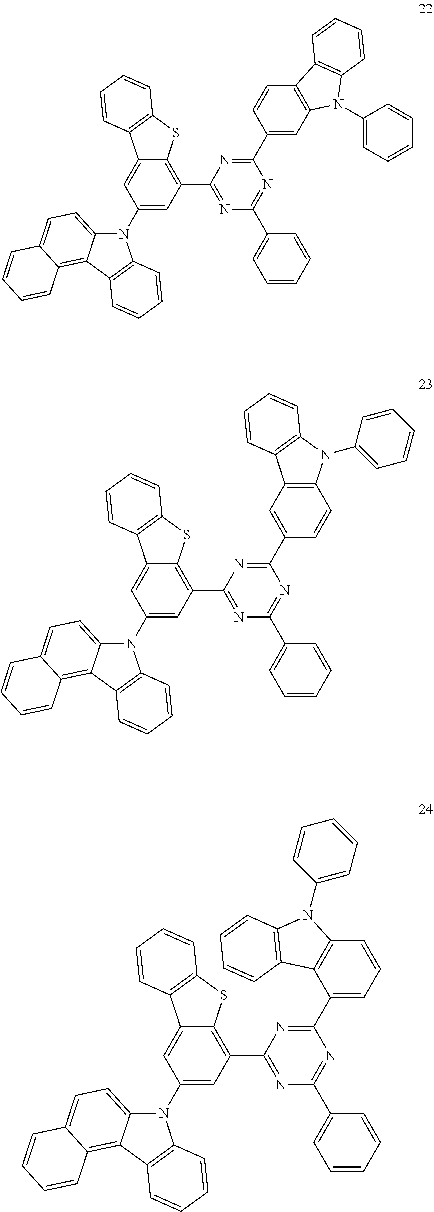 [SMILES: c1ccc(-c2nc(-c3ccc4c5ccccc5n(-c5ccccc5)c4c3)nc(-c3cc(-n4c5ccccc5c5c6ccccc6ccc54)cc4c3sc3ccccc34)n2)cc1 ; c1ccc(-c2nc(-c3ccc4c(c3)c3ccccc3n4-c3ccccc3)nc(-c3cc(-n4c5ccccc5c5c6ccccc6ccc54)cc4c3sc3ccccc34)n2)cc1 ; c1ccc(-c2nc(-c3cc(-n4c5ccccc5c5c6ccccc6ccc54)cc4c3sc3ccccc34)nc(-c3cccc4c3c3ccccc3n4-c3ccccc3)n2)cc1]

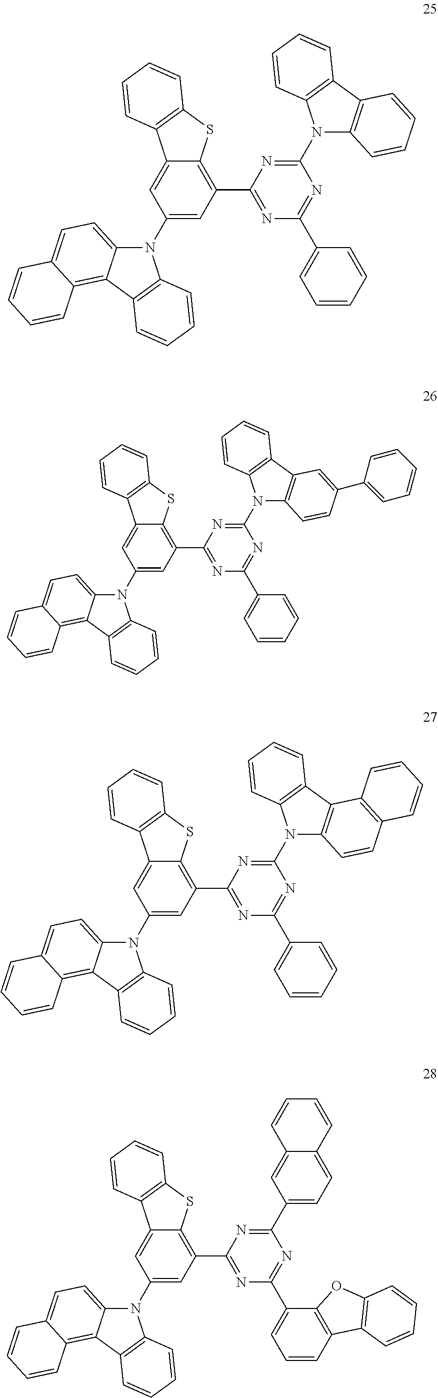 [SMILES: c1ccc(-c2nc(-c3cc(-n4c5ccccc5c5c6ccccc6ccc54)cc4c3sc3ccccc34)nc(-n3c4ccccc4c4ccccc43)n2)cc1 ; c1ccc(-c2ccc3c(c2)c2ccccc2n3-c2nc(-c3ccccc3)nc(-c3cc(-n4c5ccccc5c5c6ccccc6ccc54)cc4c3sc3ccccc34)n2)cc1 ; c1ccc(-c2nc(-c3cc(-n4c5ccccc5c5c6ccccc6ccc54)cc4c3sc3ccccc34)nc(-n3c4ccccc4c4c5ccccc5ccc43)n2)cc1 ; c1ccc2cc(-c3nc(-c4cccc5c4oc4ccccc45)nc(-c4cc(-n5c6ccccc6c6c7ccccc7ccc65)cc5c4sc4ccccc45)n3)ccc2c1]

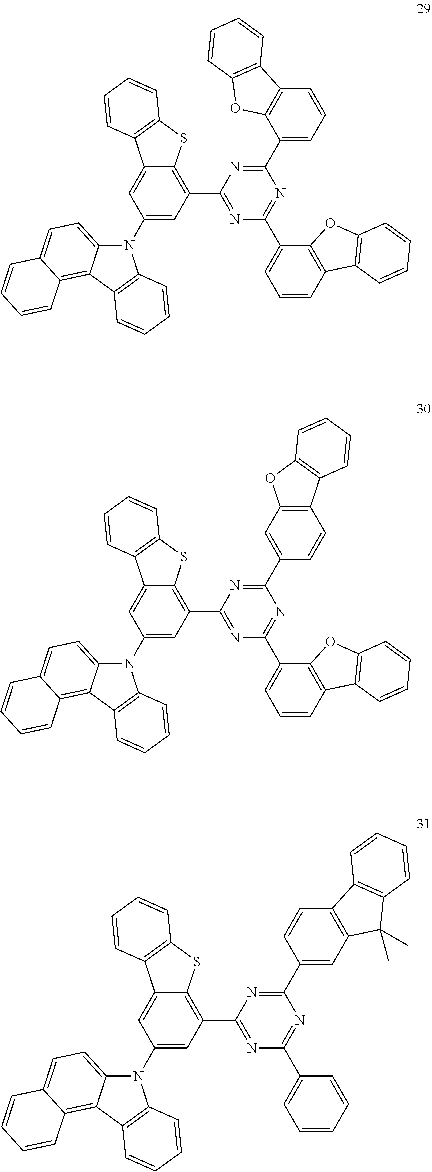 [SMILES: c1ccc2c(c1)ccc1c2c2ccccc2n1-c1cc(-c2nc(-c3cccc4c3oc3ccccc34)nc(-c3cccc4c3oc3ccccc34)n2)c2sc3ccccc3c2c1 ; c1ccc2c(c1)ccc1c2c2ccccc2n1-c1cc(-c2nc(-c3ccc4c(c3)oc3ccccc34)nc(-c3cccc4c3oc3ccccc34)n2)c2sc3ccccc3c2c1 ; CC1(C)c2ccccc2-c2ccc(-c3nc(-c4ccccc4)nc(-c4cc(-n5c6ccccc6c6c7ccccc7ccc65)cc5c4sc4ccccc45)n3)cc21]

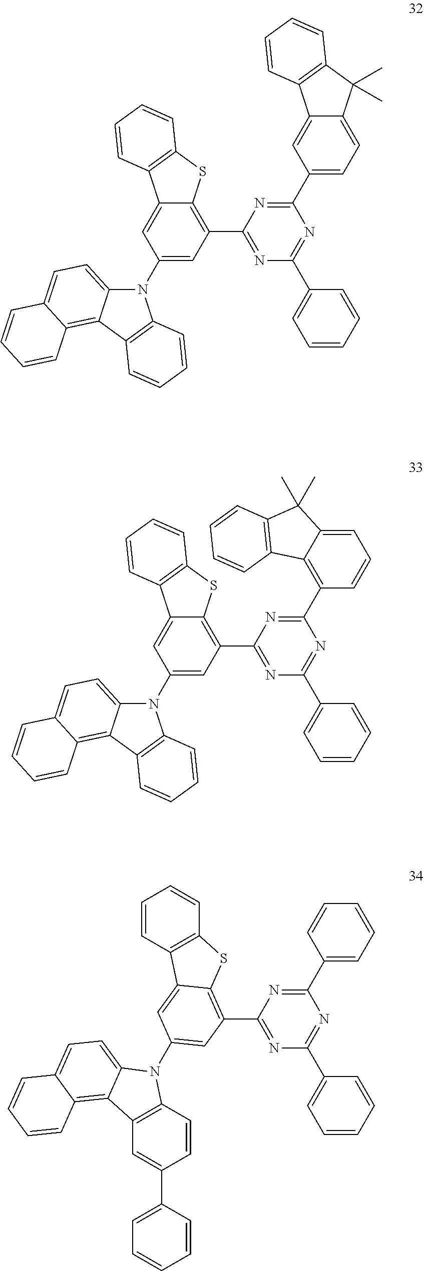 [SMILES: CC1(C)c2ccccc2-c2cc(-c3nc(-c4ccccc4)nc(-c4cc(-n5c6ccccc6c6c7ccccc7ccc65)cc5c4sc4ccccc45)n3)ccc21 ; CC1(C)c2ccccc2-c2c(-c3nc(-c4ccccc4)nc(-c4cc(-n5c6ccccc6c6c7ccccc7ccc65)cc5c4sc4ccccc45)n3)cccc21 ; c1ccc(-c2ccc3c(c2)c2c4ccccc4ccc2n3-c2cc(-c3nc(-c4ccccc4)nc(-c4ccccc4)n3)c3sc4ccccc4c3c2)cc1]

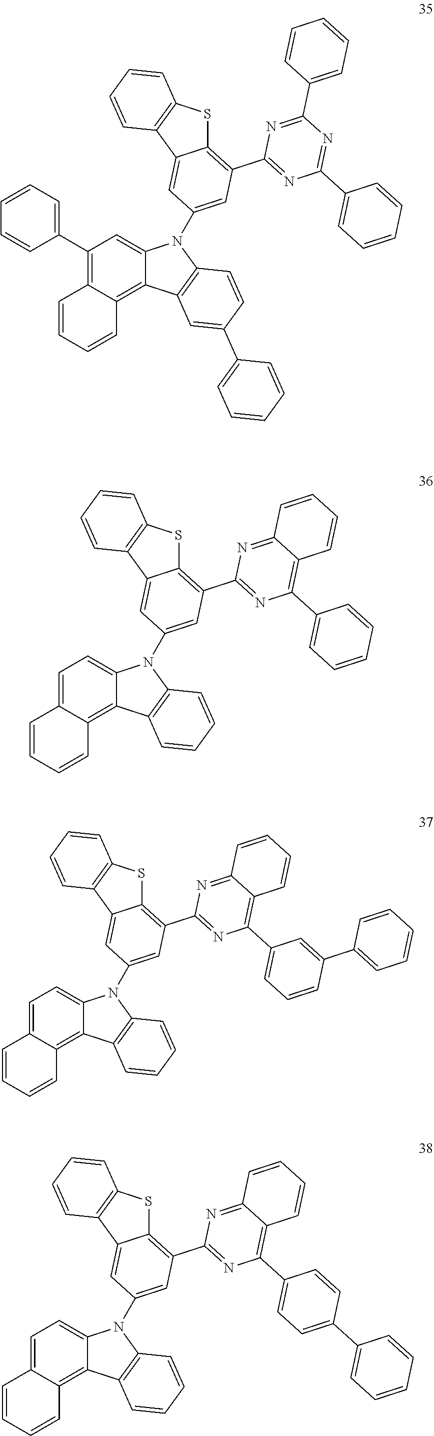 [SMILES: c1ccc(-c2ccc3c(c2)c2c4ccccc4c(-c4ccccc4)cc2n3-c2cc(-c3nc(-c4ccccc4)nc(-c4ccccc4)n3)c3sc4ccccc4c3c2)cc1 ; c1ccc(-c2nc(-c3cc(-n4c5ccccc5c5c6ccccc6ccc54)cc4c3sc3ccccc34)nc3ccccc23)cc1 ; c1ccc(-c2cccc(-c3nc(-c4cc(-n5c6ccccc6c6c7ccccc7ccc65)cc5c4sc4ccccc45)nc4ccccc34)c2)cc1 ; c1ccc(-c2ccc(-c3nc(-c4cc(-n5c6ccccc6c6c7ccccc7ccc65)cc5c4sc4ccccc45)nc4ccccc34)cc2)cc1]

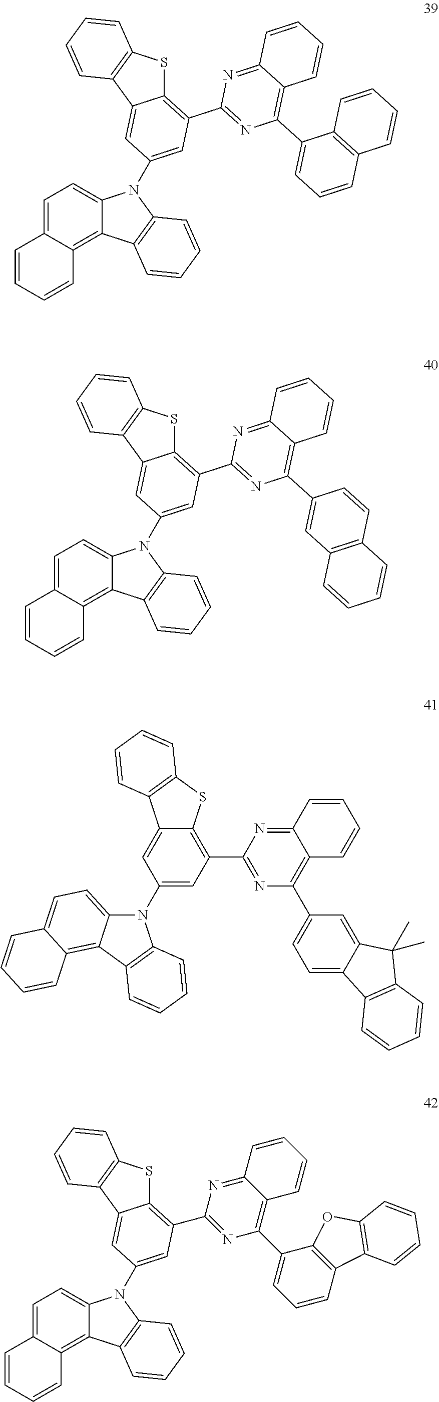 [SMILES: c1ccc2c(-c3nc(-c4cc(-n5c6ccccc6c6c7ccccc7ccc65)cc5c4sc4ccccc45)nc4ccccc34)cccc2c1 ; c1ccc2cc(-c3nc(-c4cc(-n5c6ccccc6c6c7ccccc7ccc65)cc5c4sc4ccccc45)nc4ccccc34)ccc2c1 ; CC1(C)c2ccccc2-c2ccc(-c3nc(-c4cc(-n5c6ccccc6c6c7ccccc7ccc65)cc5c4sc4ccccc45)nc4ccccc34)cc21 ; c1ccc2c(c1)ccc1c2c2ccccc2n1-c1cc(-c2nc(-c3cccc4c3oc3ccccc34)c3ccccc3n2)c2sc3ccccc3c2c1]

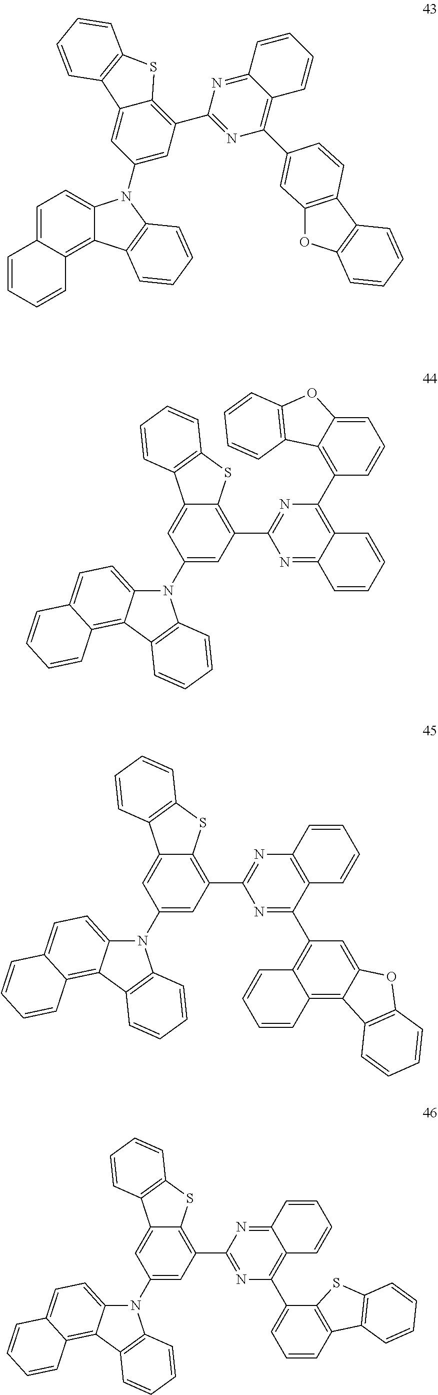 [SMILES: c1ccc2c(c1)ccc1c2c2ccccc2n1-c1cc(-c2nc(-c3ccc4c(c3)oc3ccccc34)c3ccccc3n2)c2sc3ccccc3c2c1 ; c1ccc2c(c1)ccc1c2c2ccccc2n1-c1cc(-c2nc(-c3cccc4oc5ccccc5c34)c3ccccc3n2)c2sc3ccccc3c2c1 ; c1ccc2c(c1)ccc1c2c2ccccc2n1-c1cc(-c2nc(-c3cc4oc5ccccc5c4c4ccccc34)c3ccccc3n2)c2sc3ccccc3c2c1 ; c1ccc2c(c1)ccc1c2c2ccccc2n1-c1cc(-c2nc(-c3cccc4c3sc3ccccc34)c3ccccc3n2)c2sc3ccccc3c2c1]

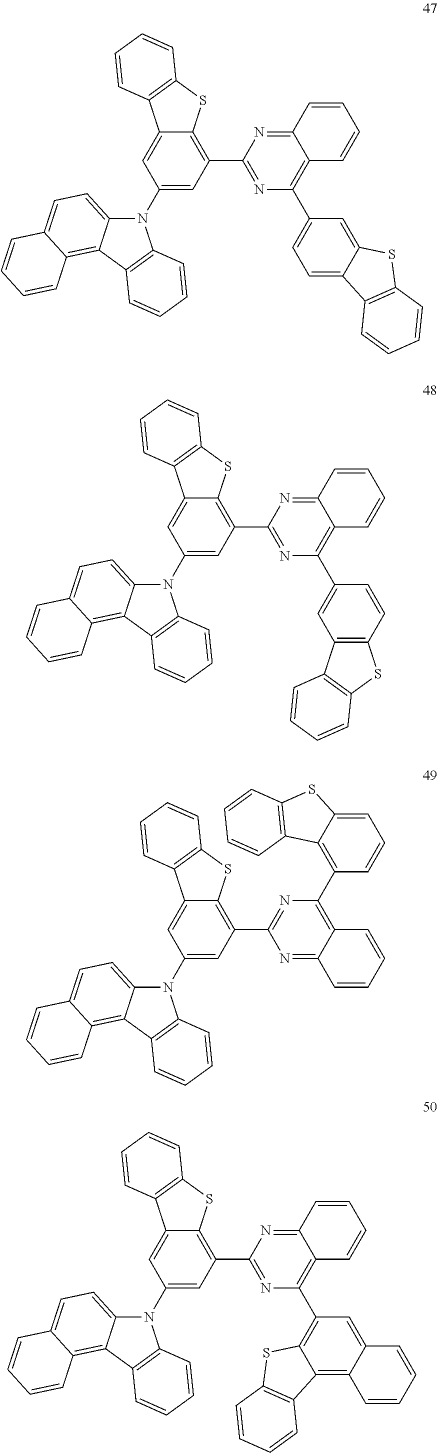 [SMILES: c1ccc2c(c1)ccc1c2c2ccccc2n1-c1cc(-c2nc(-c3ccc4c(c3)sc3ccccc34)c3ccccc3n2)c2sc3ccccc3c2c1 ; c1ccc2c(c1)ccc1c2c2ccccc2n1-c1cc(-c2nc(-c3ccc4sc5ccccc5c4c3)c3ccccc3n2)c2sc3ccccc3c2c1 ; c1ccc2c(c1)ccc1c2c2ccccc2n1-c1cc(-c2nc(-c3cccc4sc5ccccc5c34)c3ccccc3n2)c2sc3ccccc3c2c1 ; c1ccc2c(c1)cc(-c1nc(-c3cc(-n4c5ccccc5c5c6ccccc6ccc54)cc4c3sc3ccccc34)nc3ccccc13)c1sc3ccccc3c12]

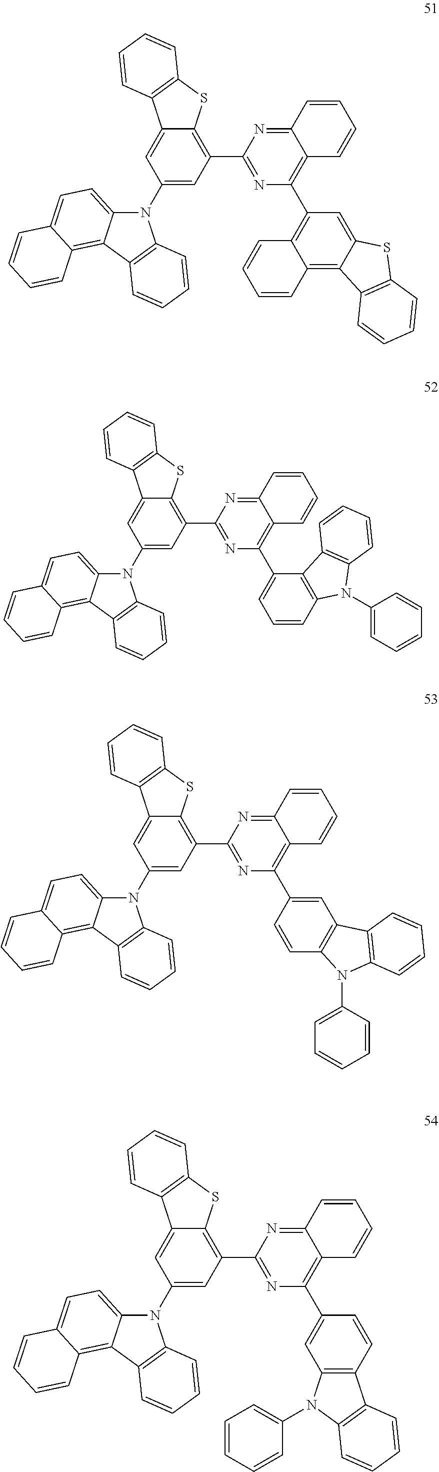 [SMILES: c1ccc2c(c1)ccc1c2c2ccccc2n1-c1cc(-c2nc(-c3cc4sc5ccccc5c4c4ccccc34)c3ccccc3n2)c2sc3ccccc3c2c1 ; c1ccc(-n2c3ccccc3c3c(-c4nc(-c5cc(-n6c7ccccc7c7c8ccccc8ccc76)cc6c5sc5ccccc56)nc5ccccc45)cccc32)cc1 ; c1ccc(-n2c3ccccc3c3cc(-c4nc(-c5cc(-n6c7ccccc7c7c8ccccc8ccc76)cc6c5sc5ccccc56)nc5ccccc45)ccc32)cc1 ; c1ccc(-n2c3ccccc3c3ccc(-c4nc(-c5cc(-n6c7ccccc7c7c8ccccc8ccc76)cc6c5sc5ccccc56)nc5ccccc45)cc32)cc1]

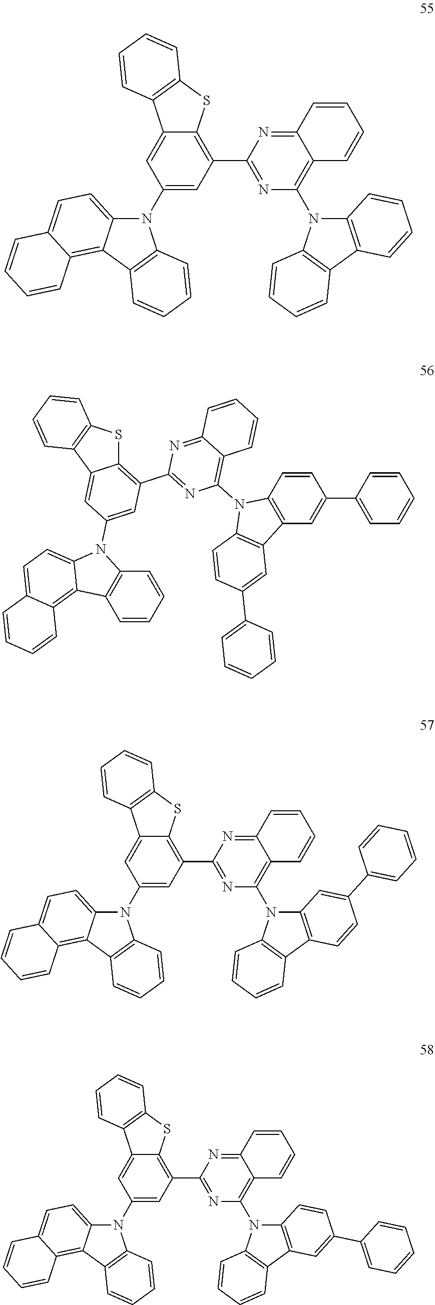 [SMILES: c1ccc2c(c1)ccc1c2c2ccccc2n1-c1cc(-c2nc(-n3c4ccccc4c4ccccc43)c3ccccc3n2)c2sc3ccccc3c2c1 ; c1ccc(-c2ccc3c(c2)c2cc(-c4ccccc4)ccc2n3-c2nc(-c3cc(-n4c5ccccc5c5c6ccccc6ccc54)cc4c3sc3ccccc34)nc3ccccc23)cc1 ; c1ccc(-c2ccc3c4ccccc4n(-c4nc(-c5cc(-n6c7ccccc7c7c8ccccc8ccc76)cc6c5sc5ccccc56)nc5ccccc45)c3c2)cc1 ; c1ccc(-c2ccc3c(c2)c2ccccc2n3-c2nc(-c3cc(-n4c5ccccc5c5c6ccccc6ccc54)cc4c3sc3ccccc34)nc3ccccc23)cc1]

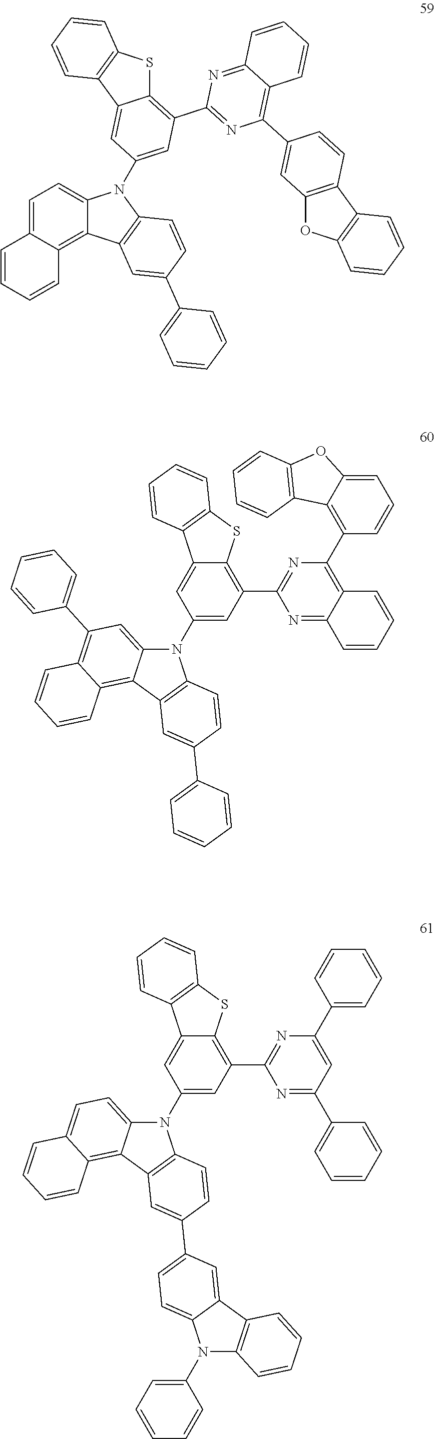 [SMILES: c1ccc(-c2ccc3c(c2)c2c4ccccc4ccc2n3-c2cc(-c3nc(-c4ccc5c(c4)oc4ccccc45)c4ccccc4n3)c3sc4ccccc4c3c2)cc1 ; c1ccc(-c2ccc3c(c2)c2c4ccccc4c(-c4ccccc4)cc2n3-c2cc(-c3nc(-c4cccc5oc6ccccc6c45)c4ccccc4n3)c3sc4ccccc4c3c2)cc1 ; c1ccc(-c2cc(-c3ccccc3)nc(-c3cc(-n4c5ccc(-c6ccc7c(c6)c6ccccc6n7-c6ccccc6)cc5c5c6ccccc6ccc54)cc4c3sc3ccccc34)n2)cc1]

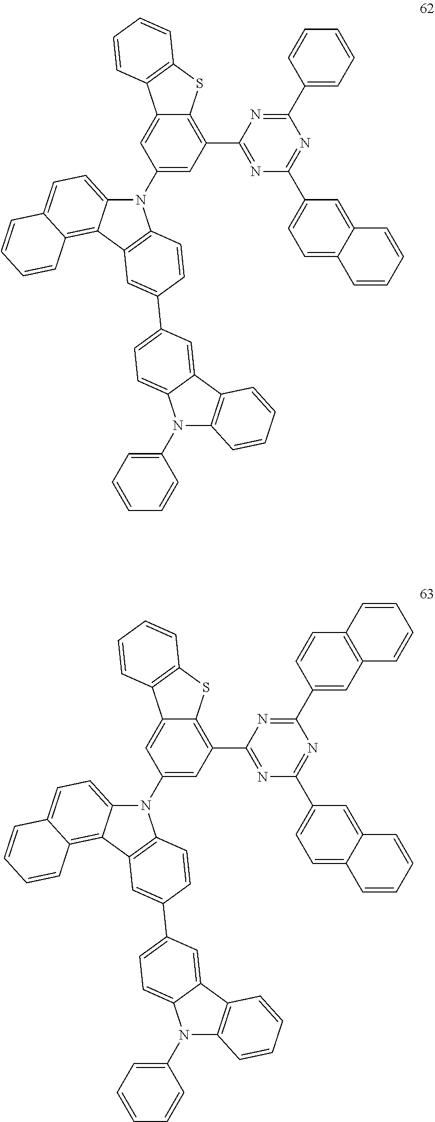 [SMILES: c1ccc(-c2nc(-c3ccc4ccccc4c3)nc(-c3cc(-n4c5ccc(-c6ccc7c(c6)c6ccccc6n7-c6ccccc6)cc5c5c6ccccc6ccc54)cc4c3sc3ccccc34)n2)cc1 ; c1ccc(-n2c3ccccc3c3cc(-c4ccc5c(c4)c4c6ccccc6ccc4n5-c4cc(-c5nc(-c6ccc7ccccc7c6)nc(-c6ccc7ccccc7c6)n5)c5sc6ccccc6c5c4)ccc32)cc1]

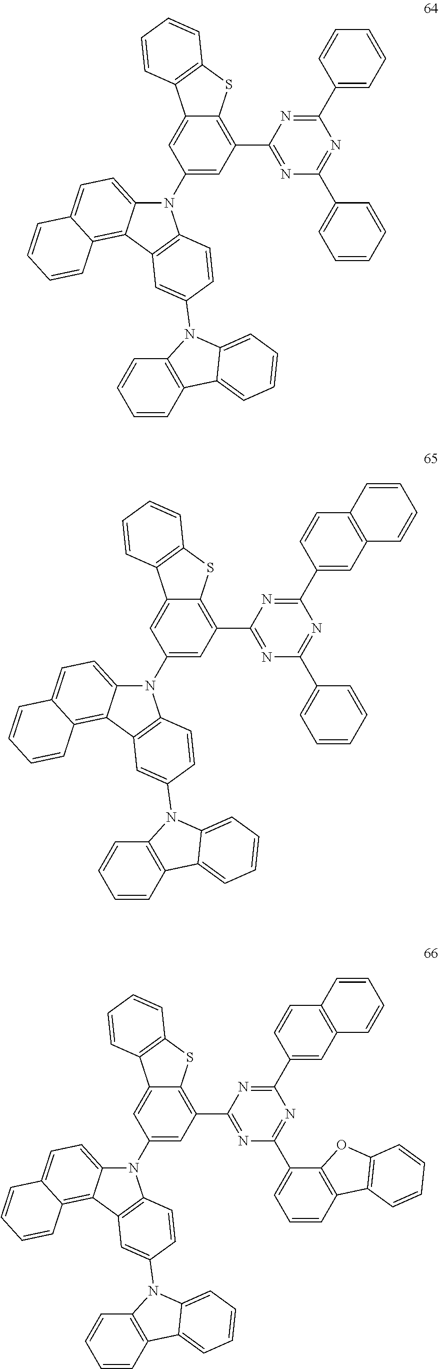 [SMILES: c1ccc(-c2nc(-c3ccccc3)nc(-c3cc(-n4c5ccc(-n6c7ccccc7c7ccccc76)cc5c5c6ccccc6ccc54)cc4c3sc3ccccc34)n2)cc1 ; c1ccc(-c2nc(-c3ccc4ccccc4c3)nc(-c3cc(-n4c5ccc(-n6c7ccccc7c7ccccc76)cc5c5c6ccccc6ccc54)cc4c3sc3ccccc34)n2)cc1 ; c1ccc2cc(-c3nc(-c4cccc5c4oc4ccccc45)nc(-c4cc(-n5c6ccc(-n7c8ccccc8c8ccccc87)cc6c6c7ccccc7ccc65)cc5c4sc4ccccc45)n3)ccc2c1]

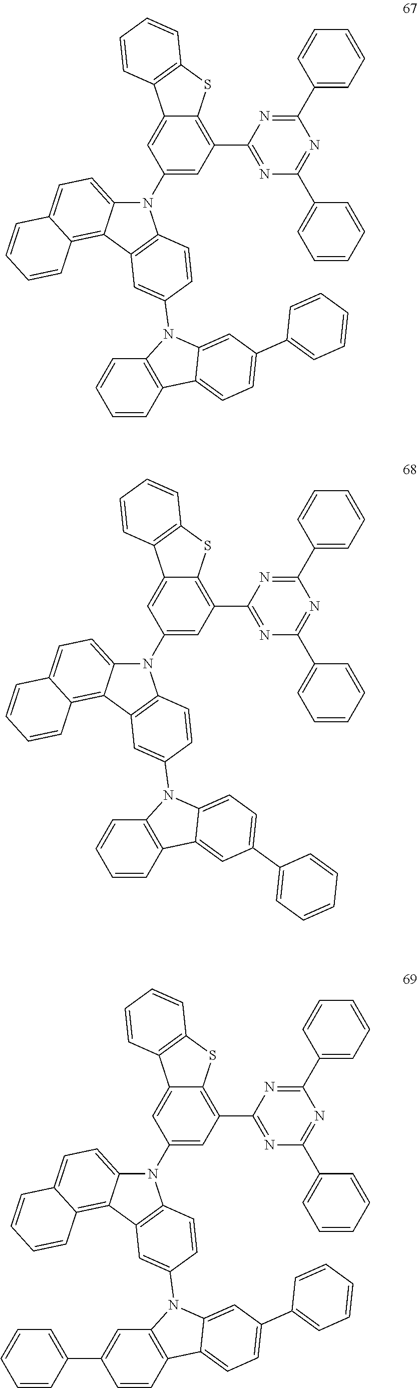 [SMILES: c1ccc(-c2ccc3c4ccccc4n(-c4ccc5c(c4)c4c6ccccc6ccc4n5-c4cc(-c5nc(-c6ccccc6)nc(-c6ccccc6)n5)c5sc6ccccc6c5c4)c3c2)cc1 ; c1ccc(-c2ccc3c(c2)c2ccccc2n3-c2ccc3c(c2)c2c4ccccc4ccc2n3-c2cc(-c3nc(-c4ccccc4)nc(-c4ccccc4)n3)c3sc4ccccc4c3c2)cc1 ; c1ccc(-c2ccc3c4ccc(-c5ccccc5)cc4n(-c4ccc5c(c4)c4c6ccccc6ccc4n5-c4cc(-c5nc(-c6ccccc6)nc(-c6ccccc6)n5)c5sc6ccccc6c5c4)c3c2)cc1]

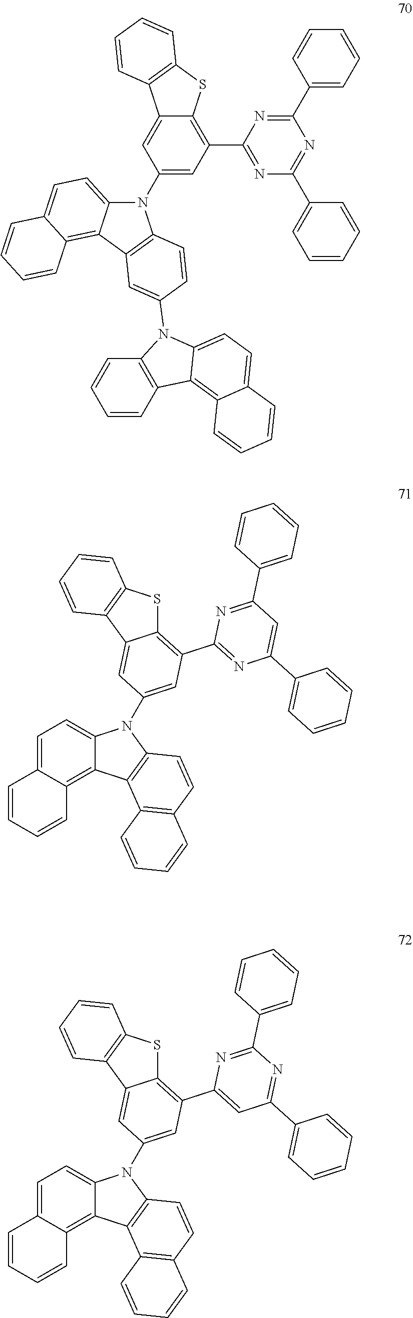 [SMILES: c1ccc(-c2nc(-c3ccccc3)nc(-c3cc(-n4c5ccc(-n6c7ccccc7c7c8ccccc8ccc76)cc5c5c6ccccc6ccc54)cc4c3sc3ccccc34)n2)cc1 ; c1ccc(-c2cc(-c3ccccc3)nc(-c3cc(-n4c5ccc6ccccc6c5c5c6ccccc6ccc54)cc4c3sc3ccccc34)n2)cc1 ; c1ccc(-c2cc(-c3cc(-n4c5ccc6ccccc6c5c5c6ccccc6ccc54)cc4c3sc3ccccc34)nc(-c3ccccc3)n2)cc1]

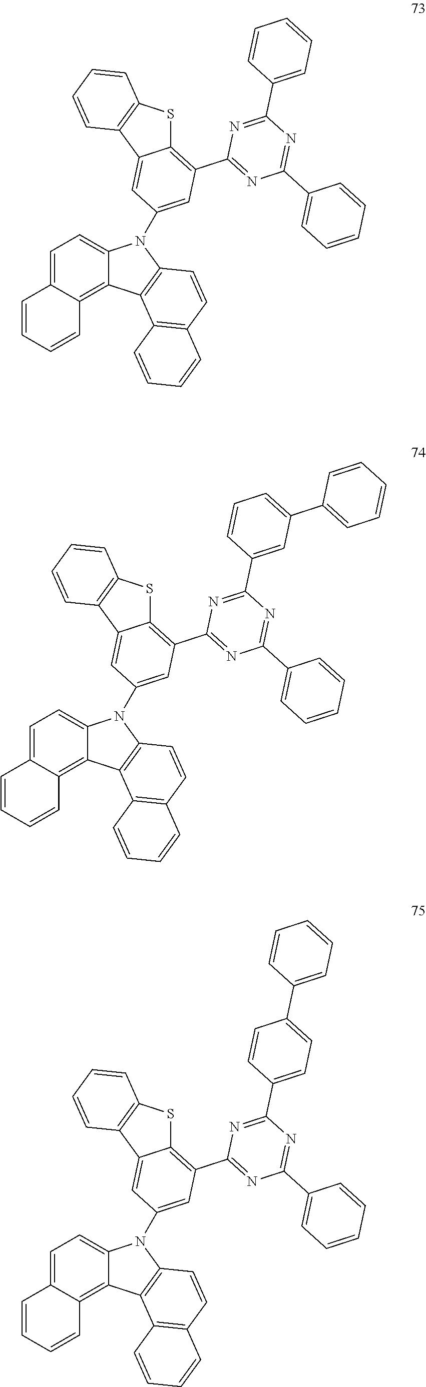 [SMILES: c1ccc(-c2nc(-c3ccccc3)nc(-c3cc(-n4c5ccc6ccccc6c5c5c6ccccc6ccc54)cc4c3sc3ccccc34)n2)cc1 ; c1ccc(-c2cccc(-c3nc(-c4ccccc4)nc(-c4cc(-n5c6ccc7ccccc7c6c6c7ccccc7ccc65)cc5c4sc4ccccc45)n3)c2)cc1 ; c1ccc(-c2ccc(-c3nc(-c4ccccc4)nc(-c4cc(-n5c6ccc7ccccc7c6c6c7ccccc7ccc65)cc5c4sc4ccccc45)n3)cc2)cc1]

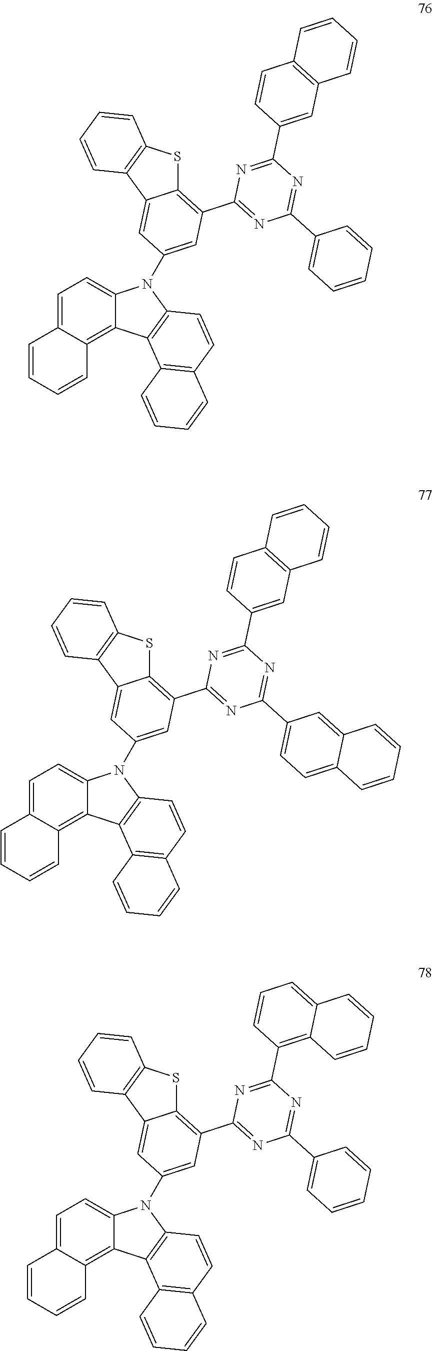 [SMILES: c1ccc(-c2nc(-c3ccc4ccccc4c3)nc(-c3cc(-n4c5ccc6ccccc6c5c5c6ccccc6ccc54)cc4c3sc3ccccc34)n2)cc1 ; c1ccc2cc(-c3nc(-c4ccc5ccccc5c4)nc(-c4cc(-n5c6ccc7ccccc7c6c6c7ccccc7ccc65)cc5c4sc4ccccc45)n3)ccc2c1 ; c1ccc(-c2nc(-c3cccc4ccccc34)nc(-c3cc(-n4c5ccc6ccccc6c5c5c6ccccc6ccc54)cc4c3sc3ccccc34)n2)cc1]

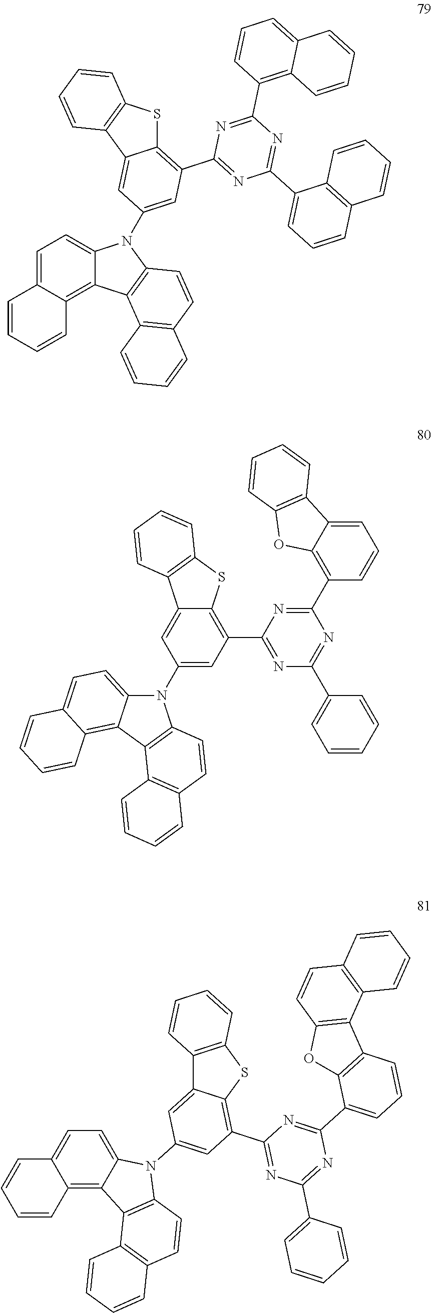 [SMILES: c1ccc2c(-c3nc(-c4cccc5ccccc45)nc(-c4cc(-n5c6ccc7ccccc7c6c6c7ccccc7ccc65)cc5c4sc4ccccc45)n3)cccc2c1 ; c1ccc(-c2nc(-c3cccc4c3oc3ccccc34)nc(-c3cc(-n4c5ccc6ccccc6c5c5c6ccccc6ccc54)cc4c3sc3ccccc34)n2)cc1 ; c1ccc(-c2nc(-c3cccc4c3oc3ccc5ccccc5c34)nc(-c3cc(-n4c5ccc6ccccc6c5c5c6ccccc6ccc54)cc4c3sc3ccccc34)n2)cc1]

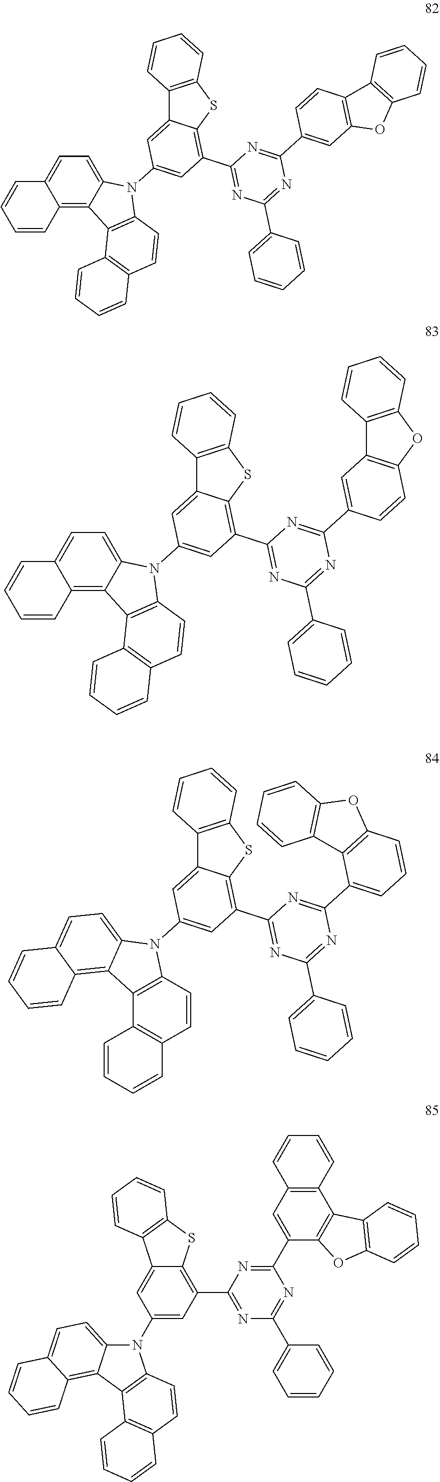 [SMILES: c1ccc(-c2nc(-c3ccc4c(c3)oc3ccccc34)nc(-c3cc(-n4c5ccc6ccccc6c5c5c6ccccc6ccc54)cc4c3sc3ccccc34)n2)cc1 ; c1ccc(-c2nc(-c3ccc4oc5ccccc5c4c3)nc(-c3cc(-n4c5ccc6ccccc6c5c5c6ccccc6ccc54)cc4c3sc3ccccc34)n2)cc1 ; c1ccc(-c2nc(-c3cc(-n4c5ccc6ccccc6c5c5c6ccccc6ccc54)cc4c3sc3ccccc34)nc(-c3cccc4oc5ccccc5c34)n2)cc1 ; c1ccc(-c2nc(-c3cc4ccccc4c4c3oc3ccccc34)nc(-c3cc(-n4c5ccc6ccccc6c5c5c6ccccc6ccc54)cc4c3sc3ccccc34)n2)cc1]

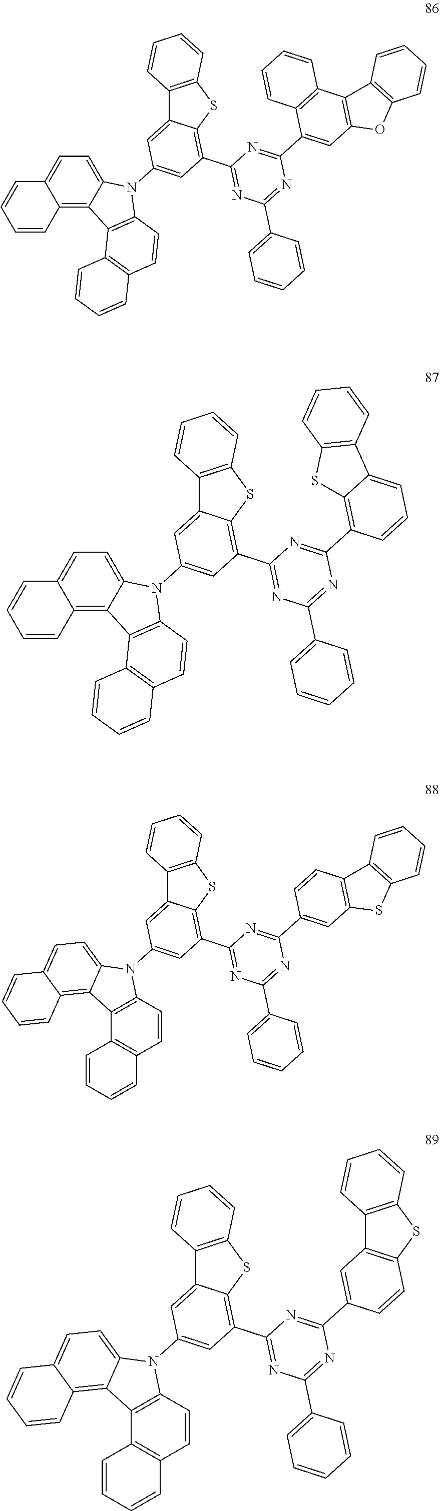 [SMILES: c1ccc(-c2nc(-c3cc4oc5ccccc5c4c4ccccc34)nc(-c3cc(-n4c5ccc6ccccc6c5c5c6ccccc6ccc54)cc4c3sc3ccccc34)n2)cc1 ; c1ccc(-c2nc(-c3cccc4c3sc3ccccc34)nc(-c3cc(-n4c5ccc6ccccc6c5c5c6ccccc6ccc54)cc4c3sc3ccccc34)n2)cc1 ; c1ccc(-c2nc(-c3ccc4c(c3)sc3ccccc34)nc(-c3cc(-n4c5ccc6ccccc6c5c5c6ccccc6ccc54)cc4c3sc3ccccc34)n2)cc1 ; c1ccc(-c2nc(-c3ccc4sc5ccccc5c4c3)nc(-c3cc(-n4c5ccc6ccccc6c5c5c6ccccc6ccc54)cc4c3sc3ccccc34)n2)cc1]

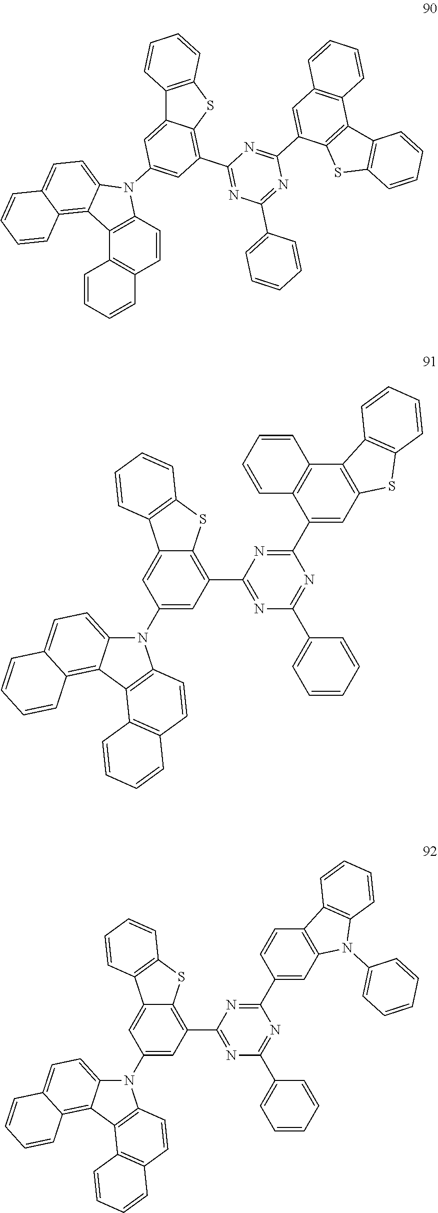 [SMILES: c1ccc(-c2nc(-c3cc(-n4c5ccc6ccccc6c5c5c6ccccc6ccc54)cc4c3sc3ccccc34)nc(-c3cc4ccccc4c4c3sc3ccccc34)n2)cc1 ; c1ccc(-c2nc(-c3cc4sc5ccccc5c4c4ccccc34)nc(-c3cc(-n4c5ccc6ccccc6c5c5c6ccccc6ccc54)cc4c3sc3ccccc34)n2)cc1 ; c1ccc(-c2nc(-c3ccc4c5ccccc5n(-c5ccccc5)c4c3)nc(-c3cc(-n4c5ccc6ccccc6c5c5c6ccccc6ccc54)cc4c3sc3ccccc34)n2)cc1]

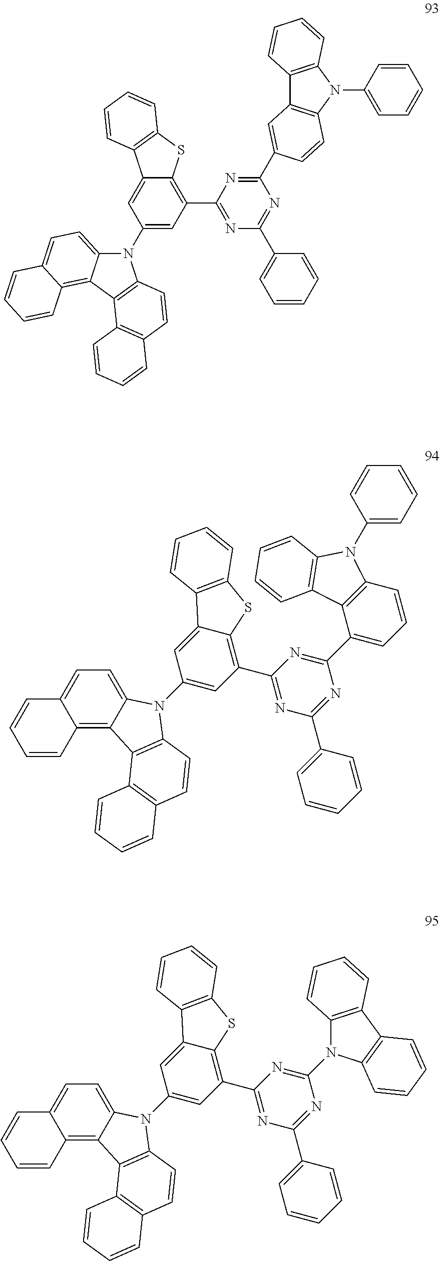 [SMILES: c1ccc(-c2nc(-c3ccc4c(c3)c3ccccc3n4-c3ccccc3)nc(-c3cc(-n4c5ccc6ccccc6c5c5c6ccccc6ccc54)cc4c3sc3ccccc34)n2)cc1 ; c1ccc(-c2nc(-c3cc(-n4c5ccc6ccccc6c5c5c6ccccc6ccc54)cc4c3sc3ccccc34)nc(-c3cccc4c3c3ccccc3n4-c3ccccc3)n2)cc1 ; c1ccc(-c2nc(-c3cc(-n4c5ccc6ccccc6c5c5c6ccccc6ccc54)cc4c3sc3ccccc34)nc(-n3c4ccccc4c4ccccc43)n2)cc1]

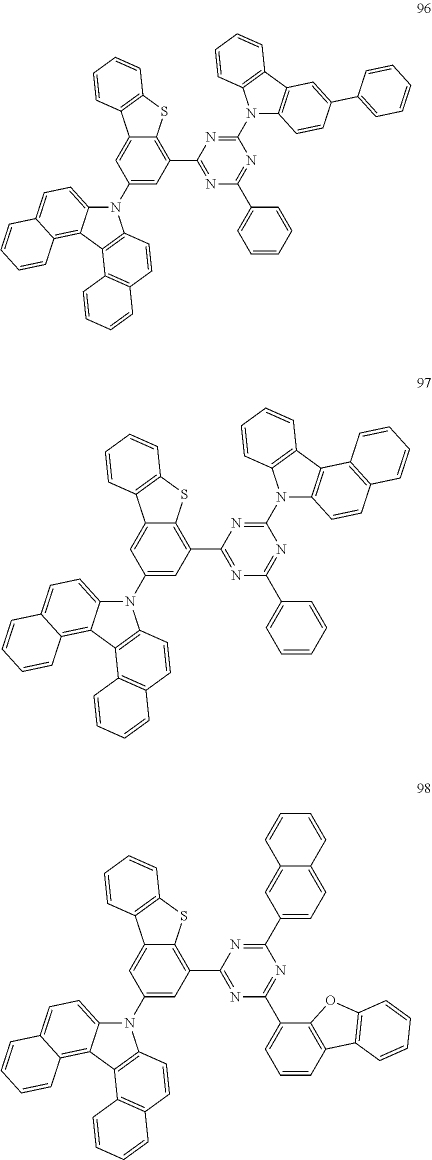 [SMILES: c1ccc(-c2ccc3c(c2)c2ccccc2n3-c2nc(-c3ccccc3)nc(-c3cc(-n4c5ccc6ccccc6c5c5c6ccccc6ccc54)cc4c3sc3ccccc34)n2)cc1 ; c1ccc(-c2nc(-c3cc(-n4c5ccc6ccccc6c5c5c6ccccc6ccc54)cc4c3sc3ccccc34)nc(-n3c4ccccc4c4c5ccccc5ccc43)n2)cc1 ; c1ccc2cc(-c3nc(-c4cccc5c4oc4ccccc45)nc(-c4cc(-n5c6ccc7ccccc7c6c6c7ccccc7ccc65)cc5c4sc4ccccc45)n3)ccc2c1]

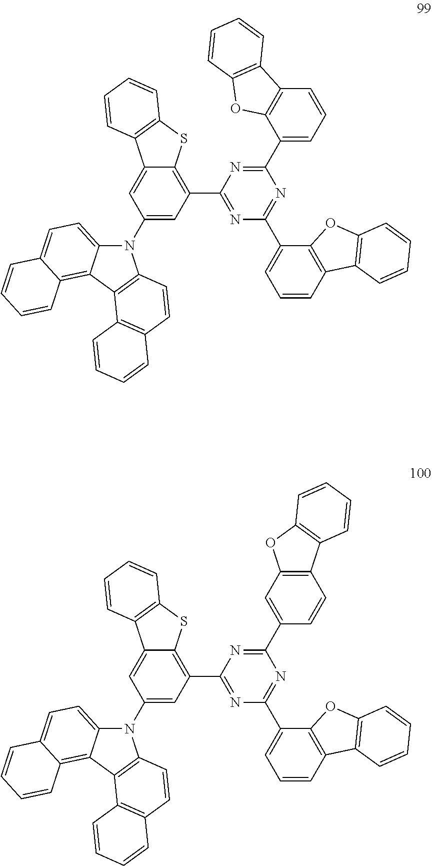 [SMILES: c1ccc2c(c1)ccc1c2c2c3ccccc3ccc2n1-c1cc(-c2nc(-c3cccc4c3oc3ccccc34)nc(-c3cccc4c3oc3ccccc34)n2)c2sc3ccccc3c2c1 ; c1ccc2c(c1)ccc1c2c2c3ccccc3ccc2n1-c1cc(-c2nc(-c3ccc4c(c3)oc3ccccc34)nc(-c3cccc4c3oc3ccccc34)n2)c2sc3ccccc3c2c1]

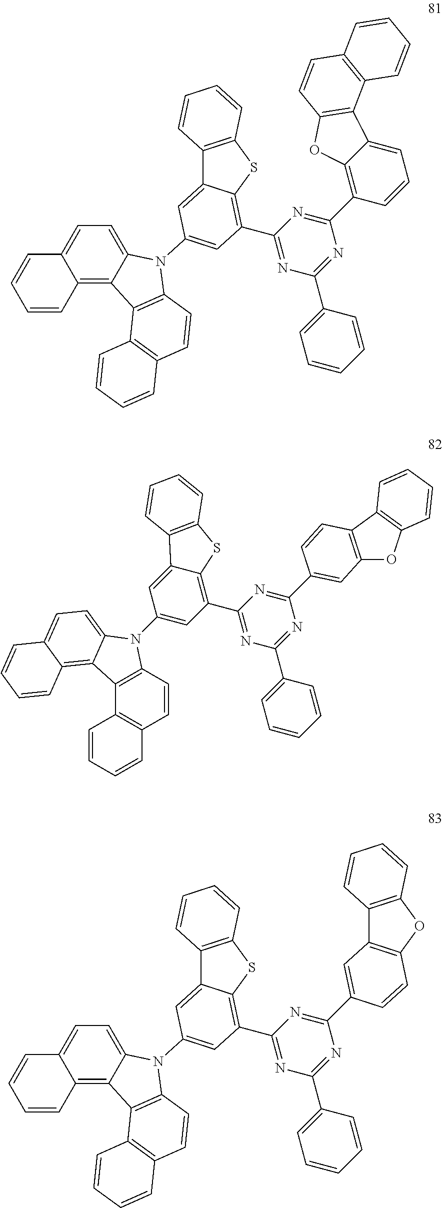 [SMILES: c1ccc(-c2nc(-c3cccc4c3oc3ccc5ccccc5c34)nc(-c3cc(-n4c5ccc6ccccc6c5c5c6ccccc6ccc54)cc4c3sc3ccccc34)n2)cc1 ; c1ccc(-c2nc(-c3ccc4c(c3)oc3ccccc34)nc(-c3cc(-n4c5ccc6ccccc6c5c5c6ccccc6ccc54)cc4c3sc3ccccc34)n2)cc1 ; c1ccc(-c2nc(-c3ccc4oc5ccccc5c4c3)nc(-c3cc(-n4c5ccc6ccccc6c5c5c6ccccc6ccc54)cc4c3sc3ccccc34)n2)cc1]

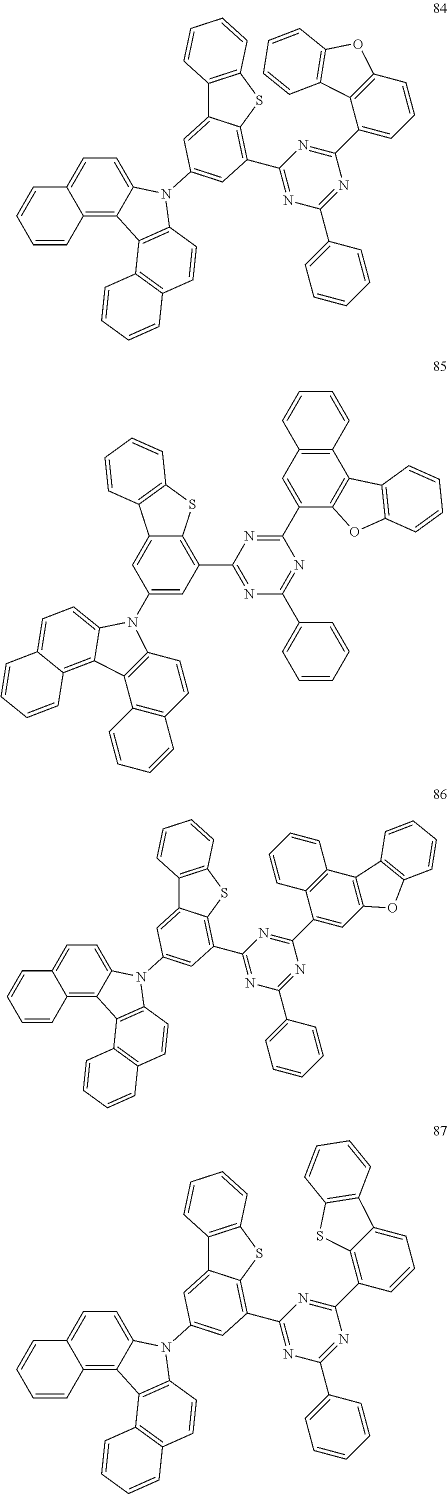 [SMILES: c1ccc(-c2nc(-c3cc(-n4c5ccc6ccccc6c5c5c6ccccc6ccc54)cc4c3sc3ccccc34)nc(-c3cccc4oc5ccccc5c34)n2)cc1 ; c1ccc(-c2nc(-c3cc4ccccc4c4c3oc3ccccc34)nc(-c3cc(-n4c5ccc6ccccc6c5c5c6ccccc6ccc54)cc4c3sc3ccccc34)n2)cc1 ; c1ccc(-c2nc(-c3cc4oc5ccccc5c4c4ccccc34)nc(-c3cc(-n4c5ccc6ccccc6c5c5c6ccccc6ccc54)cc4c3sc3ccccc34)n2)cc1 ; c1ccc(-c2nc(-c3cccc4c3sc3ccccc34)nc(-c3cc(-n4c5ccc6ccccc6c5c5c6ccccc6ccc54)cc4c3sc3ccccc34)n2)cc1]

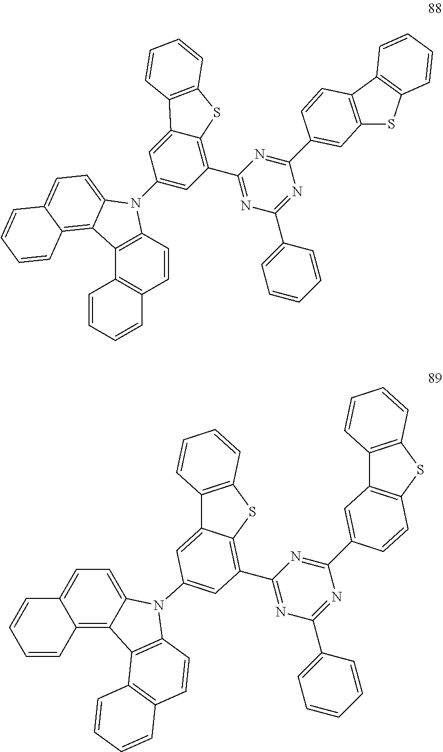 [SMILES: c1ccc(-c2nc(-c3ccc4c(c3)sc3ccccc34)nc(-c3cc(-n4c5ccc6ccccc6c5c5c6ccccc6ccc54)cc4c3sc3ccccc34)n2)cc1 ; c1ccc(-c2nc(-c3ccc4sc5ccccc5c4c3)nc(-c3cc(-n4c5ccc6ccccc6c5c5c6ccccc6ccc54)cc4c3sc3ccccc34)n2)cc1]

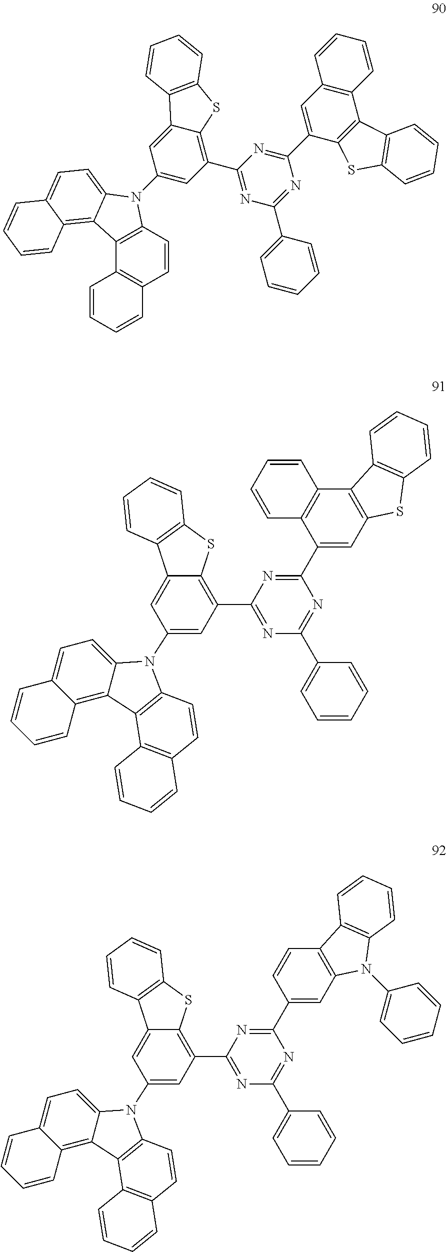 [SMILES: c1ccc(-c2nc(-c3cc(-n4c5ccc6ccccc6c5c5c6ccccc6ccc54)cc4c3sc3ccccc34)nc(-c3cc4ccccc4c4c3sc3ccccc34)n2)cc1 ; c1ccc(-c2nc(-c3cc4sc5ccccc5c4c4ccccc34)nc(-c3cc(-n4c5ccc6ccccc6c5c5c6ccccc6ccc54)cc4c3sc3ccccc34)n2)cc1 ; c1ccc(-c2nc(-c3ccc4c5ccccc5n(-c5ccccc5)c4c3)nc(-c3cc(-n4c5ccc6ccccc6c5c5c6ccccc6ccc54)cc4c3sc3ccccc34)n2)cc1]

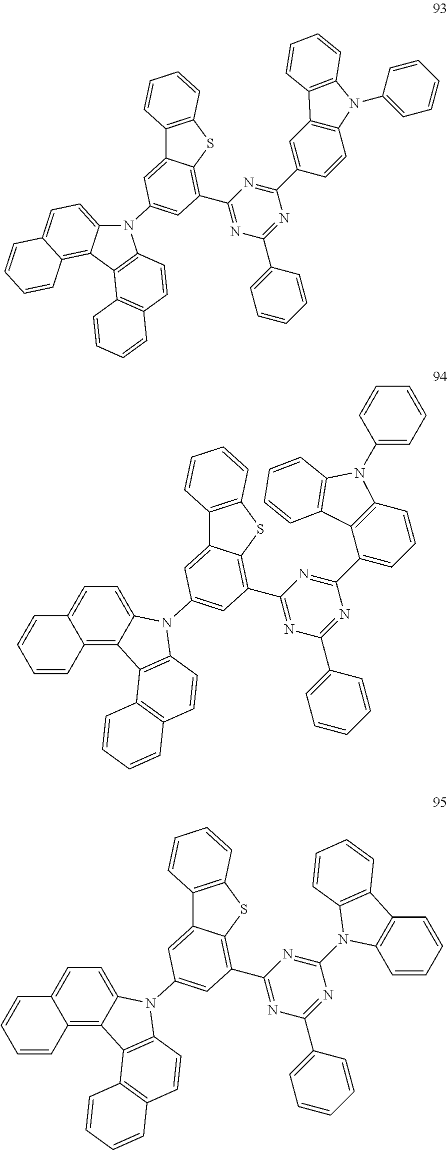 [SMILES: c1ccc(-c2nc(-c3ccc4c(c3)c3ccccc3n4-c3ccccc3)nc(-c3cc(-n4c5ccc6ccccc6c5c5c6ccccc6ccc54)cc4c3sc3ccccc34)n2)cc1 ; c1ccc(-c2nc(-c3cc(-n4c5ccc6ccccc6c5c5c6ccccc6ccc54)cc4c3sc3ccccc34)nc(-c3cccc4c3c3ccccc3n4-c3ccccc3)n2)cc1 ; c1ccc(-c2nc(-c3cc(-n4c5ccc6ccccc6c5c5c6ccccc6ccc54)cc4c3sc3ccccc34)nc(-n3c4ccccc4c4ccccc43)n2)cc1]

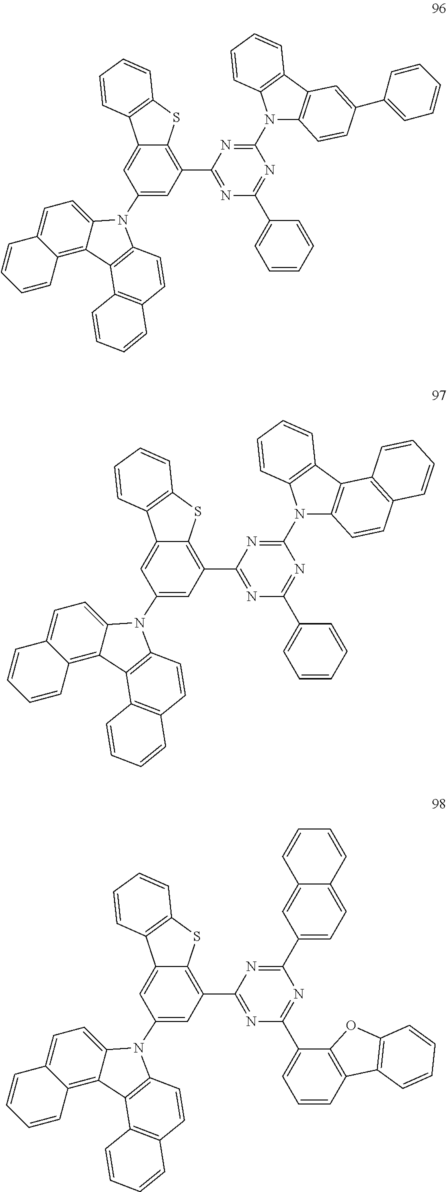 [SMILES: c1ccc(-c2ccc3c(c2)c2ccccc2n3-c2nc(-c3ccccc3)nc(-c3cc(-n4c5ccc6ccccc6c5c5c6ccccc6ccc54)cc4c3sc3ccccc34)n2)cc1 ; c1ccc(-c2nc(-c3cc(-n4c5ccc6ccccc6c5c5c6ccccc6ccc54)cc4c3sc3ccccc34)nc(-n3c4ccccc4c4c5ccccc5ccc43)n2)cc1 ; c1ccc2cc(-c3nc(-c4cccc5c4oc4ccccc45)nc(-c4cc(-n5c6ccc7ccccc7c6c6c7ccccc7ccc65)cc5c4sc4ccccc45)n3)ccc2c1]

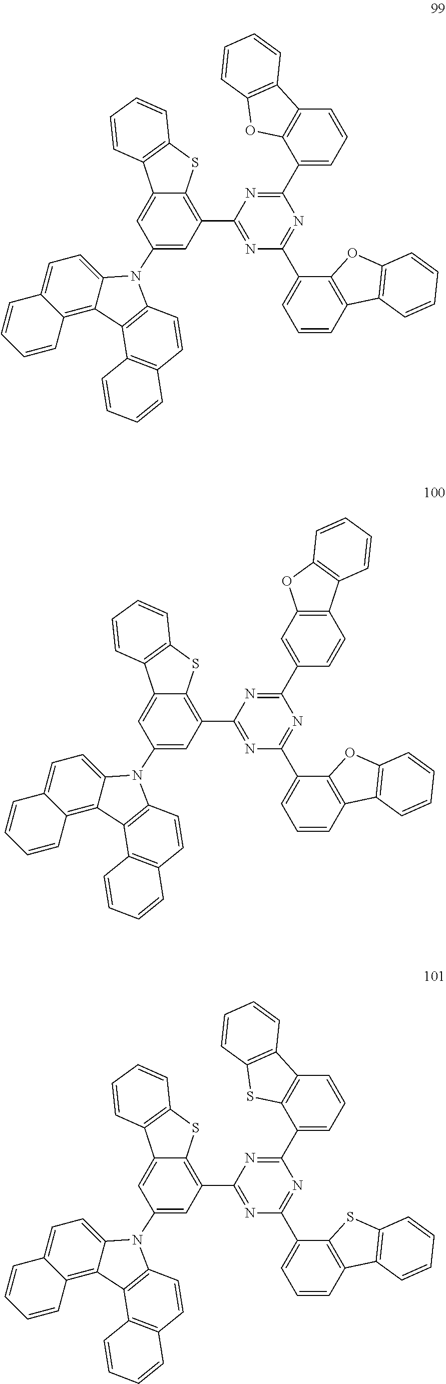 [SMILES: c1ccc2c(c1)ccc1c2c2c3ccccc3ccc2n1-c1cc(-c2nc(-c3cccc4c3oc3ccccc34)nc(-c3cccc4c3oc3ccccc34)n2)c2sc3ccccc3c2c1 ; c1ccc2c(c1)ccc1c2c2c3ccccc3ccc2n1-c1cc(-c2nc(-c3ccc4c(c3)oc3ccccc34)nc(-c3cccc4c3oc3ccccc34)n2)c2sc3ccccc3c2c1 ; c1ccc2c(c1)ccc1c2c2c3ccccc3ccc2n1-c1cc(-c2nc(-c3cccc4c3sc3ccccc34)nc(-c3cccc4c3sc3ccccc34)n2)c2sc3ccccc3c2c1]

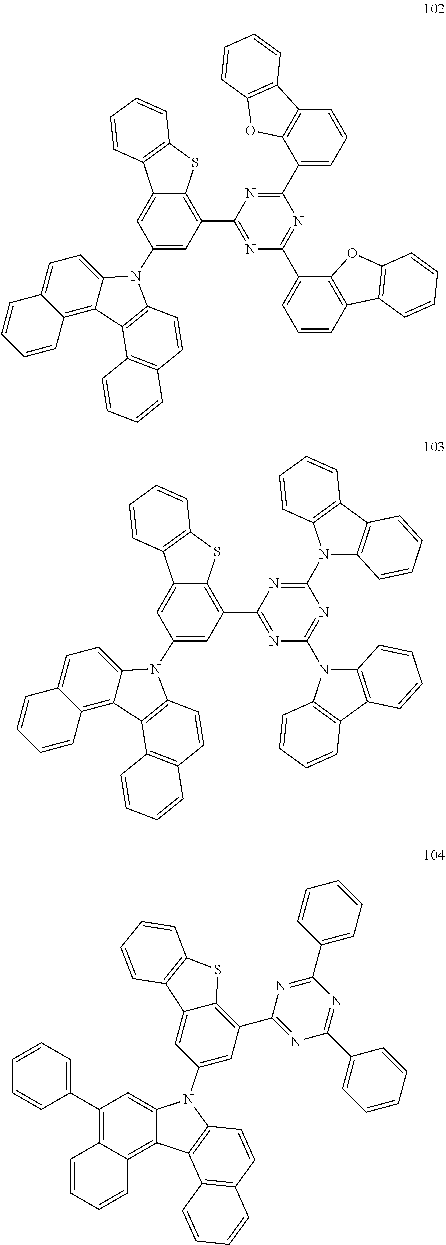 [SMILES: c1ccc2c(c1)ccc1c2c2c3ccccc3ccc2n1-c1cc(-c2nc(-c3cccc4c3oc3ccccc34)nc(-c3cccc4c3oc3ccccc34)n2)c2sc3ccccc3c2c1 ; c1ccc2c(c1)ccc1c2c2c3ccccc3ccc2n1-c1cc(-c2nc(-n3c4ccccc4c4ccccc43)nc(-n3c4ccccc4c4ccccc43)n2)c2sc3ccccc3c2c1 ; c1ccc(-c2nc(-c3ccccc3)nc(-c3cc(-n4c5ccc6ccccc6c5c5c6ccccc6c(-c6ccccc6)cc54)cc4c3sc3ccccc34)n2)cc1]

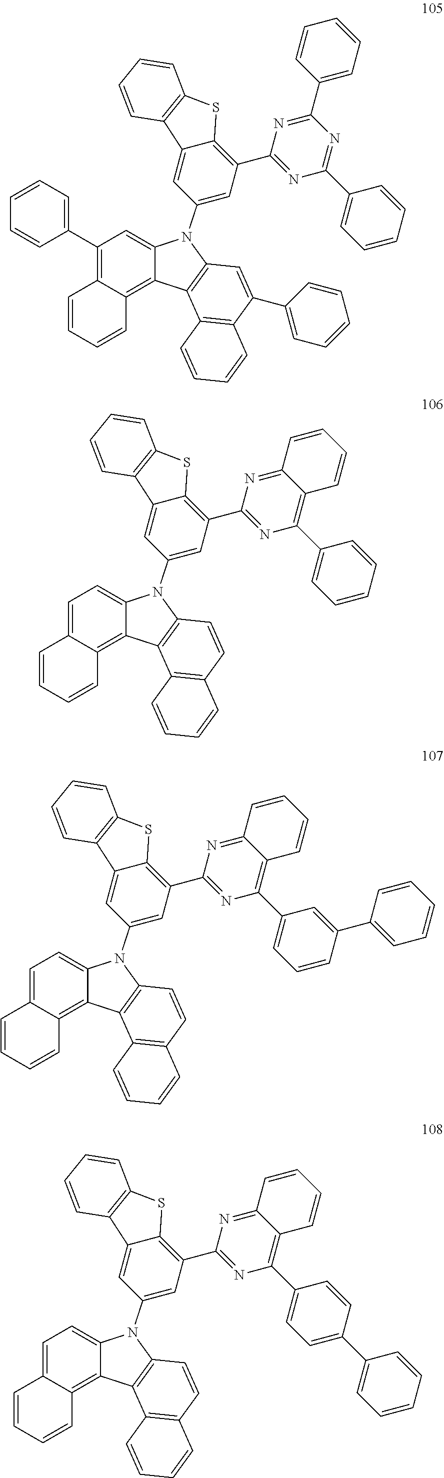 [SMILES: c1ccc(-c2nc(-c3ccccc3)nc(-c3cc(-n4c5cc(-c6ccccc6)c6ccccc6c5c5c6ccccc6c(-c6ccccc6)cc54)cc4c3sc3ccccc34)n2)cc1 ; c1ccc(-c2nc(-c3cc(-n4c5ccc6ccccc6c5c5c6ccccc6ccc54)cc4c3sc3ccccc34)nc3ccccc23)cc1 ; c1ccc(-c2cccc(-c3nc(-c4cc(-n5c6ccc7ccccc7c6c6c7ccccc7ccc65)cc5c4sc4ccccc45)nc4ccccc34)c2)cc1 ; c1ccc(-c2ccc(-c3nc(-c4cc(-n5c6ccc7ccccc7c6c6c7ccccc7ccc65)cc5c4sc4ccccc45)nc4ccccc34)cc2)cc1]

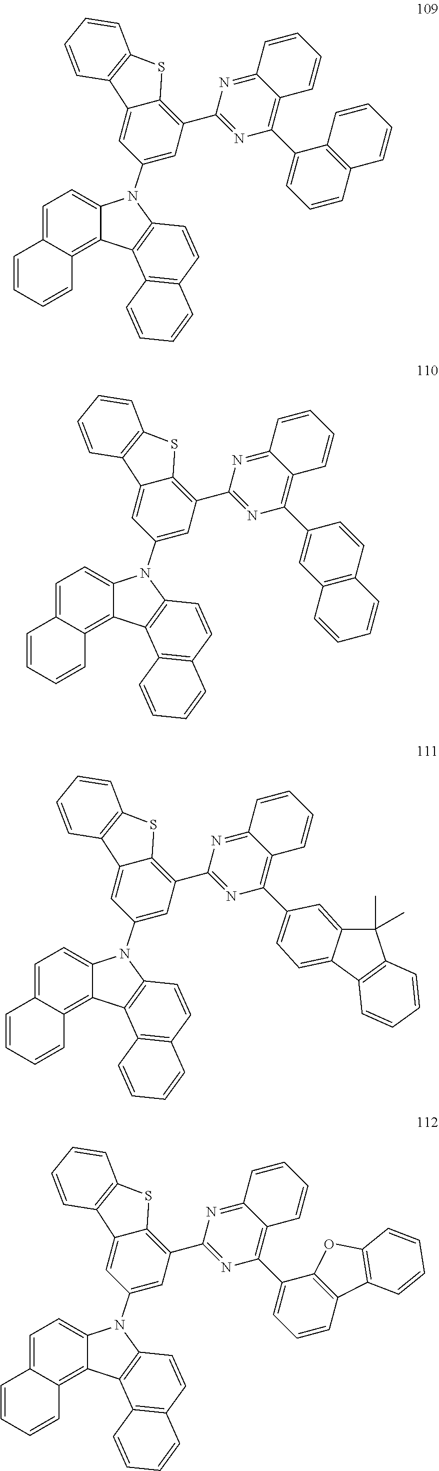 [SMILES: c1ccc2c(-c3nc(-c4cc(-n5c6ccc7ccccc7c6c6c7ccccc7ccc65)cc5c4sc4ccccc45)nc4ccccc34)cccc2c1 ; c1ccc2cc(-c3nc(-c4cc(-n5c6ccc7ccccc7c6c6c7ccccc7ccc65)cc5c4sc4ccccc45)nc4ccccc34)ccc2c1 ; CC1(C)c2ccccc2-c2ccc(-c3nc(-c4cc(-n5c6ccc7ccccc7c6c6c7ccccc7ccc65)cc5c4sc4ccccc45)nc4ccccc34)cc21 ; c1ccc2c(c1)ccc1c2c2c3ccccc3ccc2n1-c1cc(-c2nc(-c3cccc4c3oc3ccccc34)c3ccccc3n2)c2sc3ccccc3c2c1]

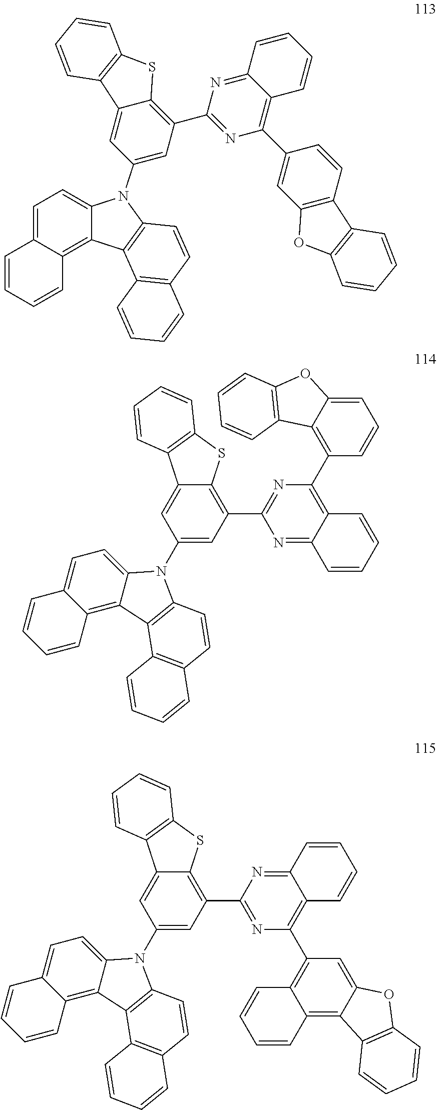

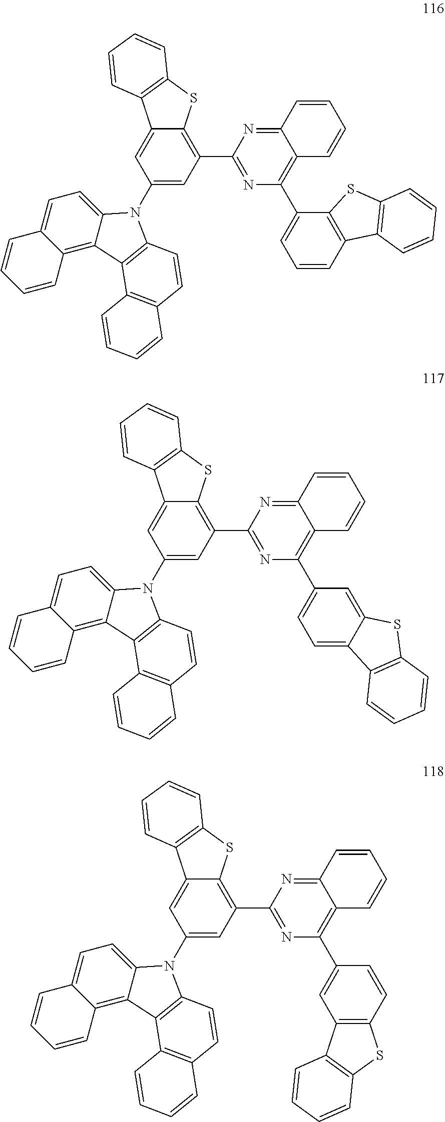

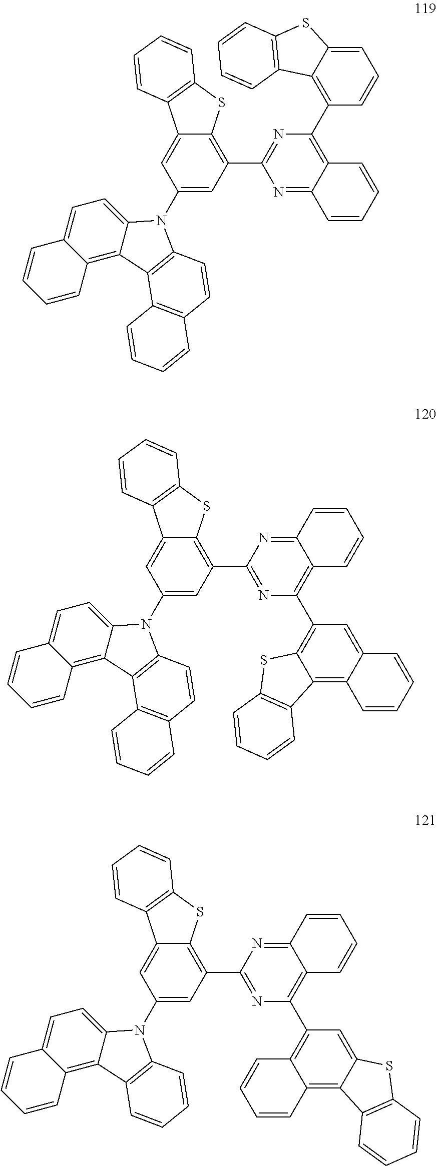 [SMILES: c1ccc2c(c1)ccc1c2c2c3ccccc3ccc2n1-c1cc(-c2nc(-c3cccc4sc5ccccc5c34)c3ccccc3n2)c2sc3ccccc3c2c1 ; c1ccc2c(c1)cc(-c1nc(-c3cc(-n4c5ccc6ccccc6c5c5c6ccccc6ccc54)cc4c3sc3ccccc34)nc3ccccc13)c1sc3ccccc3c12 ; c1ccc2c(c1)ccc1c2c2ccccc2n1-c1cc(-c2nc(-c3cc4sc5ccccc5c4c4ccccc34)c3ccccc3n2)c2sc3ccccc3c2c1]

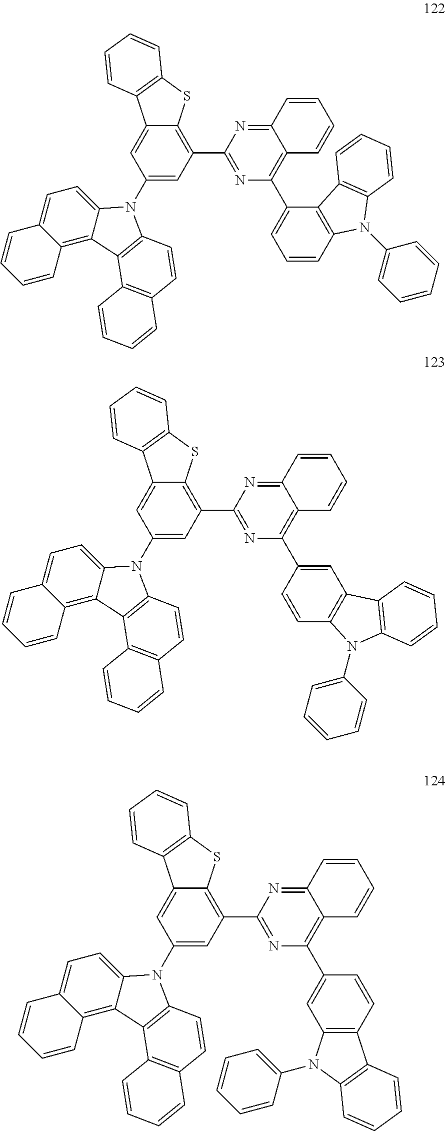

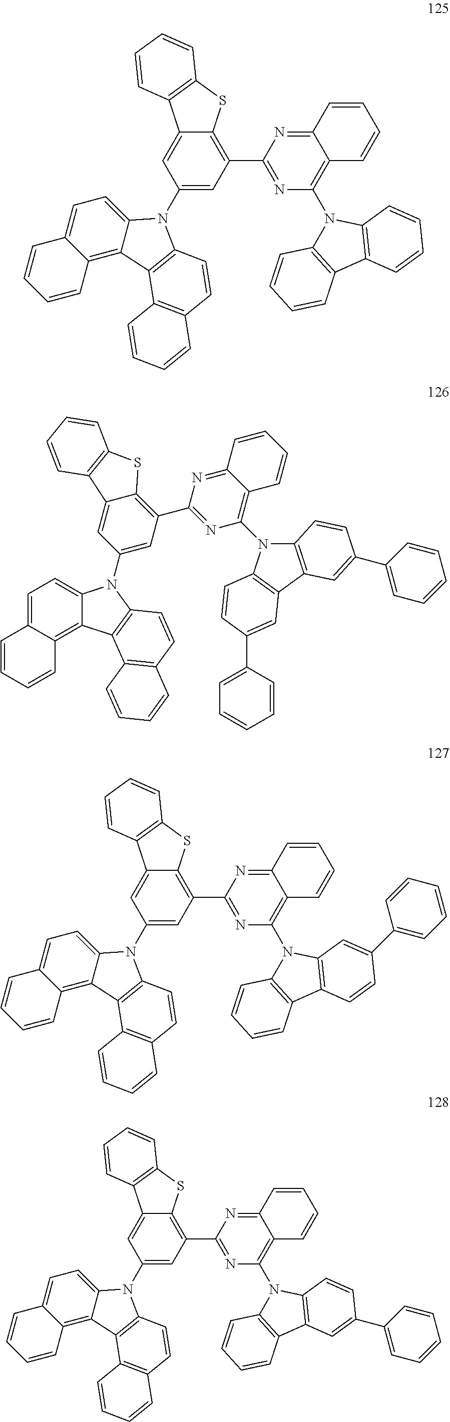

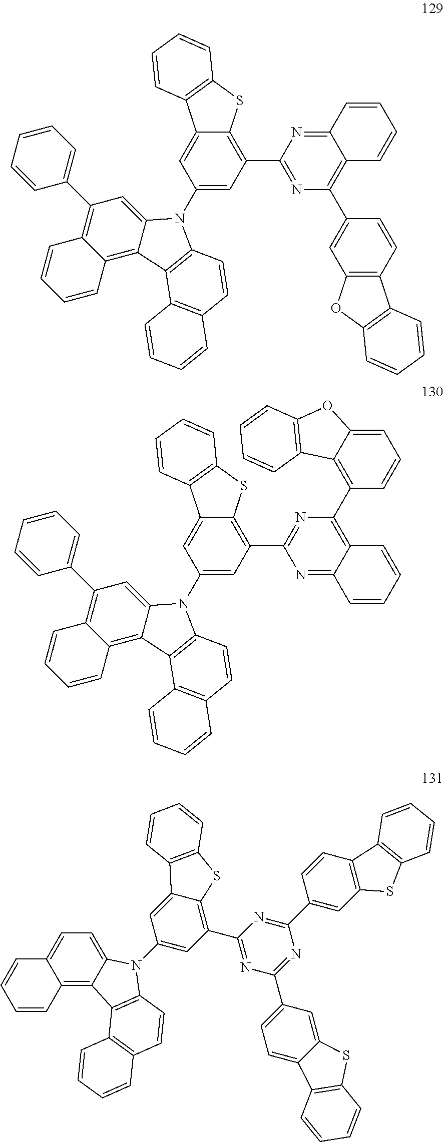 [SMILES: c1ccc(-c2cc3c(c4ccccc24)c2c4ccccc4ccc2n3-c2cc(-c3nc(-c4ccc5c(c4)oc4ccccc45)c4ccccc4n3)c3sc4ccccc4c3c2)cc1 ; c1ccc(-c2cc3c(c4ccccc24)c2c4ccccc4ccc2n3-c2cc(-c3nc(-c4cccc5oc6ccccc6c45)c4ccccc4n3)c3sc4ccccc4c3c2)cc1 ; c1ccc2c(c1)ccc1c2c2c3ccccc3ccc2n1-c1cc(-c2nc(-c3ccc4c(c3)sc3ccccc34)nc(-c3ccc4c(c3)sc3ccccc34)n2)c2sc3ccccc3c2c1]

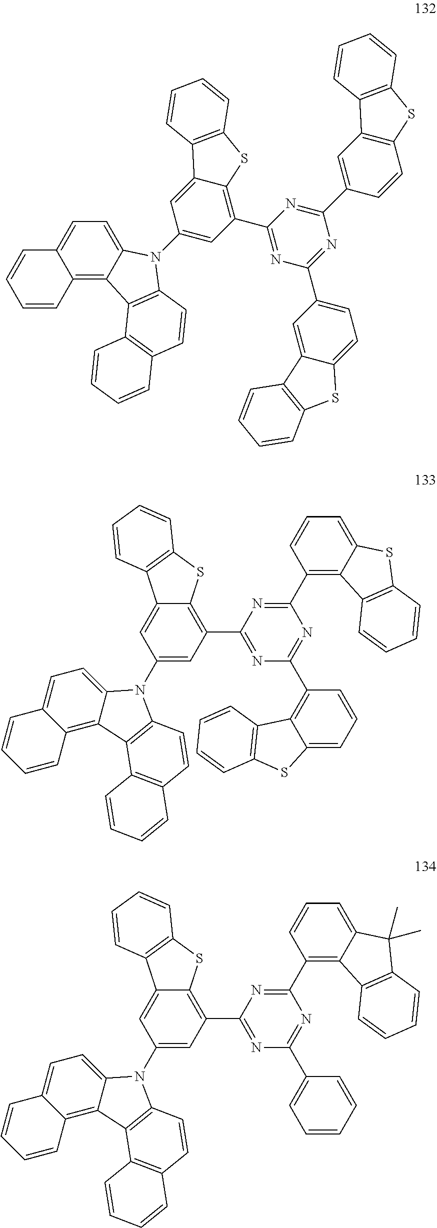 [SMILES: c1ccc2c(c1)ccc1c2c2c3ccccc3ccc2n1-c1cc(-c2nc(-c3ccc4sc5ccccc5c4c3)nc(-c3ccc4sc5ccccc5c4c3)n2)c2sc3ccccc3c2c1 ; c1ccc2c(c1)ccc1c2c2c3ccccc3ccc2n1-c1cc(-c2nc(-c3cccc4sc5ccccc5c34)nc(-c3cccc4sc5ccccc5c34)n2)c2sc3ccccc3c2c1 ; CC1(C)c2ccccc2-c2c(-c3nc(-c4ccccc4)nc(-c4cc(-n5c6ccc7ccccc7c6c6c7ccccc7ccc65)cc5c4sc4ccccc45)n3)cccc21]

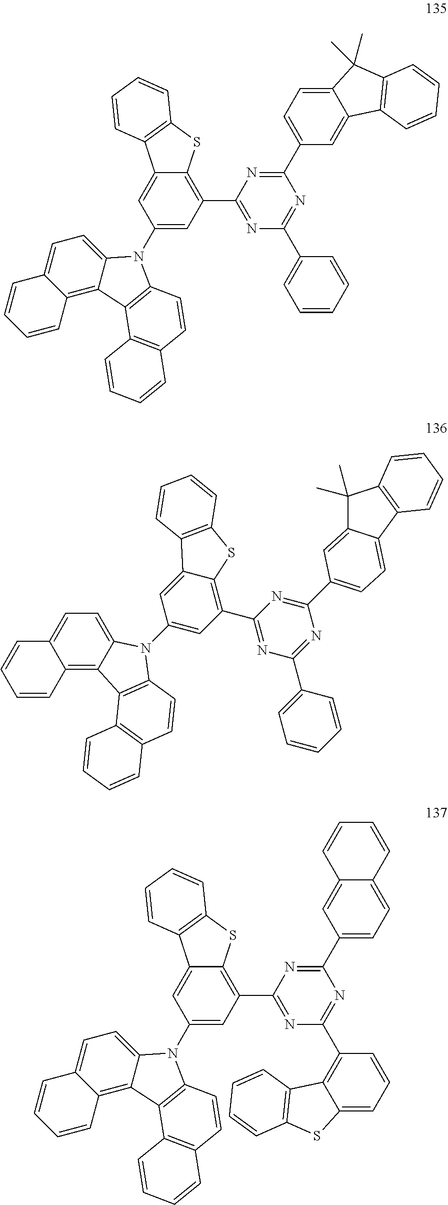 [SMILES: CC1(C)c2ccccc2-c2cc(-c3nc(-c4ccccc4)nc(-c4cc(-n5c6ccc7ccccc7c6c6c7ccccc7ccc65)cc5c4sc4ccccc45)n3)ccc21 ; CC1(C)c2ccccc2-c2ccc(-c3nc(-c4ccccc4)nc(-c4cc(-n5c6ccc7ccccc7c6c6c7ccccc7ccc65)cc5c4sc4ccccc45)n3)cc21 ; c1ccc2cc(-c3nc(-c4cc(-n5c6ccc7ccccc7c6c6c7ccccc7ccc65)cc5c4sc4ccccc45)nc(-c4cccc5sc6ccccc6c45)n3)ccc2c1]

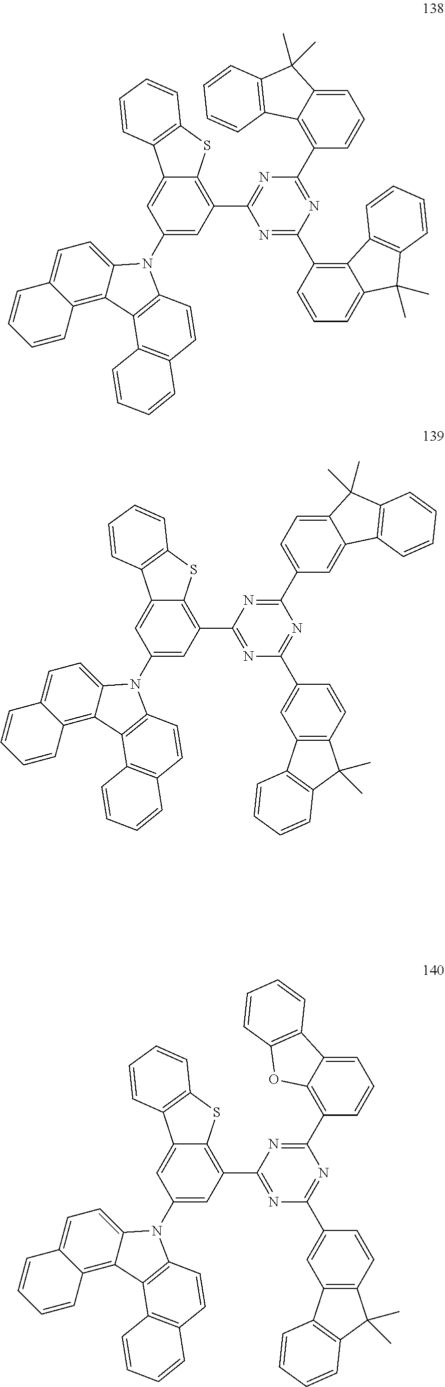 [SMILES: CC1(C)c2ccccc2-c2c(-c3nc(-c4cccc5c4-c4ccccc4C5(C)C)nc(-c4cc(-n5c6ccc7ccccc7c6c6c7ccccc7ccc65)cc5c4sc4ccccc45)n3)cccc21 ; CC1(C)c2ccccc2-c2cc(-c3nc(-c4ccc5c(c4)-c4ccccc4C5(C)C)nc(-c4cc(-n5c6ccc7ccccc7c6c6c7ccccc7ccc65)cc5c4sc4ccccc45)n3)ccc21 ; CC1(C)c2ccccc2-c2cc(-c3nc(-c4cccc5c4oc4ccccc45)nc(-c4cc(-n5c6ccc7ccccc7c6c6c7ccccc7ccc65)cc5c4sc4ccccc45)n3)ccc21]

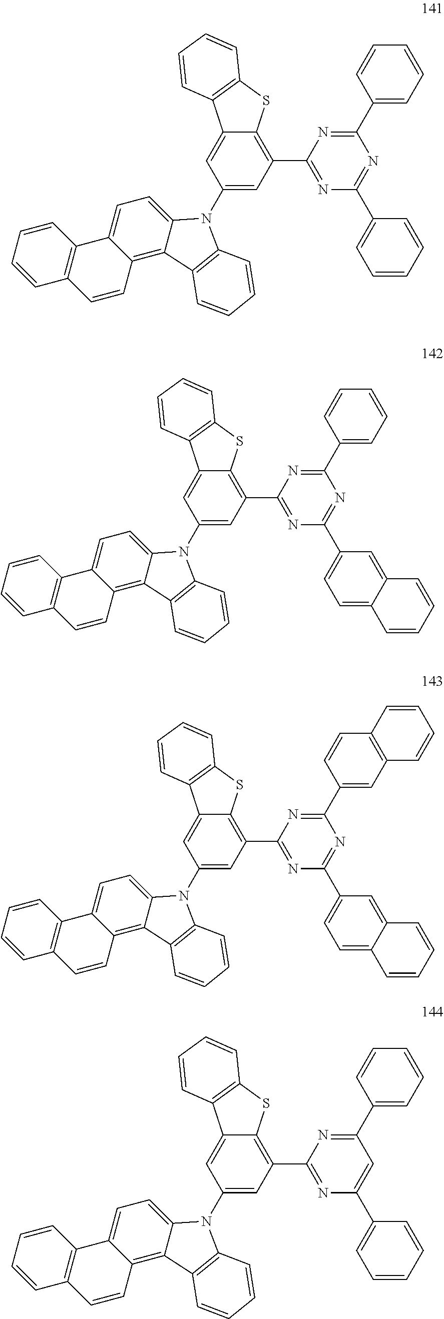 [SMILES: c1ccc(-c2nc(-c3ccccc3)nc(-c3cc(-n4c5ccccc5c5c6ccc7ccccc7c6ccc54)cc4c3sc3ccccc34)n2)cc1 ; c1ccc(-c2nc(-c3ccc4ccccc4c3)nc(-c3cc(-n4c5ccccc5c5c6ccc7ccccc7c6ccc54)cc4c3sc3ccccc34)n2)cc1 ; c1ccc2cc(-c3nc(-c4ccc5ccccc5c4)nc(-c4cc(-n5c6ccccc6c6c7ccc8ccccc8c7ccc65)cc5c4sc4ccccc45)n3)ccc2c1 ; c1ccc(-c2cc(-c3ccccc3)nc(-c3cc(-n4c5ccccc5c5c6ccc7ccccc7c6ccc54)cc4c3sc3ccccc34)n2)cc1]

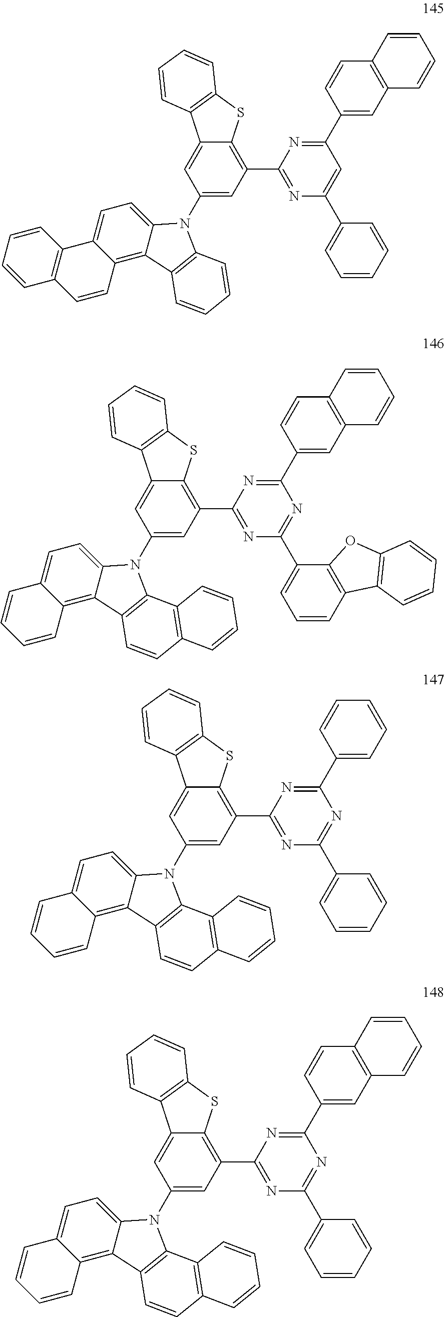

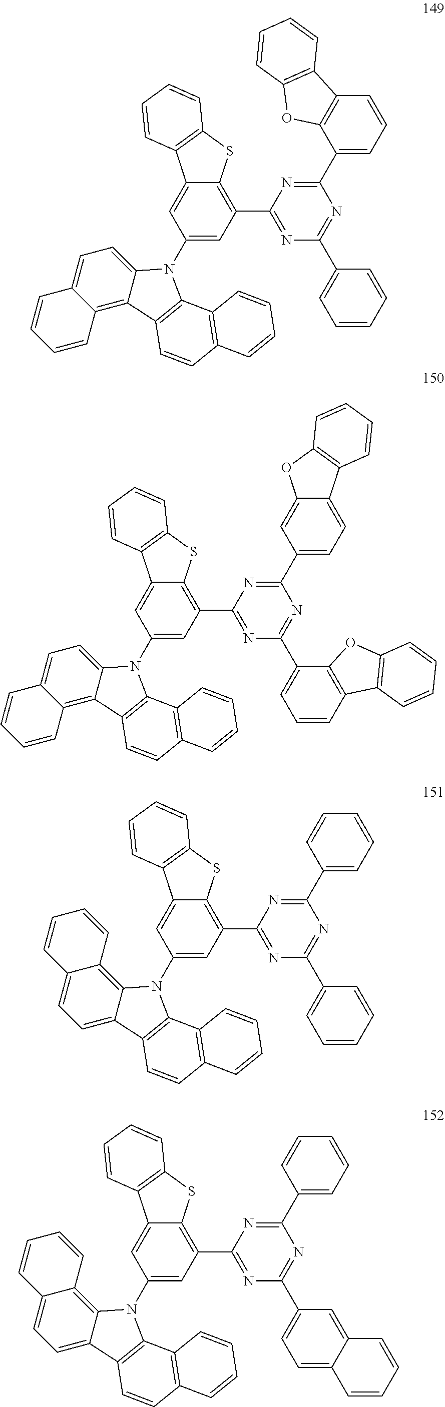

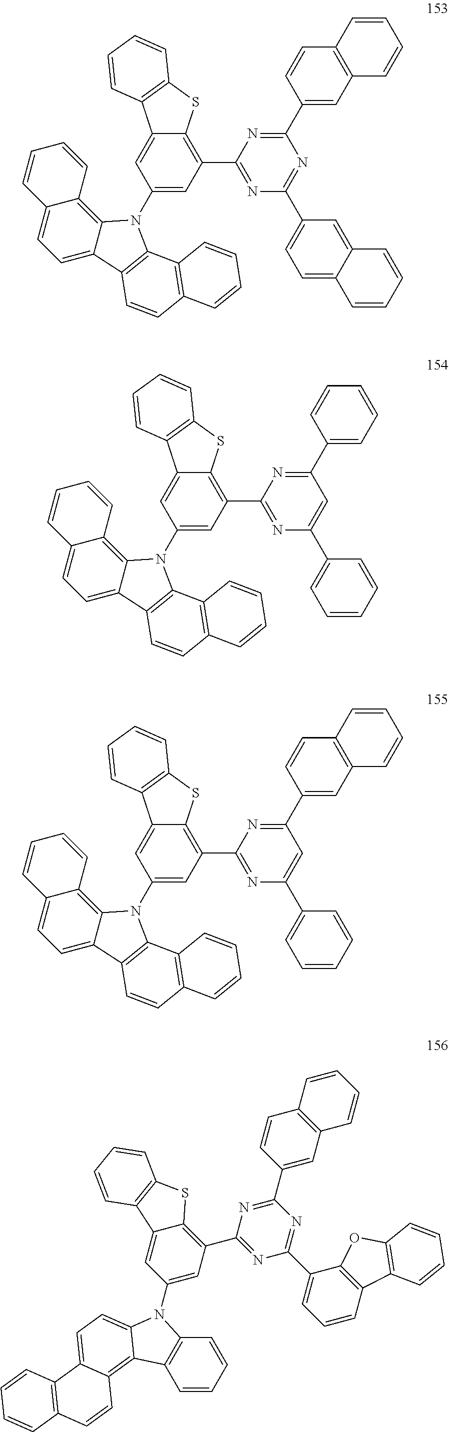 [SMILES: c1ccc2cc(-c3nc(-c4ccc5ccccc5c4)nc(-c4cc(-n5c6c7ccccc7ccc6c6ccc7ccccc7c65)cc5c4sc4ccccc45)n3)ccc2c1 ; c1ccc(-c2cc(-c3ccccc3)nc(-c3cc(-n4c5c6ccccc6ccc5c5ccc6ccccc6c54)cc4c3sc3ccccc34)n2)cc1 ; c1ccc(-c2cc(-c3ccc4ccccc4c3)nc(-c3cc(-n4c5c6ccccc6ccc5c5ccc6ccccc6c54)cc4c3sc3ccccc34)n2)cc1 ; c1ccc2cc(-c3nc(-c4cccc5c4oc4ccccc45)nc(-c4cc(-n5c6ccccc6c6c7ccc8ccccc8c7ccc65)cc5c4sc4ccccc45)n3)ccc2c1]

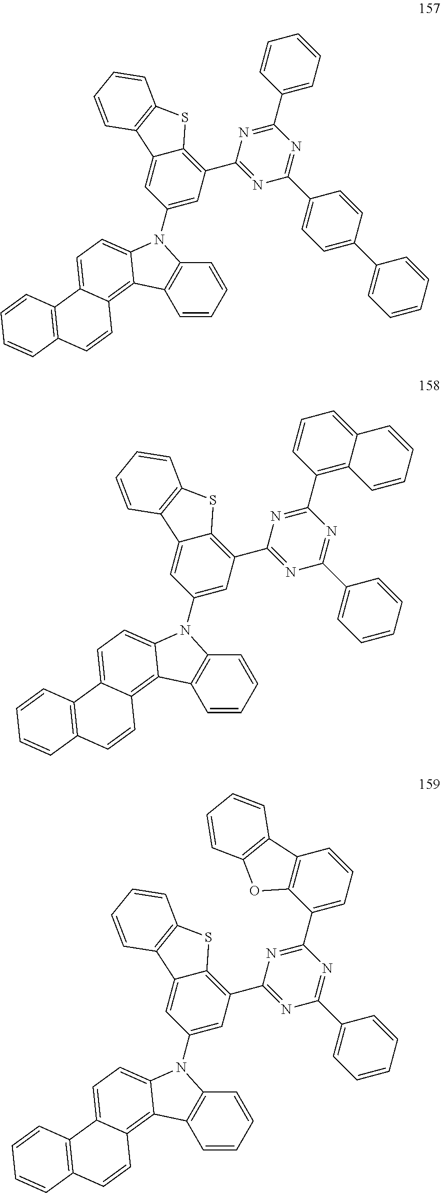 [SMILES: c1ccc(-c2ccc(-c3nc(-c4ccccc4)nc(-c4cc(-n5c6ccccc6c6c7ccc8ccccc8c7ccc65)cc5c4sc4ccccc45)n3)cc2)cc1 ; c1ccc(-c2nc(-c3cccc4ccccc34)nc(-c3cc(-n4c5ccccc5c5c6ccc7ccccc7c6ccc54)cc4c3sc3ccccc34)n2)cc1 ; c1ccc(-c2nc(-c3cccc4c3oc3ccccc34)nc(-c3cc(-n4c5ccccc5c5c6ccc7ccccc7c6ccc54)cc4c3sc3ccccc34)n2)cc1]

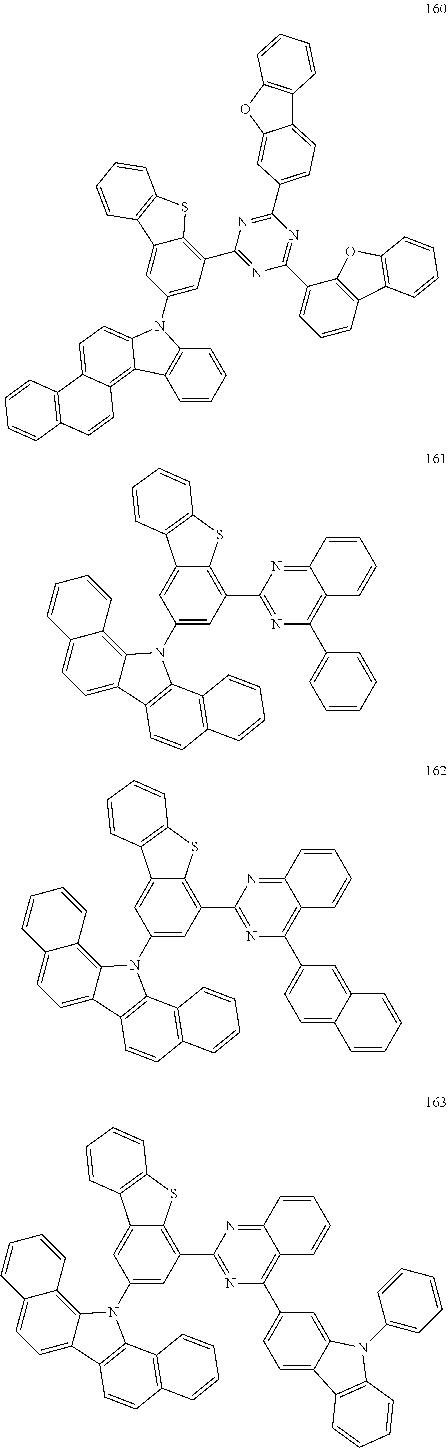

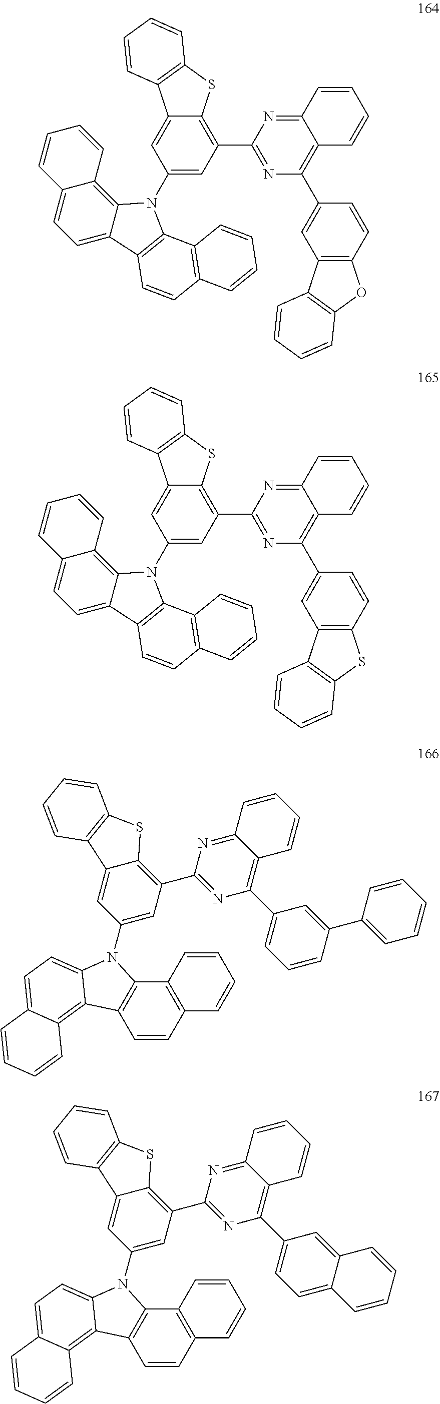 [SMILES: c1ccc2c(c1)ccc1c3ccc4ccccc4c3n(-c3cc(-c4nc(-c5ccc6oc7ccccc7c6c5)c5ccccc5n4)c4sc5ccccc5c4c3)c21 ; c1ccc2c(c1)ccc1c3ccc4ccccc4c3n(-c3cc(-c4nc(-c5ccc6sc7ccccc7c6c5)c5ccccc5n4)c4sc5ccccc5c4c3)c21 ; c1ccc(-c2cccc(-c3nc(-c4cc(-n5c6ccc7ccccc7c6c6ccc7ccccc7c65)cc5c4sc4ccccc45)nc4ccccc34)c2)cc1 ; c1ccc2cc(-c3nc(-c4cc(-n5c6ccc7ccccc7c6c6ccc7ccccc7c65)cc5c4sc4ccccc45)nc4ccccc34)ccc2c1]

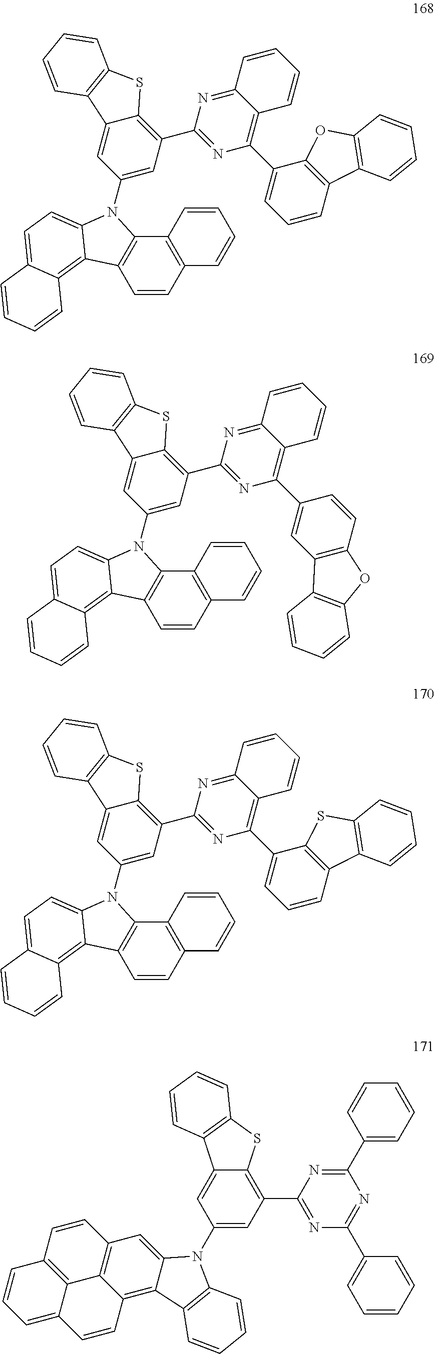 [SMILES: c1ccc2c(c1)ccc1c2c2ccc3ccccc3c2n1-c1cc(-c2nc(-c3cccc4c3oc3ccccc34)c3ccccc3n2)c2sc3ccccc3c2c1 ; c1ccc2c(c1)ccc1c2c2ccc3ccccc3c2n1-c1cc(-c2nc(-c3ccc4oc5ccccc5c4c3)c3ccccc3n2)c2sc3ccccc3c2c1 ; c1ccc2c(c1)ccc1c2c2ccc3ccccc3c2n1-c1cc(-c2nc(-c3cccc4c3sc3ccccc34)c3ccccc3n2)c2sc3ccccc3c2c1 ; c1ccc(-c2nc(-c3ccccc3)nc(-c3cc(-n4c5ccccc5c5c6ccc7cccc8ccc(cc54)c6c87)cc4c3sc3ccccc34)n2)cc1]

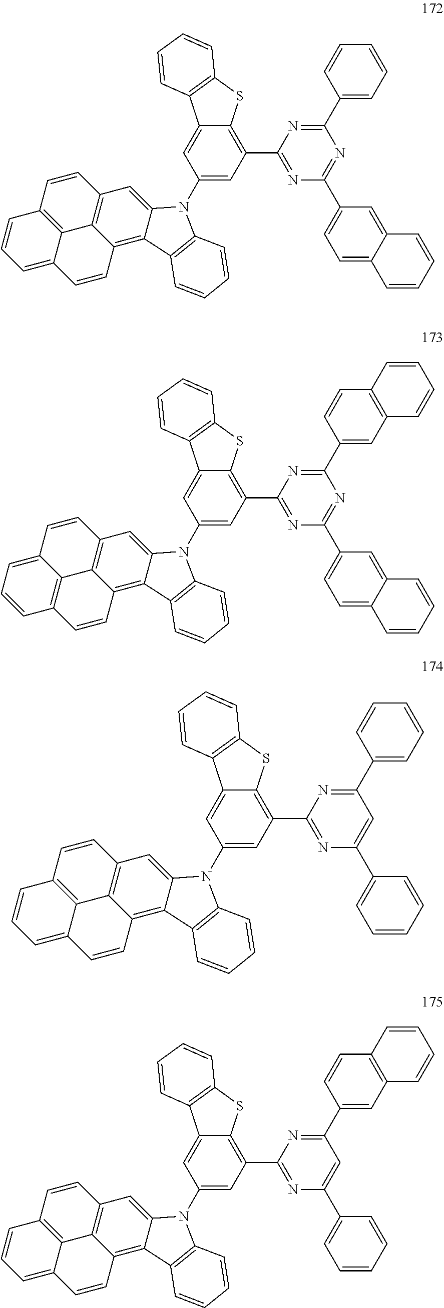 [SMILES: c1ccc(-c2nc(-c3ccc4ccccc4c3)nc(-c3cc(-n4c5ccccc5c5c6ccc7cccc8ccc(cc54)c6c87)cc4c3sc3ccccc34)n2)cc1 ; c1ccc2cc(-c3nc(-c4ccc5ccccc5c4)nc(-c4cc(-n5c6ccccc6c6c7ccc8cccc9ccc(cc65)c7c98)cc5c4sc4ccccc45)n3)ccc2c1 ; c1ccc(-c2cc(-c3ccccc3)nc(-c3cc(-n4c5ccccc5c5c6ccc7cccc8ccc(cc54)c6c87)cc4c3sc3ccccc34)n2)cc1 ; c1ccc(-c2cc(-c3ccc4ccccc4c3)nc(-c3cc(-n4c5ccccc5c5c6ccc7cccc8ccc(cc54)c6c87)cc4c3sc3ccccc34)n2)cc1]

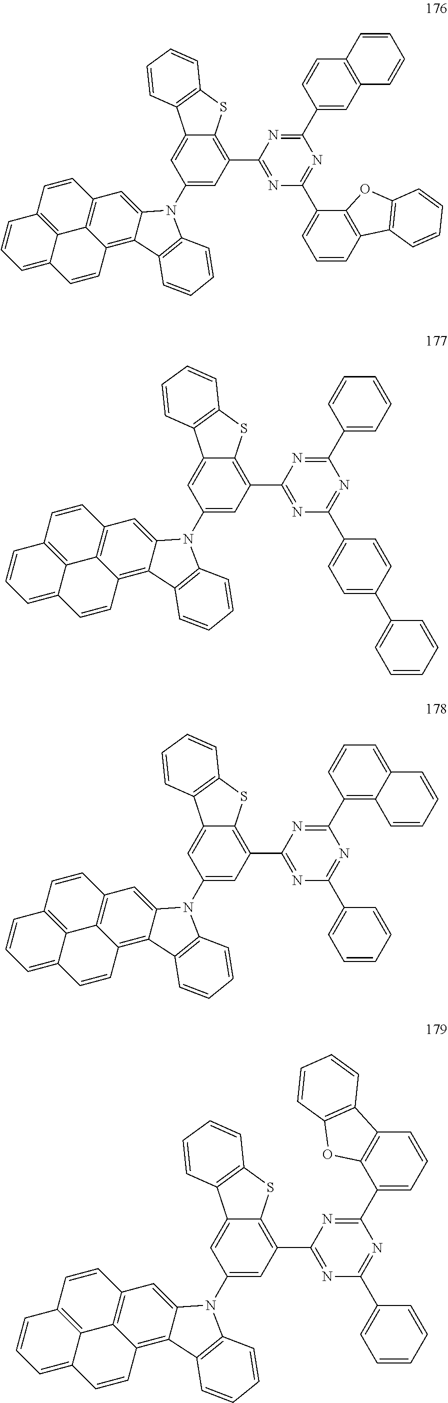 [SMILES: c1ccc2cc(-c3nc(-c4cccc5c4oc4ccccc45)nc(-c4cc(-n5c6ccccc6c6c7ccc8cccc9ccc(cc65)c7c98)cc5c4sc4ccccc45)n3)ccc2c1 ; c1ccc(-c2ccc(-c3nc(-c4ccccc4)nc(-c4cc(-n5c6ccccc6c6c7ccc8cccc9ccc(cc65)c7c98)cc5c4sc4ccccc45)n3)cc2)cc1 ; c1ccc(-c2nc(-c3cccc4ccccc34)nc(-c3cc(-n4c5ccccc5c5c6ccc7cccc8ccc(cc54)c6c87)cc4c3sc3ccccc34)n2)cc1 ; c1ccc(-c2nc(-c3cccc4c3oc3ccccc34)nc(-c3cc(-n4c5ccccc5c5c6ccc7cccc8ccc(cc54)c6c87)cc4c3sc3ccccc34)n2)cc1]

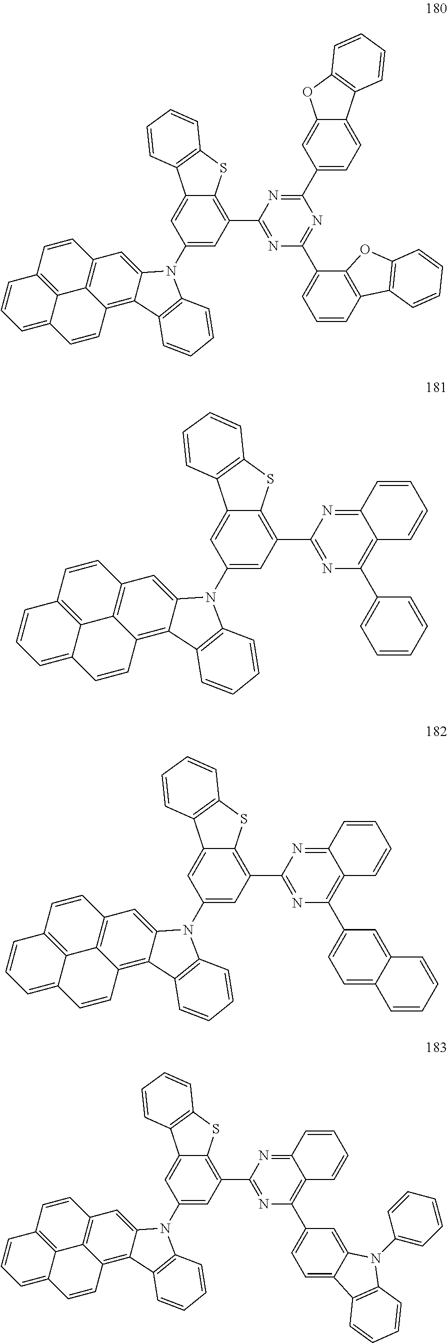 [SMILES: c1cc2ccc3cc4c(c5ccc(c1)c2c35)c1ccccc1n4-c1cc(-c2nc(-c3ccc4c(c3)oc3ccccc34)nc(-c3cccc4c3oc3ccccc34)n2)c2sc3ccccc3c2c1 ; c1ccc(-c2nc(-c3cc(-n4c5ccccc5c5c6ccc7cccc8ccc(cc54)c6c87)cc4c3sc3ccccc34)nc3ccccc23)cc1 ; c1ccc2cc(-c3nc(-c4cc(-n5c6ccccc6c6c7ccc8cccc9ccc(cc65)c7c98)cc5c4sc4ccccc45)nc4ccccc34)ccc2c1 ; c1ccc(-n2c3ccccc3c3ccc(-c4nc(-c5cc(-n6c7ccccc7c7c8ccc9cccc%10ccc(cc76)c8c%109)cc6c5sc5ccccc56)nc5ccccc45)cc32)cc1]

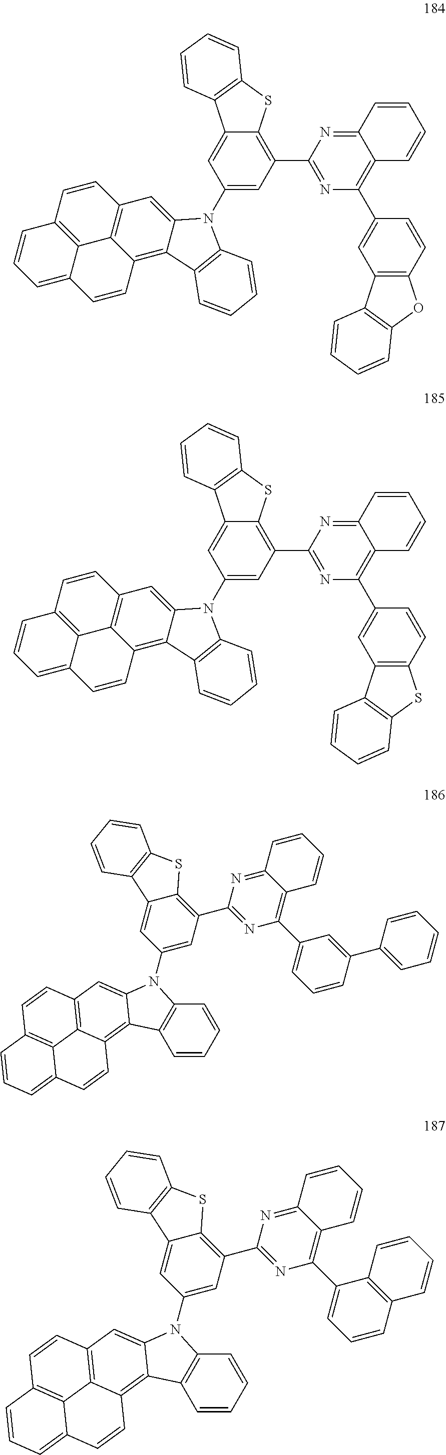 [SMILES: c1cc2ccc3cc4c(c5ccc(c1)c2c35)c1ccccc1n4-c1cc(-c2nc(-c3ccc4oc5ccccc5c4c3)c3ccccc3n2)c2sc3ccccc3c2c1 ; c1cc2ccc3cc4c(c5ccc(c1)c2c35)c1ccccc1n4-c1cc(-c2nc(-c3ccc4sc5ccccc5c4c3)c3ccccc3n2)c2sc3ccccc3c2c1 ; c1ccc(-c2cccc(-c3nc(-c4cc(-n5c6ccccc6c6c7ccc8cccc9ccc(cc65)c7c98)cc5c4sc4ccccc45)nc4ccccc34)c2)cc1 ; c1ccc2c(-c3nc(-c4cc(-n5c6ccccc6c6c7ccc8cccc9ccc(cc65)c7c98)cc5c4sc4ccccc45)nc4ccccc34)cccc2c1]

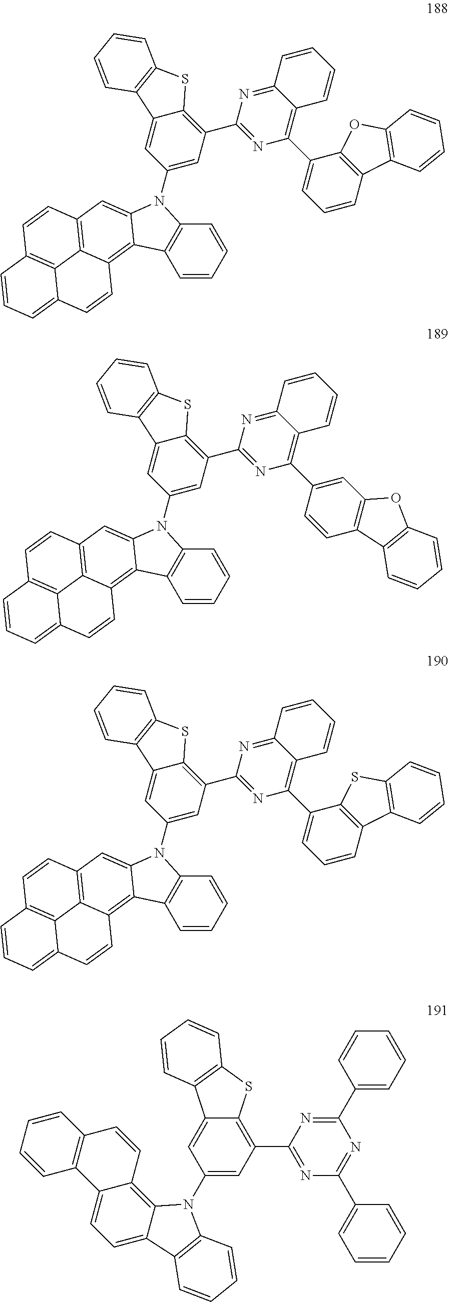 [SMILES: c1cc2ccc3cc4c(c5ccc(c1)c2c35)c1ccccc1n4-c1cc(-c2nc(-c3cccc4c3oc3ccccc34)c3ccccc3n2)c2sc3ccccc3c2c1 ; c1cc2ccc3cc4c(c5ccc(c1)c2c35)c1ccccc1n4-c1cc(-c2nc(-c3ccc4c(c3)oc3ccccc34)c3ccccc3n2)c2sc3ccccc3c2c1 ; c1cc2ccc3cc4c(c5ccc(c1)c2c35)c1ccccc1n4-c1cc(-c2nc(-c3cccc4c3sc3ccccc34)c3ccccc3n2)c2sc3ccccc3c2c1 ; c1ccc(-c2nc(-c3ccccc3)nc(-c3cc(-n4c5ccccc5c5ccc6c7ccccc7ccc6c54)cc4c3sc3ccccc34)n2)cc1]

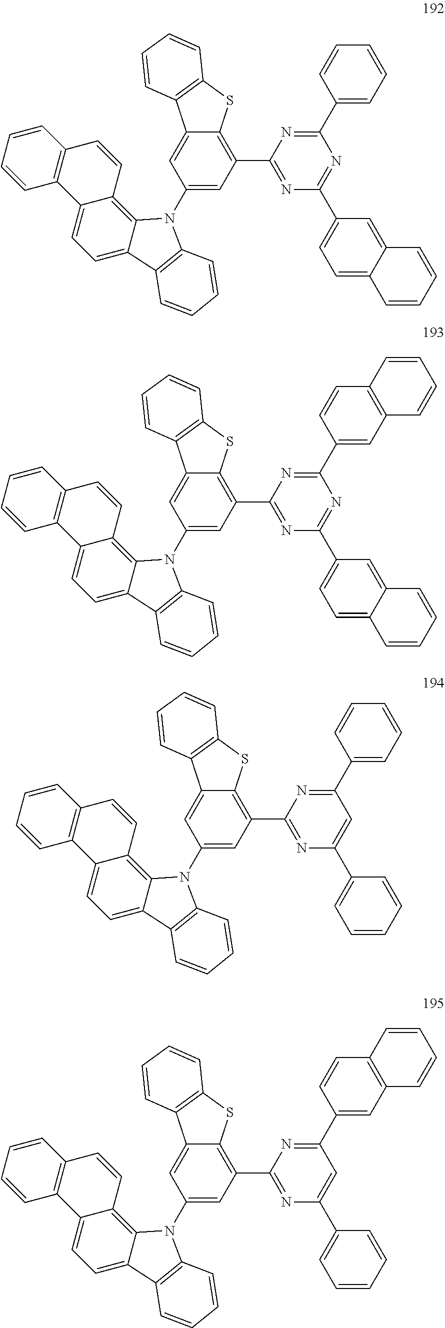

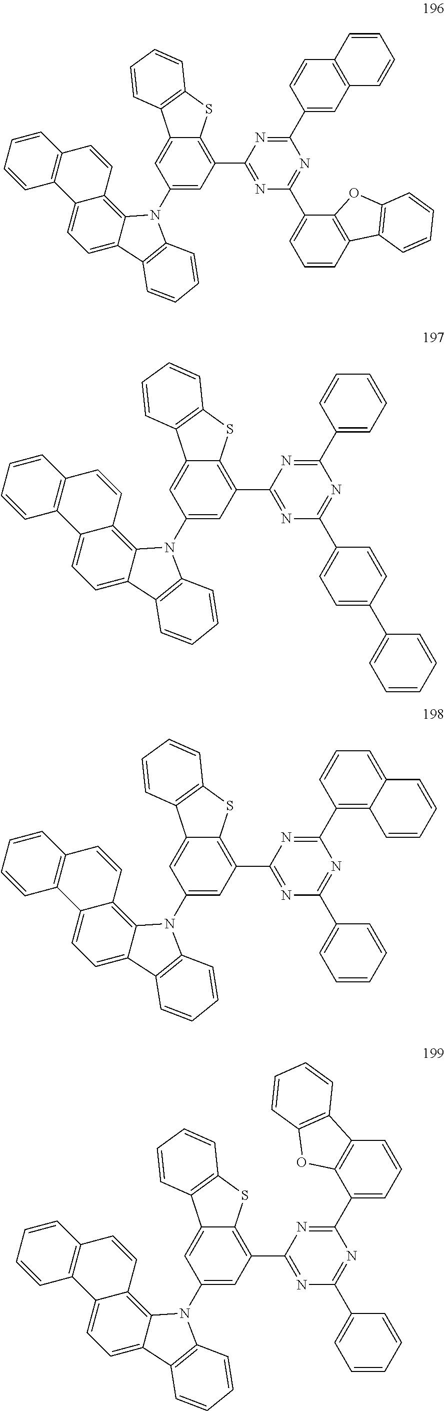 [SMILES: c1ccc2cc(-c3nc(-c4cccc5c4oc4ccccc45)nc(-c4cc(-n5c6ccccc6c6ccc7c8ccccc8ccc7c65)cc5c4sc4ccccc45)n3)ccc2c1 ; c1ccc(-c2ccc(-c3nc(-c4ccccc4)nc(-c4cc(-n5c6ccccc6c6ccc7c8ccccc8ccc7c65)cc5c4sc4ccccc45)n3)cc2)cc1 ; c1ccc(-c2nc(-c3cccc4ccccc34)nc(-c3cc(-n4c5ccccc5c5ccc6c7ccccc7ccc6c54)cc4c3sc3ccccc34)n2)cc1 ; c1ccc(-c2nc(-c3cccc4c3oc3ccccc34)nc(-c3cc(-n4c5ccccc5c5ccc6c7ccccc7ccc6c54)cc4c3sc3ccccc34)n2)cc1]

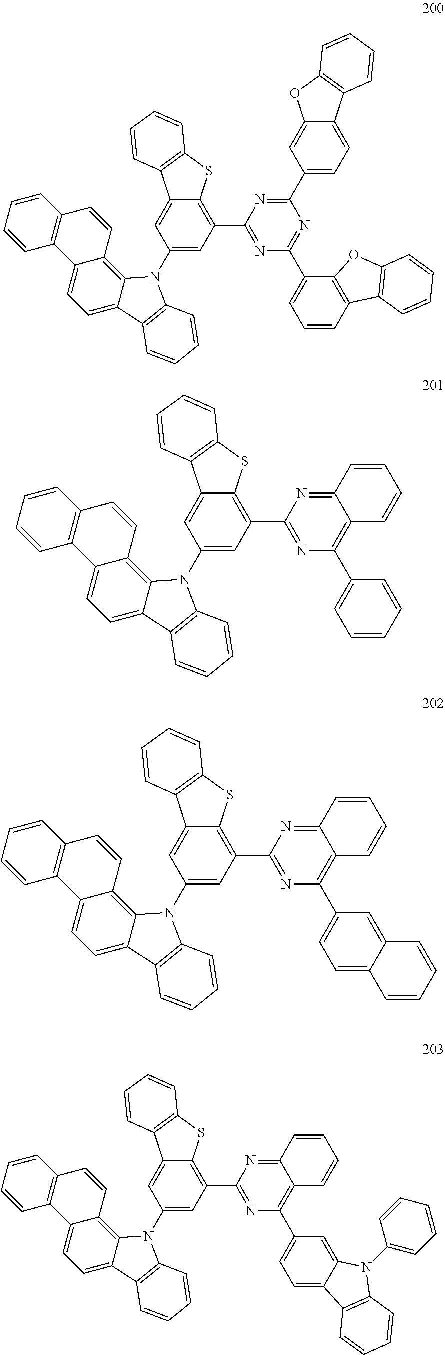

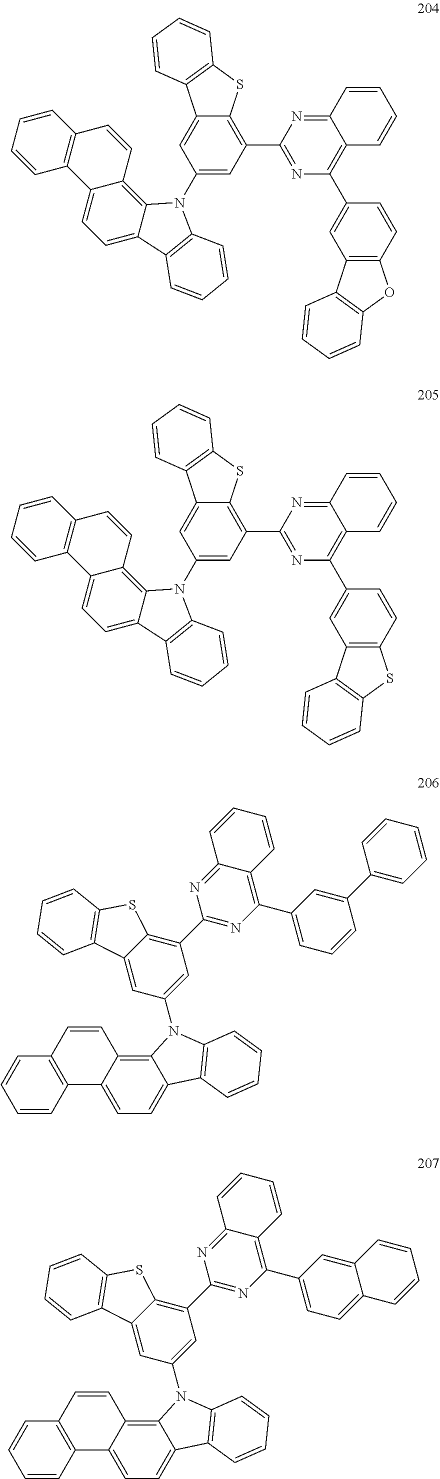 [SMILES: c1ccc2c(c1)ccc1c2ccc2c3ccccc3n(-c3cc(-c4nc(-c5ccc6oc7ccccc7c6c5)c5ccccc5n4)c4sc5ccccc5c4c3)c12 ; c1ccc2c(c1)ccc1c2ccc2c3ccccc3n(-c3cc(-c4nc(-c5ccc6sc7ccccc7c6c5)c5ccccc5n4)c4sc5ccccc5c4c3)c12 ; c1ccc(-c2cccc(-c3nc(-c4cc(-n5c6ccccc6c6ccc7c8ccccc8ccc7c65)cc5c4sc4ccccc45)nc4ccccc34)c2)cc1 ; c1ccc2cc(-c3nc(-c4cc(-n5c6ccccc6c6ccc7c8ccccc8ccc7c65)cc5c4sc4ccccc45)nc4ccccc34)ccc2c1]

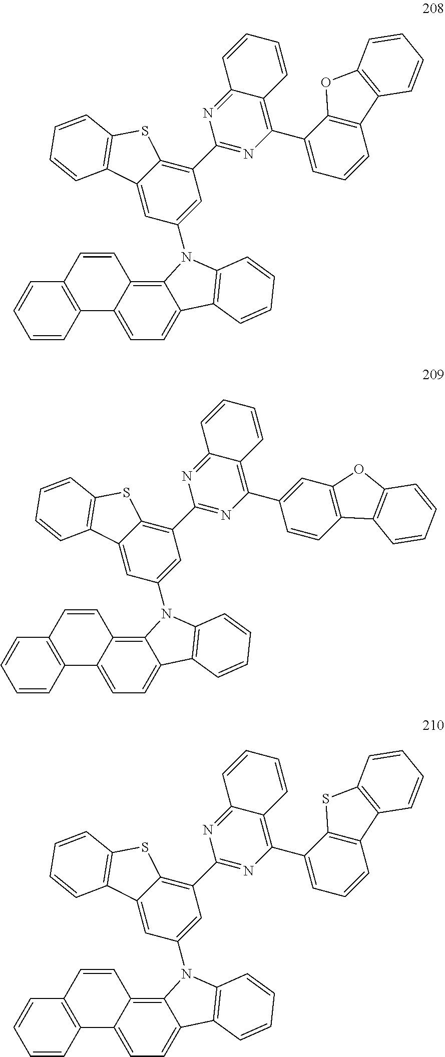 [SMILES: c1ccc2c(c1)ccc1c2ccc2c3ccccc3n(-c3cc(-c4nc(-c5cccc6c5oc5ccccc56)c5ccccc5n4)c4sc5ccccc5c4c3)c12 ; c1ccc2c(c1)ccc1c2ccc2c3ccccc3n(-c3cc(-c4nc(-c5ccc6c(c5)oc5ccccc56)c5ccccc5n4)c4sc5ccccc5c4c3)c12 ; c1ccc2c(c1)ccc1c2ccc2c3ccccc3n(-c3cc(-c4nc(-c5cccc6c5sc5ccccc56)c5ccccc5n4)c4sc5ccccc5c4c3)c12]

The compound or composition described above may be for an organic optoelectronic diode, and the compound for an organic optoelectronic diode or composition for an organic optoelectronic diode may be formed by dry film formation such as chemical vapor deposition.

Hereinafter, an organic optoelectronic diode applied the above-mentioned compound or composition for an organic optoelectronic diode is described.

The organic optoelectronic diode is not particularly limited as long as it is a diode that converts electrical energy into light energy, and vice versa, and examples thereof include an organic photoelectric diode, an organic light emitting diode, an organic solar cell, and an organic photoconductor drum.

In addition, in one embodiment of the present invention, the organic emitting diode includes: the first electrode; the second electrode provided to face the first electrode; and one or more organic material layers provided between the first electrode and the second electrode, wherein the one or more organic material layers provide the organic emitting diode including the heterocyclic compound represented by Formula 1.

In one embodiment of the present invention, the first electrode may be an anode, and the second electrode may be a cathode.

In another embodiment, the first electrode may be a cathode, and the second electrode may be an anode.

Details of the heterocyclic compound represented by Formula 1 are the same as describe above.

In one embodiment of the present application, the organic light emitting diode may be a blue organic light emitting diode, and the heterocyclic compound according to Formula 1 can be used as a material of the blue organic light emitting diode.

In one embodiment of the present application, the organic light emitting diode may be a green organic light emitting diode, and the heterocyclic compound according to Formula 1 can be used as a material of the green organic light emitting diode.

In one embodiment of the present application, the organic light emitting diode may be a red organic light emitting diode, and the heterocyclic compound according to Formula 1 can be used as a material of the red organic light emitting diode.

The organic light emitting diode of the present invention may be manufactured by a method and with materials for manufacturing a conventional organic light emitting diode, except that one or more organic material layers are formed using the heterocyclic compound described above.

The heterocyclic compound may be formed as an organic material layer by a solution coating method as well as a vacuum deposition method in the manufacture of the organic light emitting diode.

In here, the solution coating method may be spin coating, dip coating, inkjet printing, screen printing, a spray method, roll coating, and the like, but is not limited thereto.

Hereinafter, another example of the organic light emitting diode, which is an example of the organic optoelectronic diode, is described with reference to the drawings.

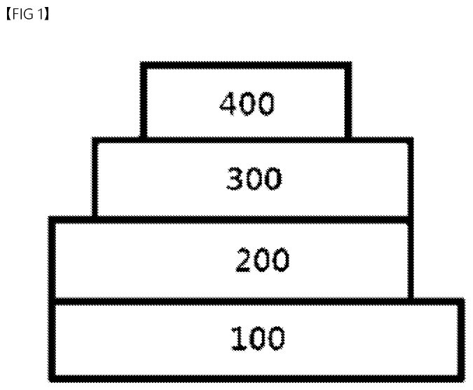

FIGS. 1 to 3 illustrate a lamination order of electrodes and organic material layers of an organic light emitting diode according to one embodiment of the present application.

However, these figures are not intended to limit the scope of the present application, and the structure of the organic optoelectronic diode in the art may be applied to the present application.

According to FIG. 1, an organic light emitting diode in which an anode 200, an organic material layer 300, and a cathode 400 are sequentially laminated on a substrate 100 is illustrated.

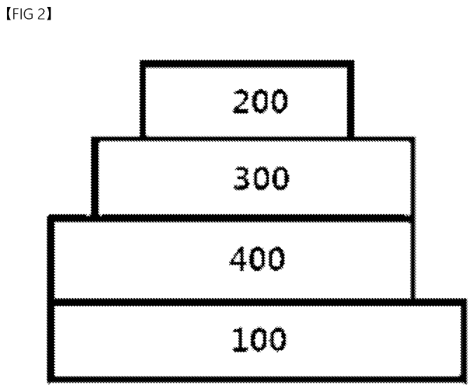

However, the present application is not limited thereto, and as illustrated in FIG. 2, an organic light emitting diode in which a cathode 400, an organic material layer 300, and an anode 200 are sequentially laminated on a substrate 100 may be provided.

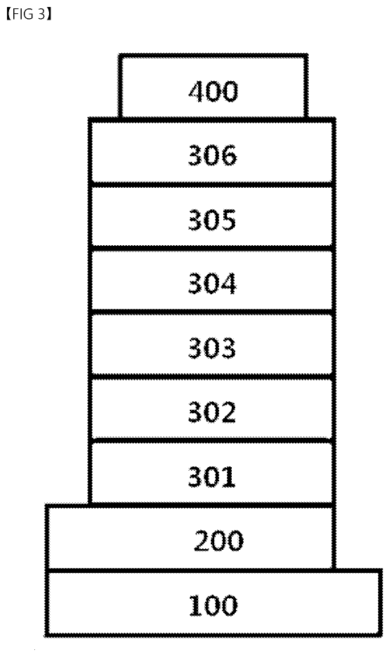

FIG. 3 illustrates a case where the organic material layer is a multilayer.

An organic light emitting diode according to FIG. 3 may include a hole injection layer 301, a hole transport layer 302, a light emitting layer 303, a hole blocking layer 304, an electron transport layer 305, and an electron injection layer 306.

However, the scope of the present application is not limited by such a laminated structure, and other layers except for the light emitting layer may be omitted, and other functional layers may be added as needed.

The compound represented by Formula 1 may be used as an electron transport layer, a hole transport layer, or a light emitting layer in an organic light emitting diode.

As the anode material, materials having a relatively large work function may be used, and a transparent conductive oxide, a metal, or a conductive polymer may be used.

Specific examples of the anode material include metals such as vanadium, chromium, copper, zinc, and gold, or alloys thereof; metal oxides such as zinc oxide, indium oxide, indium tin oxide (ITO), and indium zinc oxide (IZO); ZnO; Al or SnO₂; combinations of oxides with metals such as Sb; conductive polymers such as poly(3-methylthiophene), poly[3,4-(ethylene-1,2-dioxy)thiophene](PEDT), polypyrrole, polyaniline, and the like, but are not limited thereto.

As a cathode material, materials having a relatively low work function may be used, and metals, metal oxides, or conductive polymers may be used.

Specific examples of the cathode materials include metals such as magnesium, calcium, sodium, potassium, titanium, indium, yttrium, lithium, gadolinium, aluminum, silver, tin, and lead, or alloys thereof; multilayer structural materials such as LiF/Al, LiO₂/Al; and the like, but are not limited thereto.

As the hole injection material, well-known hole injection materials may be used, for example, a phthalocyanine compound such as copper phthalocyanine disclosed in U.S. Pat. No. 4,356,429, or a starburst type of amine derivative such as tris(4-carbazoyl)-9-ylphenyl)amine (TCTA), 4,4′,4″-tri[phenyl (m-tolyl)amino]triphenylamine (m-MTDATA), 1,3,5-tris[4-(3-methylphenylphenylamino)phenyl]benzene (m-MTDAPB) disclosed in [Advanced Material, 6, p. 677 (1994)], a soluble conductive polymer such as polyaniline/dodecylbenzenesulfonic acid, poly(3,4-ethylenedioxythiophene)/poly(4-styrenesulfonate), polyaniline/camphor sulfonic acid, polyaniline/poly(4-styrenesulfonate), and the like.

As the hole transport material, pyrazoline derivatives, arylamine derivatives, stilbene derivatives, triphenyldiamine derivatives, and the like may be used, and low molecular or high molecular materials may be used.

As the electron transport material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinomethane and derivatives thereof, fluorenone derivatives, diphenyldicyanoethylene and derivatives thereof, diphenoquinone derivatives, 8-hydroxyquinoline and derivatives thereof, and the like may be used, and high molecular materials as well as low molecular materials may be used.

As the electron injection material, for example, LiF is representatively used in the art, but the present application is not limited thereto.

As the light emitting materials, red, green, or blue light emitting materials may be used, and if necessary, two or more light emitting materials may be mixed and used.

In this case, two or more light emitting materials may be deposited by separate sources, or premixed and deposited by one source.

In addition, although fluorescent materials may be used as the light emitting materials, it can also be used as phosphorescent materials.

As the light emitting materials, materials which combine holes and electrons respectively injected from the anode and the cathode to emit light may be used, but materials in which both the host material and the dopant material are involved in light emitting may be used.

In case of mixing and using hosts of light emitting materials, hosts of the same type may be mixed and used, or hosts of different types may be mixed and used.

For example, two or more kinds of materials selected from n-type host materials and p-type host materials may be used as the host materials of the light emitting layers.

The organic light emitting diodes according to the exemplary embodiment of the present application may be top emission types, bottom emission types, or double-sided emission types according to materials used.

Hereinafter, the above-mentioned embodiments are described in more details with reference to the following examples. However, the following examples are for description purpose only, and do not limit the scope of the present application.

Hereinafter, starting materials and reactants used in the examples and synthesis examples were purchased from Sigma-Aldrich, TCI (Tokyo Chemical Industry), or P&H tech, or synthesized through known methods, unless otherwise specified.

### Preparation of Compounds for Organic Optoelectronic Diode

The main mechanism is as follows.

More details synthesis examples thereof are also described.

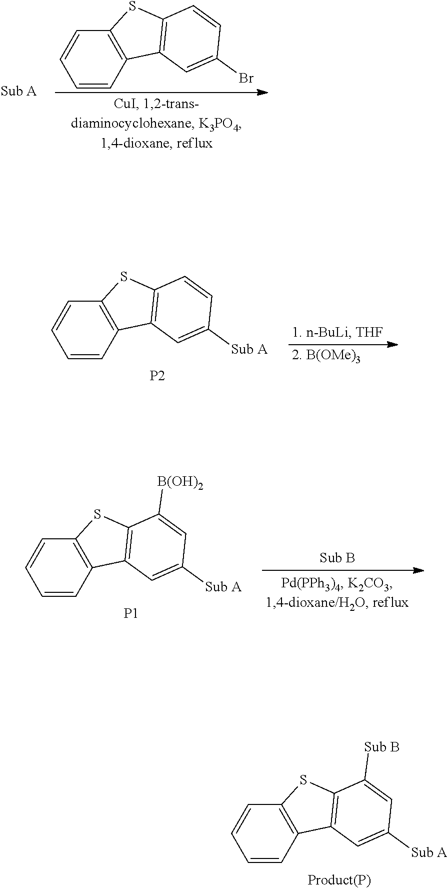

### [Synthesis Examples] Preparation of Product (P)

### a) Preparation of Compound P2

A mixture of Sub A (1 eq), 2-bromodibenzo[b,d]thiophene (1.5 eq), CuI (1 eq), trans-1,2-diaminocyclohexane (1 eq), K₃PO₄ (3 eq), and 1,4-dioxane (10 T) was stirred at reflux for 12 hours in a one neck r.b.f.

The objective compound P2 was obtained from the mixture by extracting with MC and water, drying with MgSO₄, and separating through silica gel column chromatography.

### b) Preparation of Compound P1

P2 (1 eq) and THF (10 T) were added to a one neck r.b.f, and then nitrogen-substituted and cooled to −78° C.

2.5 M n-BuLi in hexane (1.05 eq) was slowly added dropwise, followed by stirring at room temperature for 1 hour, and then B(OMe)₃ (3 eq) was added dropwise and stirred at room temperature for 3 hours.

The objective compound P1 was obtained from the mixture by extracting with MC and water, drying with MgSO₄, and separating through silica gel column chromatography.

### c) Preparation of Compound P

P1 (1 eq), Sub B (1.5 eq), Pd(PPh₃)₄ (0.05 eq), K₂CO₃ (3 eq), 1,4-dioxane/H₂O (10 T) were added and stirred at reflux for 12 hours in a one neck r.b.f.

After the reaction was completed, the precipitated solid was filtered, and the solid was dissolved in MC to obtain the object compound P by silica gel column chromatography.

Specific compounds for the Sub A and Sub B are as follows.

Specific compounds synthesized from a combination of these are shown in Table 1 below.

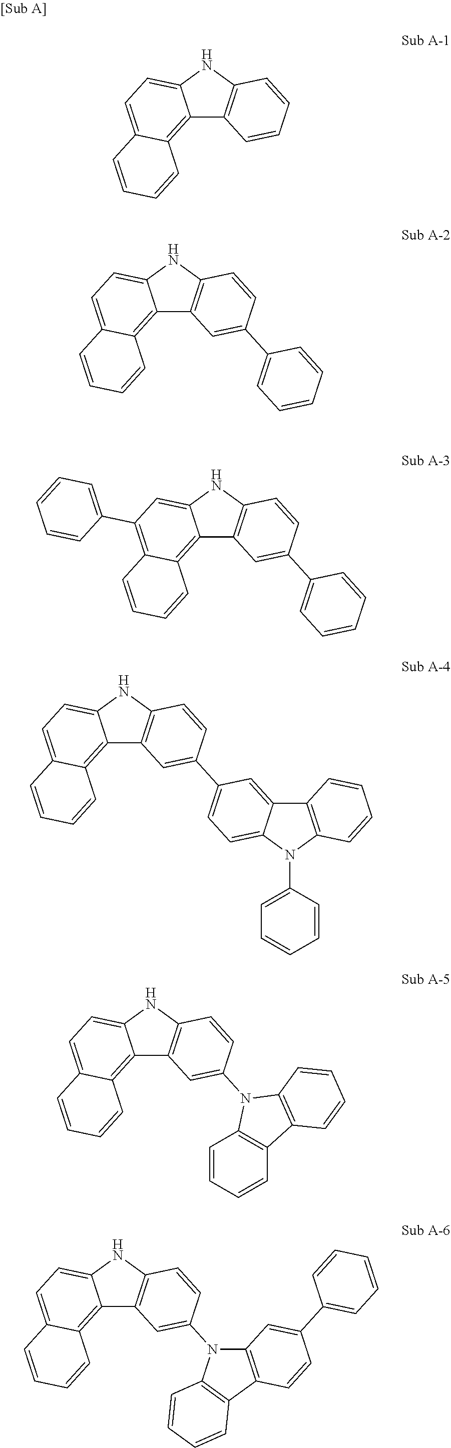 [SMILES: c1ccc2c(c1)ccc1[nH]c3ccccc3c12 ; c1ccc(-c2ccc3[nH]c4ccc5ccccc5c4c3c2)cc1 ; c1ccc(-c2ccc3[nH]c4cc(-c5ccccc5)c5ccccc5c4c3c2)cc1 ; c1ccc(-n2c3ccccc3c3cc(-c4ccc5[nH]c6ccc7ccccc7c6c5c4)ccc32)cc1 ; c1ccc2c(c1)ccc1[nH]c3ccc(-n4c5ccccc5c5ccccc54)cc3c12 ; c1ccc(-c2ccc3c4ccccc4n(-c4ccc5[nH]c6ccc7ccccc7c6c5c4)c3c2)cc1]

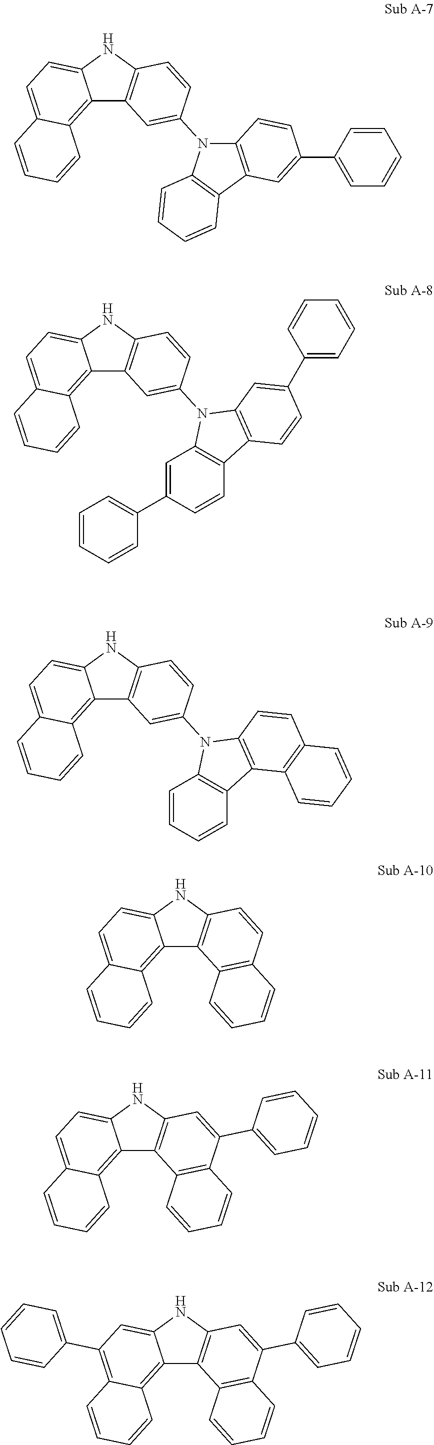 [SMILES: c1ccc(-c2ccc3c(c2)c2ccccc2n3-c2ccc3[nH]c4ccc5ccccc5c4c3c2)cc1 ; c1ccc(-c2ccc3c4ccc(-c5ccccc5)cc4n(-c4ccc5[nH]c6ccc7ccccc7c6c5c4)c3c2)cc1 ; c1ccc2c(c1)ccc1[nH]c3ccc(-n4c5ccccc5c5c6ccccc6ccc54)cc3c12 ; c1ccc2c(c1)ccc1[nH]c3ccc4ccccc4c3c12 ; c1ccc(-c2cc3[nH]c4ccc5ccccc5c4c3c3ccccc23)cc1 ; c1ccc(-c2cc3[nH]c4cc(-c5ccccc5)c5ccccc5c4c3c3ccccc23)cc1]

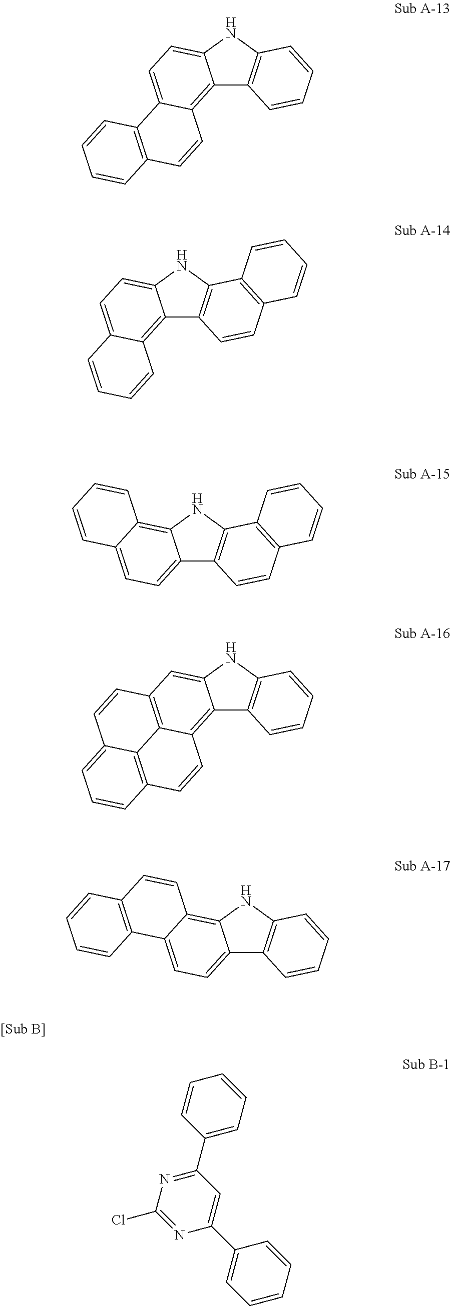 [SMILES: c1ccc2c(c1)ccc1c2ccc2[nH]c3ccccc3c21 ; c1ccc2c(c1)ccc1c2[nH]c2ccc3ccccc3c21 ; c1ccc2c(c1)ccc1c3ccc4ccccc4c3[nH]c21 ; c1cc2ccc3cc4[nH]c5ccccc5c4c4ccc(c1)c2c34 ; c1ccc2c(c1)ccc1c2ccc2c3ccccc3[nH]c21 ; Clc1nc(-c2ccccc2)cc(-c2ccccc2)n1]

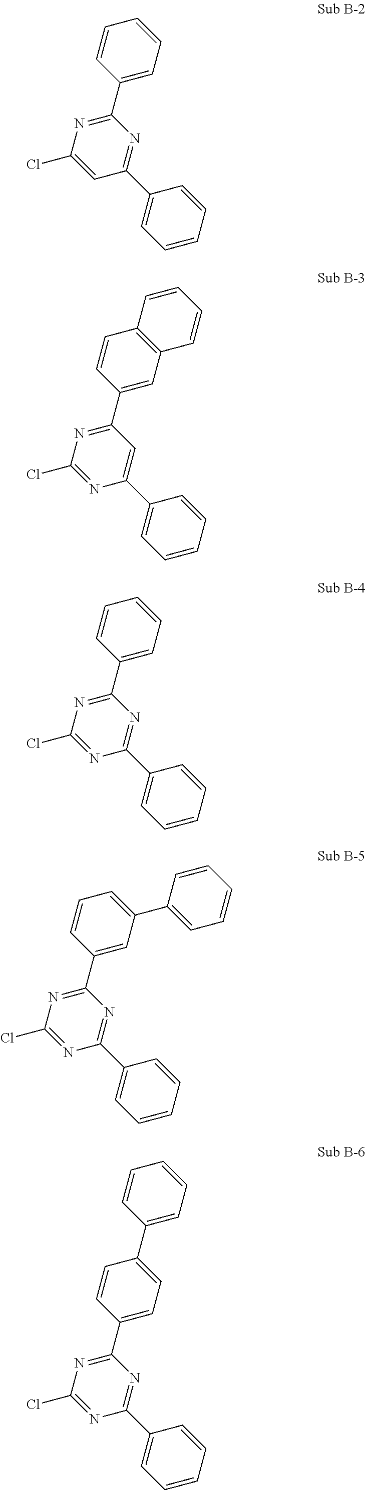

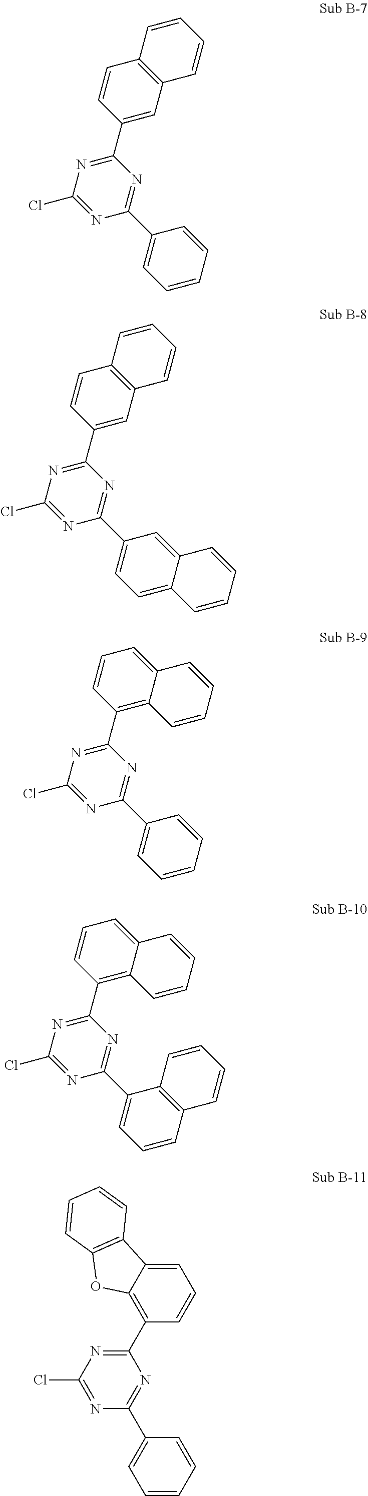

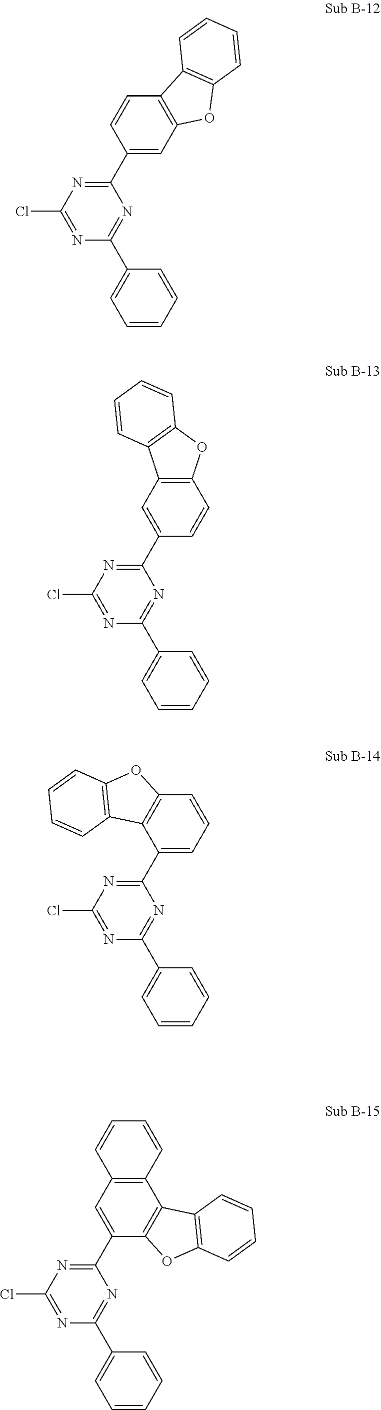 [SMILES: Clc1nc(-c2ccccc2)nc(-c2ccc3c(c2)oc2ccccc23)n1 ; Clc1nc(-c2ccccc2)nc(-c2ccc3oc4ccccc4c3c2)n1 ; Clc1nc(-c2ccccc2)nc(-c2cccc3oc4ccccc4c23)n1 ; Clc1nc(-c2ccccc2)nc(-c2cc3ccccc3c3c2oc2ccccc23)n1]

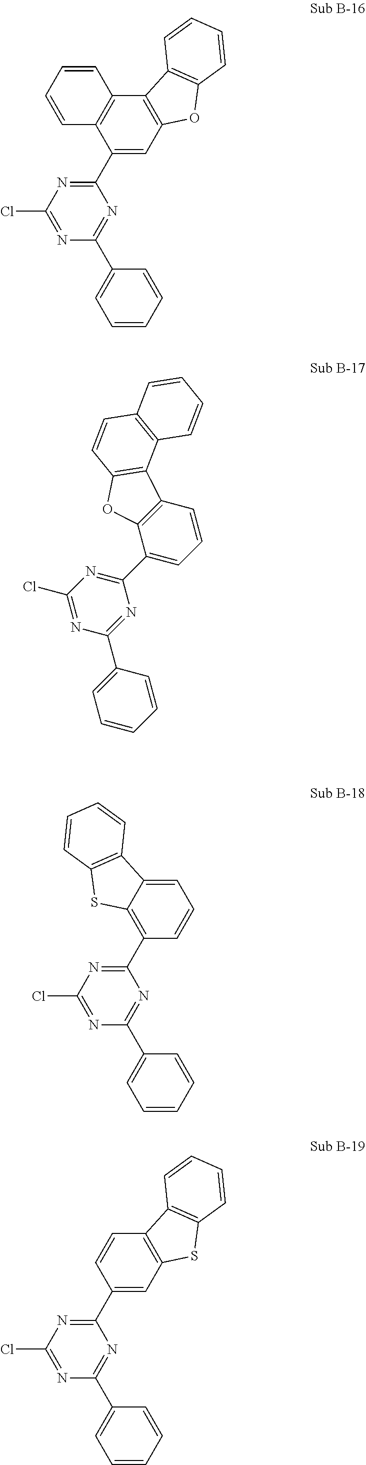 [SMILES: Clc1nc(-c2ccccc2)nc(-c2cc3oc4ccccc4c3c3ccccc23)n1 ; Clc1nc(-c2ccccc2)nc(-c2cccc3c2oc2ccc4ccccc4c23)n1 ; Clc1nc(-c2ccccc2)nc(-c2cccc3c2sc2ccccc23)n1 ; Clc1nc(-c2ccccc2)nc(-c2ccc3c(c2)sc2ccccc23)n1]

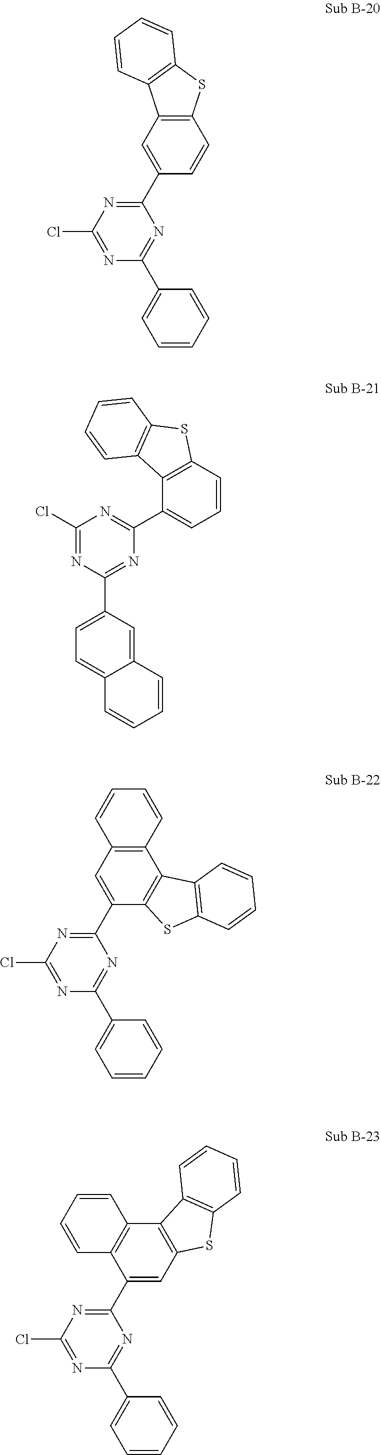 [SMILES: Clc1nc(-c2ccccc2)nc(-c2ccc3sc4ccccc4c3c2)n1 ; Clc1nc(-c2ccc3ccccc3c2)nc(-c2cccc3sc4ccccc4c23)n1 ; Clc1nc(-c2ccccc2)nc(-c2cc3ccccc3c3c2sc2ccccc23)n1 ; Clc1nc(-c2ccccc2)nc(-c2cc3sc4ccccc4c3c3ccccc23)n1]

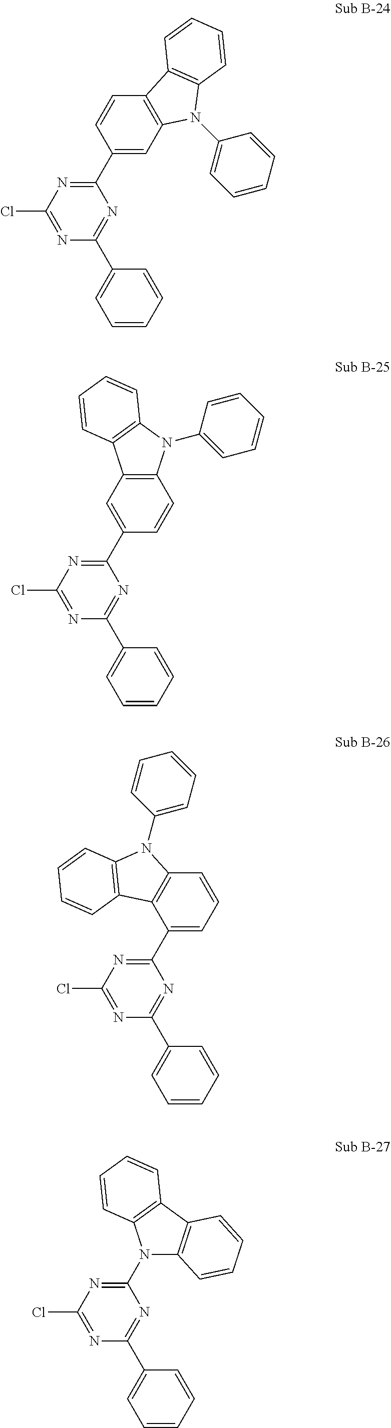 [SMILES: Clc1nc(-c2ccccc2)nc(-c2ccc3c4ccccc4n(-c4ccccc4)c3c2)n1 ; Clc1nc(-c2ccccc2)nc(-c2ccc3c(c2)c2ccccc2n3-c2ccccc2)n1 ; Clc1nc(-c2ccccc2)nc(-c2cccc3c2c2ccccc2n3-c2ccccc2)n1 ; Clc1nc(-c2ccccc2)nc(-n2c3ccccc3c3ccccc32)n1]

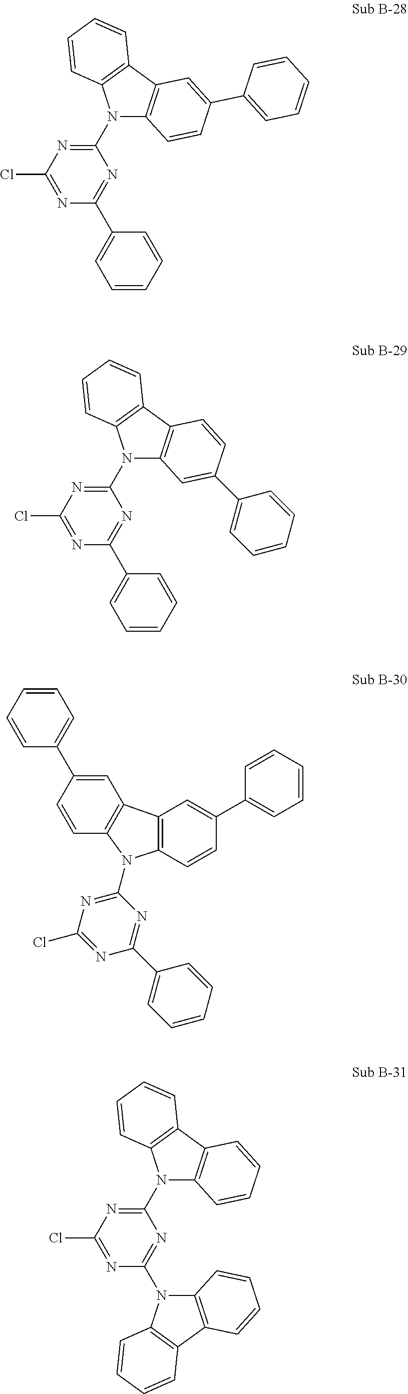 [SMILES: Clc1nc(-c2ccccc2)nc(-n2c3ccccc3c3cc(-c4ccccc4)ccc32)n1 ; Clc1nc(-c2ccccc2)nc(-n2c3ccccc3c3ccc(-c4ccccc4)cc32)n1 ; Clc1nc(-c2ccccc2)nc(-n2c3ccc(-c4ccccc4)cc3c3cc(-c4ccccc4)ccc32)n1 ; Clc1nc(-n2c3ccccc3c3ccccc32)nc(-n2c3ccccc3c3ccccc32)n1]

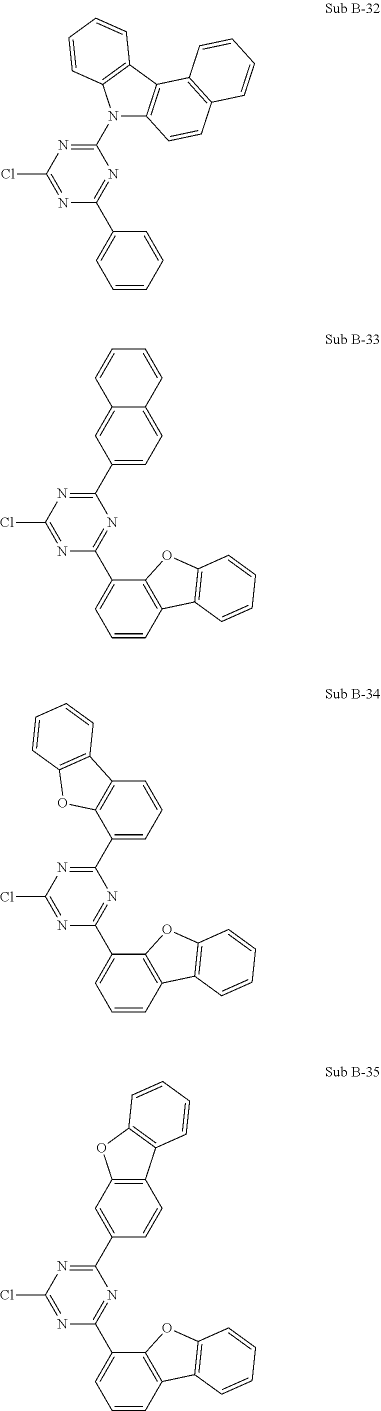 [SMILES: Clc1nc(-c2ccccc2)nc(-n2c3ccccc3c3c4ccccc4ccc32)n1 ; Clc1nc(-c2ccc3ccccc3c2)nc(-c2cccc3c2oc2ccccc23)n1 ; Clc1nc(-c2cccc3c2oc2ccccc23)nc(-c2cccc3c2oc2ccccc23)n1 ; Clc1nc(-c2ccc3c(c2)oc2ccccc23)nc(-c2cccc3c2oc2ccccc23)n1]

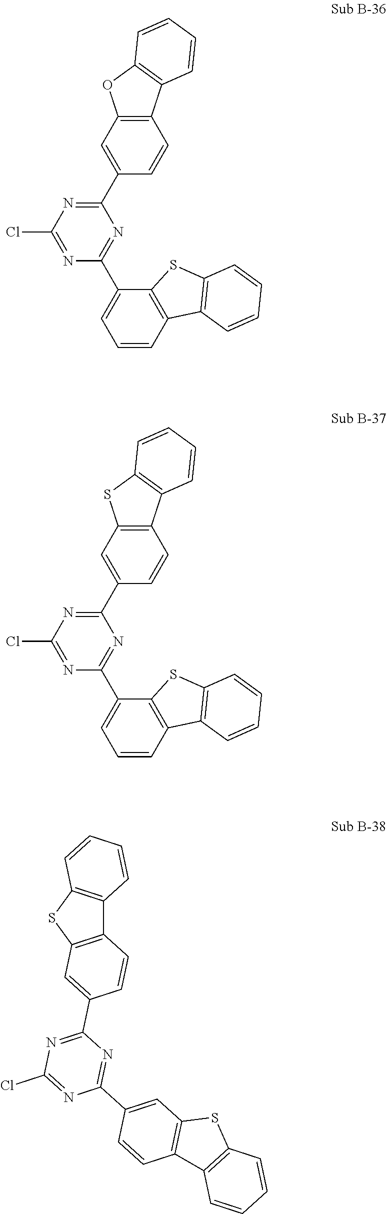 [SMILES: Clc1nc(-c2ccc3c(c2)oc2ccccc23)nc(-c2cccc3c2sc2ccccc23)n1 ; Clc1nc(-c2ccc3c(c2)sc2ccccc23)nc(-c2cccc3c2sc2ccccc23)n1 ; Clc1nc(-c2ccc3c(c2)sc2ccccc23)nc(-c2ccc3c(c2)sc2ccccc23)n1]

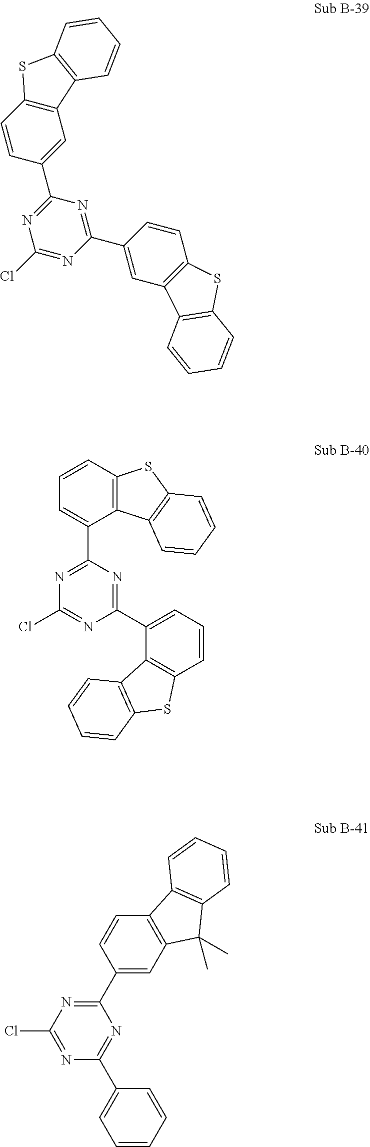 [SMILES: Clc1nc(-c2ccc3sc4ccccc4c3c2)nc(-c2ccc3sc4ccccc4c3c2)n1 ; Clc1nc(-c2cccc3sc4ccccc4c23)nc(-c2cccc3sc4ccccc4c23)n1 ; CC1(C)c2ccccc2-c2ccc(-c3nc(Cl)nc(-c4ccccc4)n3)cc21]

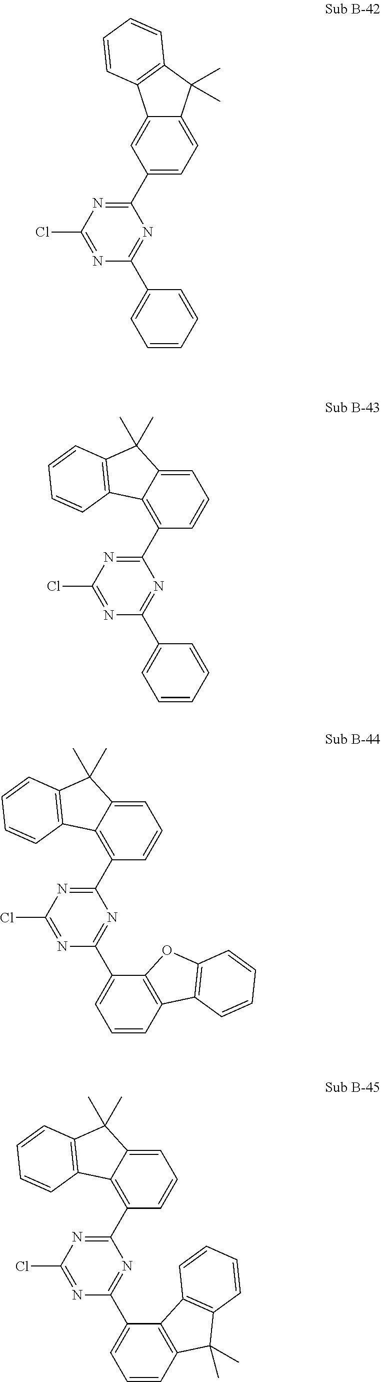 [SMILES: CC1(C)c2ccccc2-c2cc(-c3nc(Cl)nc(-c4ccccc4)n3)ccc21 ; CC1(C)c2ccccc2-c2c(-c3nc(Cl)nc(-c4ccccc4)n3)cccc21 ; CC1(C)c2ccccc2-c2c(-c3nc(Cl)nc(-c4cccc5c4oc4ccccc45)n3)cccc21 ; CC1(C)c2ccccc2-c2c(-c3nc(Cl)nc(-c4cccc5c4-c4ccccc4C5(C)C)n3)cccc21]

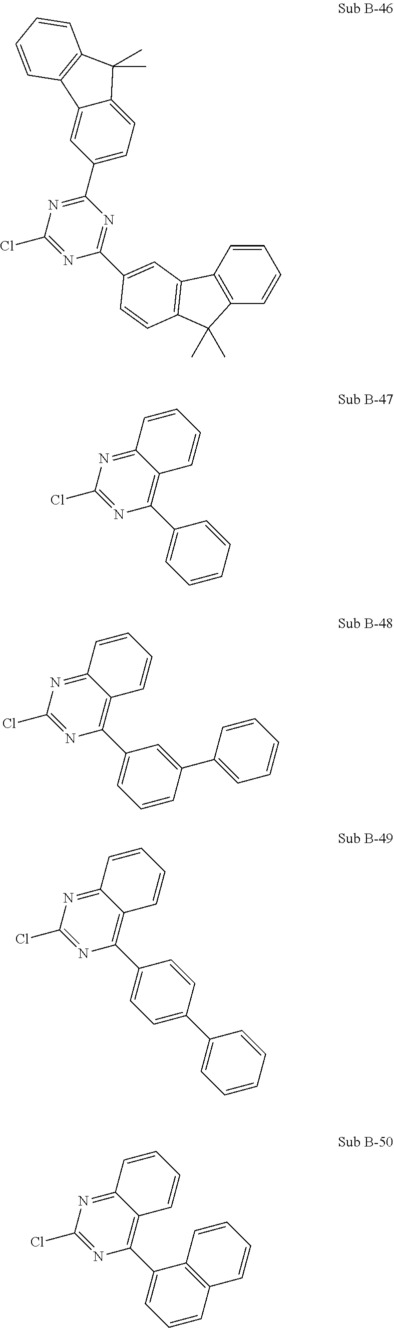

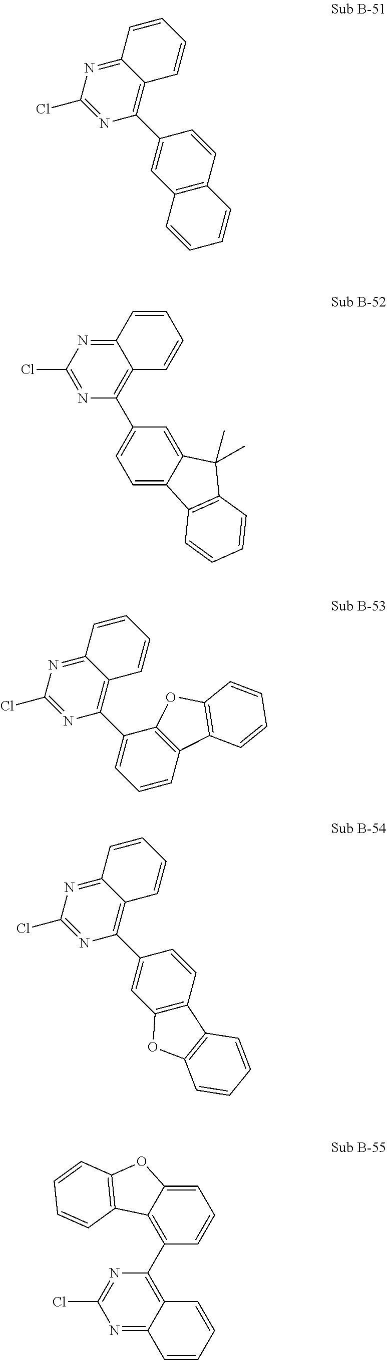

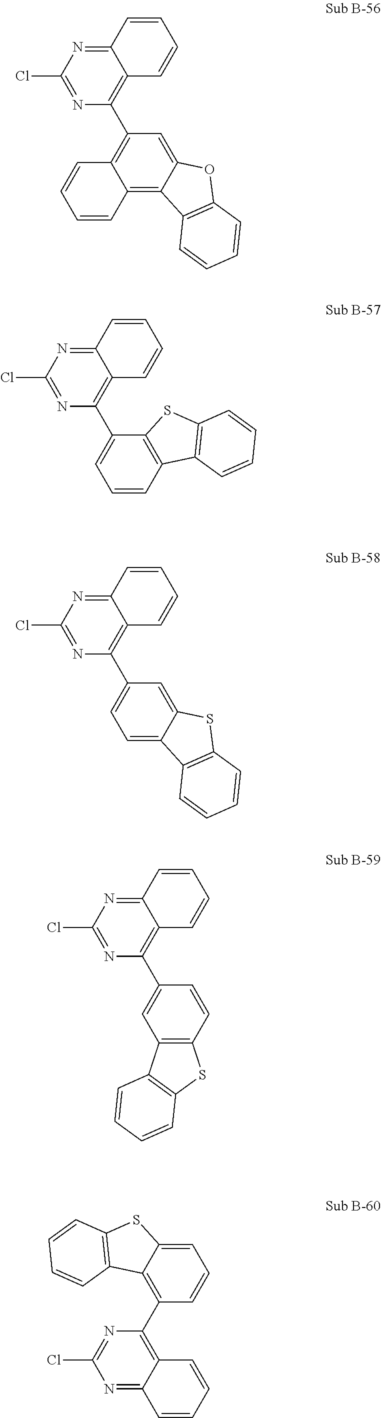 [SMILES: Clc1nc(-c2cc3oc4ccccc4c3c3ccccc23)c2ccccc2n1 ; Clc1nc(-c2cccc3c2sc2ccccc23)c2ccccc2n1 ; Clc1nc(-c2ccc3c(c2)sc2ccccc23)c2ccccc2n1 ; Clc1nc(-c2ccc3sc4ccccc4c3c2)c2ccccc2n1 ; Clc1nc(-c2cccc3sc4ccccc4c23)c2ccccc2n1]

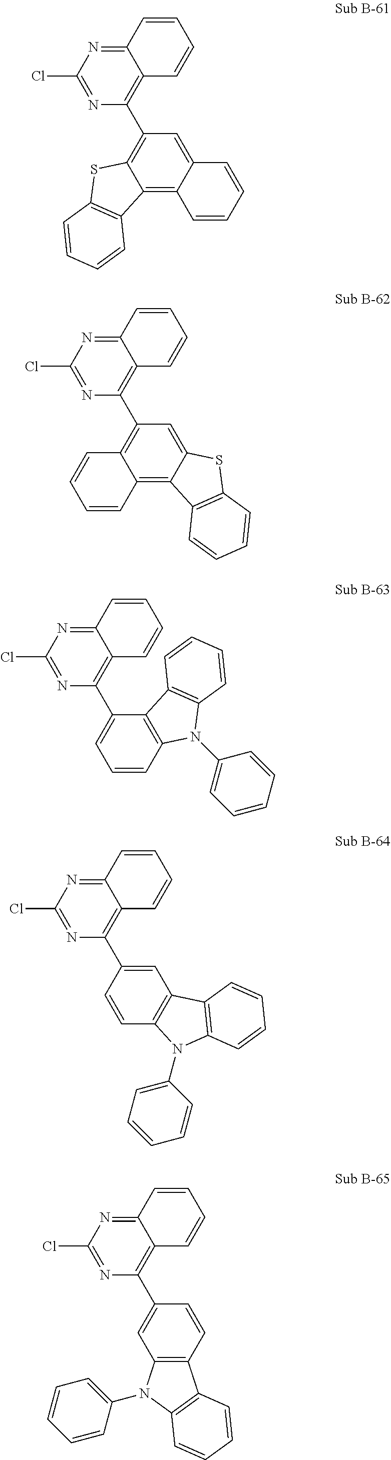

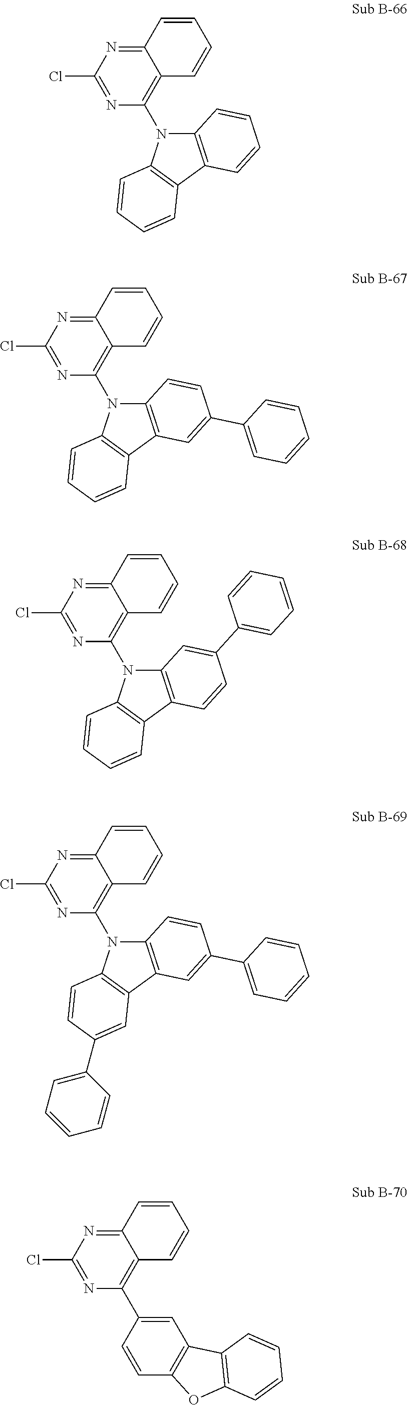 [SMILES: Clc1nc(-n2c3ccccc3c3ccccc32)c2ccccc2n1 ; Clc1nc(-n2c3ccccc3c3cc(-c4ccccc4)ccc32)c2ccccc2n1 ; Clc1nc(-n2c3ccccc3c3ccc(-c4ccccc4)cc32)c2ccccc2n1 ; Clc1nc(-n2c3ccc(-c4ccccc4)cc3c3cc(-c4ccccc4)ccc32)c2ccccc2n1 ; Clc1nc(-c2ccc3oc4ccccc4c3c2)c2ccccc2n1]

TABLE 1

| Compound | Sub A | Linker |
|---|---|---|
| 1 | 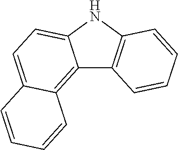 [SMILES: c1ccc2c(c1)ccc1[nH]c3ccccc3c12] | 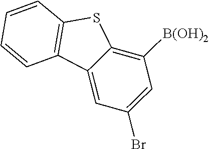 |
| 3 | 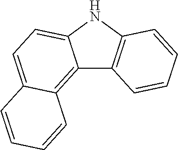 [SMILES: c1ccc2c(c1)ccc1[nH]c3ccccc3c12] | 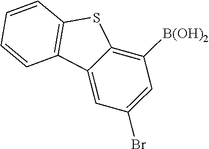 |
| 5 | 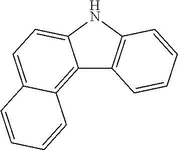 [SMILES: c1ccc2c(c1)ccc1[nH]c3ccccc3c12] | 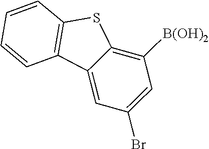 |
| 12 | 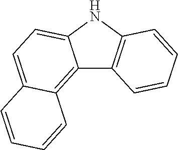 [SMILES: c1ccc2c(c1)ccc1[nH]c3ccccc3c12] | 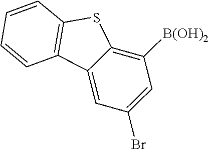 |
| 18 | 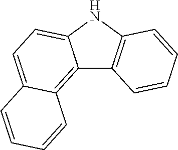 [SMILES: c1ccc2c(c1)ccc1[nH]c3ccccc3c12] | 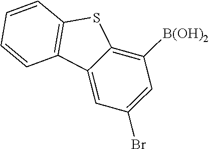 |
| 22 | 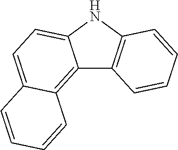 [SMILES: c1ccc2c(c1)ccc1[nH]c3ccccc3c12] | 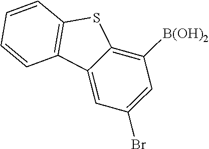 |
| 31 | 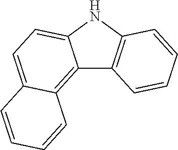 [SMILES: c1ccc2c(c1)ccc1[nH]c3ccccc3c12] | 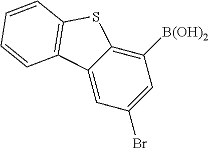 |
| 34 | 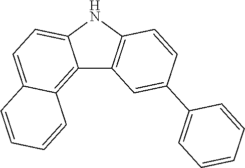 [SMILES: c1ccc(-c2ccc3[nH]c4ccc5ccccc5c4c3c2)cc1] | 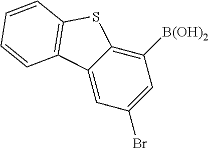 |
| 35 | 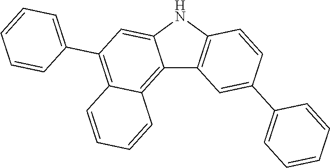 [SMILES: c1ccc(-c2ccc3[nH]c4cc(-c5ccccc5)c5ccccc5c4c3c2)cc1] | 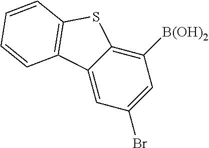 |
| 36 | 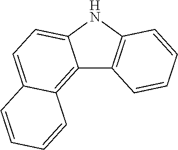 [SMILES: c1ccc2c(c1)ccc1[nH]c3ccccc3c12] | 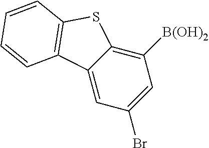 |
| 38 | 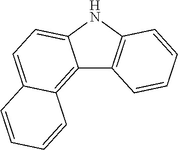 [SMILES: c1ccc2c(c1)ccc1[nH]c3ccccc3c12] | 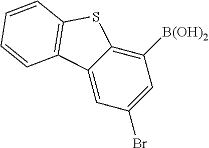 |
| 43 | 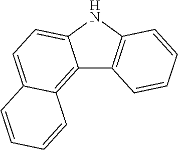 [SMILES: c1ccc2c(c1)ccc1[nH]c3ccccc3c12] | 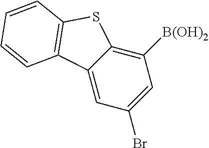 |
| 47 | 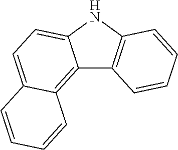 [SMILES: c1ccc2c(c1)ccc1[nH]c3ccccc3c12] | 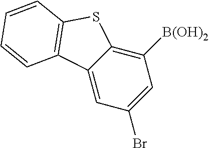 |
| 54 | 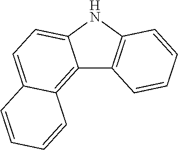 [SMILES: c1ccc2c(c1)ccc1[nH]c3ccccc3c12] | 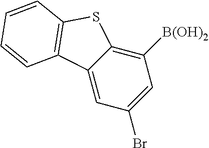 |
| 59 | 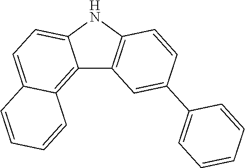 [SMILES: c1ccc(-c2ccc3[nH]c4ccc5ccccc5c4c3c2)cc1] | 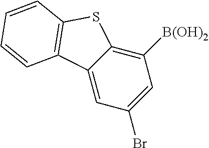 |
| 61 | 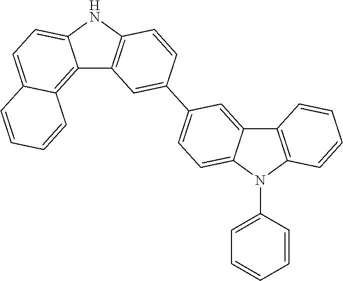 [SMILES: c1ccc(-n2c3ccccc3c3cc(-c4ccc5[nH]c6ccc7ccccc7c6c5c4)ccc32)cc1] | 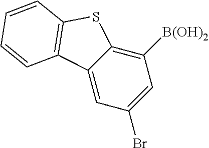 |
| 64 | 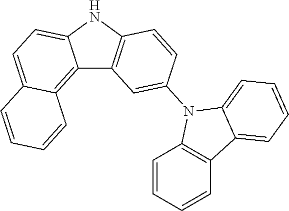 [SMILES: c1ccc2c(c1)ccc1[nH]c3ccc(-n4c5ccccc5c5ccccc54)cc3c12] | 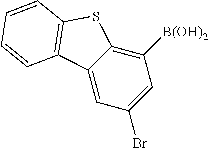 |
| 67 | 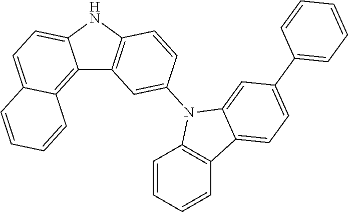 [SMILES: c1ccc(-c2ccc3c4ccccc4n(-c4ccc5[nH]c6ccc7ccccc7c6c5c4)c3c2)cc1] | 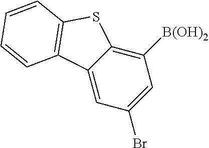 |
| 68 | 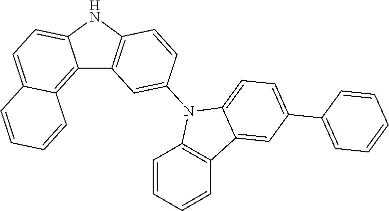 [SMILES: c1ccc(-c2ccc3c(c2)c2ccccc2n3-c2ccc3[nH]c4ccc5ccccc5c4c3c2)cc1] | 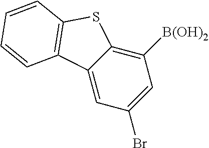 |
| 69 | 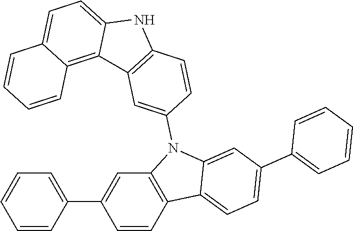 [SMILES: c1ccc(-c2ccc3c4ccc(-c5ccccc5)cc4n(-c4ccc5[nH]c6ccc7ccccc7c6c5c4)c3c2)cc1] | 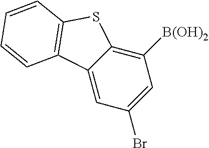 |
| 70 | 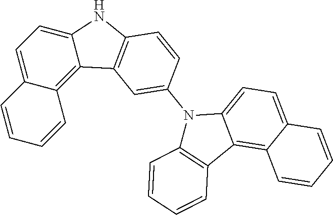 [SMILES: c1ccc2c(c1)ccc1[nH]c3ccc(-n4c5ccccc5c5c6ccccc6ccc54)cc3c12] | 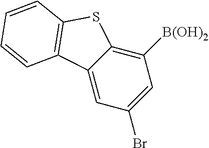 |
| 71 | 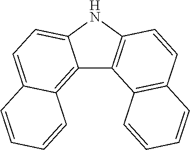 [SMILES: c1ccc2c(c1)ccc1[nH]c3ccc4ccccc4c3c12] | 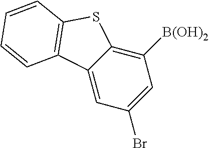 |
| 73 | 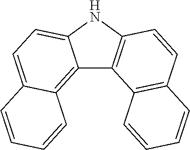 [SMILES: c1ccc2c(c1)ccc1[nH]c3ccc4ccccc4c3c12] | 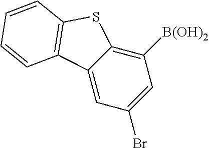 |
| 75 | 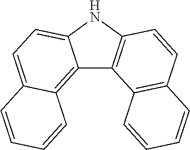 [SMILES: c1ccc2c(c1)ccc1[nH]c3ccc4ccccc4c3c12] | 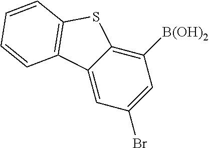 |
| 82 | 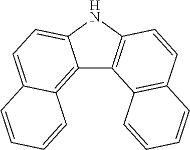 [SMILES: c1ccc2c(c1)ccc1[nH]c3ccc4ccccc4c3c12] | 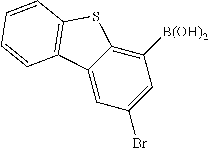 |
| 88 | 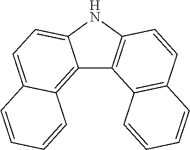 [SMILES: c1ccc2c(c1)ccc1[nH]c3ccc4ccccc4c3c12] | 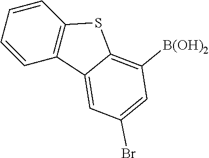 |
| 92 | 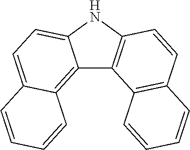 [SMILES: c1ccc2c(c1)ccc1[nH]c3ccc4ccccc4c3c12] | 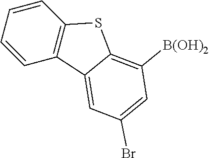 |
| 104 | 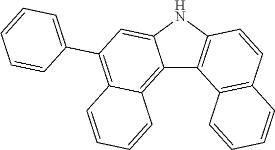 [SMILES: c1ccc(-c2cc3[nH]c4ccc5ccccc5c4c3c3ccccc23)cc1] | 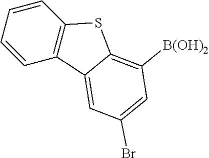 |
| 105 | 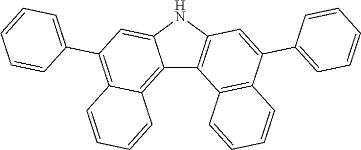 [SMILES: c1ccc(-c2cc3[nH]c4cc(-c5ccccc5)c5ccccc5c4c3c3ccccc23)cc1] | 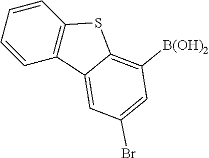 |
| 106 | 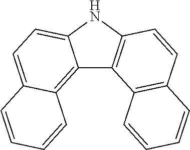 [SMILES: c1ccc2c(c1)ccc1[nH]c3ccc4ccccc4c3c12] | 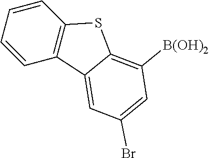 |
| 108 | 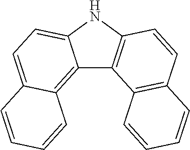 [SMILES: c1ccc2c(c1)ccc1[nH]c3ccc4ccccc4c3c12] | 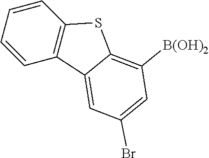 |
| 113 | 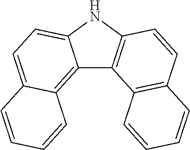 [SMILES: c1ccc2c(c1)ccc1[nH]c3ccc4ccccc4c3c12] | 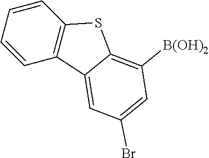 |
| 117 | 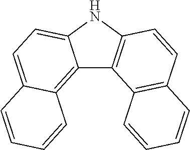 [SMILES: c1ccc2c(c1)ccc1[nH]c3ccc4ccccc4c3c12] | 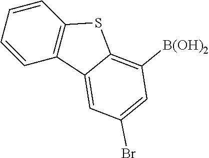 |
| 124 | 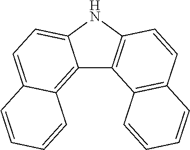 [SMILES: c1ccc2c(c1)ccc1[nH]c3ccc4ccccc4c3c12] | 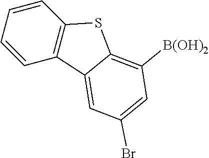 |
| 129 | 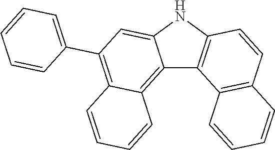 [SMILES: c1ccc(-c2cc3[nH]c4ccc5ccccc5c4c3c3ccccc23)cc1] | 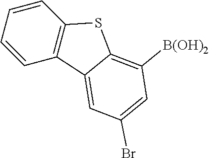 |
| 136 | 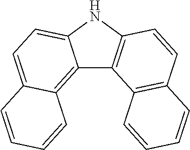 [SMILES: c1ccc2c(c1)ccc1[nH]c3ccc4ccccc4c3c12] | 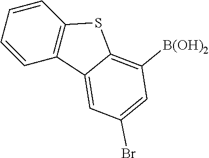 |
| 141 | 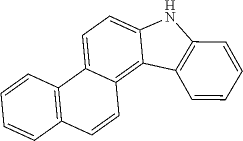 [SMILES: c1ccc2c(c1)ccc1c2ccc2[nH]c3ccccc3c21] | 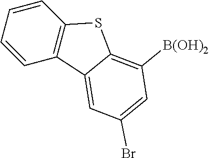 |
| 144 | 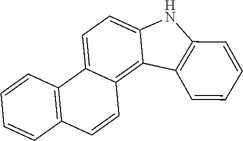 [SMILES: c1ccc2c(c1)ccc1c2ccc2[nH]c3ccccc3c21] | 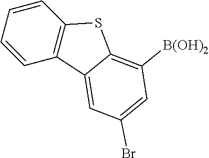 |
| 147 | 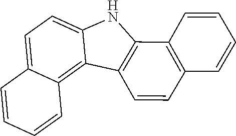 [SMILES: c1ccc2c(c1)ccc1c2[nH]c2ccc3ccccc3c21] | 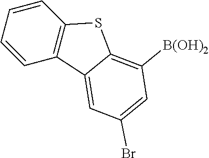 |
| 150 | 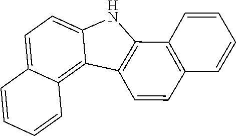 [SMILES: c1ccc2c(c1)ccc1c2[nH]c2ccc3ccccc3c21] | 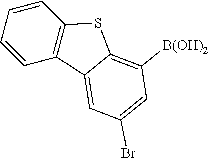 |
| 151 | 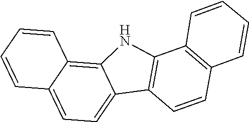 [SMILES: c1ccc2c(c1)ccc1c3ccc4ccccc4c3[nH]c21] | 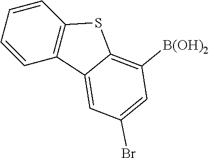 |
| 154 | 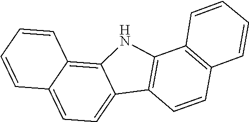 [SMILES: c1ccc2c(c1)ccc1c3ccc4ccccc4c3[nH]c21] | 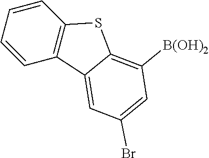 |
| 157 | 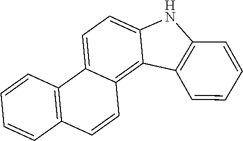 [SMILES: c1ccc2c(c1)ccc1c2ccc2[nH]c3ccccc3c21] | 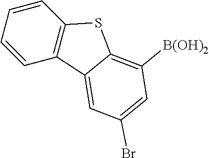 |
| 161 | 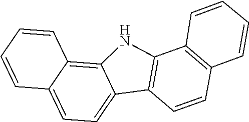 [SMILES: c1ccc2c(c1)ccc1c3ccc4ccccc4c3[nH]c21] | 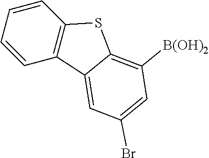 |
| 163 | 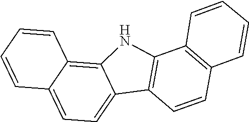 [SMILES: c1ccc2c(c1)ccc1c3ccc4ccccc4c3[nH]c21] | 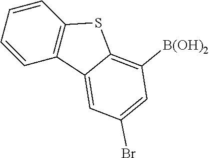 |
| 166 | 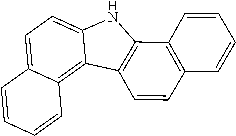 [SMILES: c1ccc2c(c1)ccc1c2[nH]c2ccc3ccccc3c21] | 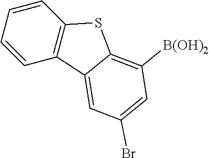 |
| 169 | 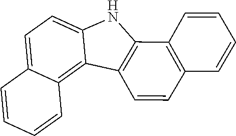 [SMILES: c1ccc2c(c1)ccc1c2[nH]c2ccc3ccccc3c21] | 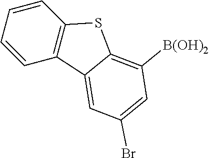 |
| 171 | 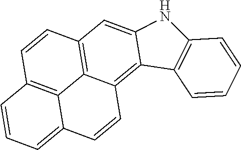 [SMILES: c1cc2ccc3cc4[nH]c5ccccc5c4c4ccc(c1)c2c34] | 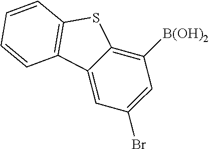 |
| 174 | 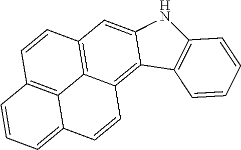 [SMILES: c1cc2ccc3cc4[nH]c5ccccc5c4c4ccc(c1)c2c34] | 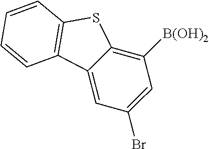 |
| 177 | 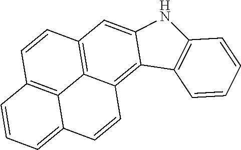 [SMILES: c1cc2ccc3cc4[nH]c5ccccc5c4c4ccc(c1)c2c34] | 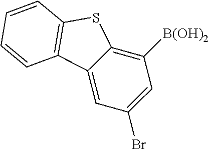 |
| 180 | 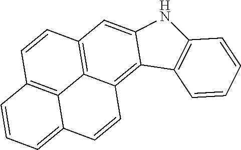 [SMILES: c1cc2ccc3cc4[nH]c5ccccc5c4c4ccc(c1)c2c34] | 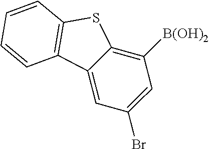 |
| 181 | 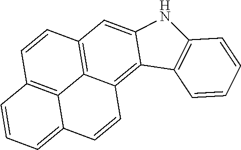 [SMILES: c1cc2ccc3cc4[nH]c5ccccc5c4c4ccc(c1)c2c34] | 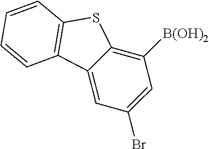 |
| 183 | 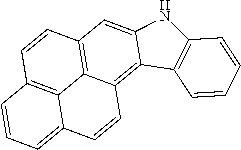 [SMILES: c1cc2ccc3cc4[nH]c5ccccc5c4c4ccc(c1)c2c34] | 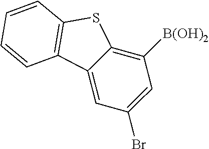 |
| 186 | 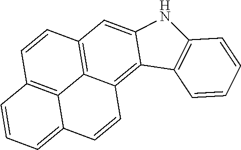 [SMILES: c1cc2ccc3cc4[nH]c5ccccc5c4c4ccc(c1)c2c34] | 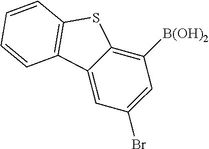 |
| 189 | 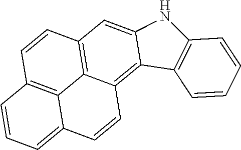 [SMILES: c1cc2ccc3cc4[nH]c5ccccc5c4c4ccc(c1)c2c34] | 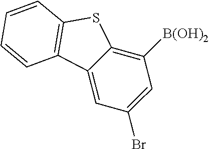 |
| 191 | 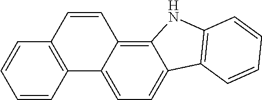 [SMILES: c1ccc2c(c1)ccc1c2ccc2c3ccccc3[nH]c21] | 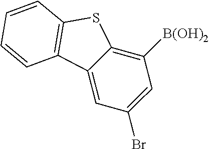 |
| 194 | 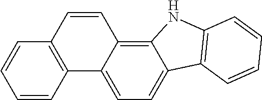 [SMILES: c1ccc2c(c1)ccc1c2ccc2c3ccccc3[nH]c21] | 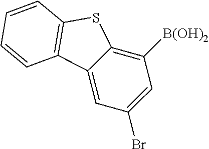 |
| 197 | 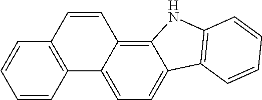 [SMILES: c1ccc2c(c1)ccc1c2ccc2c3ccccc3[nH]c21] | 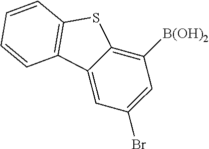 |
| 201 | 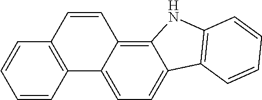 [SMILES: c1ccc2c(c1)ccc1c2ccc2c3ccccc3[nH]c21] | 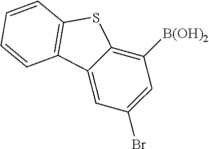 |
| 203 | 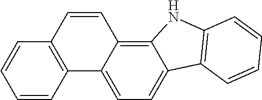 [SMILES: c1ccc2c(c1)ccc1c2ccc2c3ccccc3[nH]c21] | 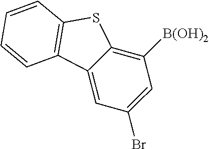 |
| 206 | 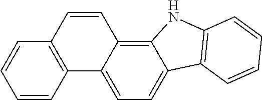 [SMILES: c1ccc2c(c1)ccc1c2ccc2c3ccccc3[nH]c21] | 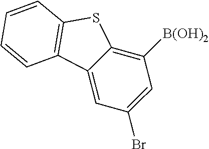 |
| 209 | 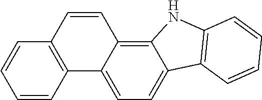 [SMILES: c1ccc2c(c1)ccc1c2ccc2c3ccccc3[nH]c21] | 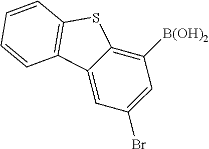 |
| 210 | 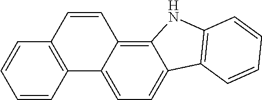 [SMILES: c1ccc2c(c1)ccc1c2ccc2c3ccccc3[nH]c21] | 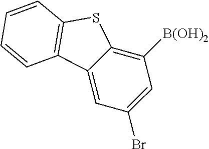 |

| Com- | | | |
|---|---|---|---|
| pound | Sub B | Product (P) | Yield |
| 1 | 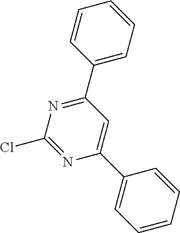 [SMILES: Clc1nc(-c2ccccc2)cc(-c2ccccc2)n1] | 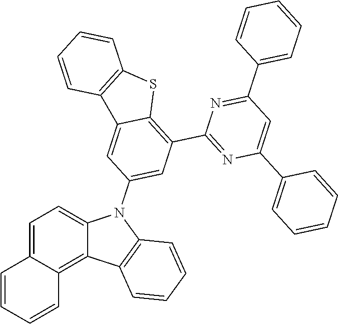 [SMILES: c1ccc(-c2cc(-c3ccccc3)nc(-c3cc(-n4c5ccccc5c5c6ccccc6ccc54)cc4c3sc3ccccc34)n2)cc1] | 54% |
| 3 | 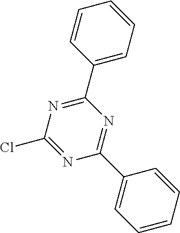 [SMILES: Clc1nc(-c2ccccc2)nc(-c2ccccc2)n1] | 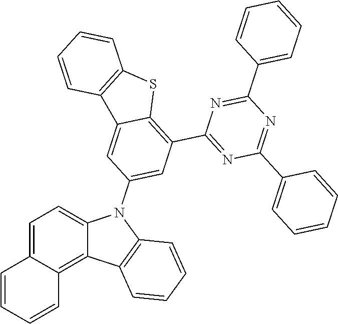 [SMILES: c1ccc(-c2nc(-c3ccccc3)nc(-c3cc(-n4c5ccccc5c5c6ccccc6ccc54)cc4c3sc3ccccc34)n2)cc1] | 68% |
| 5 | 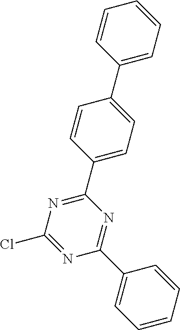 [SMILES: Clc1nc(-c2ccccc2)nc(-c2ccc(-c3ccccc3)cc2)n1] | 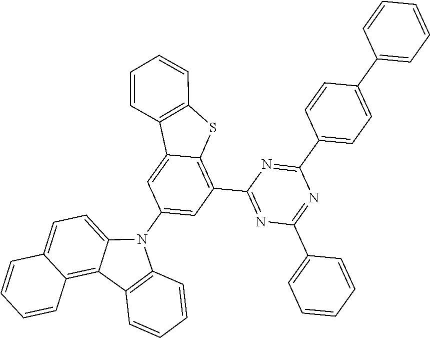 [SMILES: c1ccc(-c2ccc(-c3nc(-c4ccccc4)nc(-c4cc(-n5c6ccccc6c6c7ccccc7ccc65)cc5c4sc4ccccc45)n3)cc2)cc1] | 67% |
| 12 | 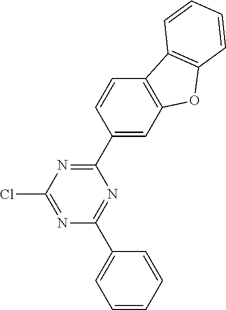 [SMILES: Clc1nc(-c2ccccc2)nc(-c2ccc3c(c2)oc2ccccc23)n1] | 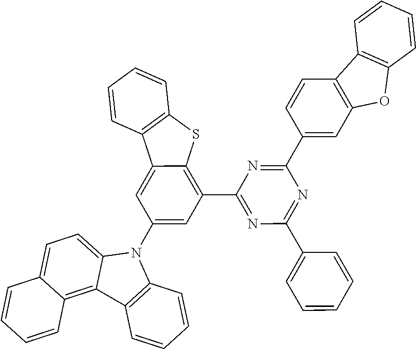 [SMILES: c1ccc(-c2nc(-c3ccc4c(c3)oc3ccccc34)nc(-c3cc(-n4c5ccccc5c5c6ccccc6ccc54)cc4c3sc3ccccc34)n2)cc1] | 63% |
| 18 | 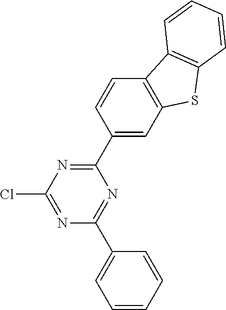 [SMILES: Clc1nc(-c2ccccc2)nc(-c2ccc3c(c2)sc2ccccc23)n1] | 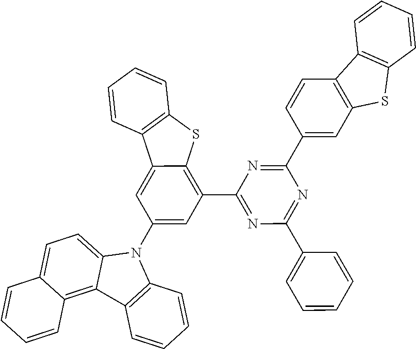 [SMILES: c1ccc(-c2nc(-c3ccc4c(c3)sc3ccccc34)nc(-c3cc(-n4c5ccccc5c5c6ccccc6ccc54)cc4c3sc3ccccc34)n2)cc1] | 59% |
| 22 | 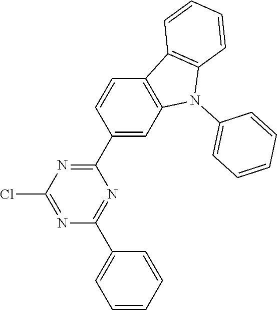 [SMILES: Clc1nc(-c2ccccc2)nc(-c2ccc3c4ccccc4n(-c4ccccc4)c3c2)n1] | 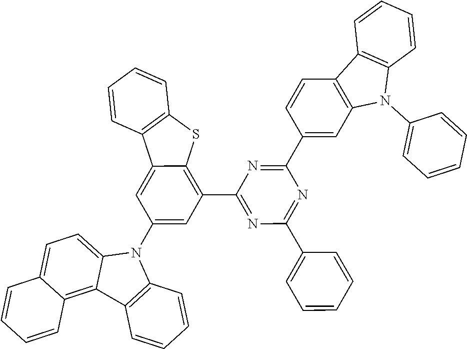 [SMILES: c1ccc(-c2nc(-c3ccc4c5ccccc5n(-c5ccccc5)c4c3)nc(-c3cc(-n4c5ccccc5c5c6ccccc6ccc54)cc4c3sc3ccccc34)n2)cc1] | 60% |
| 31 | 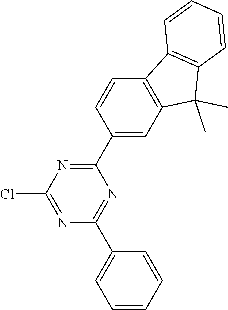 [SMILES: CC1(C)c2ccccc2-c2ccc(-c3nc(Cl)nc(-c4ccccc4)n3)cc21] | 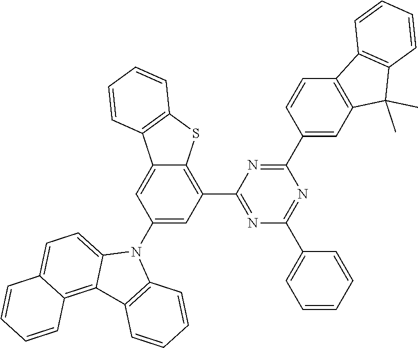 [SMILES: CC1(C)c2ccccc2-c2ccc(-c3nc(-c4ccccc4)nc(-c4cc(-n5c6ccccc6c6c7ccccc7ccc65)cc5c4sc4ccccc45)n3)cc21] | 51% |
| 34 | 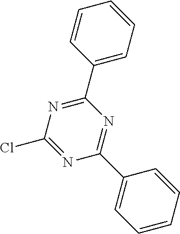 [SMILES: Clc1nc(-c2ccccc2)nc(-c2ccccc2)n1] | 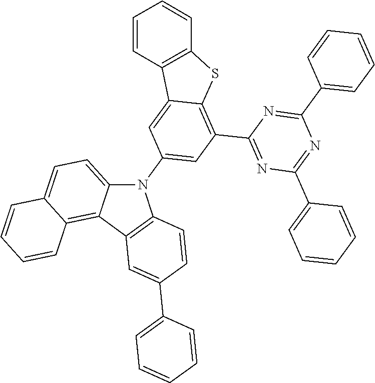 [SMILES: c1ccc(-c2ccc3c(c2)c2c4ccccc4ccc2n3-c2cc(-c3nc(-c4ccccc4)nc(-c4ccccc4)n3)c3sc4ccccc4c3c2)cc1] | 62% |
| 35 | 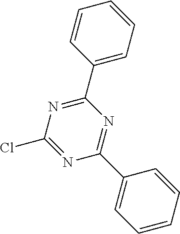 [SMILES: Clc1nc(-c2ccccc2)nc(-c2ccccc2)n1] | 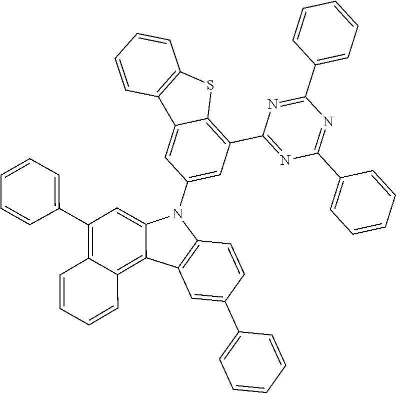 [SMILES: c1ccc(-c2ccc3c(c2)c2c4ccccc4c(-c4ccccc4)cc2n3-c2cc(-c3nc(-c4ccccc4)nc(-c4ccccc4)n3)c3sc4ccccc4c3c2)cc1] | 59% |
| 36 | 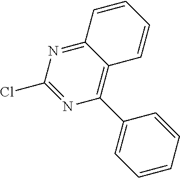 [SMILES: Clc1nc(-c2ccccc2)c2ccccc2n1] | 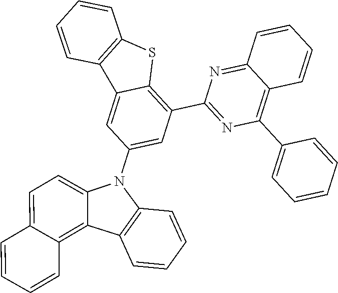 [SMILES: c1ccc(-c2nc(-c3cc(-n4c5ccccc5c5c6ccccc6ccc54)cc4c3sc3ccccc34)nc3ccccc23)cc1] | 62% |
| 38 | 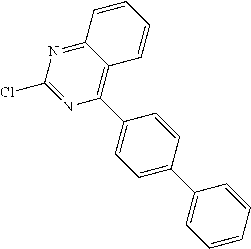 [SMILES: Clc1nc(-c2ccc(-c3ccccc3)cc2)c2ccccc2n1] | 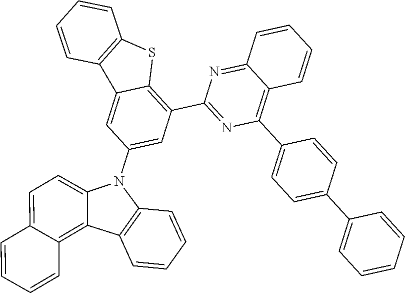 [SMILES: c1ccc(-c2ccc(-c3nc(-c4cc(-n5c6ccccc6c6c7ccccc7ccc65)cc5c4sc4ccccc45)nc4ccccc34)cc2)cc1] | 53% |
| 43 | 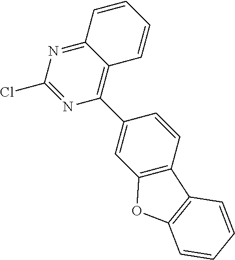 [SMILES: Clc1nc(-c2ccc3c(c2)oc2ccccc23)c2ccccc2n1] | 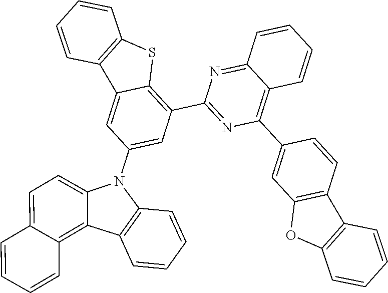 [SMILES: c1ccc2c(c1)ccc1c2c2ccccc2n1-c1cc(-c2nc(-c3ccc4c(c3)oc3ccccc34)c3ccccc3n2)c2sc3ccccc3c2c1] | 57% |
| 47 | 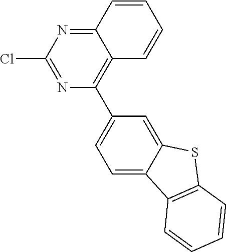 [SMILES: Clc1nc(-c2ccc3c(c2)sc2ccccc23)c2ccccc2n1] | 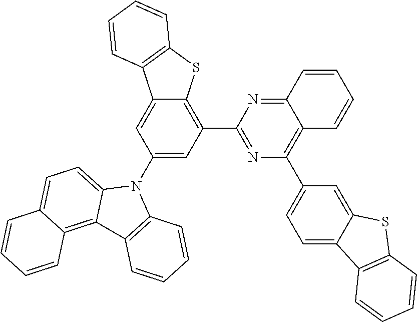 [SMILES: c1ccc2c(c1)ccc1c2c2ccccc2n1-c1cc(-c2nc(-c3ccc4c(c3)sc3ccccc34)c3ccccc3n2)c2sc3ccccc3c2c1] | 54% |
| 54 | 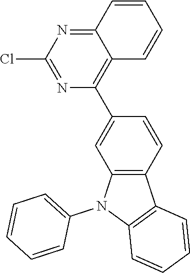 [SMILES: Clc1nc(-c2ccc3c4ccccc4n(-c4ccccc4)c3c2)c2ccccc2n1] | 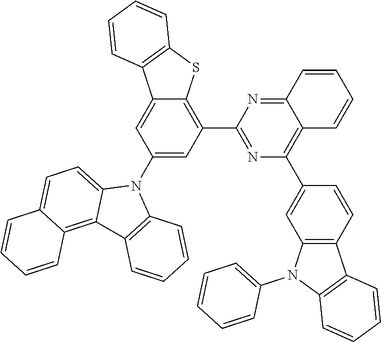 [SMILES: c1ccc(-n2c3ccccc3c3ccc(-c4nc(-c5cc(-n6c7ccccc7c7c8ccccc8ccc76)cc6c5sc5ccccc56)nc5ccccc45)cc32)cc1] | 63% |
| 59 | 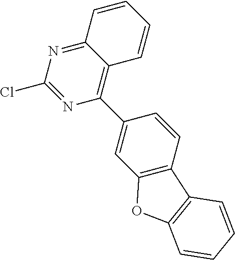 [SMILES: Clc1nc(-c2ccc3c(c2)oc2ccccc23)c2ccccc2n1] | 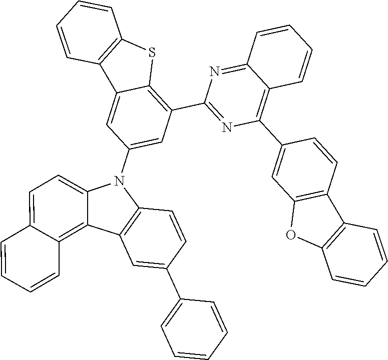 [SMILES: c1ccc(-c2ccc3c(c2)c2c4ccccc4ccc2n3-c2cc(-c3nc(-c4ccc5c(c4)oc4ccccc45)c4ccccc4n3)c3sc4ccccc4c3c2)cc1] | 59% |
| 61 | 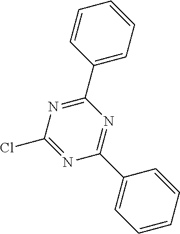 [SMILES: Clc1nc(-c2ccccc2)nc(-c2ccccc2)n1] | 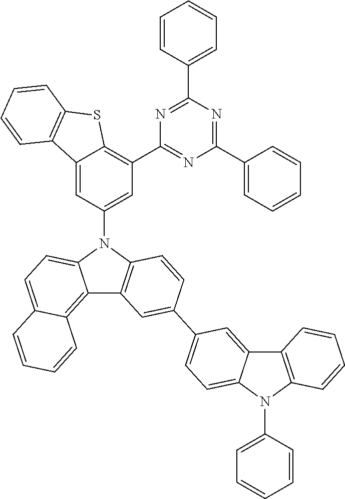 [SMILES: c1ccc(-c2nc(-c3ccccc3)nc(-c3cc(-n4c5ccc(-c6ccc7c(c6)c6ccccc6n7-c6ccccc6)cc5c5c6ccccc6ccc54)cc4c3sc3ccccc34)n2)cc1] | 51% |
| 64 | 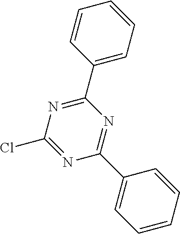 [SMILES: Clc1nc(-c2ccccc2)nc(-c2ccccc2)n1] | 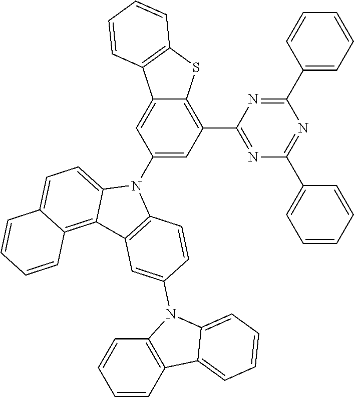 [SMILES: c1ccc(-c2nc(-c3ccccc3)nc(-c3cc(-n4c5ccc(-n6c7ccccc7c7ccccc76)cc5c5c6ccccc6ccc54)cc4c3sc3ccccc34)n2)cc1] | 53% |
| 67 | 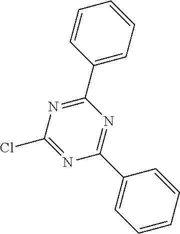 [SMILES: Clc1nc(-c2ccccc2)nc(-c2ccccc2)n1] | 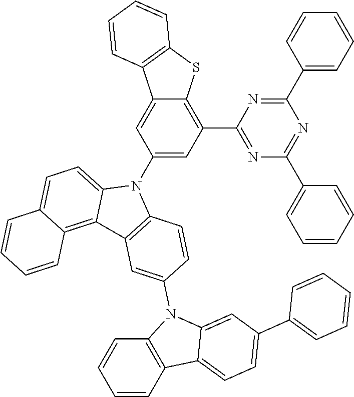 [SMILES: c1ccc(-c2ccc3c4ccccc4n(-c4ccc5c(c4)c4c6ccccc6ccc4n5-c4cc(-c5nc(-c6ccccc6)nc(-c6ccccc6)n5)c5sc6ccccc6c5c4)c3c2)cc1] | 57% |
| 68 | 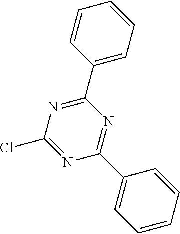 [SMILES: Clc1nc(-c2ccccc2)nc(-c2ccccc2)n1] | 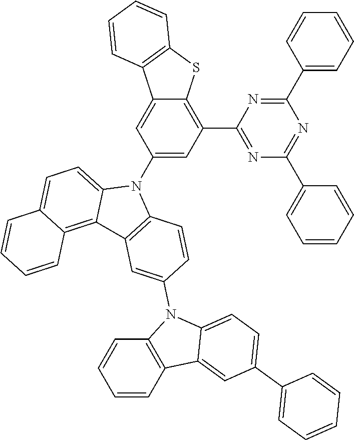 [SMILES: c1ccc(-c2ccc3c(c2)c2ccccc2n3-c2ccc3c(c2)c2c4ccccc4ccc2n3-c2cc(-c3nc(-c4ccccc4)nc(-c4ccccc4)n3)c3sc4ccccc4c3c2)cc1] | 55% |
| 69 | 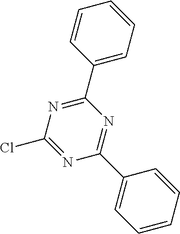 [SMILES: Clc1nc(-c2ccccc2)nc(-c2ccccc2)n1] | 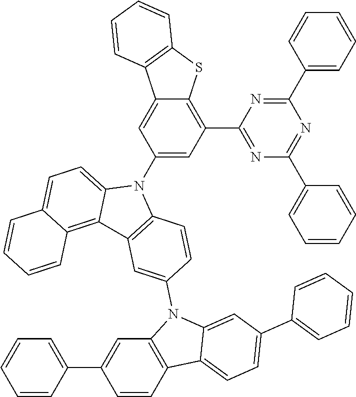 [SMILES: c1ccc(-c2ccc3c4ccc(-c5ccccc5)cc4n(-c4ccc5c(c4)c4c6ccccc6ccc4n5-c4cc(-c5nc(-c6ccccc6)nc(-c6ccccc6)n5)c5sc6ccccc6c5c4)c3c2)cc1] | 56% |
| 70 | 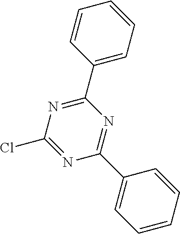 [SMILES: Clc1nc(-c2ccccc2)nc(-c2ccccc2)n1] | 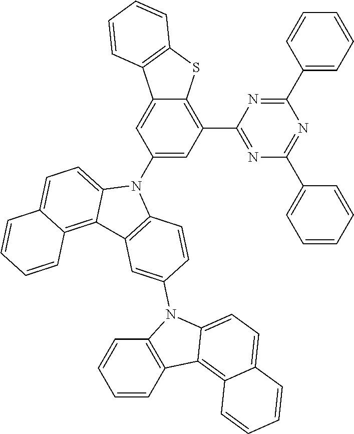 [SMILES: c1ccc(-c2nc(-c3ccccc3)nc(-c3cc(-n4c5ccc(-n6c7ccccc7c7c8ccccc8ccc76)cc5c5c6ccccc6ccc54)cc4c3sc3ccccc34)n2)cc1] | 61% |
| 71 | 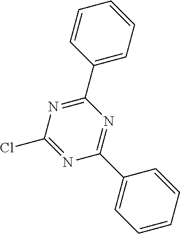 [SMILES: Clc1nc(-c2ccccc2)nc(-c2ccccc2)n1] | 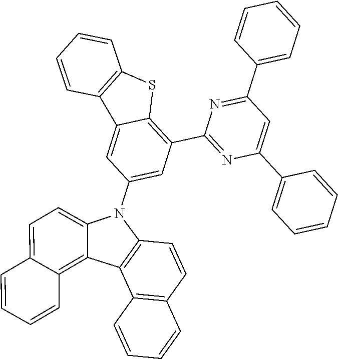 [SMILES: c1ccc(-c2cc(-c3ccccc3)nc(-c3cc(-n4c5ccc6ccccc6c5c5c6ccccc6ccc54)cc4c3sc3ccccc34)n2)cc1] | 58% |
| 73 | 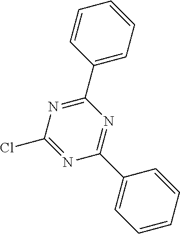 [SMILES: Clc1nc(-c2ccccc2)nc(-c2ccccc2)n1] | 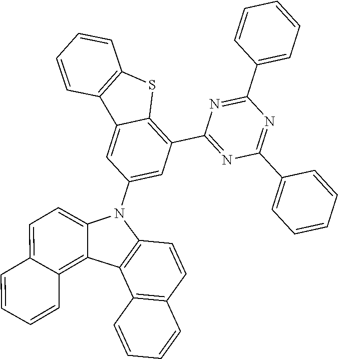 [SMILES: c1ccc(-c2nc(-c3ccccc3)nc(-c3cc(-n4c5ccc6ccccc6c5c5c6ccccc6ccc54)cc4c3sc3ccccc34)n2)cc1] | 55% |
| 75 | 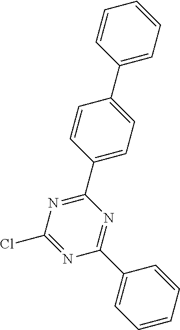 [SMILES: Clc1nc(-c2ccccc2)nc(-c2ccc(-c3ccccc3)cc2)n1] | 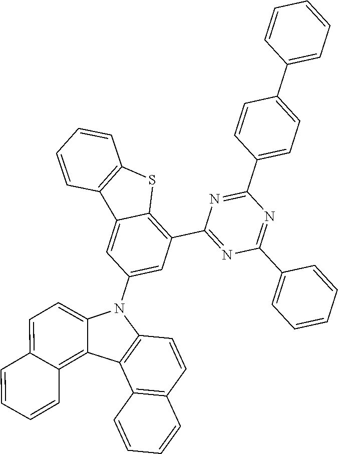 [SMILES: c1ccc(-c2ccc(-c3nc(-c4ccccc4)nc(-c4cc(-n5c6ccc7ccccc7c6c6c7ccccc7ccc65)cc5c4sc4ccccc45)n3)cc2)cc1] | 62% |
| 82 | 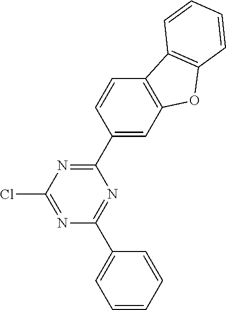 [SMILES: Clc1nc(-c2ccccc2)nc(-c2ccc3c(c2)oc2ccccc23)n1] | 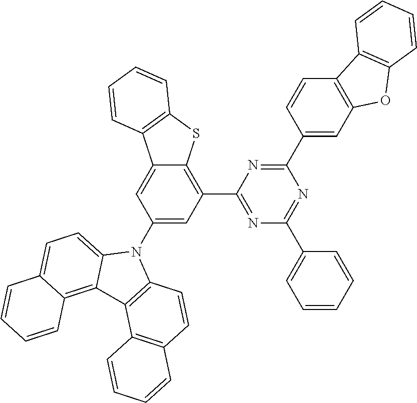 [SMILES: c1ccc(-c2nc(-c3ccc4c(c3)oc3ccccc34)nc(-c3cc(-n4c5ccc6ccccc6c5c5c6ccccc6ccc54)cc4c3sc3ccccc34)n2)cc1] | 60% |
| 88 | 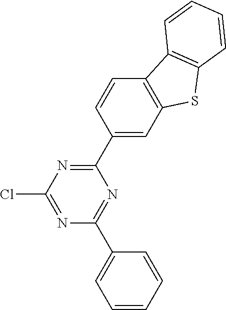 [SMILES: Clc1nc(-c2ccccc2)nc(-c2ccc3c(c2)sc2ccccc23)n1] | 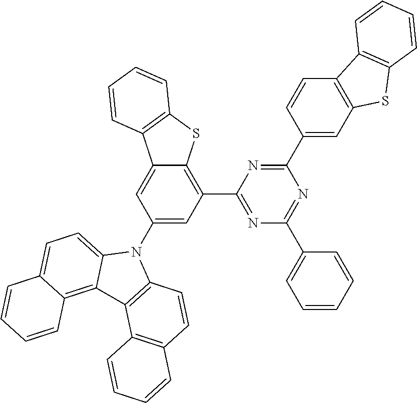 [SMILES: c1ccc(-c2nc(-c3ccc4c(c3)sc3ccccc34)nc(-c3cc(-n4c5ccc6ccccc6c5c5c6ccccc6ccc54)cc4c3sc3ccccc34)n2)cc1] | 59% |
| 92 | 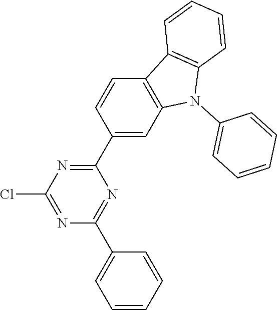 [SMILES: Clc1nc(-c2ccccc2)nc(-c2ccc3c4ccccc4n(-c4ccccc4)c3c2)n1] | 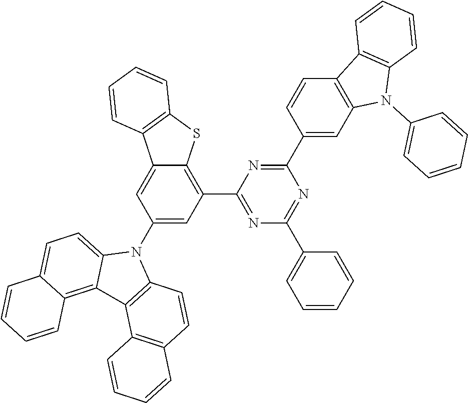 [SMILES: c1ccc(-c2nc(-c3ccc4c5ccccc5n(-c5ccccc5)c4c3)nc(-c3cc(-n4c5ccc6ccccc6c5c5c6ccccc6ccc54)cc4c3sc3ccccc34)n2)cc1] | 49% |
| 104 | 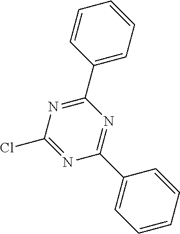 [SMILES: Clc1nc(-c2ccccc2)nc(-c2ccccc2)n1] | 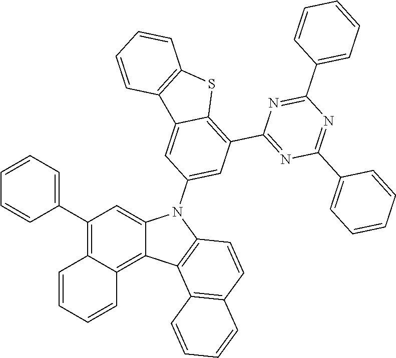 [SMILES: c1ccc(-c2nc(-c3ccccc3)nc(-c3cc(-n4c5ccc6ccccc6c5c5c6ccccc6c(-c6ccccc6)cc54)cc4c3sc3ccccc34)n2)cc1] | 67% |
| 105 | 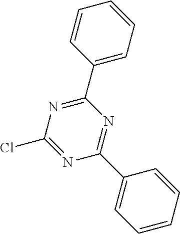 [SMILES: Clc1nc(-c2ccccc2)nc(-c2ccccc2)n1] | 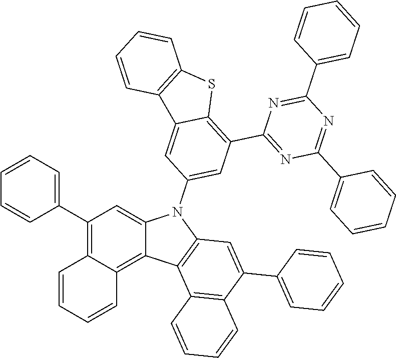 [SMILES: c1ccc(-c2nc(-c3ccccc3)nc(-c3cc(-n4c5cc(-c6ccccc6)c6ccccc6c5c5c6ccccc6c(-c6ccccc6)cc54)cc4c3sc3ccccc34)n2)cc1] | 63% |
| 106 | 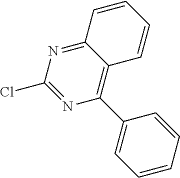 [SMILES: Clc1nc(-c2ccccc2)c2ccccc2n1] | 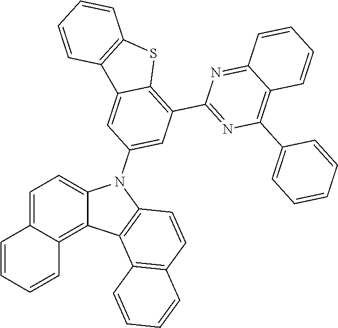 [SMILES: c1ccc(-c2nc(-c3cc(-n4c5ccc6ccccc6c5c5c6ccccc6ccc54)cc4c3sc3ccccc34)nc3ccccc23)cc1] | 56% |
| 108 | 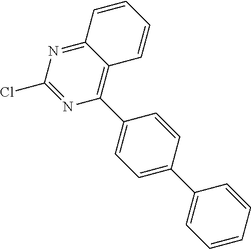 [SMILES: Clc1nc(-c2ccc(-c3ccccc3)cc2)c2ccccc2n1] | 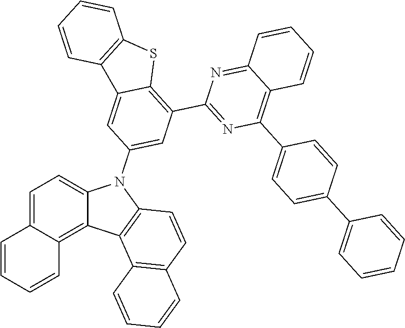 [SMILES: c1ccc(-c2ccc(-c3nc(-c4cc(-n5c6ccc7ccccc7c6c6c7ccccc7ccc65)cc5c4sc4ccccc45)nc4ccccc34)cc2)cc1] | 61% |
| 113 | 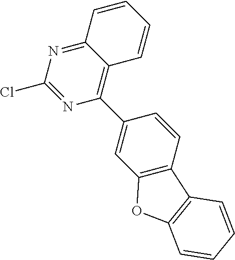 [SMILES: Clc1nc(-c2ccc3c(c2)oc2ccccc23)c2ccccc2n1] | 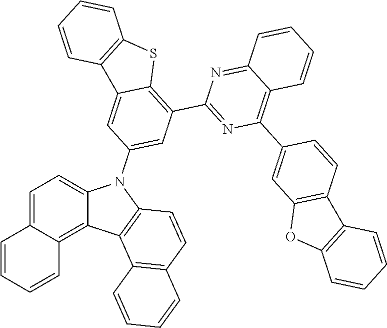 [SMILES: c1ccc2c(c1)ccc1c2c2c3ccccc3ccc2n1-c1cc(-c2nc(-c3ccc4c(c3)oc3ccccc34)c3ccccc3n2)c2sc3ccccc3c2c1] | 67% |
| 117 | 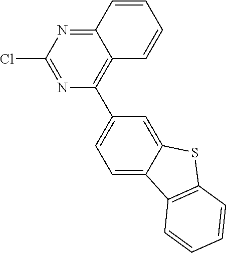 [SMILES: Clc1nc(-c2ccc3c(c2)sc2ccccc23)c2ccccc2n1] | 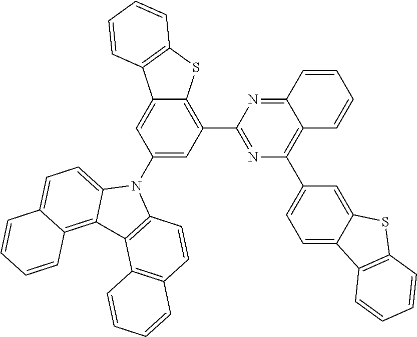 [SMILES: c1ccc2c(c1)ccc1c2c2c3ccccc3ccc2n1-c1cc(-c2nc(-c3ccc4c(c3)sc3ccccc34)c3ccccc3n2)c2sc3ccccc3c2c1] | 64% |
| 124 | 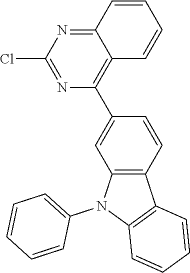 [SMILES: Clc1nc(-c2ccc3c4ccccc4n(-c4ccccc4)c3c2)c2ccccc2n1] | 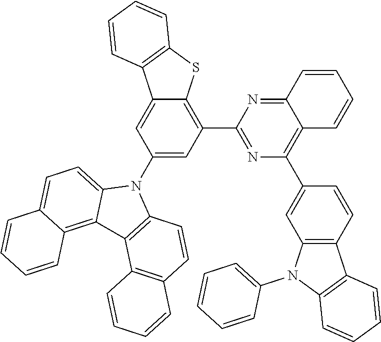 [SMILES: c1ccc(-n2c3ccccc3c3ccc(-c4nc(-c5cc(-n6c7ccc8ccccc8c7c7c8ccccc8ccc76)cc6c5sc5ccccc56)nc5ccccc45)cc32)cc1] | 51% |
| 129 | 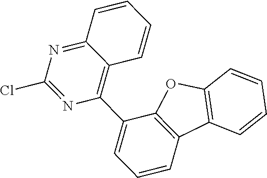 [SMILES: Clc1nc(-c2cccc3c2oc2ccccc23)c2ccccc2n1] | 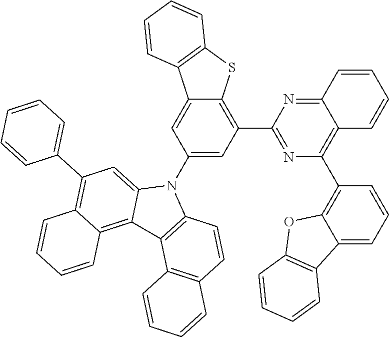 [SMILES: c1ccc(-c2cc3c(c4ccccc24)c2c4ccccc4ccc2n3-c2cc(-c3nc(-c4cccc5c4oc4ccccc45)c4ccccc4n3)c3sc4ccccc4c3c2)cc1] | 58% |
| 136 | 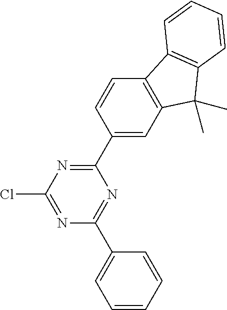 [SMILES: CC1(C)c2ccccc2-c2ccc(-c3nc(Cl)nc(-c4ccccc4)n3)cc21] | 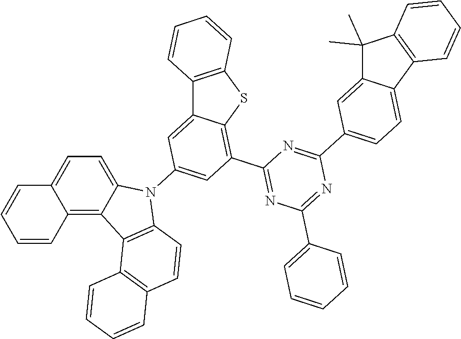 [SMILES: CC1(C)c2ccccc2-c2ccc(-c3nc(-c4ccccc4)nc(-c4cc(-n5c6ccc7ccccc7c6c6c7ccccc7ccc65)cc5c4sc4ccccc45)n3)cc21] | 50% |
| 141 | 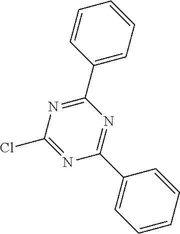 [SMILES: Clc1nc(-c2ccccc2)nc(-c2ccccc2)n1] | 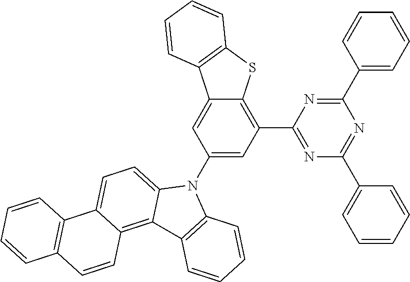 [SMILES: c1ccc(-c2nc(-c3ccccc3)nc(-c3cc(-n4c5ccccc5c5c6ccc7ccccc7c6ccc54)cc4c3sc3ccccc34)n2)cc1] | 56% |
| 144 | 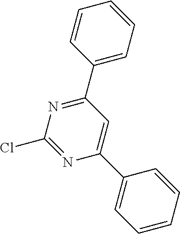 [SMILES: Clc1nc(-c2ccccc2)cc(-c2ccccc2)n1] | 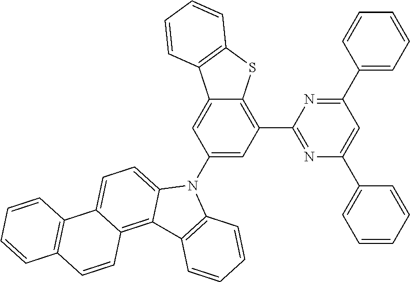 [SMILES: c1ccc(-c2cc(-c3ccccc3)nc(-c3cc(-n4c5ccccc5c5c6ccc7ccccc7c6ccc54)cc4c3sc3ccccc34)n2)cc1] | 58% |
| 147 | 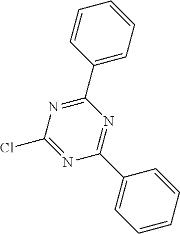 [SMILES: Clc1nc(-c2ccccc2)nc(-c2ccccc2)n1] | 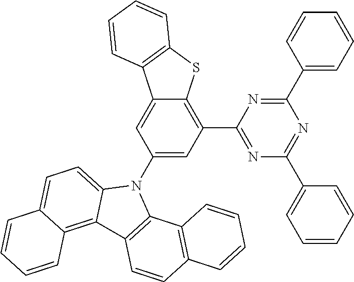 [SMILES: c1ccc(-c2nc(-c3ccccc3)nc(-c3cc(-n4c5ccc6ccccc6c5c5ccc6ccccc6c54)cc4c3sc3ccccc34)n2)cc1] | 55% |
| 150 | 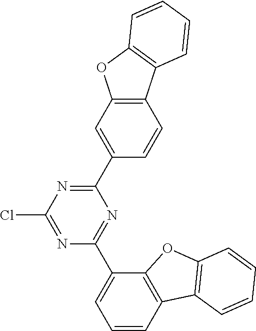 [SMILES: Clc1nc(-c2ccc3c(c2)oc2ccccc23)nc(-c2cccc3c2oc2ccccc23)n1] | 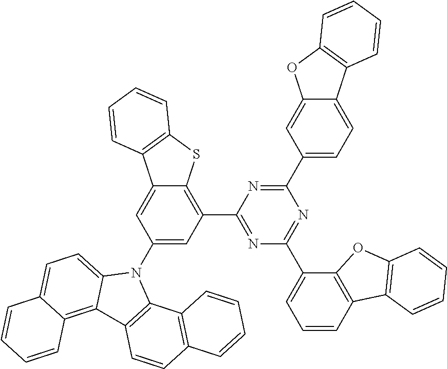 [SMILES: c1ccc2c(c1)ccc1c2c2ccc3ccccc3c2n1-c1cc(-c2nc(-c3ccc4c(c3)oc3ccccc34)nc(-c3cccc4c3oc3ccccc34)n2)c2sc3ccccc3c2c1] | 52% |
| 151 | 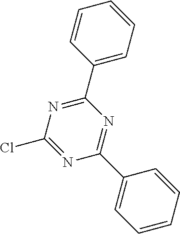 [SMILES: Clc1nc(-c2ccccc2)nc(-c2ccccc2)n1] | 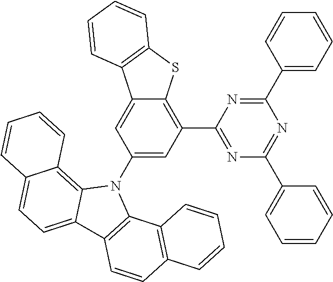 [SMILES: c1ccc(-c2nc(-c3ccccc3)nc(-c3cc(-n4c5c6ccccc6ccc5c5ccc6ccccc6c54)cc4c3sc3ccccc34)n2)cc1] | 67% |
| 154 | 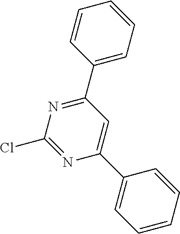 [SMILES: Clc1nc(-c2ccccc2)cc(-c2ccccc2)n1] | 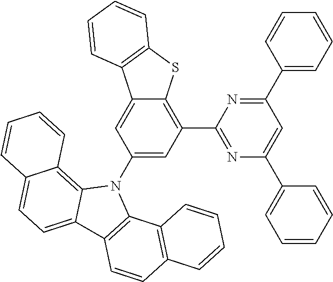 [SMILES: c1ccc(-c2cc(-c3ccccc3)nc(-c3cc(-n4c5c6ccccc6ccc5c5ccc6ccccc6c54)cc4c3sc3ccccc34)n2)cc1] | 63% |
| 157 | 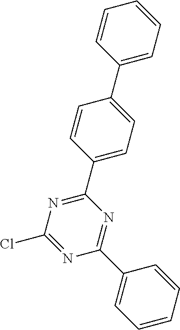 [SMILES: Clc1nc(-c2ccccc2)nc(-c2ccc(-c3ccccc3)cc2)n1] | 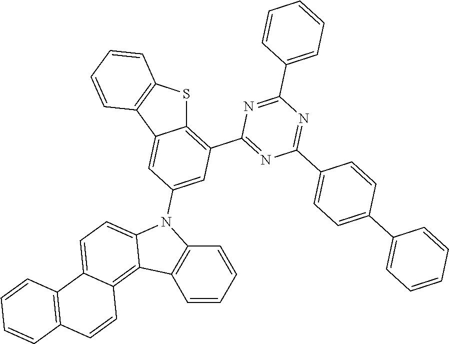 [SMILES: c1ccc(-c2ccc(-c3nc(-c4ccccc4)nc(-c4cc(-n5c6ccccc6c6c7ccc8ccccc8c7ccc65)cc5c4sc4ccccc45)n3)cc2)cc1] | 66% |
| 161 | 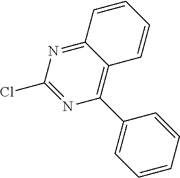 [SMILES: Clc1nc(-c2ccccc2)c2ccccc2n1] | 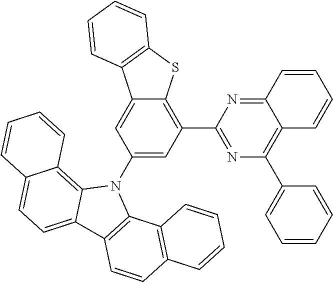 [SMILES: c1ccc(-c2nc(-c3cc(-n4c5c6ccccc6ccc5c5ccc6ccccc6c54)cc4c3sc3ccccc34)nc3ccccc23)cc1] | 61% |
| 163 | 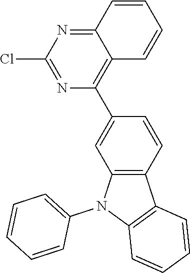 [SMILES: Clc1nc(-c2ccc3c4ccccc4n(-c4ccccc4)c3c2)c2ccccc2n1] | 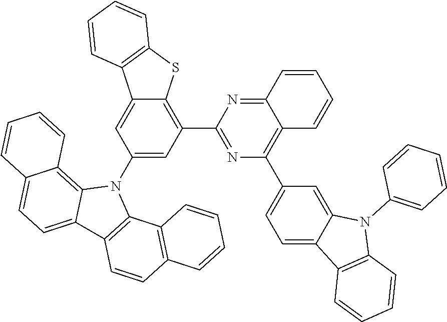 [SMILES: c1ccc(-n2c3ccccc3c3ccc(-c4nc(-c5cc(-n6c7c8ccccc8ccc7c7ccc8ccccc8c76)cc6c5sc5ccccc56)nc5ccccc45)cc32)cc1] | 52% |
| 166 | 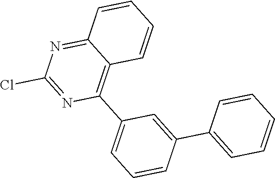 [SMILES: Clc1nc(-c2cccc(-c3ccccc3)c2)c2ccccc2n1] | 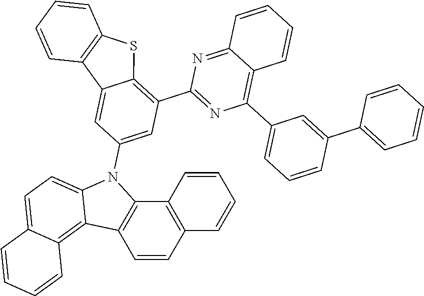 [SMILES: c1ccc(-c2cccc(-c3nc(-c4cc(-n5c6ccc7ccccc7c6c6ccc7ccccc7c65)cc5c4sc4ccccc45)nc4ccccc34)c2)cc1] | 67% |
| 169 | 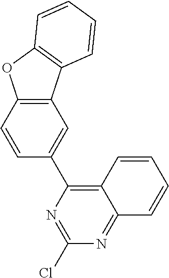 [SMILES: Clc1nc(-c2ccc3oc4ccccc4c3c2)c2ccccc2n1] | 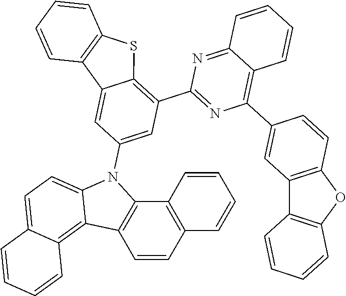 [SMILES: c1ccc2c(c1)ccc1c2c2ccc3ccccc3c2n1-c1cc(-c2nc(-c3ccc4oc5ccccc5c4c3)c3ccccc3n2)c2sc3ccccc3c2c1] | 52% |
| 171 | 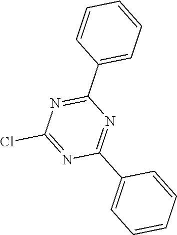 [SMILES: Clc1nc(-c2ccccc2)nc(-c2ccccc2)n1] | 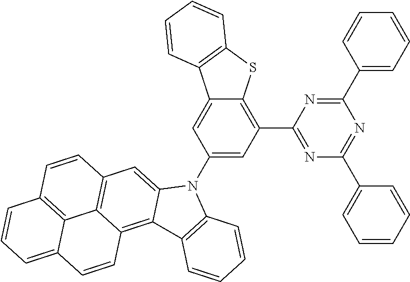 [SMILES: c1ccc(-c2nc(-c3ccccc3)nc(-c3cc(-n4c5ccccc5c5c6ccc7cccc8ccc(cc54)c6c87)cc4c3sc3ccccc34)n2)cc1] | 64% |
| 174 | 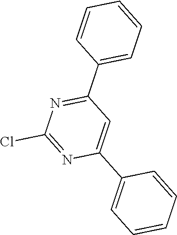 [SMILES: Clc1nc(-c2ccccc2)cc(-c2ccccc2)n1] | 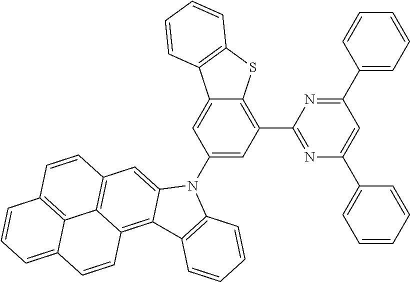 [SMILES: c1ccc(-c2cc(-c3ccccc3)nc(-c3cc(-n4c5ccccc5c5c6ccc7cccc8ccc(cc54)c6c87)cc4c3sc3ccccc34)n2)cc1] | 69% |
| 177 | 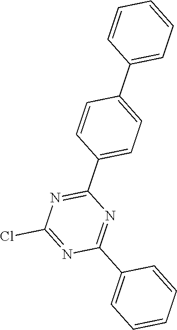 [SMILES: Clc1nc(-c2ccccc2)nc(-c2ccc(-c3ccccc3)cc2)n1] | 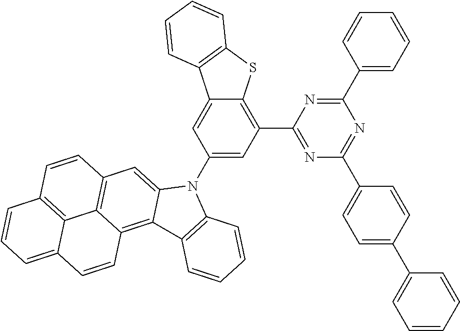 [SMILES: c1ccc(-c2ccc(-c3nc(-c4ccccc4)nc(-c4cc(-n5c6ccccc6c6c7ccc8cccc9ccc(cc65)c7c98)cc5c4sc4ccccc45)n3)cc2)cc1] | 71% |
| 180 | 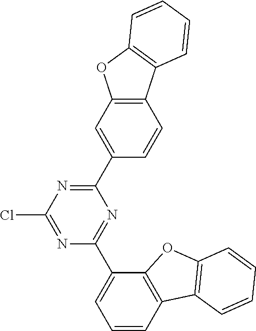 [SMILES: Clc1nc(-c2ccc3c(c2)oc2ccccc23)nc(-c2cccc3c2oc2ccccc23)n1] | 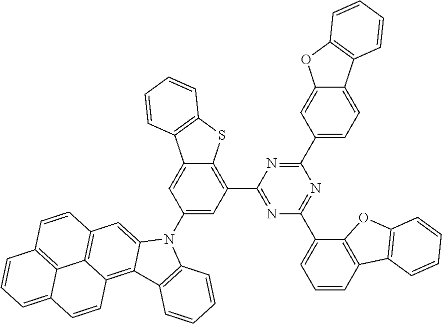 [SMILES: c1cc2ccc3cc4c(c5ccc(c1)c2c35)c1ccccc1n4-c1cc(-c2nc(-c3ccc4c(c3)oc3ccccc34)nc(-c3cccc4c3oc3ccccc34)n2)c2sc3ccccc3c2c1] | 52% |
| 181 | 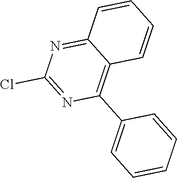 [SMILES: Clc1nc(-c2ccccc2)c2ccccc2n1] | 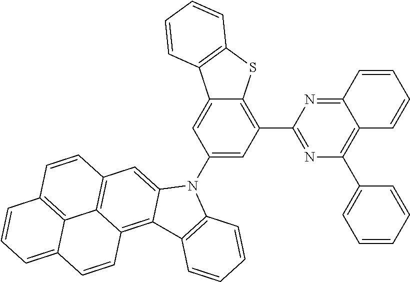 [SMILES: c1ccc(-c2nc(-c3cc(-n4c5ccccc5c5c6ccc7cccc8ccc(cc54)c6c87)cc4c3sc3ccccc34)nc3ccccc23)cc1] | 53% |
| 183 | 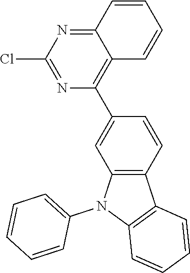 [SMILES: Clc1nc(-c2ccc3c4ccccc4n(-c4ccccc4)c3c2)c2ccccc2n1] | 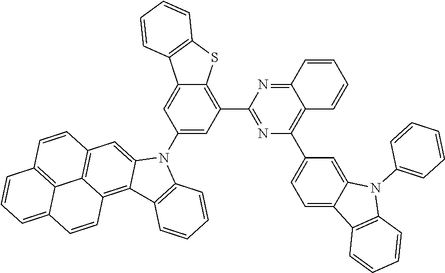 [SMILES: c1ccc(-n2c3ccccc3c3ccc(-c4nc(-c5cc(-n6c7ccccc7c7c8ccc9cccc%10ccc(cc76)c8c%109)cc6c5sc5ccccc56)nc5ccccc45)cc32)cc1] | 49% |
| 186 | 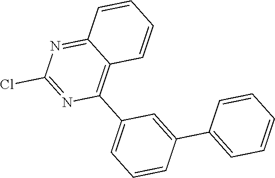 [SMILES: Clc1nc(-c2cccc(-c3ccccc3)c2)c2ccccc2n1] | 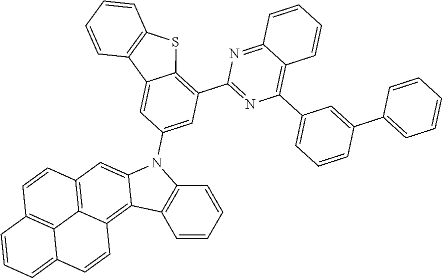 [SMILES: c1ccc(-c2cccc(-c3nc(-c4cc(-n5c6ccccc6c6c7ccc8cccc9ccc(cc65)c7c98)cc5c4sc4ccccc45)nc4ccccc34)c2)cc1] | 57% |
| 189 | 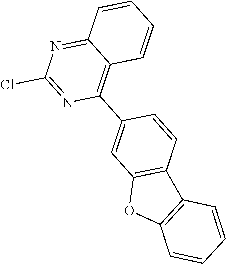 [SMILES: Clc1nc(-c2ccc3c(c2)oc2ccccc23)c2ccccc2n1] | 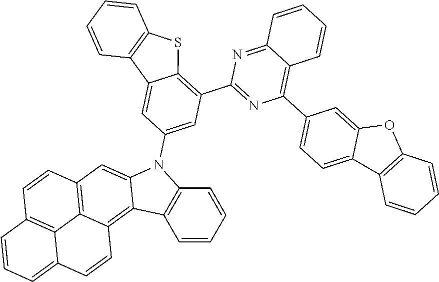 [SMILES: c1cc2ccc3cc4c(c5ccc(c1)c2c35)c1ccccc1n4-c1cc(-c2nc(-c3ccc4c(c3)oc3ccccc34)c3ccccc3n2)c2sc3ccccc3c2c1] | 54% |
| 191 | 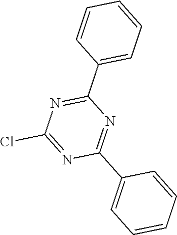 [SMILES: Clc1nc(-c2ccccc2)nc(-c2ccccc2)n1] | 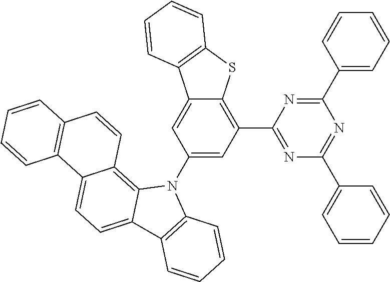 [SMILES: c1ccc(-c2nc(-c3ccccc3)nc(-c3cc(-n4c5ccccc5c5ccc6c7ccccc7ccc6c54)cc4c3sc3ccccc34)n2)cc1] | 55% |
| 194 | 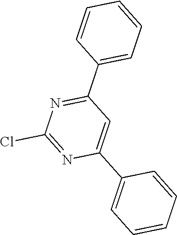 [SMILES: Clc1nc(-c2ccccc2)cc(-c2ccccc2)n1] | 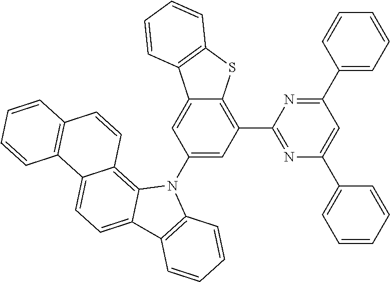 [SMILES: c1ccc(-c2cc(-c3ccccc3)nc(-c3cc(-n4c5ccccc5c5ccc6c7ccccc7ccc6c54)cc4c3sc3ccccc34)n2)cc1] | 59% |
| 197 | 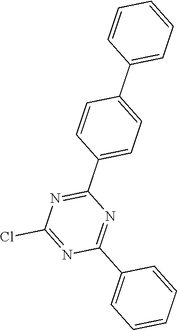 [SMILES: Clc1nc(-c2ccccc2)nc(-c2ccc(-c3ccccc3)cc2)n1] | 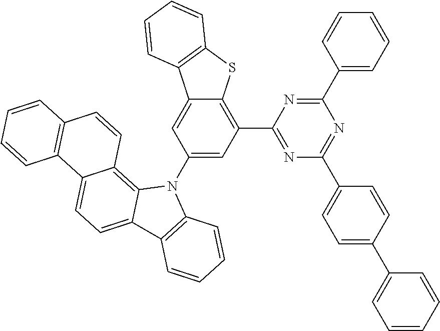 [SMILES: c1ccc(-c2ccc(-c3nc(-c4ccccc4)nc(-c4cc(-n5c6ccccc6c6ccc7c8ccccc8ccc7c65)cc5c4sc4ccccc45)n3)cc2)cc1] | 64% |
| 201 | 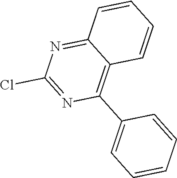 [SMILES: Clc1nc(-c2ccccc2)c2ccccc2n1] | 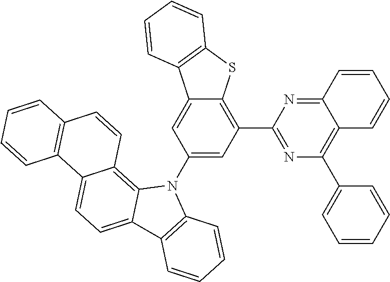 [SMILES: c1ccc(-c2nc(-c3cc(-n4c5ccccc5c5ccc6c7ccccc7ccc6c54)cc4c3sc3ccccc34)nc3ccccc23)cc1] | 53% |
| 203 | 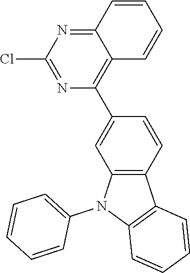 [SMILES: Clc1nc(-c2ccc3c4ccccc4n(-c4ccccc4)c3c2)c2ccccc2n1] | 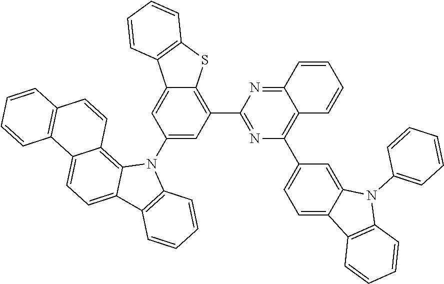 [SMILES: c1ccc(-n2c3ccccc3c3ccc(-c4nc(-c5cc(-n6c7ccccc7c7ccc8c9ccccc9ccc8c76)cc6c5sc5ccccc56)nc5ccccc45)cc32)cc1] | 47% |
| 206 | 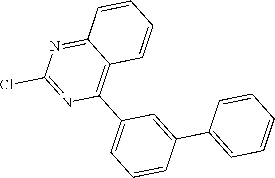 [SMILES: Clc1nc(-c2cccc(-c3ccccc3)c2)c2ccccc2n1] | 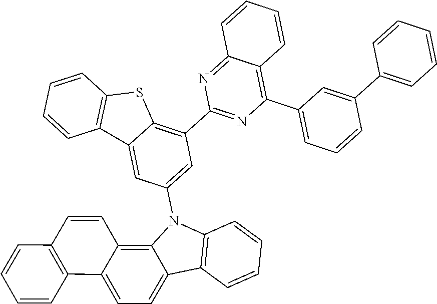 [SMILES: c1ccc(-c2cccc(-c3nc(-c4cc(-n5c6ccccc6c6ccc7c8ccccc8ccc7c65)cc5c4sc4ccccc45)nc4ccccc34)c2)cc1] | 70% |
| 209 | 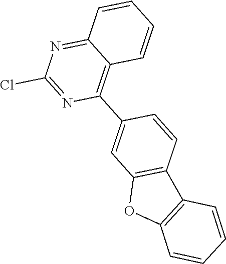 [SMILES: Clc1nc(-c2ccc3c(c2)oc2ccccc23)c2ccccc2n1] | 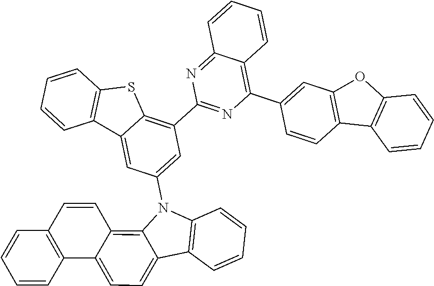 [SMILES: c1ccc2c(c1)ccc1c2ccc2c3ccccc3n(-c3cc(-c4nc(-c5ccc6c(c5)oc5ccccc56)c5ccccc5n4)c4sc5ccccc5c4c3)c12] | 52% |
| 210 | 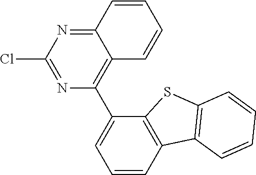 [SMILES: Clc1nc(-c2cccc3c2sc2ccccc23)c2ccccc2n1] | 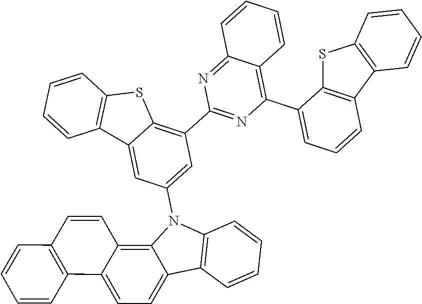 [SMILES: c1ccc2c(c1)ccc1c2ccc2c3ccccc3n(-c3cc(-c4nc(-c5cccc6c5sc5ccccc56)c5ccccc5n4)c4sc5ccccc5c4c3)c12] | 59% |

As a comparative example, the following compounds were used.

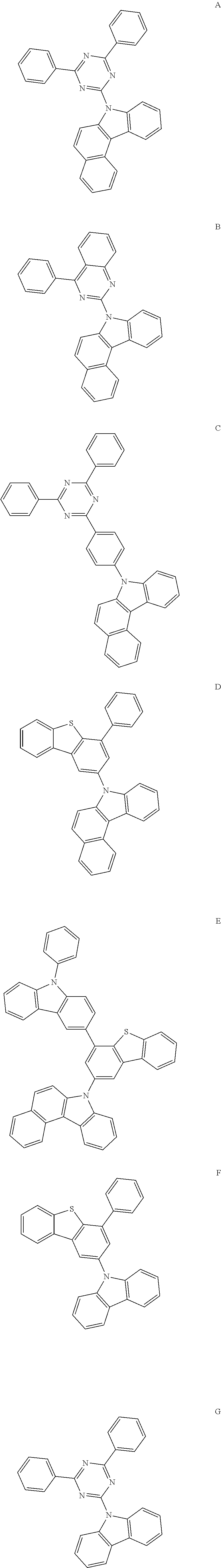

The prepared compound was confirmed from Mass results.

TABLE 2

| Com- | | Com- | |
|---|---|---|---|
| pound | FD-Mass | pound | FD-Mass |
| 1 | m/z = 629.78(C₄₄H₂₇N₃S = 629.19) | 2 | m/z = 629.78(C₄₄H₂₇N₃S = 629.19) |
| 3 | m/z = 630.77(C₄₃H₂₆N₄S = 630.19) | 4 | m/z = 706.87(C₄₉H₃₀N₄S = 706.22) |
| 5 | m/z = 706.87(C₄₉H₃₀N₄S = 706.22) | 6 | m/z = 680.83(C₄₇H₂₈N₄S = 680.20) |
| 7 | m/z = 730.89(C₅₁H₃₀N₄S = 730.22) | 8 | m/z = 680.83(C₄₇H₂₈N₄S = 680.20) |
| 9 | m/z = 730.89(C₅₁H₃₀N₄S = 730.22) | 10 | m/z = 720.85(C₄₉H₂₈N₄OS = 720.20) |
| 11 | m/z = 770.91(C₅₃H₃₀N₄OS = 770.21) | 12 | m/z = 720.85(C₄₉H₂₈N₄OS = 720.20) |
| 13 | m/z = 720.85(C₄₉H₂₈N₄OS = 720.20) | 14 | m/z = 720.85(C₄₉H₂₈N₄OS = 720.20) |
| 15 | m/z = 770.91(C₅₃H₃₀N₄OS = 770.21) | 16 | m/z = 770.91(C₅₃H₃₀N₄OS = 770.21) |
| 17 | m/z = 736.91(C₄₉H₂₈N₄S₂ = 736.18) | 18 | m/z = 736.91(C₄₉H₂₈N₄S₂ = 736.18) |
| 19 | m/z = 736.91(C₄₉H₂₈N₄S₂ = 736.18) | 20 | m/z = 786.97(C₅₃H₃₀N₄S₂ = 786.19) |
| 21 | m/z = 786.97(C₅₃H₃₀N₄S₂ = 786.19) | 22 | m/z = 795.96(C₅₅H₃₃N₅S = 795.25) |
| 23 | m/z = 795.96(C₅₅H₃₃N₅S = 795.25) | 24 | m/z = 795.96(C₅₅H₃₃N₅S = 795.25) |
| 25 | m/z = 719.87(C₄₉H₂₉N₅S = 719.21) | 26 | m/z = 795.96(C₅₅H₃₃N₅S = 795.25) |
| 27 | m/z = 769.93(C₅₃H₃₁N₅S = 769.23) | 28 | m/z = 770.91(C₅₃H₃₀N₄OS = 770.21) |
| 29 | m/z = 810.93(C₅₅H₃₀N₄O₂S = 810.21) | 30 | m/z = 810.93(C₅₅H₃₀N₄O₂S = 810.21) |
| 31 | m/z = 746.93(C₅₂H₃₄N₄S = 746.25) | 32 | m/z = 746.93(C₅₂H₃₄N₄S = 746.25) |
| 33 | m/z = 746.93(C₅₂H₃₄N₄S = 746.25) | 34 | m/z = 706.87(C₄₉H₃₀N₄S = 706.22) |
| 35 | m/z = 782.96(C₅₅H₃₄N₄S = 782.25) | 36 | m/z = 603.74(C₄₂H₂₅N₃S = 603.18) |
| 37 | m/z = 679.84(C₄₈H₂₉N₃S = 679.21) | 38 | m/z = 679.84(C₄₈H₂₉N₃S = 679.21) |
| 39 | m/z = 653.80(C₄₆H₂₇N₃S = 653.19) | 40 | m/z = 653.80(C₄₆H₂₇N₃S = 653.19) |
| 41 | m/z = 719.91(C₅₁H₃₃N₃S = 719.24) | 42 | m/z = 693.82(C₄₈H₂₇N₃OS = 693.19) |
| 43 | m/z = 693.82(C₄₈H₂₇N₃OS = 693.19) | 44 | m/z = 693.82(C₄₈H₂₇N₃OS = 693.19) |
| 45 | m/z = 743.88(C₅₂H₂₉N₃OS = 743.20) | 46 | m/z = 709.88(C₄₈H₂₇N₃S₂ = 709.16) |
| 47 | m/z = 709.88(C₄₈H₂₇N₃S₂ = 709.16) | 48 | m/z = 709.88(C₄₈H₂₇N₃S₂ = 709.16) |
| 49 | m/z = 709.88(C₄₈H₂₇N₃S₂ = 709.16) | 50 | m/z = 759.94(C₅₂H₂₉N₃S₂ = 759.18) |
| 51 | m/z = 759.94(C₅₂H₂₉N₃S₂ = 759.18) | 52 | m/z = 768.94(C₅₄H₃₂N₄S = 768.23) |
| 53 | m/z = 768.94(C₅₄H₃₂N₄S = 768.23) | 54 | m/z = 768.94(C₅₄H₃₂N₄S = 768.23) |
| 55 | m/z = 692.84(C₄₈H₂₈N₄S = 692.20) | 56 | m/z = 845.04(C₆₀H₃₆N₄S = 844.27) |
| 57 | m/z = 768.94(C₅₄H₃₂N₄S = 768.23) | 58 | m/z = 768.94(C₅₄H₃₂N₄S = 768.23) |
| 59 | m/z = 769.92(C₅₄H₃₁N₃OS = 769.22) | 60 | m/z = 846.02(C₆₀H₃₅N₃OS = 845.25) |
| 61 | m/z = 872.06(C₆₁H₃₇N₅S = 871.28) | 62 | m/z = 922.12(C₆₅H₃₉N₅S = 921.29) |
| 63 | m/z = 972.18(C₆₉H₄₁N₅S = 971.31) | 64 | m/z = 795.96(C₅₅H₃₃N₅S = 795.25) |
| 65 | m/z = 846.02(C₅₉H₃₅N₅S = 845.26) | 66 | m/z = 936.11(C₆₅H₃₇N₅OS = 935.27) |
| 67 | m/z = 872.06(C₆₁H₃₇N₅S = 871.28) | 68 | m/z = 872.06(C₆₁H₃₇N₅S = 871.28) |
| 69 | m/z = 948.16(C₆₇H₄₁N₅S = 947.31) | 70 | m/z = 846.02(C₅₉H₃₅N₅S = 845.26) |
| 71 | m/z = 679.84(C₄₈H₂₉N₃S = 679.21) | 72 | m/z = 679.84(C₄₈H₂₉N₃S = 679.21) |
| 73 | m/z = 680.83(C₄₇H₂₈N₄S = 680.20) | 74 | m/z = 756.93(C₅₃H₃₂N₄S = 756.23) |
| 75 | m/z = 756.93(C₅₃H₃₂N₄S = 756.23) | 76 | m/z = 730.89(C₅₁H₃₀N₄S = 730.22) |
| 77 | m/z = 780.95(C₅₅H₃₂N₄S = 780.23) | 78 | m/z = 730.89(C₅₁H₃₀N₄S = 730.22) |
| 79 | m/z = 780.95(C₅₅H₃₂N₄S = 780.23) | 90 | m/z = 770.91(C₅₃H₃₀N₄OS = 770.21) |
| 81 | m/z = 820.97(C₅₇H₃₂N₄OS = 820.23) | 82 | m/z = 770.91(C₅₃H₃₀N₄OS = 770.21) |
| 83 | m/z = 770.91(C₅₃H₃₀N₄OS = 770.21) | 84 | m/z = 770.91(C₅₃H₃₀N₄OS = 770.21) |
| 85 | m/z = 820.97(C₅₇H₃₂N₄OS = 820.23) | 86 | m/z = 820.97(C₅₇H₃₂N₄OS = 820.23) |
| 87 | m/z = 786.97(C₅₃H₃₀N₄S₂ = 786.19) | 88 | m/z = 786.97(C₅₃H₃₀N₄S₂ = 786.19) |
| 89 | m/z = 786.97(C₅₃H₃₀N₄S₂ = 786.19) | 90 | m/z = 837.03(C₅₇H₃₂N₄S₂ = 836.21) |
| 91 | m/z = 837.03(C₅₇H₃₂N₄S₂ = 836.21) | 92 | m/z = 846.02(C₅₉H₃₅N₅S = 845.26) |
| 93 | m/z = 846.02(C₅₉H₃₅N₅S = 845.26) | 94 | m/z = 846.02(C₅₉H₃₅N₅S = 845.26) |
| 95 | m/z = 769.93(C₅₃H₃₁N₅S = 769.23) | 96 | m/z = 846.02(C₅₉H₃₅N₅S = 845.26) |
| 97 | m/z = 819.99(C₅₇H₃₃N₅S = 819.25) | 98 | m/z = 820.97(C₅₇H₃₂N₄OS = 820.23) |
| 99 | m/z = 860.99(C₅₉H₃₂N₄O₂S = 860.22) | 100 | m/z = 860.99(C₅₉H₃₂N₄₀O₂S₅ = 860.22) |
| 101 | m/z = 893.11(C₅₉H₃₂N₄S₃ = 892.18) | 102 | m/z = 877.05(C₅₉H₃₂N_{4O}S₂ = 876.20) |
| 103 | m/z = 859.02(C₅₉H₃₄N₆S = 858.26) | 104 | m/z = 756.93(C₅₃H₃₂N₄S = 756.23) |
| 105 | m/z = 833.03(C₅₉H₃₆N₄S = 832.27) | 106 | m/z = 653.80(C₄₆H₂₇N₃S = 653.19) |
| 107 | m/z = 729.90(C₅₂H₃₁N₃S = 729.22) | 108 | m/z = 729.90(C₅₂H₃₁N₃S = 729.22) |
| 109 | m/z = 703.86(C₅₀H₂₉N₃S = 703.21) | 110 | m/z = 703.86(C₅₀H₂₉N₃S = 703.21) |
| 111 | m/z = 769.97(C₅₅H₃₅N₃S = 769.26) | 112 | m/z = 743.88(C₅₂H₂₉N₃OS = 743.20) |
| 113 | m/z = 743.88(C₅₂H₂₉N₃OS = 743.20) | 114 | m/z = 743.88(C₅₂H₂₉N₃OS = 743.20) |
| 115 | m/z = 793.94(C₅₆H₃₁N₃OS = 793.22) | 116 | m/z = 759.94(C₅₂H₂₉N₃S₂ = 759.18) |
| 117 | m/z = 759.94(C₅₂H₂₉N₃S₂ = 759.18) | 118 | m/z = 759.94(C₅₂H₂₉N₃S₂ = 759.18) |
| 119 | m/z = 759.94(C₅₂H₂₉N₃S₂ = 759.18) | 120 | m/z = 810.01(C₅₆H₃₁N₃S₂ = 809.20) |
| 121 | m/z = 759.94(C₅₂H₂₉N₃S₂ = 759.18) | 122 | m/z = 819.00(C₅₈H₃₄N₄S = 818.25) |
| 123 | m/z = 819.00(C₅₈H₃₄N₄S = 818.25) | 124 | m/z = 819.00(C₅₈H₃₄N₄S = 818.25) |
| 125 | m/z = 742.90(C₅₂H₃₀N₄S = 742.22) | 126 | m/z = 895.10(C₆₄H₃₈N₄S = 894.28) |
| 127 | m/z = 819.00(C₅₈H₃₄N₄S = 818.25) | 128 | m/z = 819.00(C₅₈H₃₄N₄S = 818.25) |
| 129 | m/z = 819.98(C₅₈H₃₃N₃OS = 819.23) | 130 | m/z = 819.98(C₅₈H₃₃N₃OS = 819.23) |
| 131 | m/z = 893.11(C₅₉H₃₂N₄S₃ = 892.18) | 132 | m/z = 893.11(C₅₉H₃₂N₄S₃ = 892.18) |
| 133 | m/z = 893.11(C₅₉H₃₂N₄S₃ = 892.18) | 134 | m/z = 796.99(C₅₆H₃₆N₄S = 796.27) |
| 135 | m/z = 796.99(C₅₆H₃₆N₄S = 796.27) | 136 | m/z = 796.99(C₅₆H₃₆N₄S = 796.27) |
| 137 | m/z = 837.03(C₅₇H₃₂N₄S₂ = 836.21) | 138 | m/z = 913.15(C₆₅H₄₄N₄S = 912.33) |
| 139 | m/z = 680.83(C₄₇H₂₈N₄S = 680.20) | 140 | m/z = 887.07(C₆₂H₃₈N₄OS = 886.28) |
| 141 | m/z = 913.15(C₆₅H₄₄N₄S = 912.33) | 142 | m/z = 730.89(C₅₁H₃₀N₄S = 730.22) |
| 143 | m/z = 780.95(C₅₅H₃₂N₄S = 780.23) | 144 | m/z = 679.84(C₄₈H₂₉N₃S = 679.21) |
| 145 | m/z = 729.90(C₅₂H₃₁ N₃S = 729.22) | 146 | m/z = 820.97(C₅₇H₃₂N₄OS = 820.23) |
| 147 | m/z = 680.83(C₄₇H₂₈N₄S = 680.20) | 148 | m/z = 730.89(C₅₁H₃₀N₄S = 730.22) |
| 149 | m/z = 770.91(C₅₃H₃₀N₄OS = 770.21) | 150 | m/z = 860.99(C₅₉H₃₂N₄O₂S = 860.22) |
| 151 | m/z = 680.83(C₄₇H₂₈N₄S = 680.20) | 152 | m/z = 730.89(C₅₁H₃₀N₄S = 730.22) |
| 153 | m/z = 780.95(C₅₅H₃₂N₄S = 780.23) | 154 | m/z = 679.84(C₄₈H₂₉N₃S =679.21) |
| 155 | m/z = 729.90(C₅₂H₃₁ N₃S = 729.22) | 156 | m/z = 820.97(C₅₇H₃₂N₄OS = 820.23) |
| 157 | m/z = 756.93(C₅₃H₃₂N4S = 756.23) | 158 | m/z = 730.89(C₅₁H₃₀N₄S = 730.22) |
| 159 | m/z = 770.91 (C₅₃H₃₀N₄OS = 770.21) | 160 | m/z = 860.99(C₅₉H₃₂N₄O₂S = 860.22) |
| 161 | m/z = 653.80(C₄₆H₂₇N₃S = 653.19) | 162 | m/z = 703.86(C₅₀H₂₉N₃S = 703.21) |
| 163 | m/z = 819.00(C₅₀H₃₄N₄S = 818.25) | 164 | m/z = 743.88(C₅₂H₂₉N₃OS = 743.20) |
| 165 | m/z = 759.94(C₅₂H₂₉N₃S₂ = 759.18) | 166 | m/z = 729.90(C₅₂H₃₁N₃S = 729.22) |
| 167 | m/z = 703.86(C₅₀H₂₉N₃S = 703.21) | 168 | m/z = 743.88(C₅₂H₂₉N₃OS = 743.20) |
| 169 | m/z = 743.88(C₅₂H₂₉N₃OS = 743.20) | 170 | m/z = 759.94(C₅₂H₂₉N₃S₂ = 759.18) |
| 171 | m/z = 704.85(C₄₉H₂₀N₄S = 704.20) | 172 | m/z = 754.91(C₅₃H₃₀N₄S = 754.22) |
| 173 | m/z = 804.97(C₅₇H₃₂N₄S = 804.23) | 174 | m/z = 703.86(C₅₀H₂₉N₃S = 703.21) |
| 175 | m/z = 753.92(C₅₄H₃₁N₃S = 753.22) | 176 | m/z = 844.99(C₅₉H₃₂N₄OS = 844.23) |
| 177 | m/z = 780.95(C₅₅H₃₂N₄S = 780.23) | 178 | m/z = 754.91(C₅₃H₃₀N₄S = 754.22) |
| 179 | m/z = 794.93(C₅₅H₃₀N₄OS = 794.21) | 180 | m/z = 885.01(C₆₁H₃₂N₄O₂S = 884.22) |
| 181 | m/z = 677.83(C₄₈H₂₇N₃S = 677.19) | 182 | m/z = 727.88(C₅₂H₂₉N₃S = 727.21) |
| 183 | m/z = 843.02(C₆₀H₃₄N₄S = 842.25) | 184 | m/z = 767.91(C₅₄H₂₉N₃OS = 767.20) |
| 185 | m/z = 783.97(C₅₄H₂₉N₃S₂ = 783.18) | 186 | m/z = 753.92(C₅₄H₃₁N₃S = 753.22) |
| 187 | m/z = 727.88(C₅₂H₂₉N₃S = 727.21) | 188 | m/z = 767.91(C₅₄H₂₉N₃OS = 767.20) |
| 189 | m/z = 767.91(C₅₄H₂₉N₃OS = 767.20) | 190 | m/z = 783.97(C₅₄H₂₉N₃S₂ = 783.18) |
| 191 | m/z = 680.83(C₄₇H₂₈N₄S = 680.20) | 192 | m/z = 730.89(C₅₁H₃₀N₄S = 730.22) |
| 193 | m/z = 780.95(C₅₅H₃₂N₄S = 780.23) | 194 | m/z = 679.84(C₄₈H₂₉N₃S = 679.21) |
| 195 | m/z = 729.90(C₅₂H₃₁N₃S = 729.22) | 196 | m/z = 820.97(C₅₇H₃₂N₄OS = 820.23) |
| 197 | m/z = 756.93(C₅₃H₃₂N₄S = 756.23) | 198 | m/z = 730.89(C₅₁H₃₀N₄S = 730.22) |
| 199 | m/z = 770.91(C₅₃H₃₀N₄OS = 770.21) | 200 | m/z = 860.99(C₅₉H₃₂N₄O₂S = 860.22) |
| 201 | m/z = 653.80(C₄₆H₂₇N₃S = 653.19) | 202 | m/z = 703.86(C₅₀H₂₉N₃S = 703.21) |
| 203 | m/z = 819.00(C₅₀H₃₄N₄S = 818.25) | 204 | m/z = 743.88(C₅₂H₂₉N₃OS = 743.20) |
| 205 | m/z = 759.94(C₅₂H₂₉N₃S₂ = 759.18) | 206 | m/z = 729.90(C₅₂H₃₁N₃S = 729.22) |
| 207 | m/z = 703.86(C₅₀H₂₉N₃S = 703.21) | 208 | m/z = 743.88(C₅₂H₂₉N₃OS = 743.20) |
| 209 | m/z = 743.88(C₅₂H₂₉N₃OS = 743.20) | 210 | m/z = 759.94(C₅₂H₂₉N₃S₂ = 759.18) |

### Preparation of Organic Light Emitting Diode—Red Host

A glass substrate coated with ITO (indium tin oxide) as a 1500 Å-thick thin film was washed with distilled water.

After washing with the distilled water, the glass substrate was ultrasonic wave-washed with a solvent such as acetone, methanol, isopropyl alcohol, and the like and dried, and then the glass substrate was UVO treated for 5 minutes using UV in a UV cleaner.

Subsequently, the substrate was moved to a plasma cleaner (PT), and then plasma-treated in a vacuum atmosphere to remove the ITO work function and residual film, and moved to a thermal depositor for organic deposition.

On the ITO transparent electrode (anode), 2-TNATA(4,4′,4″-tris[2-naphthyl(phenyl)amino] triphenylamine) as the hole injection layer and NPB(N,N′-di(1-naphthyl)-N,N′-diphenyl-(1,1-biphenyl)-4,4′-diamine) as a hole transport layer were formed as common layers.

The light emitting layer was deposited thereon by thermal evaporation as follows.

A 500 Å-thick light emitting layer was deposited by doping 3% (piq)₂(Ir)(acac) to the host, using a compound shown in the table below as a red host, and using (piq)₂(Ir)(acac) as a red phosphorescent dopant.

Thereafter, 60 Å-thick BCP was deposited as the hole blocking layer, and 200 Å-thick Alq₃ was deposited thereon as the electron transport layer.

Finally, 10 Å-thick lithium fluoride (LiF) was deposited on the electron transport layer to form the electron injection layer, and then a 1,200 Å-thick aluminum (Al) cathode was deposited on the electron injection layer to form the cathode, manufacturing an organic electroluminescent diode.

Meanwhile, all the organic compounds needed to manufacture the OLED diode were used for OLED manufacturing by vacuum sublimation purifying independently for each of the organic compounds under 10⁻⁶˜10⁻⁸ torr.

Driving Voltage and Luminous Efficiency of Organic Electroluminescence Diode

The electroluminescence (EL) characteristics of the organic electroluminescent diode manufactured as described above were measured by the Mcscience M7000 system. And the T90 was measured when the reference luminance was 6,000 cd/m² from the EL measurement result using a life span measuring instrument (M6000) manufactured by Mcscience.

The properties of the organic electroluminescent diode of the present invention are shown in the following table.

TABLE 3

| | | Driving | Efficiency | Color | Lifespan |
|---|---|---|---|---|---|
| | Compound | Voltage (V) | (cd/A) | Coordination (x, y) | (T₉₀) |
| Comparative | A | 5.68 | 14.2 | 0.672, 0.327 | 38 |
| Example 1 | | | | | |
| Comparative | B | 5.62 | 12.1 | 0.688, 0.312 | 40 |
| Example 2 | | | | | |
| Comparative | C | 5.42 | 14.1 | 0.691, 0.309 | 53 |
| Example 3 | | | | | |
| Comparative | D | 5.22 | 16.2 | 0.689, 0.310 | 28 |
| Example 4 | | | | | |
| Comparative | E | 5.52 | 15.2 | 0.689, 0.310 | 11.5 |
| Example 5 | | | | | |
| Comparative | F | 5.69 | 11.9 | 0.687, 0.313 | 32 |
| Example 6 | | | | | |
| Comparative | G | 5.72 | 12.3 | 0.673, 0.326 | 31 |
| Example 7 | | | | | |
| Example 1 | Compound 1 | 4.29 | 24.2 | 0.676, 0.324 | 138 |
| Example 2 | Compound 3 | 4.20 | 22.0 | 0.678, 0.322 | 109 |
| Example 3 | Compound 5 | 4.28 | 25.7 | 0.679, 0.321 | 161 |
| Example 4 | Compound 12 | 4.25 | 25.2 | 0.682, 0.317 | 159 |
| Example 5 | Compound 18 | 4.32 | 19.2 | 0.685, 0.314 | 124 |
| Example 6 | Compound 22 | 4.08 | 28.6 | 0.678, 0.321 | 187 |
| Example 7 | Compound 31 | 4.19 | 24.0 | 0.687, 0.313 | 130 |
| Example 8 | Compound 34 | 4.12 | 24.2 | 0.688, 0.312 | 120 |
| Example 9 | Compound 35 | 4.48 | 18.4 | 0.689, 0.310 | 109 |
| Example 10 | Compound 36 | 4.39 | 18.7 | 0.691, 0.309 | 98 |
| Example 11 | Compound 38 | 4.37 | 19.2 | 0.674, 0.325 | 103 |
| Example 12 | Compound 43 | 4.55 | 17.2 | 0.678, 0.321 | 100 |
| Example 13 | Compound 47 | 4.59 | 17.1 | 0.685, 0.314 | 97 |
| Example 14 | Compound 54 | 4.57 | 18.0 | 0.684, 0.316 | 99 |
| Example 15 | Compound 59 | 4.25 | 25.2 | 0.680, 0.319 | 131 |
| Example 16 | Compound 61 | 4.23 | 22.6 | 0.684, 0.315 | 106 |
| Example 17 | Compound 64 | 4.22 | 25.5 | 0.683, 0.317 | 158 |
| Example 18 | Compound 67 | 4.21 | 24.9 | 0.682, 0.317 | 153 |
| Example 19 | Compound 68 | 4.35 | 19.8 | 0.682, 0.317 | 120 |
| Example 20 | Compound 69 | 4.10 | 28.0 | 0.680, 0.319 | 177 |
| Example 21 | Compound 70 | 4.20 | 24.6 | 0.682, 0.318 | 133 |
| Example 22 | Compound 71 | 4.15 | 24.5 | 0.681, 0.319 | 122 |
| Example 23 | Compound 73 | 4.42 | 17.9 | 0.680, 0.319 | 119 |
| Example 24 | Compound 75 | 4.33 | 17.2 | 0.681, 0.319 | 101 |
| Example 25 | Compound 82 | 4.35 | 18.2 | 0.687, 0.313 | 93 |
| Example 26 | Compound 88 | 4.50 | 17.5 | 0.688, 0.312 | 103 |
| Example 27 | Compound 92 | 4.51 | 17.0 | 0.689, 0.310 | 99 |
| Example 28 | Compound 104 | 4.52 | 18.9 | 0.691, 0.309 | 94 |
| Example 29 | Compound 105 | 4.30 | 24.2 | 0.674, 0.325 | 140 |
| Example 30 | Compound 106 | 4.28 | 21.6 | 0.672, 0.327 | 117 |
| Example 31 | Compound 108 | 4.27 | 24.5 | 0.676, 0.324 | 169 |
| Example 32 | Compound 113 | 4.26 | 23.9 | 0.678, 0.322 | 164 |
| Example 33 | Compound 117 | 4.40 | 18.8 | 0.679, 0.321 | 131 |
| Example 34 | Compound 124 | 4.15 | 27.0 | 0.682, 0.317 | 175 |
| Example 35 | Compound 129 | 4.25 | 23.6 | 0.685, 0.314 | 144 |
| Example 36 | Compound 136 | 4.20 | 23.5 | 0.678, 0.321 | 133 |
| Example 37 | Compound 141 | 4.42 | 16.9 | 0.681, 0.319 | 120 |
| Example 38 | Compound 144 | 4.23 | 20.2 | 0.678, 0.321 | 108 |
| Example 39 | Compound 147 | 4.21 | 22.0 | 0.685, 0.314 | 99 |
| Example 40 | Compound 150 | 4.25 | 23.7 | 0.684, 0.316 | 96 |
| Example 41 | Compound 151 | 4.32 | 21.2 | 0.680, 0.319 | 120 |
| Example 42 | Compound 154 | 4.30 | 19.2 | 0.684, 0.315 | 98 |
| Example 43 | Compound 157 | 4.28 | 18.6 | 0.683, 0.317 | 100 |
| Example 44 | Compound 161 | 4.28 | 20.2 | 0.682, 0.317 | 108 |
| Example 45 | Compound 163 | 4.25 | 20.0 | 0.682, 0.317 | 102 |
| Example 46 | Compound 166 | 4.27 | 21.2 | 0.680, 0.319 | 99 |
| Example 47 | Compound 169 | 4.42 | 20.2 | 0.682, 0.318 | 125 |
| Example 48 | Compound 171 | 4.30 | 18.2 | 0.681, 0.319 | 99 |
| Example 49 | Compound 174 | 4.28 | 19.6 | 0.680, 0.319 | 97 |
| Example 50 | Compound 177 | 4.33 | 18.2 | 0.687, 0.313 | 98 |
| Example 51 | Compound 180 | 4.29 | 19.0 | 0.688, 0.312 | 101 |
| Example 52 | Compound 181 | 4.25 | 20.3 | 0.689, 0.310 | 92 |
| Example 53 | Compound 183 | 4.31 | 19.8 | 0.691, 0.309 | 112 |
| Example 54 | Compound 186 | 4.35 | 18.9 | 0.674, 0.325 | 92 |
| Example 55 | Compound 189 | 4.38 | 20.6 | 0.672, 0.327 | 88 |
| Example 56 | Compound 191 | 4.38 | 22.9 | 0.676, 0.324 | 90 |
| Example 57 | Compound 194 | 4.39 | 21.9 | 0.678, 0.322 | 103 |
| Example 58 | Compound 197 | 4.30 | 21.7 | 0.679, 0.321 | 80 |
| Example 59 | Compound 201 | 4.29 | 19.9 | 0.682, 0.317 | 89 |
| Example 60 | Compound 203 | 4.49 | 21.9 | 0.685, 0.314 | 121 |
| Example 61 | Compound 206 | 4.32 | 19.7 | 0.678, 0.321 | 98 |
| Example 62 | Compound 209 | 4.32 | 19.0 | 0.681, 0.319 | 111 |
| Example 63 | Compound 210 | 4.29 | 17.8 | 0.678, 0.321 | 119 |

Referring to Table 3, when the material using dibenzofurane which is a linker of the compound of the present invention is used as the red light emitting layer host, in the organic light emitting diode, the examples have a lower driving voltage, and significantly improved efficiency and life span compared to Comparative Examples A to G.

Referring to Table 3, the dibenzofurane linker is introduced between Sub A and Sub B of a compound to manufacture the compound having a suitable bandgap as the red host, and the compound can satisfy the requirements of the light emitting layer.

As a result, the electron transfer capability is improved, resulting in an excellent effect on driving and efficiency, while also improving thermal stability and life span properties.

In addition, the compounds have improved driving, efficiency, and life span, compared to the structure in which Sub B is directly bonded to N of carbazole (Sub A) without a linker (Comparative Example A, Comparative Example B).

While the preferred embodiments of the present invention have been described in detail, the scope of the present invention is not limited thereto, and various modifications and improvements of those skilled in the art using the basic concepts of the present invention as defined in the following claims are also within the scope of the present invention.

### DESCRIPTION OF SYMBOLS

100: Substrate
200: Anode
300: Organic layer
301: Hole injection layer
302: Hole transfer layer
303: Light emitting layer
304: Hole blocking layer
305: Electron transport layer
306: Electron injection layer
400: Cathode

1. A compound represented by Formula 1-1 and Formula 2-1: 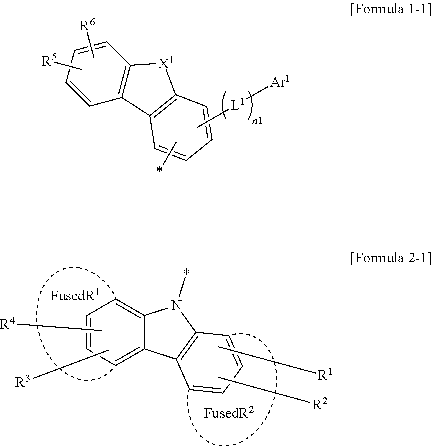
wherein, in Formula 1-1 and Formula 2-1,
X¹ is —O— or —S—,
Ar¹ is a substituent having electron characteristics or a substituent having hole characteristics,
R¹ to R⁶ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof,
L¹ is a single bond, a substituted or unsubstituted C6 to C60 arylene group, or a substituted or unsubstituted C2 to C60 heteroarylene group,
n1 is one of integers of 0 to 2,
* is a linking point of Formula 1-1 and Formula 2-1, and
FuseR¹ and FusedR² are each independently a substituted or unsubstituted C3 to C60 fused ring.

2. The compound of claim 1,
wherein Formula 1-1 is represented by Formula 1-2: 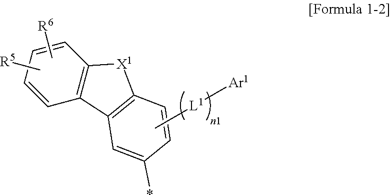
wherein, in Formula 1-2,
X¹ is —O— or —S—,
Ar¹ is a substituent having electron characteristics or a substituent having hole characteristics,
R⁵ and R⁶ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof,
L¹ is a single bond, a substituted or unsubstituted C6 to C60 arylene group, or a substituted or unsubstituted C2 to C60 heteroarylene group,
n1 is one of integers of 0 to 2, and
* is a linking point of Formula 1-2 and Formula 2-1.

3. The compound of claim 1,
wherein Formula 2-1 is represented by Formula 2-2: 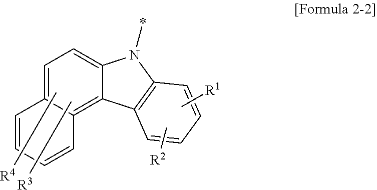
wherein, in Formula 2-2,
R¹ to R⁴ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and
* is a linking point of Formula 1-1 and Formula 2-2.

4. The compound of claim 1,
wherein Formula 2-1 is represented by Formula 2-3: 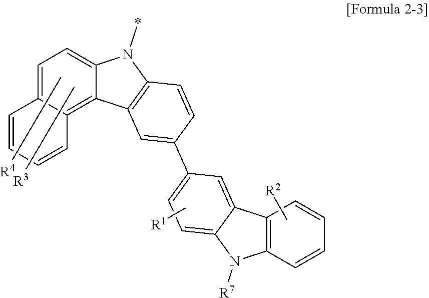
wherein, in Formula 2-3,
R¹ to R⁴ and R⁷ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and
* is a linking point of Formula 1-1 and Formula 2-3.

5. The compound of claim 1,
wherein Formula 2-1 is represented by Formula 2-4: 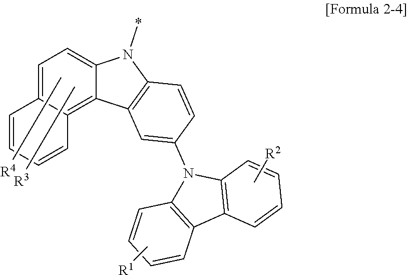
wherein, in Formula 2-4,
R¹ to R⁴ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and
* is a linking point of Formula 1-1 and Formula 2-4.

6. The compound of claim 1,
wherein Formula 2-1 is represented by Formula 2-5: 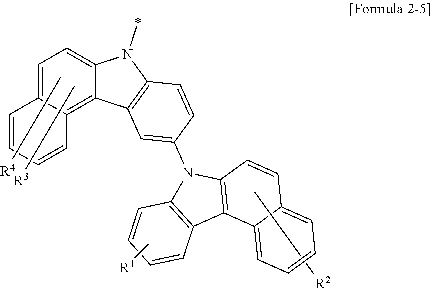
wherein, in Formula 2-5,
R¹ to R⁴ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and
* is a linking point of Formula 1-1 and Formula 2-5.

7. The compound of claim 1,
wherein Formula 2-1 is represented by Formula 2-6: 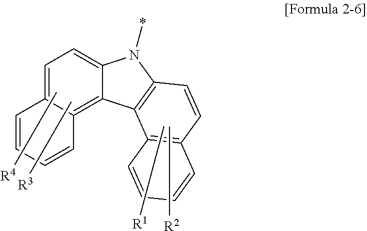
wherein, in Formula 2-6,
R¹ to R⁴ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and
* is a linking point of Formula 1-1 and Formula 2-6.

8. The compound of claim 1,
wherein Formula 2-1 is represented by Formula 2-7: 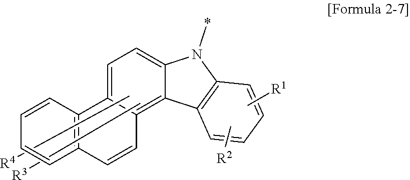
wherein, in Formula 2-7,
R¹ to R⁴ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and
* is a linking point of Formula 1-1 and Formula 2-7.

9. The compound of claim 1,
wherein Formula 2-1 is represented by Formula 2-8: 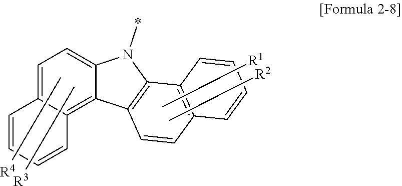
wherein, in Formula 2-8,
R¹ to R⁴ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and
* is a linking point of Formula 1-1 and Formula 2-8.

10. The compound of claim 1,
wherein Formula 2-1 is represented by Formula 2-9: 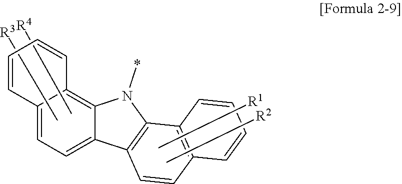
wherein, in Formula 2-9,
R¹ to R⁴ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and
* is a linking point of Formula 1-1 and Formula 2-9.

11. The compound of claim 1,
wherein Formula 2-1 is represented by Formula 2-10: 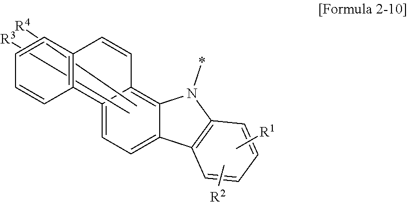
wherein, in Formula 2-10,
R¹ to R⁴ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and
* is a linking point of Formula 1-1 and Formula 2-10.

12. The compound of claim 1,
wherein Formula 2-1 is represented by Formula 2-11: 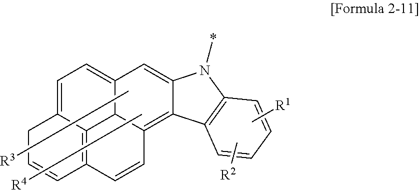
wherein, in Formula 2-11,
R¹ to R⁴ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and
* is a linking point of Formula 1-1 and Formula 2-11.

13. The compound of claim 1, wherein
the Ar¹ is a substituted or unsubstituted C6 to C60 aryl group, or a substituted or unsubstituted C2 to C60 heteroaryl group.

14. The compound of claim 1, wherein
the Ar¹ is Formula 3-1 or Formula 3-2: 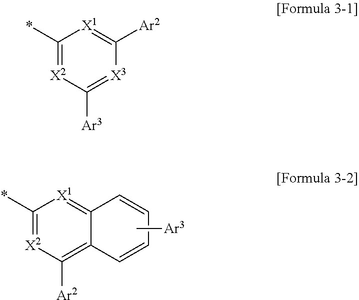
wherein, in Formula 3-1 and Formula 3-2,
the X¹ to X³ are —CR′— or —N—, at least one of X¹ to X³ is —N—,
Ar² and Ar³ are each independently hydrogen, deuterium, a cyano group, a substituted or unsubstituted C1 to C60 alkyl group, a substituted or unsubstituted C6 to C60 aryl group, a substituted or unsubstituted C2 to C60 heteroaryl group, or a combination thereof, and
R′ is hydrogen, deuterium, a cyano group, or a substituted or unsubstituted C1 to C60 alkyl group.

15. The compound of claim 14,
wherein at least one of Ar² and Ar³ is any one of Formula 4-1 to Formula 4-5: 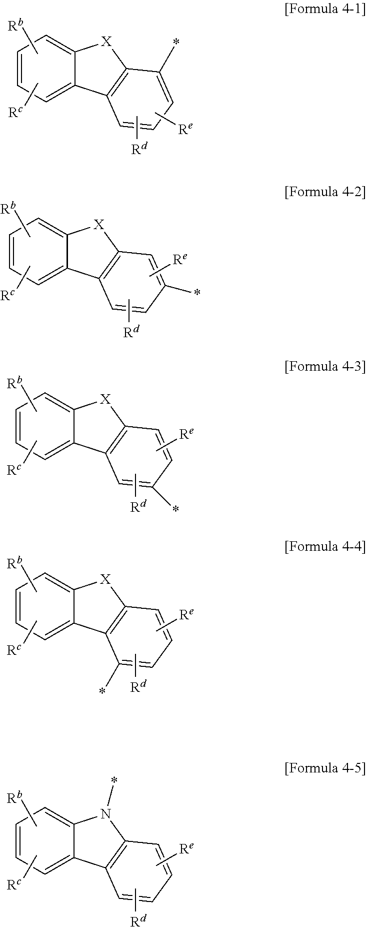
wherein, in Formula 4-1 to Formula 4-5,
X is —NR^{x}—, —O—, —S—, or —CR^{x}R^{y}—
R^{x} and R^{y} are each independently hydrogen, deuterium, a cyano group, a substituted or unsubstituted C1 to C60 alkyl group, or a C6 to C60 aryl group, and
R^{b} to R^{e} are each independently hydrogen, deuterium, a cyano group, a substituted or unsubstituted C1 to C60 alkyl group, or a C6 to C60 aryl group.

16. The compound of claim 1, wherein
the compound is any one of the compounds of Group II:

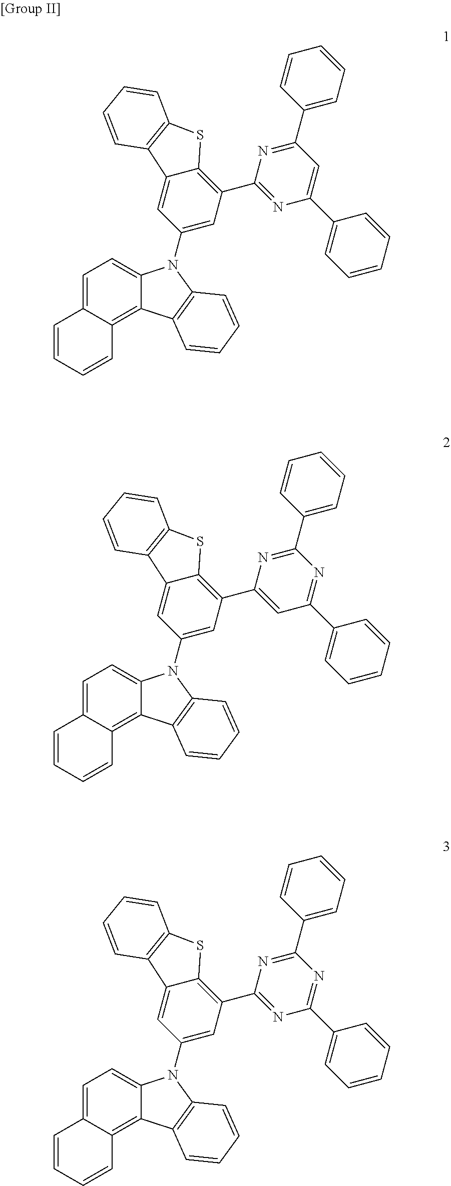 [SMILES: c1ccc(-c2cc(-c3ccccc3)nc(-c3cc(-n4c5ccccc5c5c6ccccc6ccc54)cc4c3sc3ccccc34)n2)cc1 ; c1ccc(-c2cc(-c3cc(-n4c5ccccc5c5c6ccccc6ccc54)cc4c3sc3ccccc34)nc(-c3ccccc3)n2)cc1 ; c1ccc(-c2nc(-c3ccccc3)nc(-c3cc(-n4c5ccccc5c5c6ccccc6ccc54)cc4c3sc3ccccc34)n2)cc1]

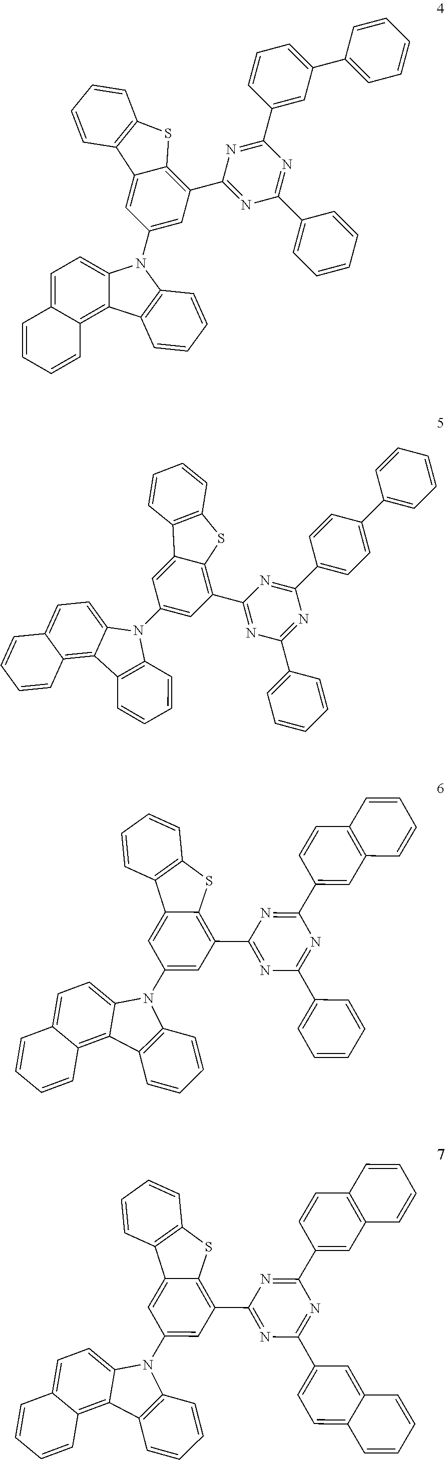 [SMILES: c1ccc(-c2cccc(-c3nc(-c4ccccc4)nc(-c4cc(-n5c6ccccc6c6c7ccccc7ccc65)cc5c4sc4ccccc45)n3)c2)cc1 ; c1ccc(-c2ccc(-c3nc(-c4ccccc4)nc(-c4cc(-n5c6ccccc6c6c7ccccc7ccc65)cc5c4sc4ccccc45)n3)cc2)cc1 ; c1ccc(-c2nc(-c3ccc4ccccc4c3)nc(-c3cc(-n4c5ccccc5c5c6ccccc6ccc54)cc4c3sc3ccccc34)n2)cc1 ; c1ccc2cc(-c3nc(-c4ccc5ccccc5c4)nc(-c4cc(-n5c6ccccc6c6c7ccccc7ccc65)cc5c4sc4ccccc45)n3)ccc2c1]

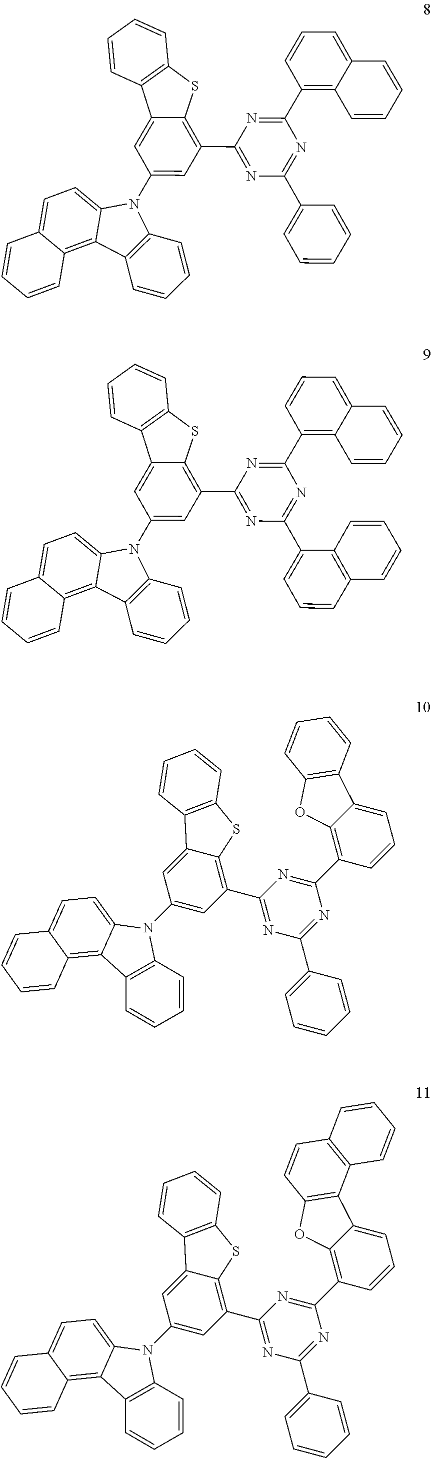 [SMILES: c1ccc(-c2nc(-c3cccc4ccccc34)nc(-c3cc(-n4c5ccccc5c5c6ccccc6ccc54)cc4c3sc3ccccc34)n2)cc1 ; c1ccc2c(-c3nc(-c4cccc5ccccc45)nc(-c4cc(-n5c6ccccc6c6c7ccccc7ccc65)cc5c4sc4ccccc45)n3)cccc2c1 ; c1ccc(-c2nc(-c3cccc4c3oc3ccccc34)nc(-c3cc(-n4c5ccccc5c5c6ccccc6ccc54)cc4c3sc3ccccc34)n2)cc1 ; c1ccc(-c2nc(-c3cccc4c3oc3ccc5ccccc5c34)nc(-c3cc(-n4c5ccccc5c5c6ccccc6ccc54)cc4c3sc3ccccc34)n2)cc1]

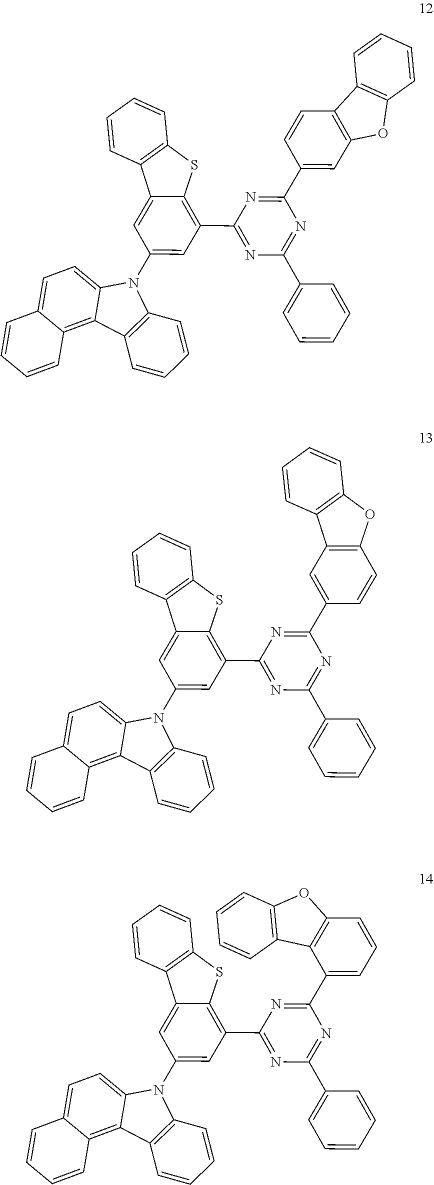 [SMILES: c1ccc(-c2nc(-c3ccc4c(c3)oc3ccccc34)nc(-c3cc(-n4c5ccccc5c5c6ccccc6ccc54)cc4c3sc3ccccc34)n2)cc1 ; c1ccc(-c2nc(-c3ccc4oc5ccccc5c4c3)nc(-c3cc(-n4c5ccccc5c5c6ccccc6ccc54)cc4c3sc3ccccc34)n2)cc1 ; c1ccc(-c2nc(-c3cc(-n4c5ccccc5c5c6ccccc6ccc54)cc4c3sc3ccccc34)nc(-c3cccc4oc5ccccc5c34)n2)cc1]

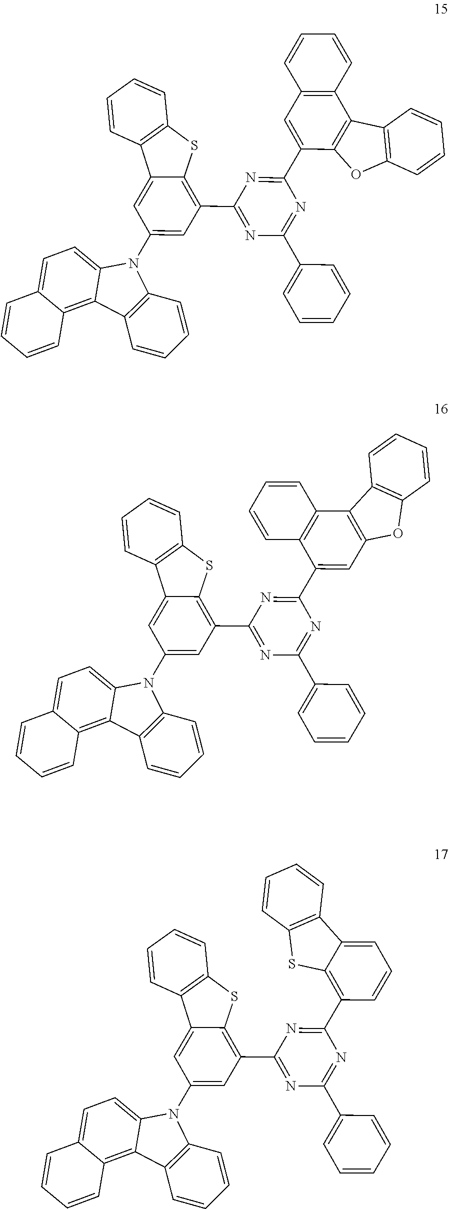 [SMILES: c1ccc(-c2nc(-c3cc4ccccc4c4c3oc3ccccc34)nc(-c3cc(-n4c5ccccc5c5c6ccccc6ccc54)cc4c3sc3ccccc34)n2)cc1 ; c1ccc(-c2nc(-c3cc4oc5ccccc5c4c4ccccc34)nc(-c3cc(-n4c5ccccc5c5c6ccccc6ccc54)cc4c3sc3ccccc34)n2)cc1 ; c1ccc(-c2nc(-c3cccc4c3sc3ccccc34)nc(-c3cc(-n4c5ccccc5c5c6ccccc6ccc54)cc4c3sc3ccccc34)n2)cc1]

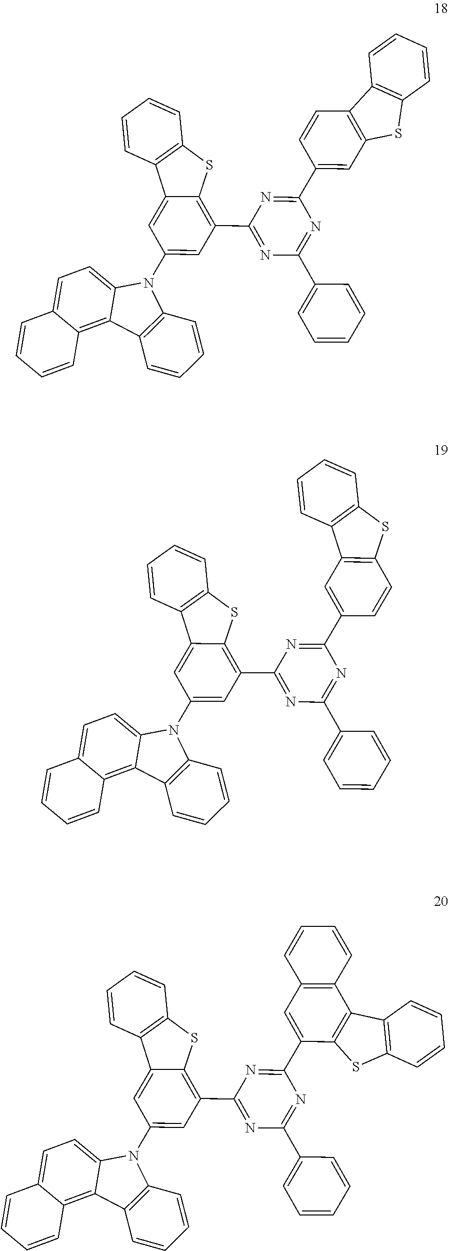 [SMILES: c1ccc(-c2nc(-c3ccc4c(c3)sc3ccccc34)nc(-c3cc(-n4c5ccccc5c5c6ccccc6ccc54)cc4c3sc3ccccc34)n2)cc1 ; c1ccc(-c2nc(-c3ccc4sc5ccccc5c4c3)nc(-c3cc(-n4c5ccccc5c5c6ccccc6ccc54)cc4c3sc3ccccc34)n2)cc1 ; c1ccc(-c2nc(-c3cc(-n4c5ccccc5c5c6ccccc6ccc54)cc4c3sc3ccccc34)nc(-c3cc4ccccc4c4c3sc3ccccc34)n2)cc1]

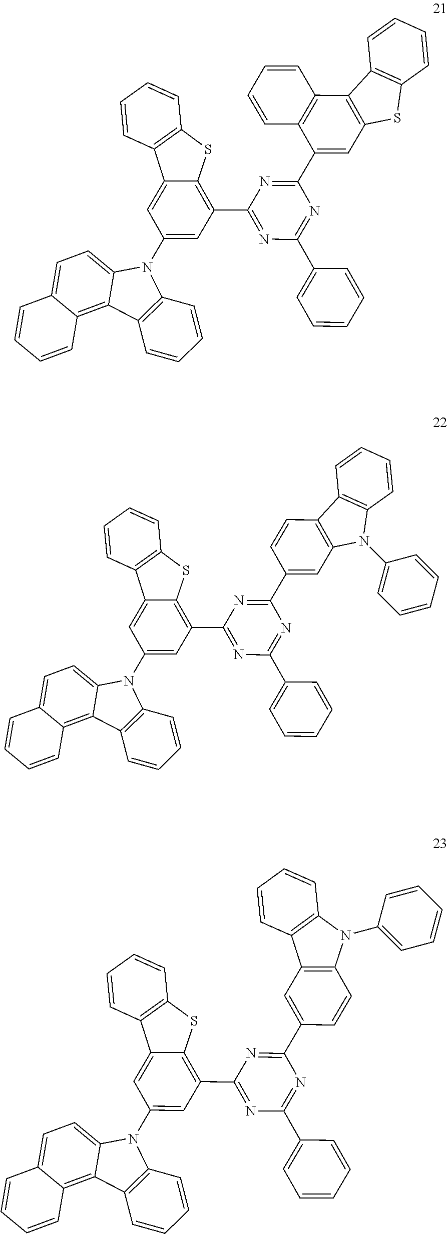 [SMILES: c1ccc(-c2nc(-c3cc4sc5ccccc5c4c4ccccc34)nc(-c3cc(-n4c5ccccc5c5c6ccccc6ccc54)cc4c3sc3ccccc34)n2)cc1 ; c1ccc(-c2nc(-c3ccc4c5ccccc5n(-c5ccccc5)c4c3)nc(-c3cc(-n4c5ccccc5c5c6ccccc6ccc54)cc4c3sc3ccccc34)n2)cc1 ; c1ccc(-c2nc(-c3ccc4c(c3)c3ccccc3n4-c3ccccc3)nc(-c3cc(-n4c5ccccc5c5c6ccccc6ccc54)cc4c3sc3ccccc34)n2)cc1]

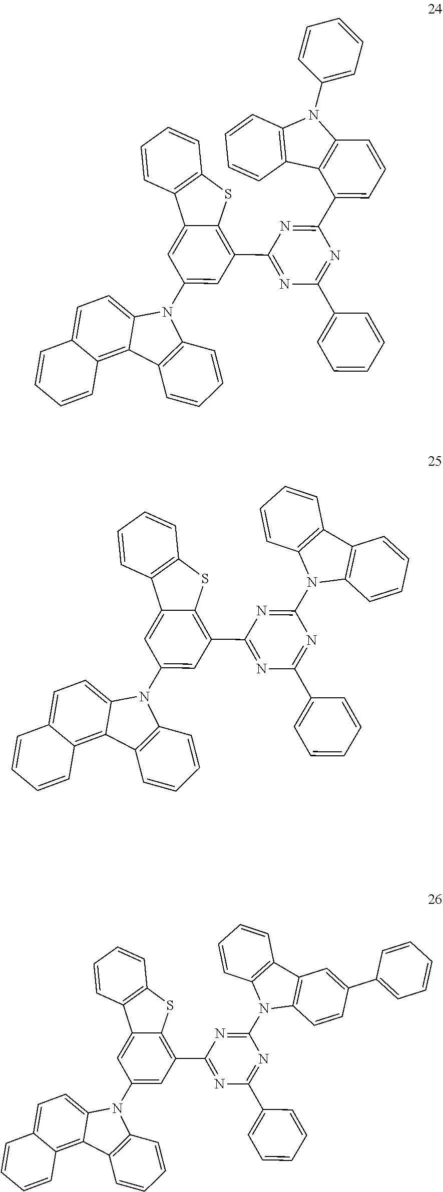 [SMILES: c1ccc(-c2nc(-c3cc(-n4c5ccccc5c5c6ccccc6ccc54)cc4c3sc3ccccc34)nc(-c3cccc4c3c3ccccc3n4-c3ccccc3)n2)cc1 ; c1ccc(-c2nc(-c3cc(-n4c5ccccc5c5c6ccccc6ccc54)cc4c3sc3ccccc34)nc(-n3c4ccccc4c4ccccc43)n2)cc1 ; c1ccc(-c2ccc3c(c2)c2ccccc2n3-c2nc(-c3ccccc3)nc(-c3cc(-n4c5ccccc5c5c6ccccc6ccc54)cc4c3sc3ccccc34)n2)cc1]

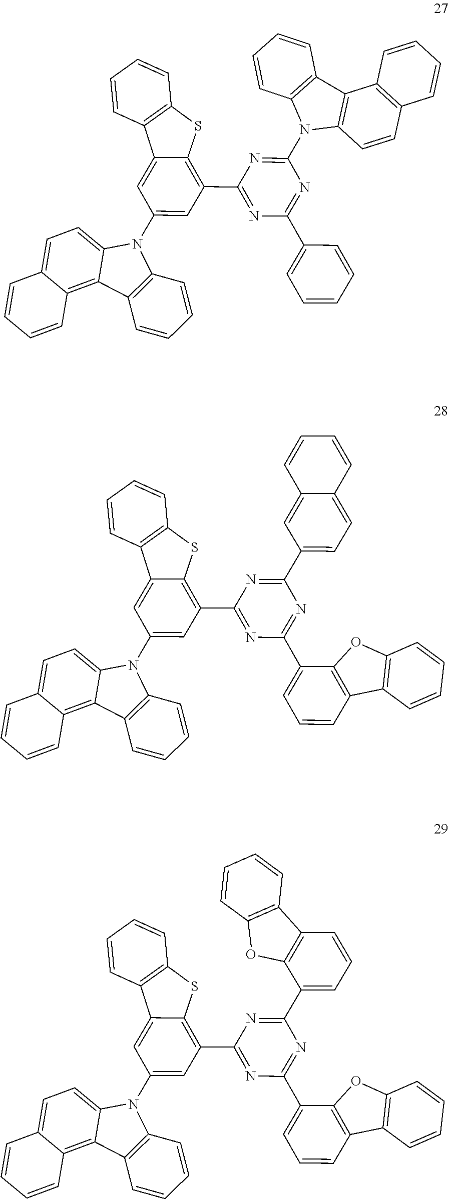 [SMILES: c1ccc(-c2nc(-c3cc(-n4c5ccccc5c5c6ccccc6ccc54)cc4c3sc3ccccc34)nc(-n3c4ccccc4c4c5ccccc5ccc43)n2)cc1 ; c1ccc2cc(-c3nc(-c4cccc5c4oc4ccccc45)nc(-c4cc(-n5c6ccccc6c6c7ccccc7ccc65)cc5c4sc4ccccc45)n3)ccc2c1 ; c1ccc2c(c1)ccc1c2c2ccccc2n1-c1cc(-c2nc(-c3cccc4c3oc3ccccc34)nc(-c3cccc4c3oc3ccccc34)n2)c2sc3ccccc3c2c1]

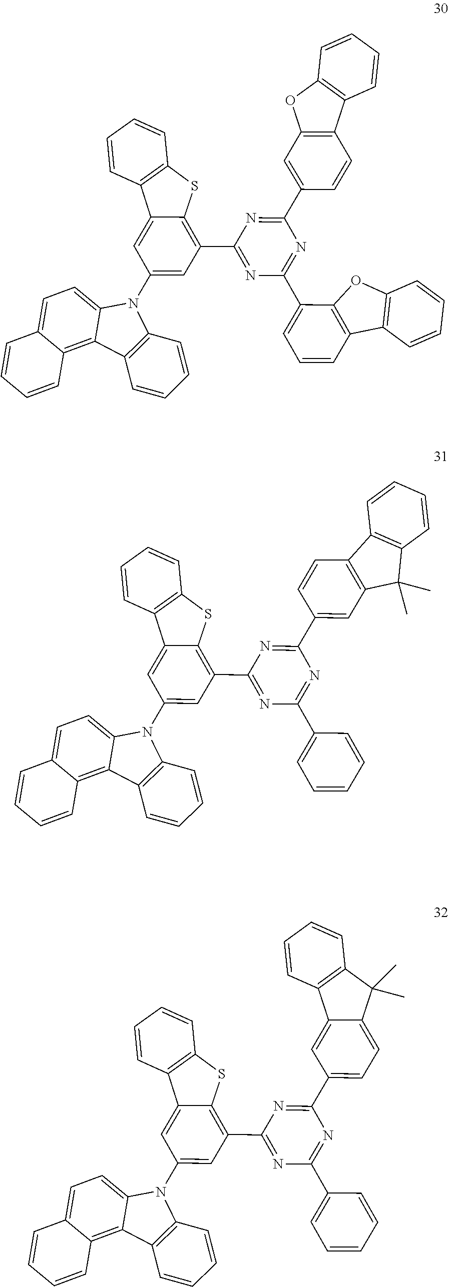 [SMILES: c1ccc2c(c1)ccc1c2c2ccccc2n1-c1cc(-c2nc(-c3ccc4c(c3)oc3ccccc34)nc(-c3cccc4c3oc3ccccc34)n2)c2sc3ccccc3c2c1 ; CC1(C)c2ccccc2-c2ccc(-c3nc(-c4ccccc4)nc(-c4cc(-n5c6ccccc6c6c7ccccc7ccc65)cc5c4sc4ccccc45)n3)cc21 ; CC1(C)c2ccccc2-c2cc(-c3nc(-c4ccccc4)nc(-c4cc(-n5c6ccccc6c6c7ccccc7ccc65)cc5c4sc4ccccc45)n3)ccc21]

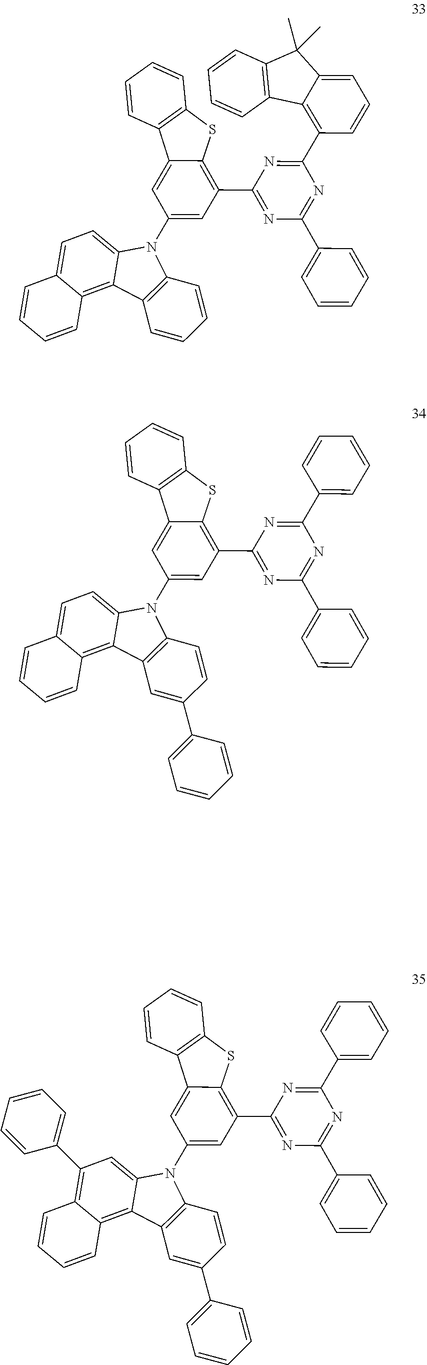 [SMILES: CC1(C)c2ccccc2-c2c(-c3nc(-c4ccccc4)nc(-c4cc(-n5c6ccccc6c6c7ccccc7ccc65)cc5c4sc4ccccc45)n3)cccc21 ; c1ccc(-c2ccc3c(c2)c2c4ccccc4ccc2n3-c2cc(-c3nc(-c4ccccc4)nc(-c4ccccc4)n3)c3sc4ccccc4c3c2)cc1 ; c1ccc(-c2ccc3c(c2)c2c4ccccc4c(-c4ccccc4)cc2n3-c2cc(-c3nc(-c4ccccc4)nc(-c4ccccc4)n3)c3sc4ccccc4c3c2)cc1]

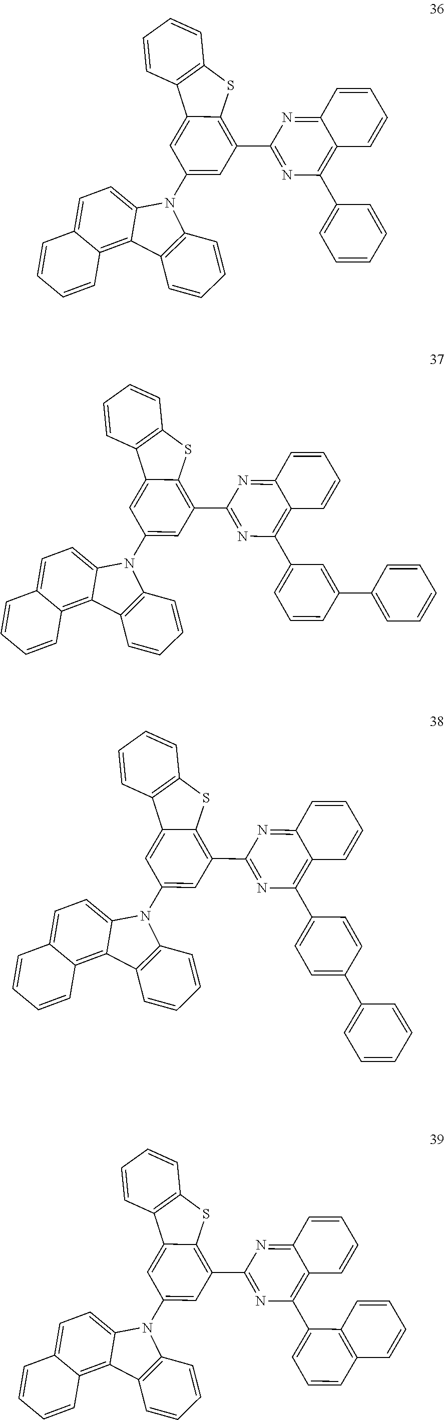 [SMILES: c1ccc(-c2nc(-c3cc(-n4c5ccccc5c5c6ccccc6ccc54)cc4c3sc3ccccc34)nc3ccccc23)cc1 ; c1ccc(-c2cccc(-c3nc(-c4cc(-n5c6ccccc6c6c7ccccc7ccc65)cc5c4sc4ccccc45)nc4ccccc34)c2)cc1 ; c1ccc(-c2ccc(-c3nc(-c4cc(-n5c6ccccc6c6c7ccccc7ccc65)cc5c4sc4ccccc45)nc4ccccc34)cc2)cc1 ; c1ccc2c(-c3nc(-c4cc(-n5c6ccccc6c6c7ccccc7ccc65)cc5c4sc4ccccc45)nc4ccccc34)cccc2c1]

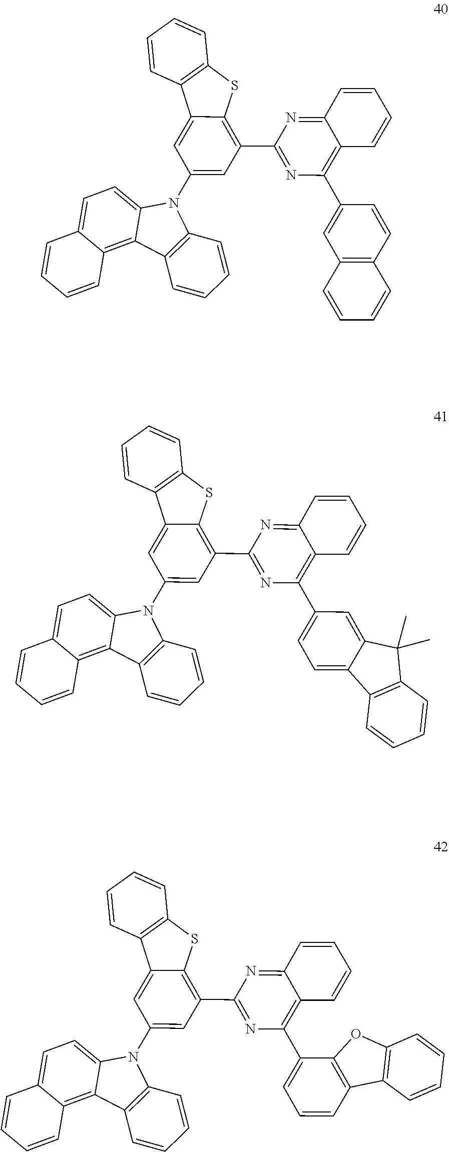 [SMILES: c1ccc2cc(-c3nc(-c4cc(-n5c6ccccc6c6c7ccccc7ccc65)cc5c4sc4ccccc45)nc4ccccc34)ccc2c1 ; CC1(C)c2ccccc2-c2ccc(-c3nc(-c4cc(-n5c6ccccc6c6c7ccccc7ccc65)cc5c4sc4ccccc45)nc4ccccc34)cc21 ; c1ccc2c(c1)ccc1c2c2ccccc2n1-c1cc(-c2nc(-c3cccc4c3oc3ccccc34)c3ccccc3n2)c2sc3ccccc3c2c1]

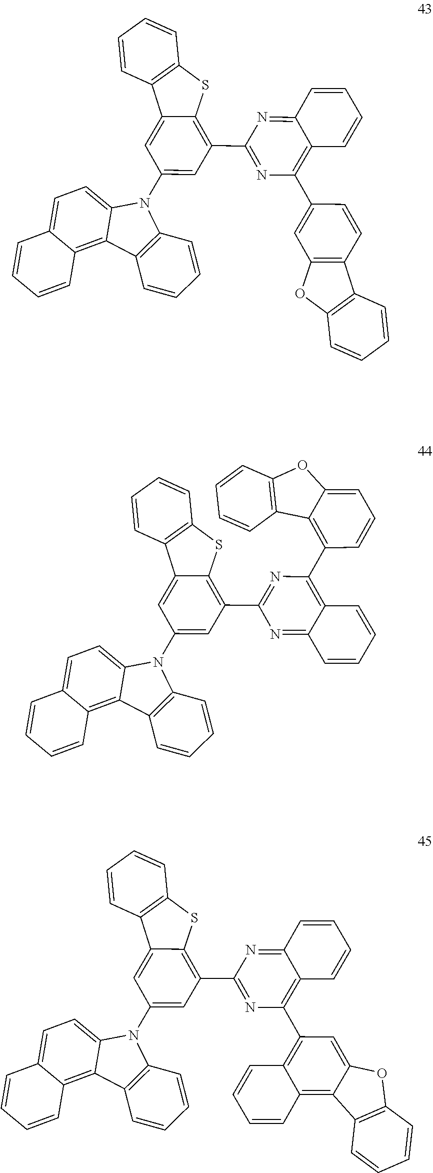 [SMILES: c1ccc2c(c1)ccc1c2c2ccccc2n1-c1cc(-c2nc(-c3ccc4c(c3)oc3ccccc34)c3ccccc3n2)c2sc3ccccc3c2c1 ; c1ccc2c(c1)ccc1c2c2ccccc2n1-c1cc(-c2nc(-c3cccc4oc5ccccc5c34)c3ccccc3n2)c2sc3ccccc3c2c1 ; c1ccc2c(c1)ccc1c2c2ccccc2n1-c1cc(-c2nc(-c3cc4oc5ccccc5c4c4ccccc34)c3ccccc3n2)c2sc3ccccc3c2c1]

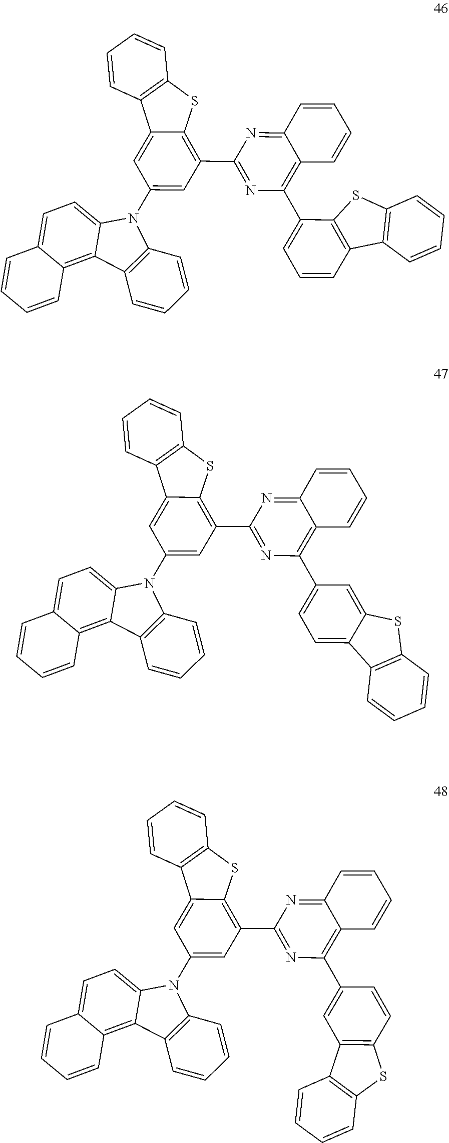 [SMILES: c1ccc2c(c1)ccc1c2c2ccccc2n1-c1cc(-c2nc(-c3cccc4c3sc3ccccc34)c3ccccc3n2)c2sc3ccccc3c2c1 ; c1ccc2c(c1)ccc1c2c2ccccc2n1-c1cc(-c2nc(-c3ccc4c(c3)sc3ccccc34)c3ccccc3n2)c2sc3ccccc3c2c1 ; c1ccc2c(c1)ccc1c2c2ccccc2n1-c1cc(-c2nc(-c3ccc4sc5ccccc5c4c3)c3ccccc3n2)c2sc3ccccc3c2c1]

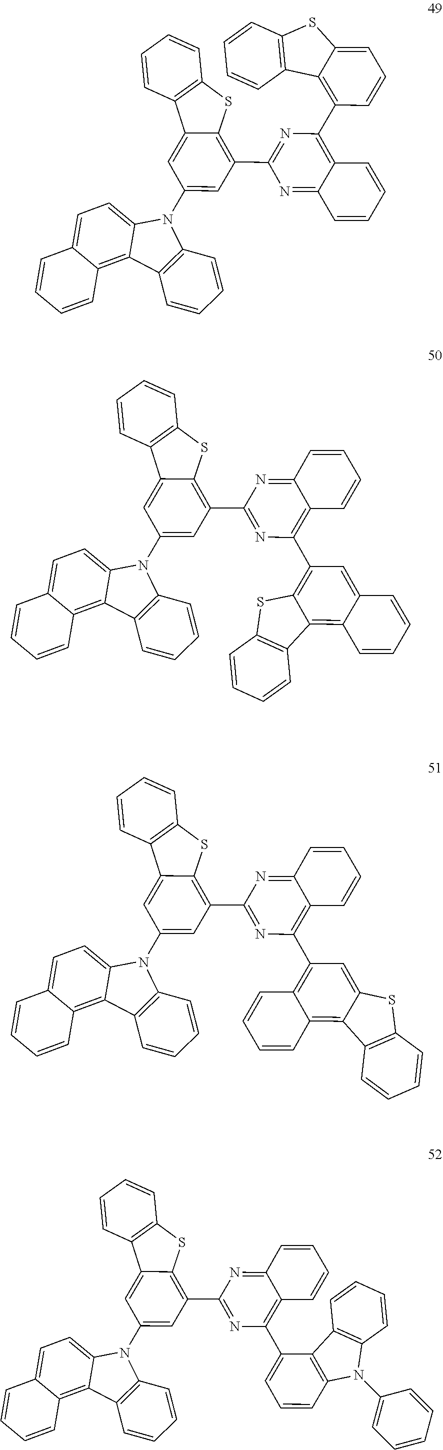 [SMILES: c1ccc2c(c1)ccc1c2c2ccccc2n1-c1cc(-c2nc(-c3cccc4sc5ccccc5c34)c3ccccc3n2)c2sc3ccccc3c2c1 ; c1ccc2c(c1)cc(-c1nc(-c3cc(-n4c5ccccc5c5c6ccccc6ccc54)cc4c3sc3ccccc34)nc3ccccc13)c1sc3ccccc3c12 ; c1ccc2c(c1)ccc1c2c2ccccc2n1-c1cc(-c2nc(-c3cc4sc5ccccc5c4c4ccccc34)c3ccccc3n2)c2sc3ccccc3c2c1 ; c1ccc(-n2c3ccccc3c3c(-c4nc(-c5cc(-n6c7ccccc7c7c8ccccc8ccc76)cc6c5sc5ccccc56)nc5ccccc45)cccc32)cc1]

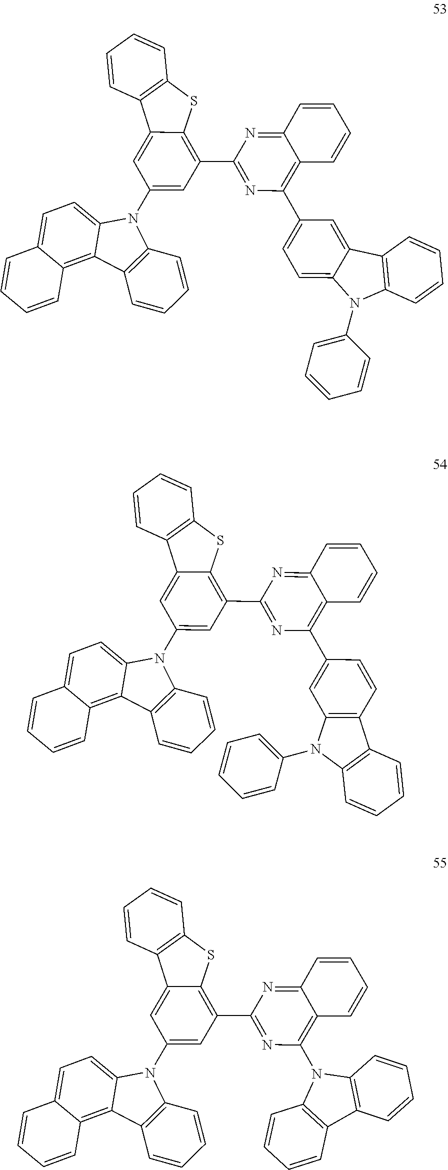 [SMILES: c1ccc(-n2c3ccccc3c3cc(-c4nc(-c5cc(-n6c7ccccc7c7c8ccccc8ccc76)cc6c5sc5ccccc56)nc5ccccc45)ccc32)cc1 ; c1ccc(-n2c3ccccc3c3ccc(-c4nc(-c5cc(-n6c7ccccc7c7c8ccccc8ccc76)cc6c5sc5ccccc56)nc5ccccc45)cc32)cc1 ; c1ccc2c(c1)ccc1c2c2ccccc2n1-c1cc(-c2nc(-n3c4ccccc4c4ccccc43)c3ccccc3n2)c2sc3ccccc3c2c1]

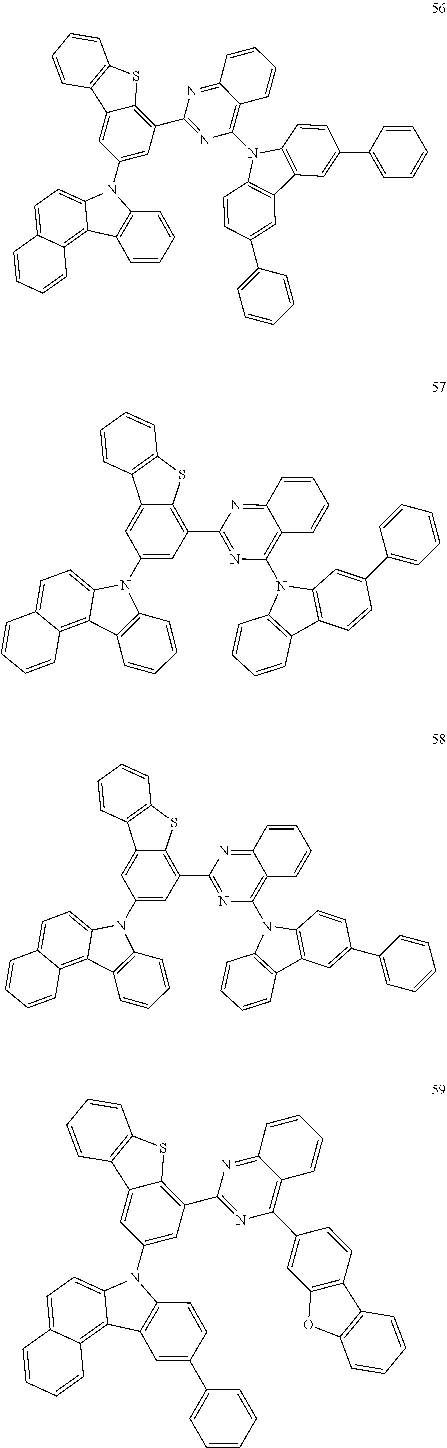 [SMILES: c1ccc(-c2ccc3c(c2)c2cc(-c4ccccc4)ccc2n3-c2nc(-c3cc(-n4c5ccccc5c5c6ccccc6ccc54)cc4c3sc3ccccc34)nc3ccccc23)cc1 ; c1ccc(-c2ccc3c4ccccc4n(-c4nc(-c5cc(-n6c7ccccc7c7c8ccccc8ccc76)cc6c5sc5ccccc56)nc5ccccc45)c3c2)cc1 ; c1ccc(-c2ccc3c(c2)c2ccccc2n3-c2nc(-c3cc(-n4c5ccccc5c5c6ccccc6ccc54)cc4c3sc3ccccc34)nc3ccccc23)cc1 ; c1ccc(-c2ccc3c(c2)c2c4ccccc4ccc2n3-c2cc(-c3nc(-c4ccc5c(c4)oc4ccccc45)c4ccccc4n3)c3sc4ccccc4c3c2)cc1]

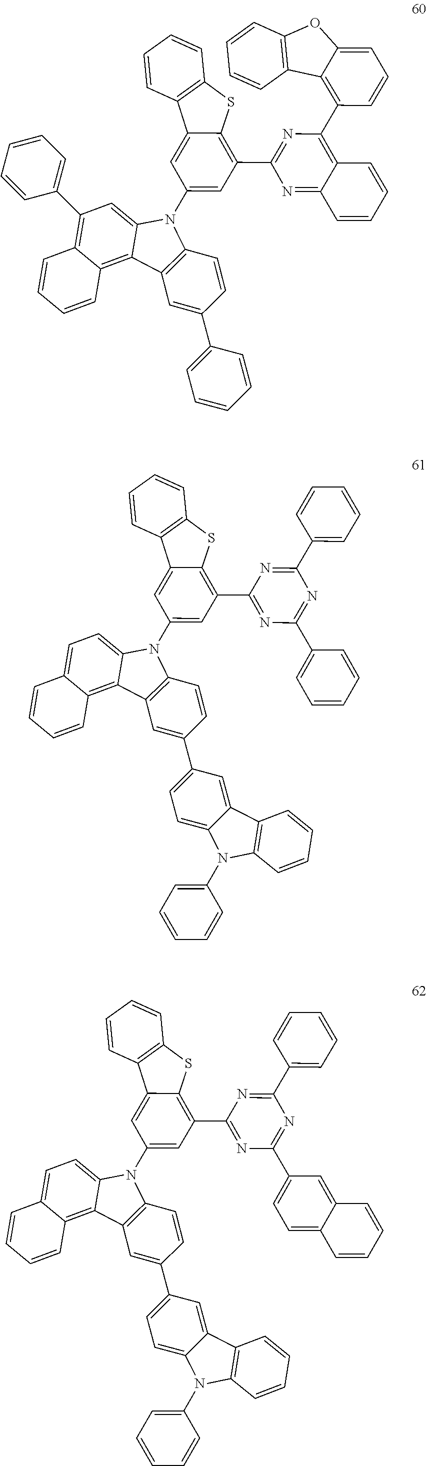 [SMILES: c1ccc(-c2ccc3c(c2)c2c4ccccc4c(-c4ccccc4)cc2n3-c2cc(-c3nc(-c4cccc5oc6ccccc6c45)c4ccccc4n3)c3sc4ccccc4c3c2)cc1 ; c1ccc(-c2nc(-c3ccccc3)nc(-c3cc(-n4c5ccc(-c6ccc7c(c6)c6ccccc6n7-c6ccccc6)cc5c5c6ccccc6ccc54)cc4c3sc3ccccc34)n2)cc1 ; c1ccc(-c2nc(-c3ccc4ccccc4c3)nc(-c3cc(-n4c5ccc(-c6ccc7c(c6)c6ccccc6n7-c6ccccc6)cc5c5c6ccccc6ccc54)cc4c3sc3ccccc34)n2)cc1]

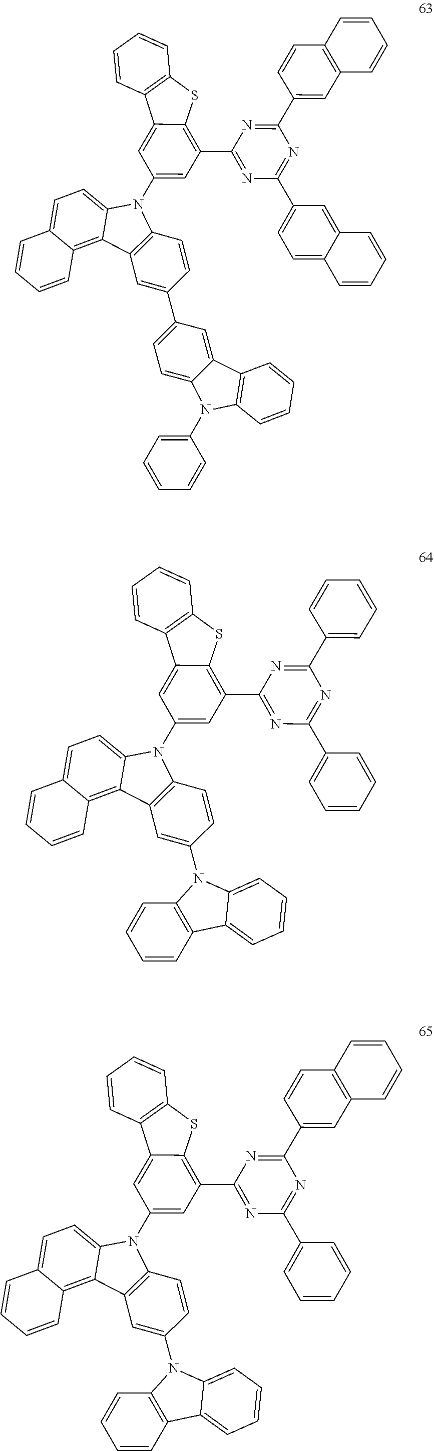 [SMILES: c1ccc(-n2c3ccccc3c3cc(-c4ccc5c(c4)c4c6ccccc6ccc4n5-c4cc(-c5nc(-c6ccc7ccccc7c6)nc(-c6ccc7ccccc7c6)n5)c5sc6ccccc6c5c4)ccc32)cc1 ; c1ccc(-c2nc(-c3ccccc3)nc(-c3cc(-n4c5ccc(-n6c7ccccc7c7ccccc76)cc5c5c6ccccc6ccc54)cc4c3sc3ccccc34)n2)cc1 ; c1ccc(-c2nc(-c3ccc4ccccc4c3)nc(-c3cc(-n4c5ccc(-n6c7ccccc7c7ccccc76)cc5c5c6ccccc6ccc54)cc4c3sc3ccccc34)n2)cc1]

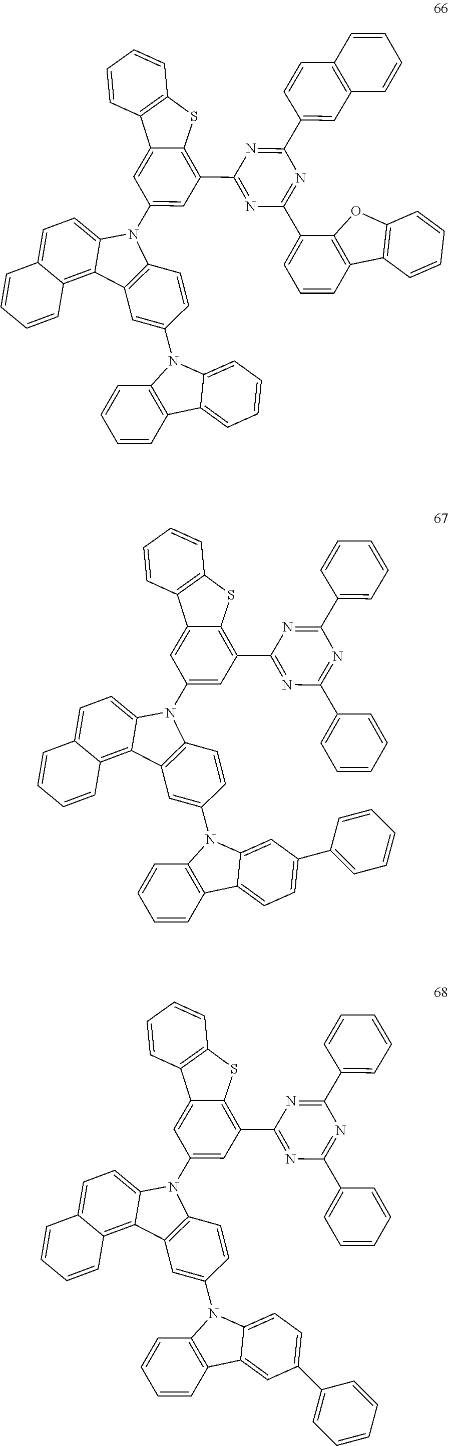 [SMILES: c1ccc2cc(-c3nc(-c4cccc5c4oc4ccccc45)nc(-c4cc(-n5c6ccc(-n7c8ccccc8c8ccccc87)cc6c6c7ccccc7ccc65)cc5c4sc4ccccc45)n3)ccc2c1 ; c1ccc(-c2ccc3c4ccccc4n(-c4ccc5c(c4)c4c6ccccc6ccc4n5-c4cc(-c5nc(-c6ccccc6)nc(-c6ccccc6)n5)c5sc6ccccc6c5c4)c3c2)cc1 ; c1ccc(-c2ccc3c(c2)c2ccccc2n3-c2ccc3c(c2)c2c4ccccc4ccc2n3-c2cc(-c3nc(-c4ccccc4)nc(-c4ccccc4)n3)c3sc4ccccc4c3c2)cc1]

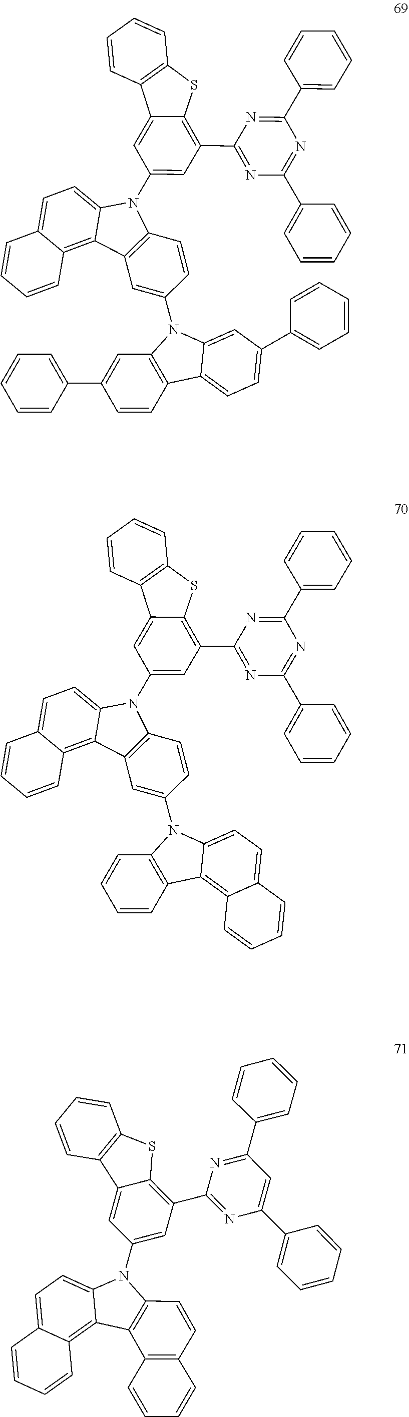 [SMILES: c1ccc(-c2ccc3c4ccc(-c5ccccc5)cc4n(-c4ccc5c(c4)c4c6ccccc6ccc4n5-c4cc(-c5nc(-c6ccccc6)nc(-c6ccccc6)n5)c5sc6ccccc6c5c4)c3c2)cc1 ; c1ccc(-c2nc(-c3ccccc3)nc(-c3cc(-n4c5ccc(-n6c7ccccc7c7c8ccccc8ccc76)cc5c5c6ccccc6ccc54)cc4c3sc3ccccc34)n2)cc1 ; c1ccc(-c2cc(-c3ccccc3)nc(-c3cc(-n4c5ccc6ccccc6c5c5c6ccccc6ccc54)cc4c3sc3ccccc34)n2)cc1]

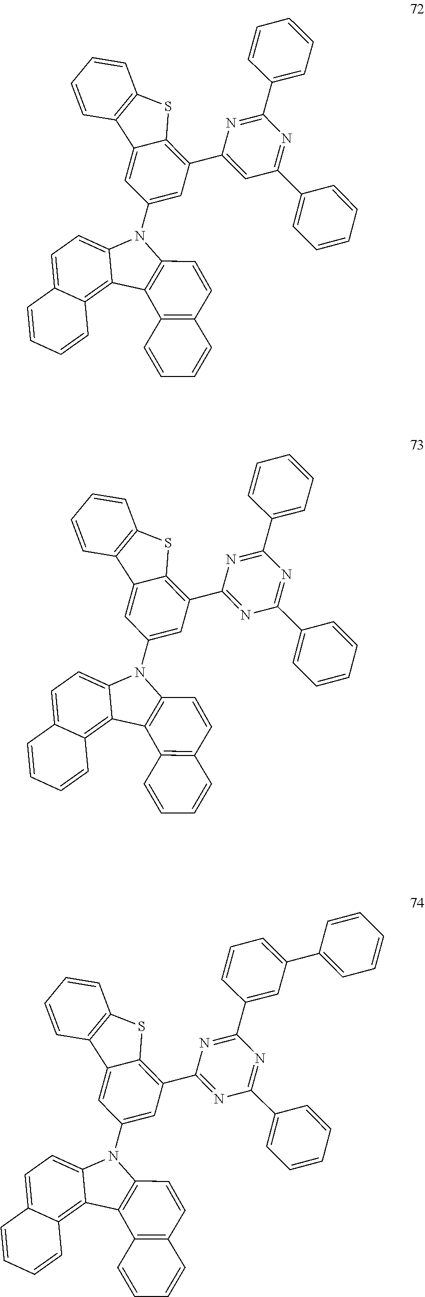 [SMILES: c1ccc(-c2cc(-c3cc(-n4c5ccc6ccccc6c5c5c6ccccc6ccc54)cc4c3sc3ccccc34)nc(-c3ccccc3)n2)cc1 ; c1ccc(-c2nc(-c3ccccc3)nc(-c3cc(-n4c5ccc6ccccc6c5c5c6ccccc6ccc54)cc4c3sc3ccccc34)n2)cc1 ; c1ccc(-c2cccc(-c3nc(-c4ccccc4)nc(-c4cc(-n5c6ccc7ccccc7c6c6c7ccccc7ccc65)cc5c4sc4ccccc45)n3)c2)cc1]

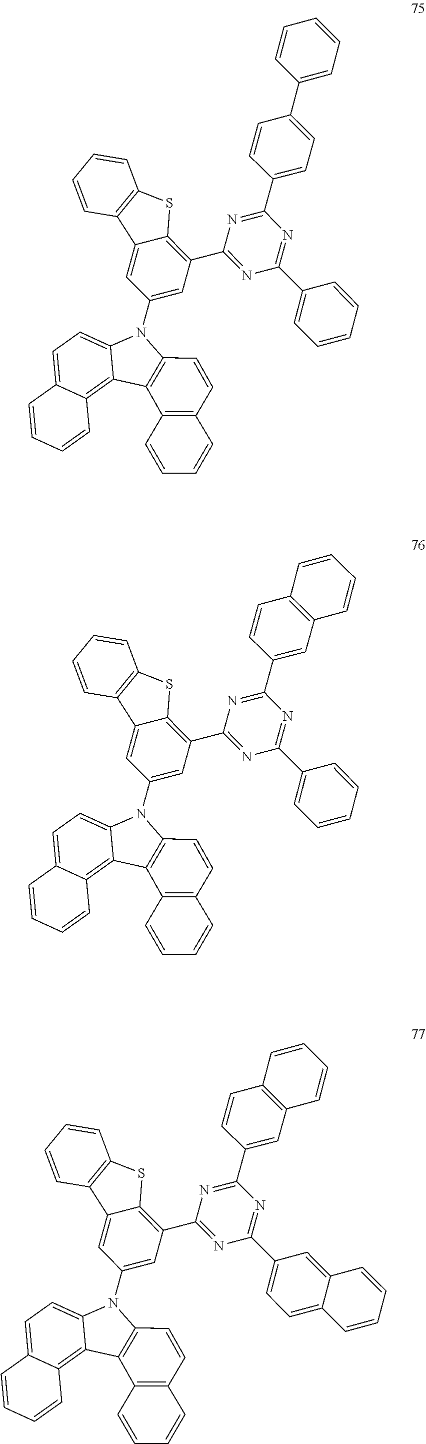 [SMILES: c1ccc(-c2ccc(-c3nc(-c4ccccc4)nc(-c4cc(-n5c6ccc7ccccc7c6c6c7ccccc7ccc65)cc5c4sc4ccccc45)n3)cc2)cc1 ; c1ccc(-c2nc(-c3ccc4ccccc4c3)nc(-c3cc(-n4c5ccc6ccccc6c5c5c6ccccc6ccc54)cc4c3sc3ccccc34)n2)cc1 ; c1ccc2cc(-c3nc(-c4ccc5ccccc5c4)nc(-c4cc(-n5c6ccc7ccccc7c6c6c7ccccc7ccc65)cc5c4sc4ccccc45)n3)ccc2c1]

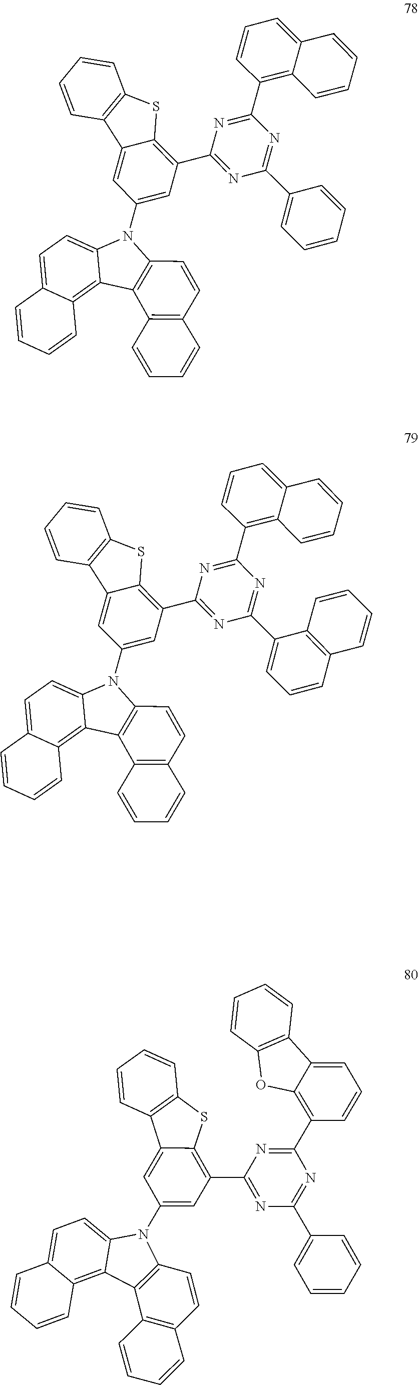 [SMILES: c1ccc(-c2nc(-c3cccc4ccccc34)nc(-c3cc(-n4c5ccc6ccccc6c5c5c6ccccc6ccc54)cc4c3sc3ccccc34)n2)cc1 ; c1ccc2c(-c3nc(-c4cccc5ccccc45)nc(-c4cc(-n5c6ccc7ccccc7c6c6c7ccccc7ccc65)cc5c4sc4ccccc45)n3)cccc2c1 ; c1ccc(-c2nc(-c3cccc4c3oc3ccccc34)nc(-c3cc(-n4c5ccc6ccccc6c5c5c6ccccc6ccc54)cc4c3sc3ccccc34)n2)cc1]

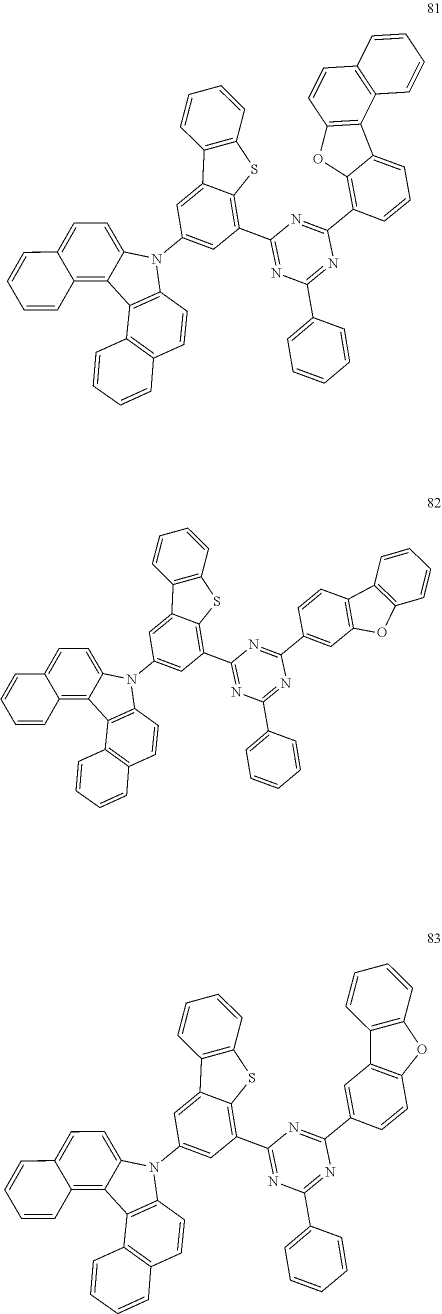 [SMILES: c1ccc(-c2nc(-c3cccc4c3oc3ccc5ccccc5c34)nc(-c3cc(-n4c5ccc6ccccc6c5c5c6ccccc6ccc54)cc4c3sc3ccccc34)n2)cc1 ; c1ccc(-c2nc(-c3ccc4c(c3)oc3ccccc34)nc(-c3cc(-n4c5ccc6ccccc6c5c5c6ccccc6ccc54)cc4c3sc3ccccc34)n2)cc1 ; c1ccc(-c2nc(-c3ccc4oc5ccccc5c4c3)nc(-c3cc(-n4c5ccc6ccccc6c5c5c6ccccc6ccc54)cc4c3sc3ccccc34)n2)cc1]

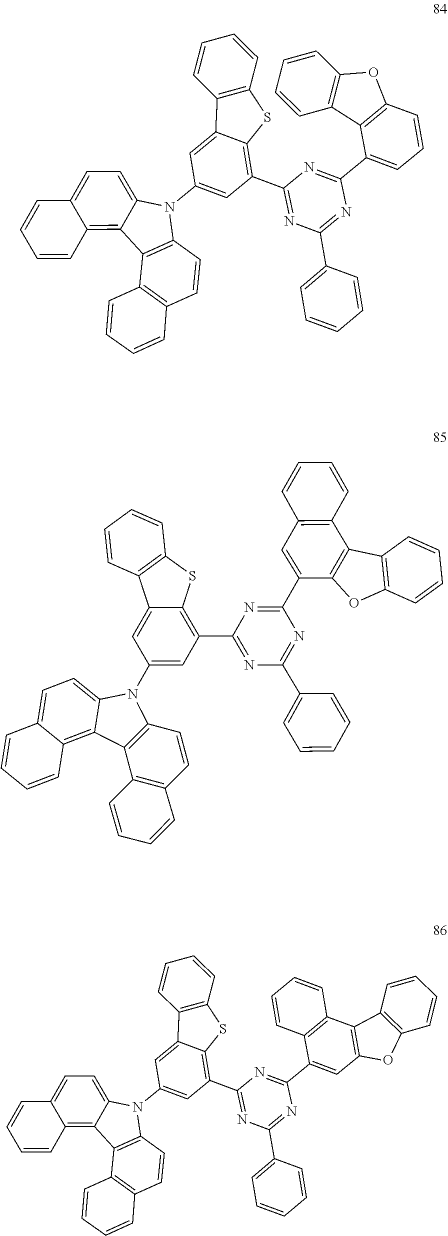 [SMILES: c1ccc(-c2nc(-c3cc(-n4c5ccc6ccccc6c5c5c6ccccc6ccc54)cc4c3sc3ccccc34)nc(-c3cccc4oc5ccccc5c34)n2)cc1 ; c1ccc(-c2nc(-c3cc4ccccc4c4c3oc3ccccc34)nc(-c3cc(-n4c5ccc6ccccc6c5c5c6ccccc6ccc54)cc4c3sc3ccccc34)n2)cc1 ; c1ccc(-c2nc(-c3cc4oc5ccccc5c4c4ccccc34)nc(-c3cc(-n4c5ccc6ccccc6c5c5c6ccccc6ccc54)cc4c3sc3ccccc34)n2)cc1]

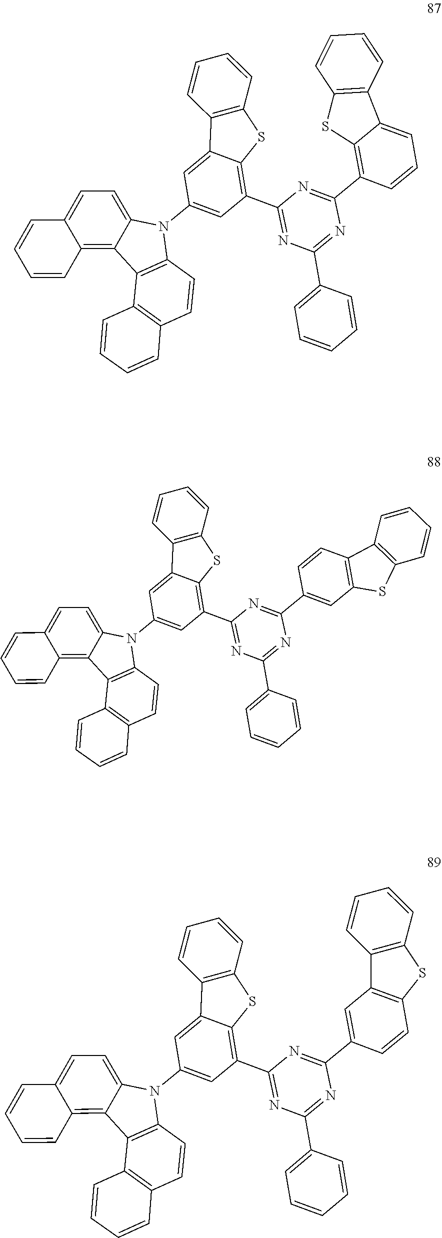 [SMILES: c1ccc(-c2nc(-c3cccc4c3sc3ccccc34)nc(-c3cc(-n4c5ccc6ccccc6c5c5c6ccccc6ccc54)cc4c3sc3ccccc34)n2)cc1 ; c1ccc(-c2nc(-c3ccc4c(c3)sc3ccccc34)nc(-c3cc(-n4c5ccc6ccccc6c5c5c6ccccc6ccc54)cc4c3sc3ccccc34)n2)cc1 ; c1ccc(-c2nc(-c3ccc4sc5ccccc5c4c3)nc(-c3cc(-n4c5ccc6ccccc6c5c5c6ccccc6ccc54)cc4c3sc3ccccc34)n2)cc1]

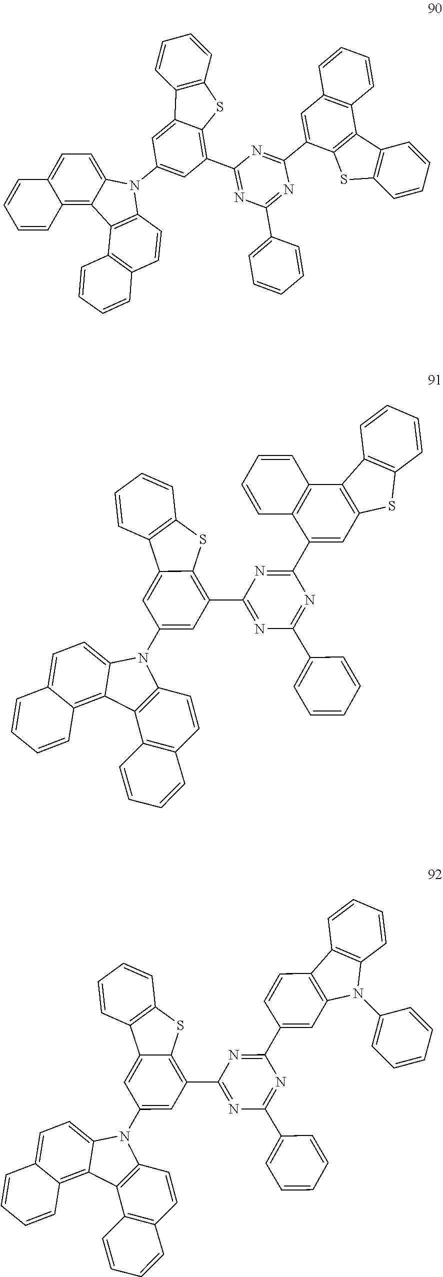 [SMILES: c1ccc(-c2nc(-c3cc(-n4c5ccc6ccccc6c5c5c6ccccc6ccc54)cc4c3sc3ccccc34)nc(-c3cc4ccccc4c4c3sc3ccccc34)n2)cc1 ; c1ccc(-c2nc(-c3cc4sc5ccccc5c4c4ccccc34)nc(-c3cc(-n4c5ccc6ccccc6c5c5c6ccccc6ccc54)cc4c3sc3ccccc34)n2)cc1 ; c1ccc(-c2nc(-c3ccc4c5ccccc5n(-c5ccccc5)c4c3)nc(-c3cc(-n4c5ccc6ccccc6c5c5c6ccccc6ccc54)cc4c3sc3ccccc34)n2)cc1]

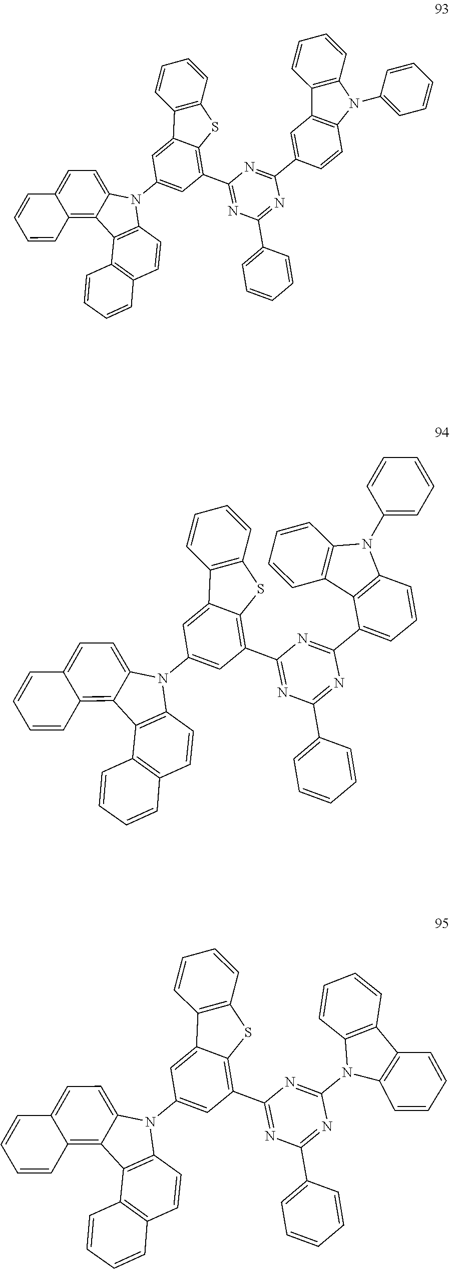 [SMILES: c1ccc(-c2nc(-c3ccc4c(c3)c3ccccc3n4-c3ccccc3)nc(-c3cc(-n4c5ccc6ccccc6c5c5c6ccccc6ccc54)cc4c3sc3ccccc34)n2)cc1 ; c1ccc(-c2nc(-c3cc(-n4c5ccc6ccccc6c5c5c6ccccc6ccc54)cc4c3sc3ccccc34)nc(-c3cccc4c3c3ccccc3n4-c3ccccc3)n2)cc1 ; c1ccc(-c2nc(-c3cc(-n4c5ccc6ccccc6c5c5c6ccccc6ccc54)cc4c3sc3ccccc34)nc(-n3c4ccccc4c4ccccc43)n2)cc1]

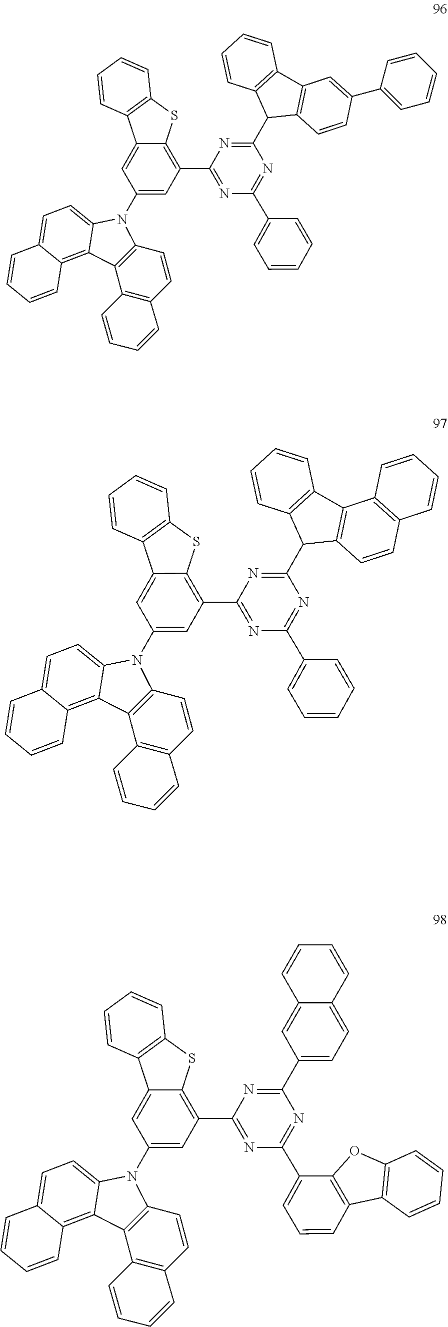 [SMILES: c1ccc(-c2ccc3c(c2)-c2ccccc2C3c2nc(-c3ccccc3)nc(-c3cc(-n4c5ccc6ccccc6c5c5c6ccccc6ccc54)cc4c3sc3ccccc34)n2)cc1 ; c1ccc(-c2nc(-c3cc(-n4c5ccc6ccccc6c5c5c6ccccc6ccc54)cc4c3sc3ccccc34)nc(C3c4ccccc4-c4c3ccc3ccccc43)n2)cc1 ; c1ccc2cc(-c3nc(-c4cccc5c4oc4ccccc45)nc(-c4cc(-n5c6ccc7ccccc7c6c6c7ccccc7ccc65)cc5c4sc4ccccc45)n3)ccc2c1]

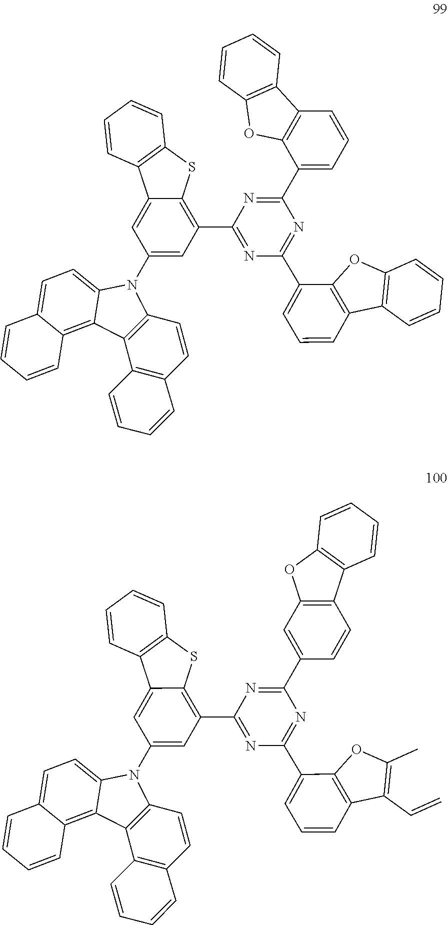 [SMILES: c1ccc2c(c1)ccc1c2c2c3ccccc3ccc2n1-c1cc(-c2nc(-c3cccc4c3oc3ccccc34)nc(-c3cccc4c3oc3ccccc34)n2)c2sc3ccccc3c2c1 ; C=Cc1c(C)oc2c(-c3nc(-c4ccc5c(c4)oc4ccccc45)nc(-c4cc(-n5c6ccc7ccccc7c6c6c7ccccc7ccc65)cc5c4sc4ccccc45)n3)cccc12]

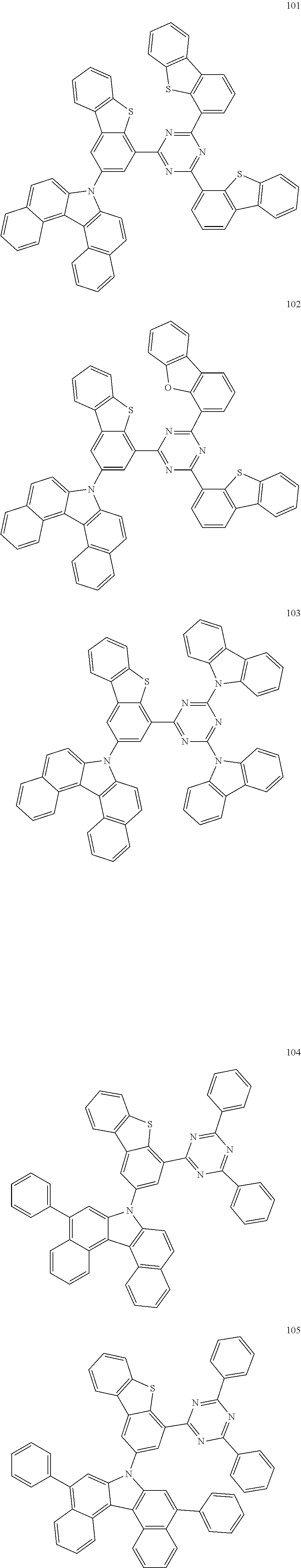

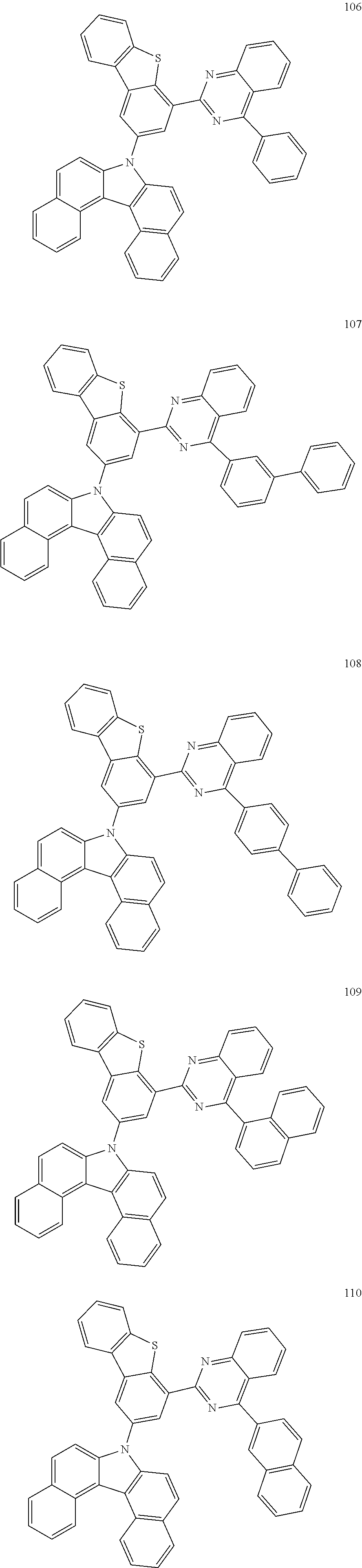 [SMILES: c1ccc(-c2nc(-c3cc(-n4c5ccc6ccccc6c5c5c6ccccc6ccc54)cc4c3sc3ccccc34)nc3ccccc23)cc1 ; c1ccc(-c2cccc(-c3nc(-c4cc(-n5c6ccc7ccccc7c6c6c7ccccc7ccc65)cc5c4sc4ccccc45)nc4ccccc34)c2)cc1 ; c1ccc(-c2ccc(-c3nc(-c4cc(-n5c6ccc7ccccc7c6c6c7ccccc7ccc65)cc5c4sc4ccccc45)nc4ccccc34)cc2)cc1 ; c1ccc2c(-c3nc(-c4cc(-n5c6ccc7ccccc7c6c6c7ccccc7ccc65)cc5c4sc4ccccc45)nc4ccccc34)cccc2c1 ; c1ccc2cc(-c3nc(-c4cc(-n5c6ccc7ccccc7c6c6c7ccccc7ccc65)cc5c4sc4ccccc45)nc4ccccc34)ccc2c1]

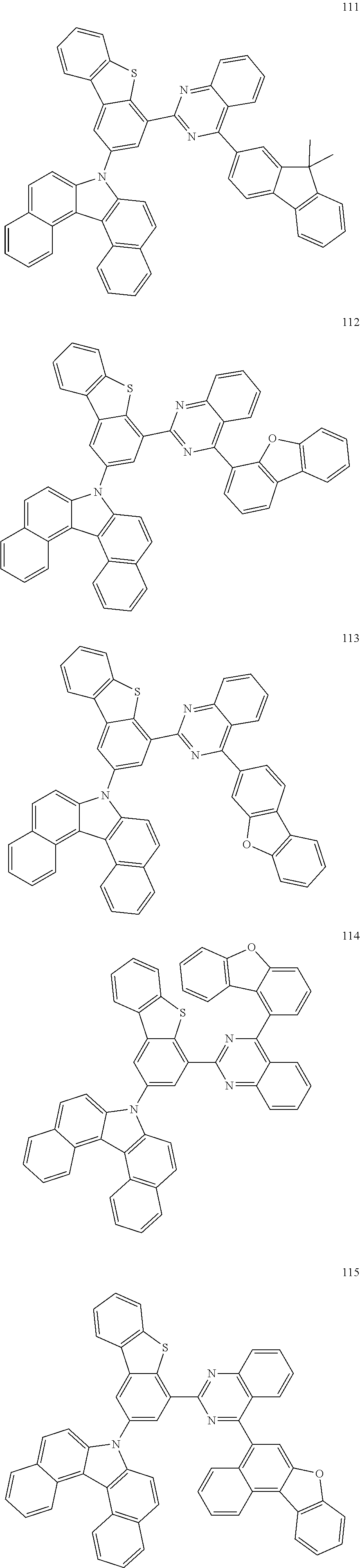 [SMILES: CC1(C)c2ccccc2-c2ccc(-c3nc(-c4cc(-n5c6ccc7ccccc7c6c6c7ccccc7ccc65)cc5c4sc4ccccc45)nc4ccccc34)cc21 ; c1ccc2c(c1)ccc1c2c2c3ccccc3ccc2n1-c1cc(-c2nc(-c3cccc4c3oc3ccccc34)c3ccccc3n2)c2sc3ccccc3c2c1 ; c1ccc2c(c1)ccc1c2c2c3ccccc3ccc2n1-c1cc(-c2nc(-c3ccc4c(c3)oc3ccccc34)c3ccccc3n2)c2sc3ccccc3c2c1 ; c1ccc2c(c1)ccc1c2c2c3ccccc3ccc2n1-c1cc(-c2nc(-c3cccc4oc5ccccc5c34)c3ccccc3n2)c2sc3ccccc3c2c1 ; c1ccc2c(c1)ccc1c2c2c3ccccc3ccc2n1-c1cc(-c2nc(-c3cc4oc5ccccc5c4c4ccccc34)c3ccccc3n2)c2sc3ccccc3c2c1]

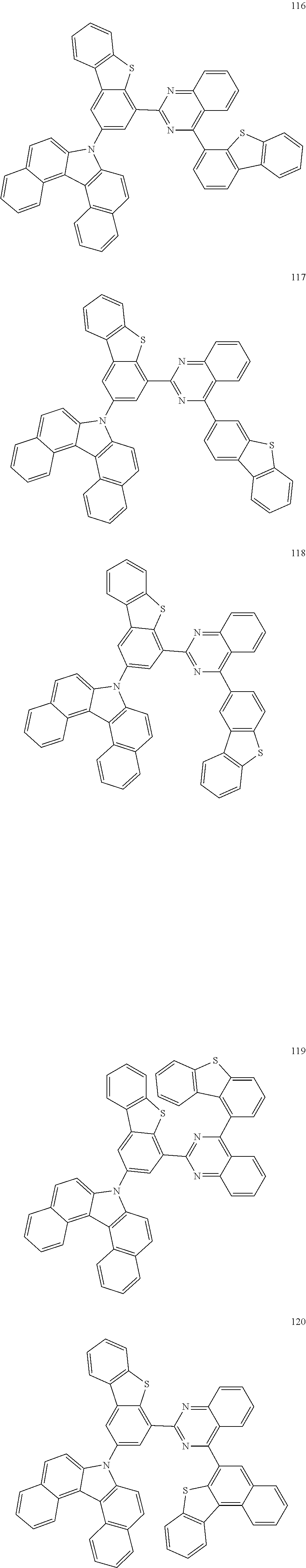 [SMILES: c1ccc2c(c1)ccc1c2c2c3ccccc3ccc2n1-c1cc(-c2nc(-c3cccc4c3sc3ccccc34)c3ccccc3n2)c2sc3ccccc3c2c1 ; c1ccc2c(c1)ccc1c2c2c3ccccc3ccc2n1-c1cc(-c2nc(-c3ccc4c(c3)sc3ccccc34)c3ccccc3n2)c2sc3ccccc3c2c1 ; c1ccc2c(c1)ccc1c2c2c3ccccc3ccc2n1-c1cc(-c2nc(-c3ccc4sc5ccccc5c4c3)c3ccccc3n2)c2sc3ccccc3c2c1 ; c1ccc2c(c1)ccc1c2c2c3ccccc3ccc2n1-c1cc(-c2nc(-c3cccc4sc5ccccc5c34)c3ccccc3n2)c2sc3ccccc3c2c1 ; c1ccc2c(c1)cc(-c1nc(-c3cc(-n4c5ccc6ccccc6c5c5c6ccccc6ccc54)cc4c3sc3ccccc34)nc3ccccc13)c1sc3ccccc3c12]

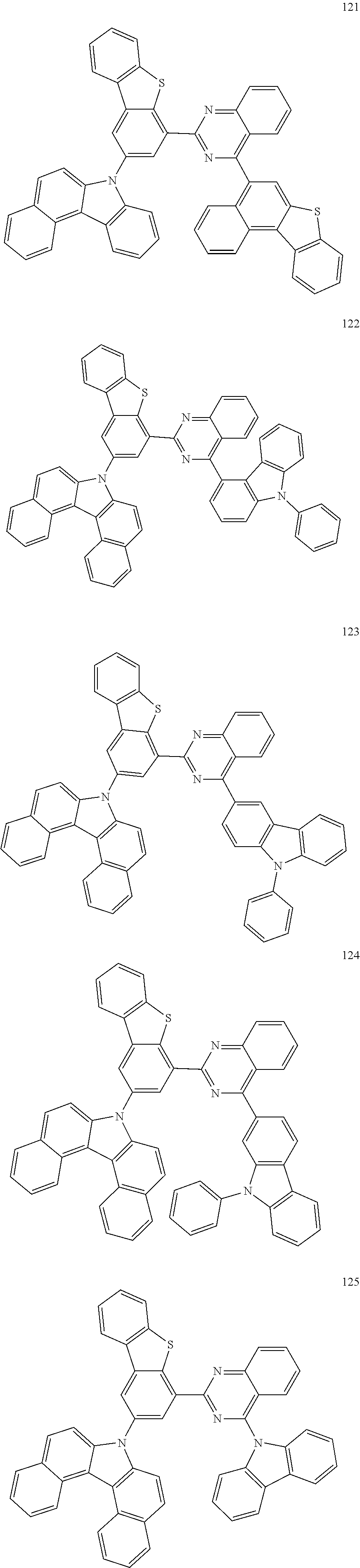

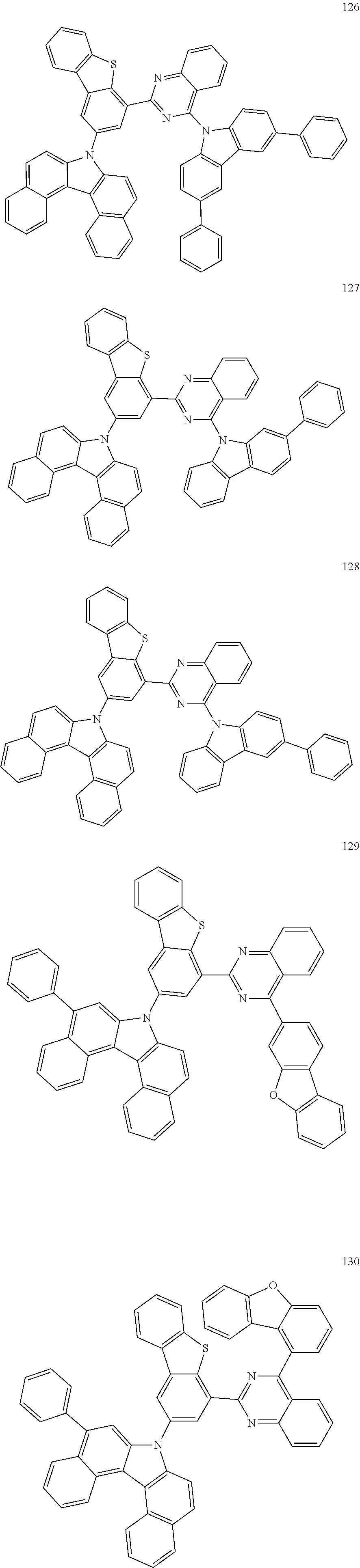

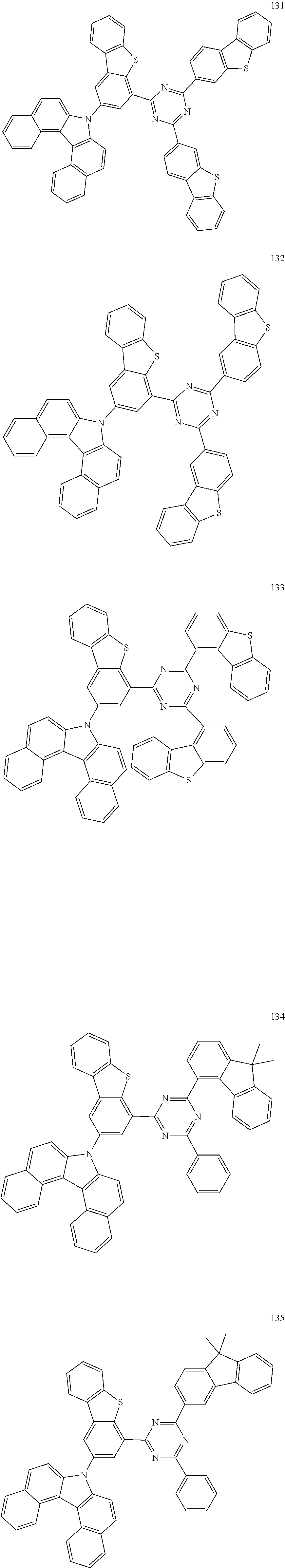

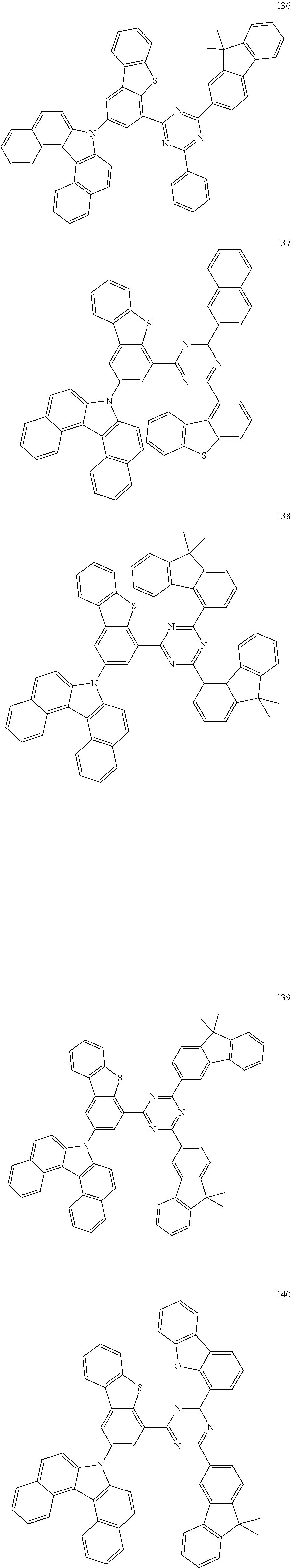

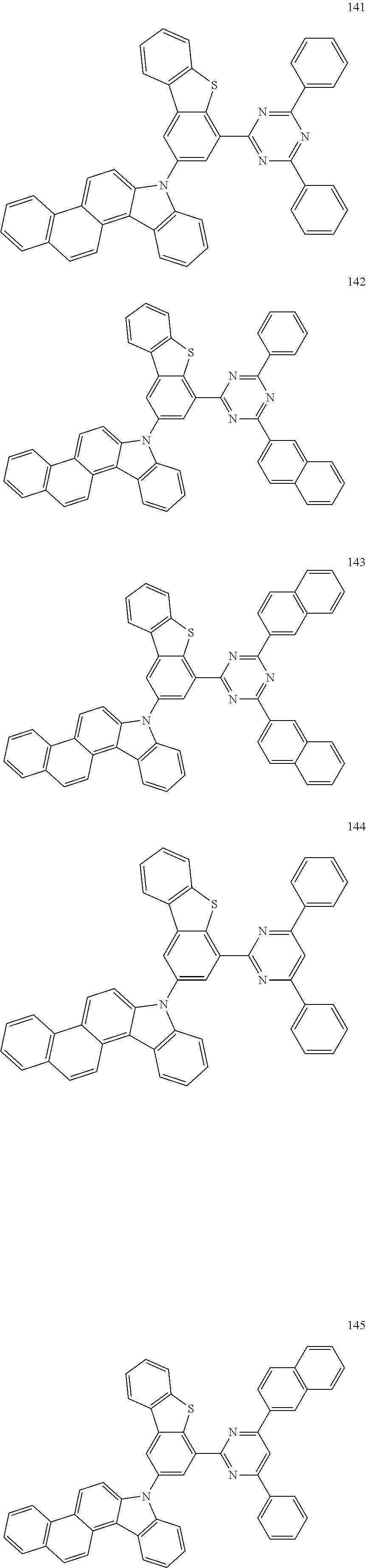 [SMILES: c1ccc(-c2nc(-c3ccccc3)nc(-c3cc(-n4c5ccccc5c5c6ccc7ccccc7c6ccc54)cc4c3sc3ccccc34)n2)cc1 ; c1ccc(-c2nc(-c3ccc4ccccc4c3)nc(-c3cc(-n4c5ccccc5c5c6ccc7ccccc7c6ccc54)cc4c3sc3ccccc34)n2)cc1 ; c1ccc2cc(-c3nc(-c4ccc5ccccc5c4)nc(-c4cc(-n5c6ccccc6c6c7ccc8ccccc8c7ccc65)cc5c4sc4ccccc45)n3)ccc2c1 ; c1ccc(-c2cc(-c3ccccc3)nc(-c3cc(-n4c5ccccc5c5c6ccc7ccccc7c6ccc54)cc4c3sc3ccccc34)n2)cc1 ; c1ccc(-c2cc(-c3ccc4ccccc4c3)nc(-c3cc(-n4c5ccccc5c5c6ccc7ccccc7c6ccc54)cc4c3sc3ccccc34)n2)cc1]

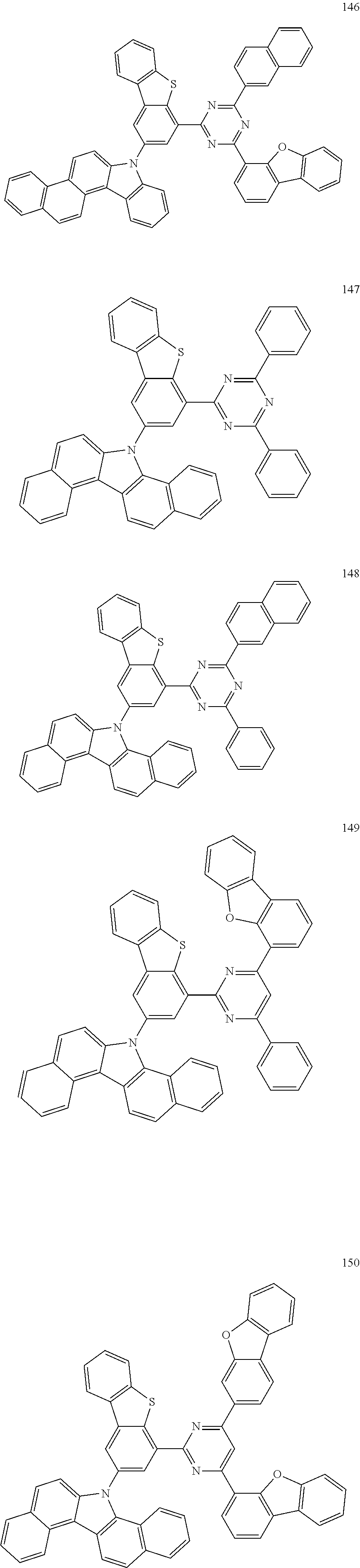 [SMILES: c1ccc2cc(-c3nc(-c4cccc5c4oc4ccccc45)nc(-c4cc(-n5c6ccccc6c6c7ccc8ccccc8c7ccc65)cc5c4sc4ccccc45)n3)ccc2c1 ; c1ccc(-c2nc(-c3ccccc3)nc(-c3cc(-n4c5ccc6ccccc6c5c5ccc6ccccc6c54)cc4c3sc3ccccc34)n2)cc1 ; c1ccc(-c2nc(-c3ccc4ccccc4c3)nc(-c3cc(-n4c5ccc6ccccc6c5c5ccc6ccccc6c54)cc4c3sc3ccccc34)n2)cc1 ; c1ccc(-c2cc(-c3cccc4c3oc3ccccc34)nc(-c3cc(-n4c5ccc6ccccc6c5c5ccc6ccccc6c54)cc4c3sc3ccccc34)n2)cc1 ; c1ccc2c(c1)ccc1c2c2ccc3ccccc3c2n1-c1cc(-c2nc(-c3ccc4c(c3)oc3ccccc34)cc(-c3cccc4c3oc3ccccc34)n2)c2sc3ccccc3c2c1]

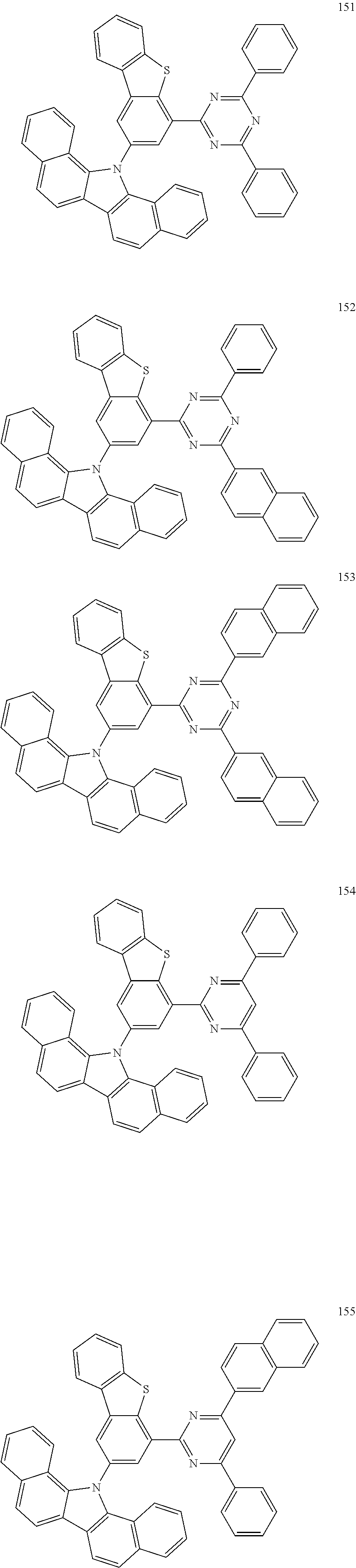 [SMILES: c1ccc(-c2nc(-c3ccccc3)nc(-c3cc(-n4c5c6ccccc6ccc5c5ccc6ccccc6c54)cc4c3sc3ccccc34)n2)cc1 ; c1ccc(-c2nc(-c3ccc4ccccc4c3)nc(-c3cc(-n4c5c6ccccc6ccc5c5ccc6ccccc6c54)cc4c3sc3ccccc34)n2)cc1 ; c1ccc2cc(-c3nc(-c4ccc5ccccc5c4)nc(-c4cc(-n5c6c7ccccc7ccc6c6ccc7ccccc7c65)cc5c4sc4ccccc45)n3)ccc2c1 ; c1ccc(-c2cc(-c3ccccc3)nc(-c3cc(-n4c5c6ccccc6ccc5c5ccc6ccccc6c54)cc4c3sc3ccccc34)n2)cc1 ; c1ccc(-c2cc(-c3ccc4ccccc4c3)nc(-c3cc(-n4c5c6ccccc6ccc5c5ccc6ccccc6c54)cc4c3sc3ccccc34)n2)cc1]

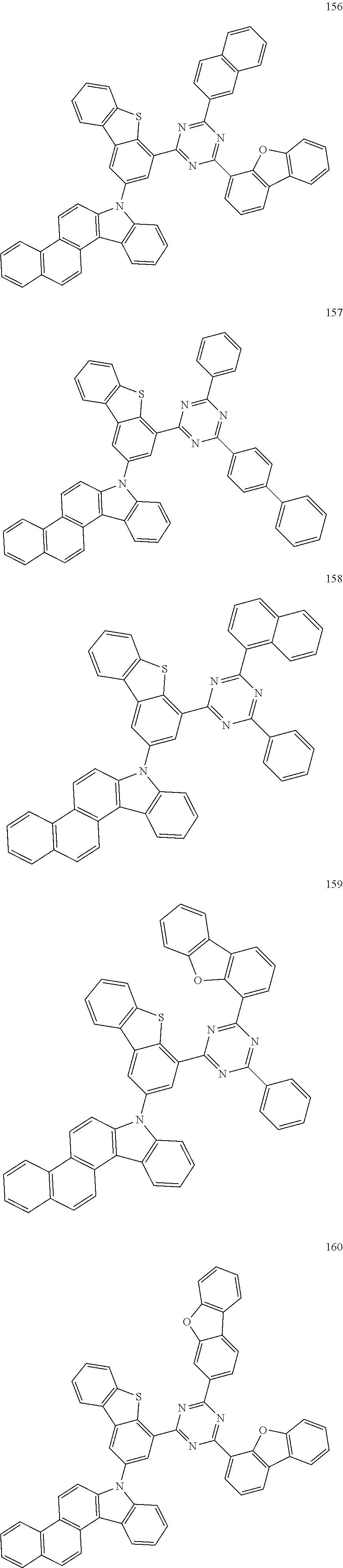

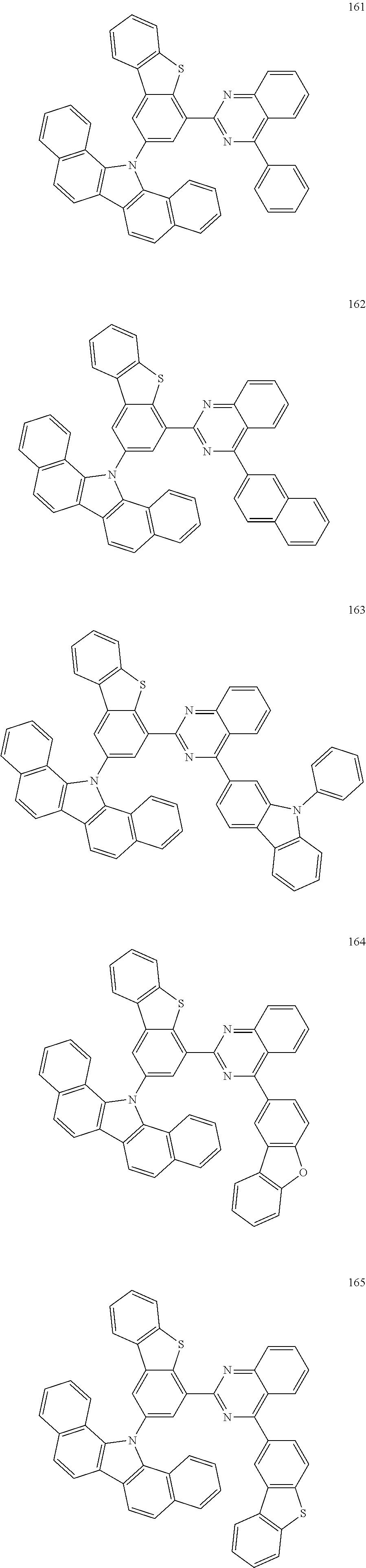 [SMILES: c1ccc(-c2nc(-c3cc(-n4c5c6ccccc6ccc5c5ccc6ccccc6c54)cc4c3sc3ccccc34)nc3ccccc23)cc1 ; c1ccc2cc(-c3nc(-c4cc(-n5c6c7ccccc7ccc6c6ccc7ccccc7c65)cc5c4sc4ccccc45)nc4ccccc34)ccc2c1 ; c1ccc(-n2c3ccccc3c3ccc(-c4nc(-c5cc(-n6c7c8ccccc8ccc7c7ccc8ccccc8c76)cc6c5sc5ccccc56)nc5ccccc45)cc32)cc1 ; c1ccc2c(c1)ccc1c3ccc4ccccc4c3n(-c3cc(-c4nc(-c5ccc6oc7ccccc7c6c5)c5ccccc5n4)c4sc5ccccc5c4c3)c21 ; c1ccc2c(c1)ccc1c3ccc4ccccc4c3n(-c3cc(-c4nc(-c5ccc6sc7ccccc7c6c5)c5ccccc5n4)c4sc5ccccc5c4c3)c21]

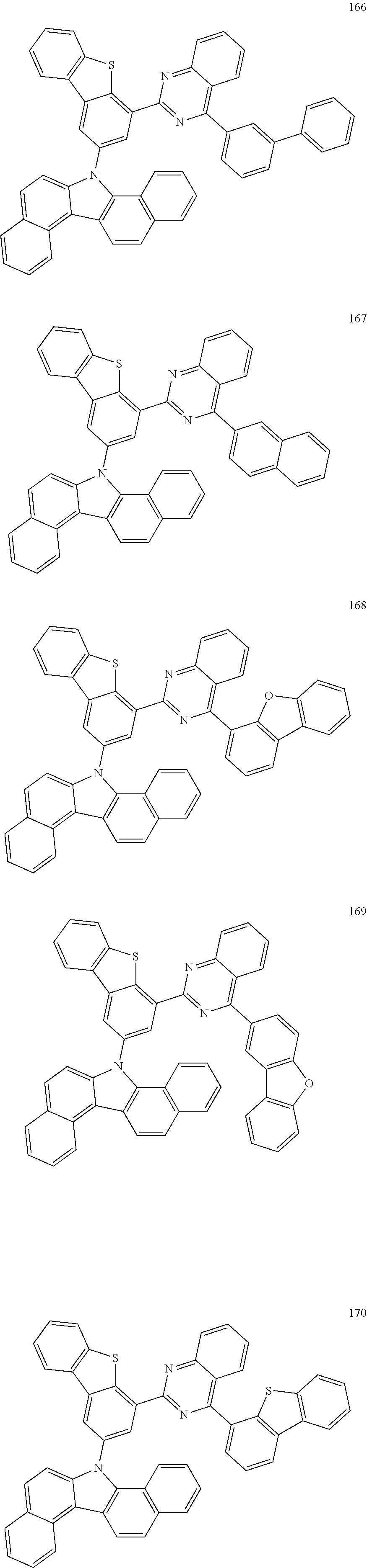 [SMILES: c1ccc(-c2cccc(-c3nc(-c4cc(-n5c6ccc7ccccc7c6c6ccc7ccccc7c65)cc5c4sc4ccccc45)nc4ccccc34)c2)cc1 ; c1ccc2cc(-c3nc(-c4cc(-n5c6ccc7ccccc7c6c6ccc7ccccc7c65)cc5c4sc4ccccc45)nc4ccccc34)ccc2c1 ; c1ccc2c(c1)ccc1c2c2ccc3ccccc3c2n1-c1cc(-c2nc(-c3cccc4c3oc3ccccc34)c3ccccc3n2)c2sc3ccccc3c2c1 ; c1ccc2c(c1)ccc1c2c2ccc3ccccc3c2n1-c1cc(-c2nc(-c3ccc4oc5ccccc5c4c3)c3ccccc3n2)c2sc3ccccc3c2c1 ; c1ccc2c(c1)ccc1c2c2ccc3ccccc3c2n1-c1cc(-c2nc(-c3cccc4c3sc3ccccc34)c3ccccc3n2)c2sc3ccccc3c2c1]

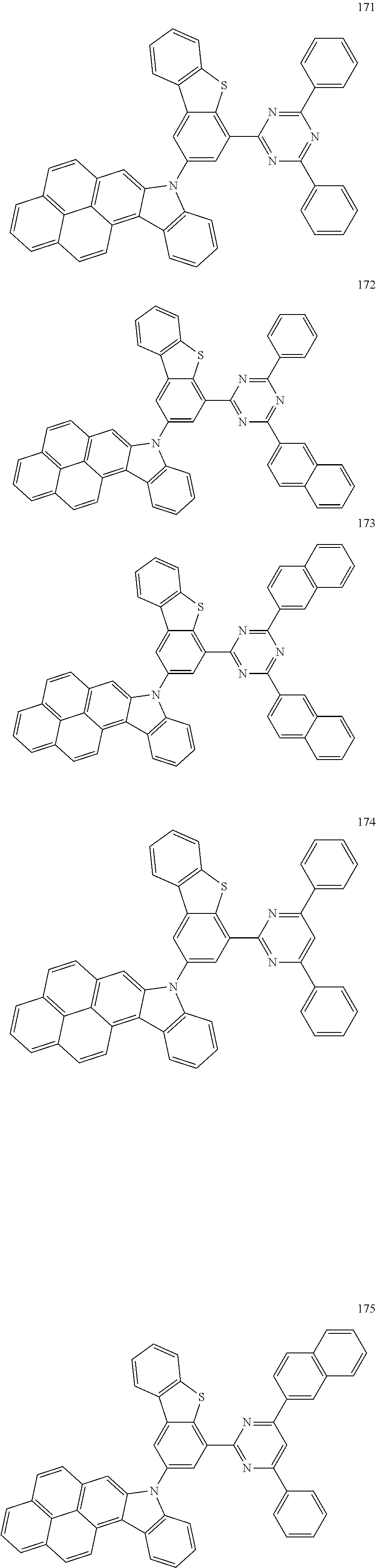

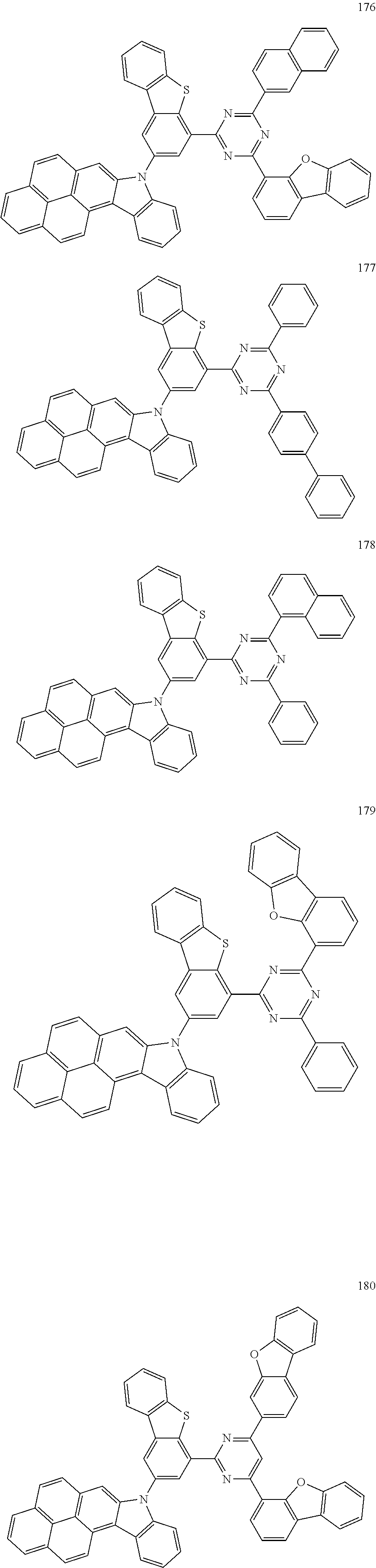

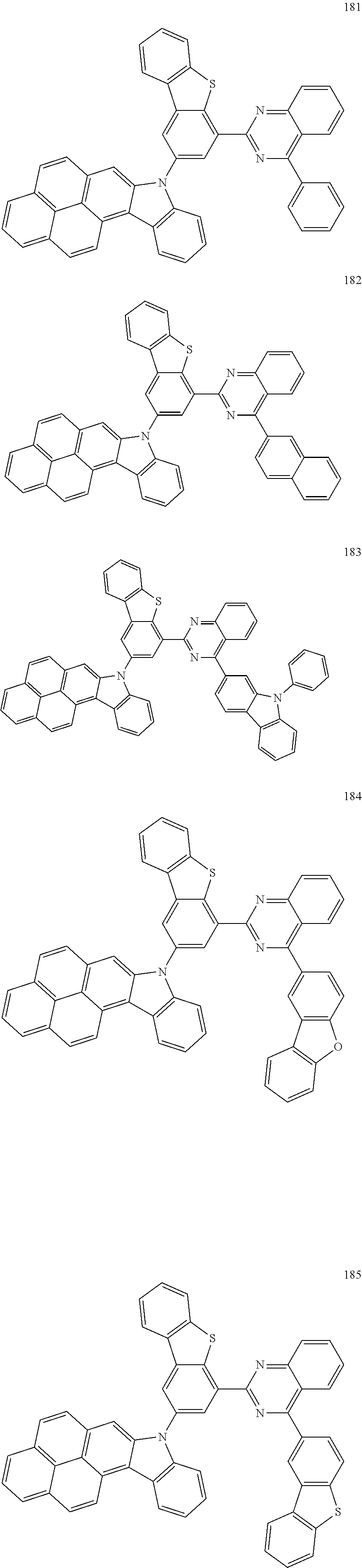

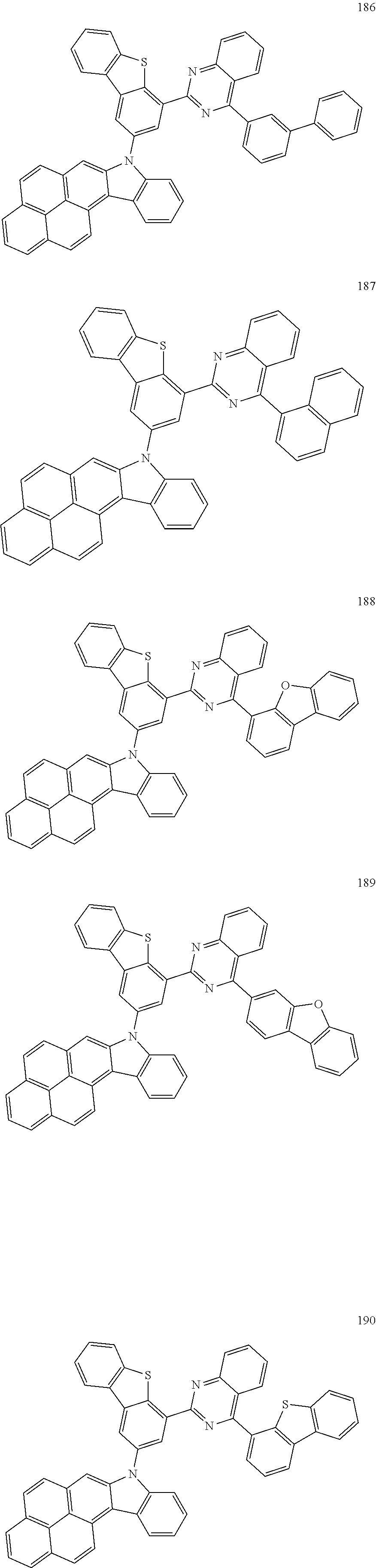 [SMILES: c1ccc(-c2cccc(-c3nc(-c4cc(-n5c6ccccc6c6c7ccc8cccc9ccc(cc65)c7c98)cc5c4sc4ccccc45)nc4ccccc34)c2)cc1 ; c1ccc2c(-c3nc(-c4cc(-n5c6ccccc6c6c7ccc8cccc9ccc(cc65)c7c98)cc5c4sc4ccccc45)nc4ccccc34)cccc2c1 ; c1cc2ccc3cc4c(c5ccc(c1)c2c35)c1ccccc1n4-c1cc(-c2nc(-c3cccc4c3oc3ccccc34)c3ccccc3n2)c2sc3ccccc3c2c1 ; c1cc2ccc3cc4c(c5ccc(c1)c2c35)c1ccccc1n4-c1cc(-c2nc(-c3ccc4c(c3)oc3ccccc34)c3ccccc3n2)c2sc3ccccc3c2c1 ; c1cc2ccc3cc4c(c5ccc(c1)c2c35)c1ccccc1n4-c1cc(-c2nc(-c3cccc4c3sc3ccccc34)c3ccccc3n2)c2sc3ccccc3c2c1]

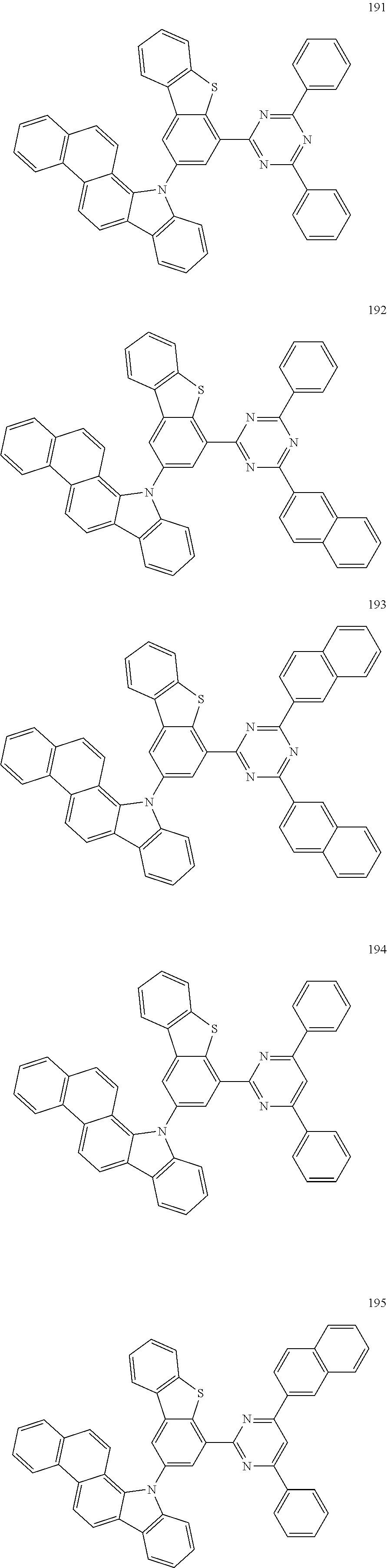 [SMILES: c1ccc(-c2nc(-c3ccccc3)nc(-c3cc(-n4c5ccccc5c5ccc6c7ccccc7ccc6c54)cc4c3sc3ccccc34)n2)cc1 ; c1ccc(-c2nc(-c3ccc4ccccc4c3)nc(-c3cc(-n4c5ccccc5c5ccc6c7ccccc7ccc6c54)cc4c3sc3ccccc34)n2)cc1 ; c1ccc2cc(-c3nc(-c4ccc5ccccc5c4)nc(-c4cc(-n5c6ccccc6c6ccc7c8ccccc8ccc7c65)cc5c4sc4ccccc45)n3)ccc2c1 ; c1ccc(-c2cc(-c3ccccc3)nc(-c3cc(-n4c5ccccc5c5ccc6c7ccccc7ccc6c54)cc4c3sc3ccccc34)n2)cc1 ; c1ccc(-c2cc(-c3ccc4ccccc4c3)nc(-c3cc(-n4c5ccccc5c5ccc6c7ccccc7ccc6c54)cc4c3sc3ccccc34)n2)cc1]

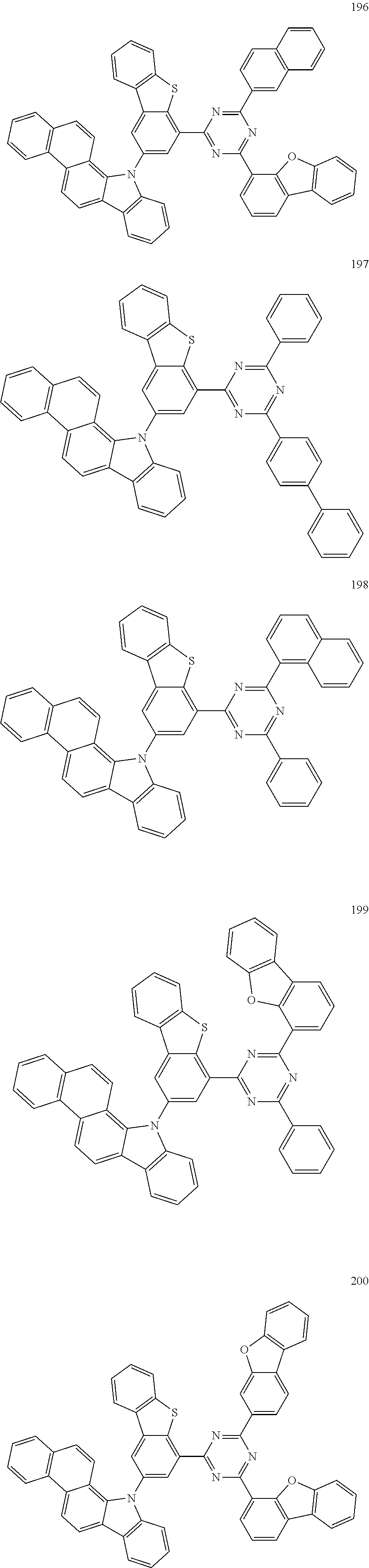

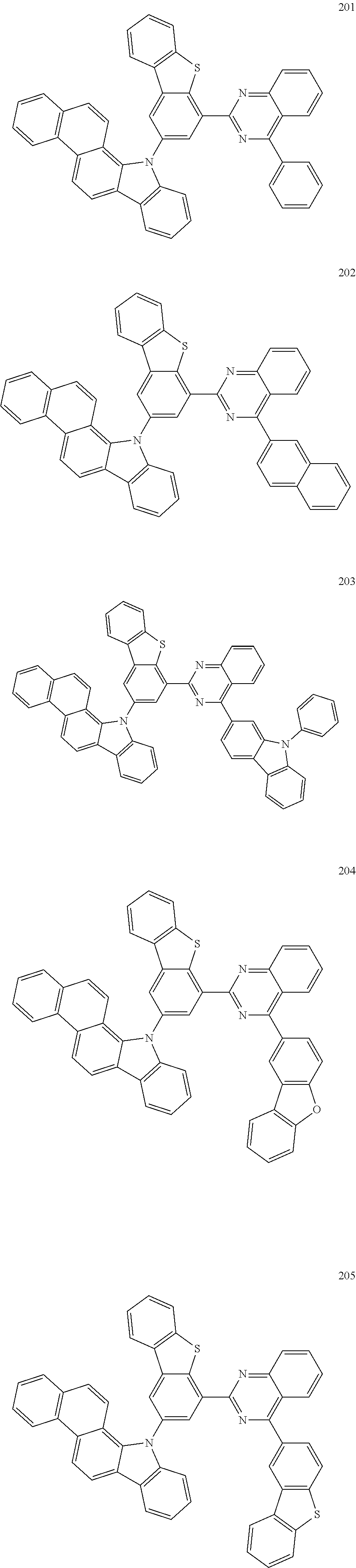 [SMILES: c1ccc(-c2nc(-c3cc(-n4c5ccccc5c5ccc6c7ccccc7ccc6c54)cc4c3sc3ccccc34)nc3ccccc23)cc1 ; c1ccc2cc(-c3nc(-c4cc(-n5c6ccccc6c6ccc7c8ccccc8ccc7c65)cc5c4sc4ccccc45)nc4ccccc34)ccc2c1 ; c1ccc(-n2c3ccccc3c3ccc(-c4nc(-c5cc(-n6c7ccccc7c7ccc8c9ccccc9ccc8c76)cc6c5sc5ccccc56)nc5ccccc45)cc32)cc1 ; c1ccc2c(c1)ccc1c2ccc2c3ccccc3n(-c3cc(-c4nc(-c5ccc6oc7ccccc7c6c5)c5ccccc5n4)c4sc5ccccc5c4c3)c12 ; c1ccc2c(c1)ccc1c2ccc2c3ccccc3n(-c3cc(-c4nc(-c5ccc6sc7ccccc7c6c5)c5ccccc5n4)c4sc5ccccc5c4c3)c12]

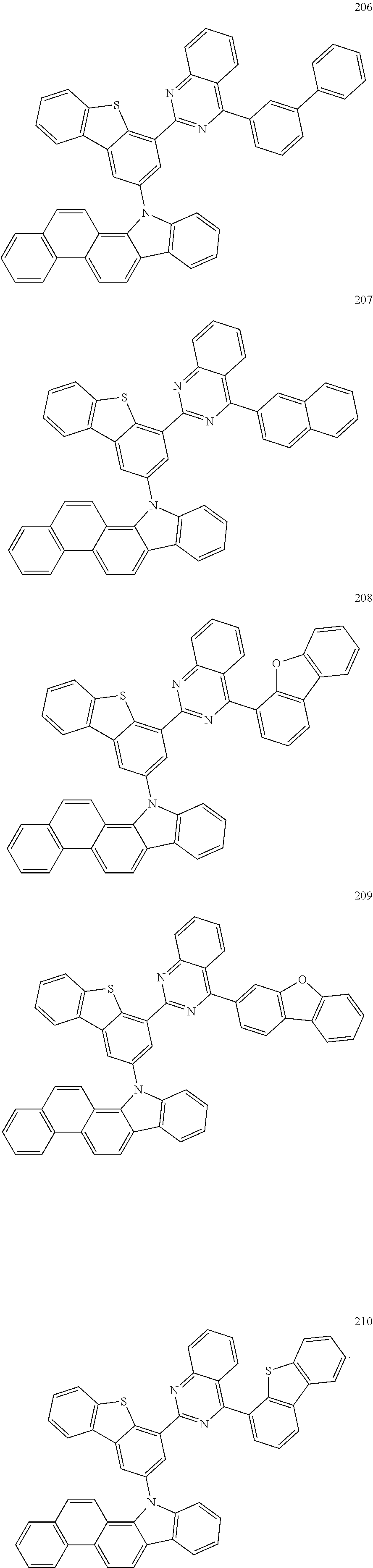 [SMILES: c1ccc(-c2cccc(-c3nc(-c4cc(-n5c6ccccc6c6ccc7c8ccccc8ccc7c65)cc5c4sc4ccccc45)nc4ccccc34)c2)cc1 ; c1ccc2cc(-c3nc(-c4cc(-n5c6ccccc6c6ccc7c8ccccc8ccc7c65)cc5c4sc4ccccc45)nc4ccccc34)ccc2c1 ; c1ccc2c(c1)ccc1c2ccc2c3ccccc3n(-c3cc(-c4nc(-c5cccc6c5oc5ccccc56)c5ccccc5n4)c4sc5ccccc5c4c3)c12 ; c1ccc2c(c1)ccc1c2ccc2c3ccccc3n(-c3cc(-c4nc(-c5ccc6c(c5)oc5ccccc56)c5ccccc5n4)c4sc5ccccc5c4c3)c12 ; c1ccc2c(c1)ccc1c2ccc2c3ccccc3n(-c3cc(-c4nc(-c5cccc6c5sc5ccccc56)c5ccccc5n4)c4sc5ccccc5c4c3)c12]

17. An organic optoelectronic diode, comprising
an anode and a cathode facing each other,
with at least one organic layer disposed between the anode and the cathode,
wherein the organic layer comprises the compound of claim 1.

18. The organic optoelectronic diode of claim 17, wherein
the organic layer comprises a light emitting layer, and
the light emitting layer comprises the compound.

19. A display device comprising the organic optoelectronic diode of claim 17.
